(19) United States
(12) Patent Application Publication
(10) Pub. No.: US 2026/0356893 A1
(43) Pub. Date: Nov. 12, 2026

(54) ENDOGENOUS GAG-BASED AND PNMA FAMILY CAPSIDS AND USES THEREOF

(71) Applicant: AERA THERAPEUTICS, INC., Cambridge, MA (US)

(72) Inventors: Adam FRAITES, Cambridge, MA (US); Colin MALONE, Cambridge, MA (US)

(21) Appl. No.: 18/998,948

(22) PCT Filed: Jul. 25, 2023
(86) PCT No.: PCT/US2023/070915

§ 371 Date: Jan. 27, 2025

Related U.S. Application Data
(60) Provisional application No. 63/369,646, filed on Jul. 27, 2022.
(60) Provisional application No. 63/369,647, filed on Jul. 27, 2022.

(51) Int. Cl. C07K 14/005; A61K 47/42

(52) U.S. Cl. CPC C07K 14/005; A61K 47/42

(57) ABSTRACT

Disclosed herein, in certain embodiments, are recombinant PNMA family and endogenous Gag polypeptides, capsids comprising the recombinant PNMA or endogenous Gag polypeptides, and methods of making and using recombinant PNMA and endogenous Gag polypeptides.

This application is the U.S. national phase of International Application No. PCT/US2023/070915, filed Jul. 25, 2023, which claims the benefit of U.S. Patent Application Nos. 63/369,646, filed Jul. 27, 2022 and 63/369,647, filed Jul. 27, 2022, each of which is hereby incorporated by reference.

### FIELD OF THE INVENTION

Disclosed herein, in certain embodiments, are recombinant PNMA family and endogenous Gag polypeptides, capsids comprising the recombinant PNMA or endogenous Gag polypeptides, and methods of making and using recombinant PNMA and endogenous Gag polypeptides.

### BACKGROUND OF THE INVENTION

There is a continuing need for improved delivery systems for nucleic acids and therapeutic agents.

### SUMMARY

Administering diagnostic or therapeutic agents to a site of interest with precision has presented an ongoing challenge. Available methods of delivering nucleic acids to cells suffer from a number of limitations. For example, AAV viral vectors often used for gene therapy are immunogenic, have a limited payload capacity, suffer from poor bio-distribution, can only be administered by direct injection, and pose a risk of disrupting host genes by integration. Some studies have suggested that a significant proportion (e.g., ≥50%) of the population have pre-existing immunity to viral vectors such as AAV, which could severely limit their effectiveness in therapeutic applications. Thus, there is a need for new and improved compositions and methods for delivering therapeutic payloads. Capsids (or virus-like particles, VLPs) disclosed herein have the potential to address many of these shortcomings. Disclosed herein, in certain embodiments, are endogenous retroviral capsid polypeptides. In some embodiments, endogenous Gag (endo-Gag) polypeptides of the disclosure, such as PNMA family polypeptides and retrotransposon Gag-like (RTL) family polypeptides, assemble into capsids for delivery of a cargo of interest. Endo-Gag polypeptides of the disclosure exhibit surprising and unexpected advantages over existing and alternate capsid-forming polypeptides, such as improved efficiency in capsid assembly, capsid disassembly, and/or capsid reassembly, e.g., reassembly with a heterologous cargo. In additional embodiments, described herein are capsids, e.g., PEG10-based, RTL10-based, PNMA-based or endo-Gag-based capsids, for delivery of a cargo of interest. Also disclosed herein are engineered endo-Gag polypeptides, such as PEG10-based, RTL10-based, other endo-Gag-based, or PNMA family polypeptides, with additional modifications of particular advantage and utility.

The present inventors have discovered that expressing the endogenous Gag polypeptide as a conjugate with, for example, a bulky solubility tag, prevents premature capsid formation and increases the efficiency of forming capsids with desired cargos.

One embodiment is a method of preparing a capsid comprising (a) expressing an endogenous Gag polypeptide conjugate in a host cell or a cell-free expression system, the endogenous Gag polypeptide conjugated to a N-terminal component which prevents, inhibits, or suppresses oligomerization of the endogenous Gag polypeptide; (b) optionally, isolating the endogenous Gag polypeptide conjugate; (c) cleaving the N-terminal tag from the endogenous Gag polypeptide conjugate to form cleaved endogenous Gag polypeptide; and (d) treating the cleaved endogenous Gag polypeptide with an assembly buffer. In one embodiment, the method further comprises contacting the endogenous Gag polypeptide with a cargo during step (d), where at least a subset of the endogenous Gag capsids comprise the cargo. In another embodiment, the method further comprises treating the isolated endogenous Gag polypeptide with a disassembly buffer prior to step (d), thereby producing or maintaining the endogenous Gag polypeptide in a disassembled state. In yet another embodiment, less than 20%, less than 10%, or less than 5% of the endogenous Gag polypeptide conjugate forms a capsid prior to step (c).

Another embodiment is a method of preparing a capsid comprising (a) cleaving the N-terminal tag from an endogenous Gag polypeptide conjugate to form cleaved endogenous Gag polypeptide, where the endogenous Gag polypeptide conjugate comprises an endogenous Gag polypeptide conjugated to a N-terminal component which prevents, inhibits, or suppresses oligomerization of the endogenous Gag polypeptide; (b) contacting the cleaved endogenous Gag polypeptide with a cargo; and (c) treating the cleaved endogenous Gag polypeptide with an assembly buffer, thereby assembling the cleaved endogenous Gag polypeptide into endogenous Gag capsids, where at least a subset of the endogenous Gag capsids comprise the cargo. In one embodiment, the method further comprises treating the endogenous Gag polypeptide with a disassembly buffer prior to step (b) or (c), thereby producing or maintaining the endogenous Gag polypeptide in a disassembled state. In another embodiment, less than 20%, less than 10%, or less than 5% of the endogenous Gag polypeptide conjugate forms a capsid prior to step (a).

In any of the methods of preparation described herein, the N-terminal component of the endogenous Gag polypeptide conjugate may comprise (i) a cleavage site directly attached to the endogenous Gag polypeptide and (ii) a bulky solubility tag which prevents, inhibits, or suppresses oligomerization of the endogenous Gag polypeptide. In one embodiment, the bulky solubility tag comprises a polypeptide. In another embodiment, the bulky solubility tag increases the solubility of the endogenous Gag polypeptide. In yet another embodiment, the bulky solubility tag sterically blocks assembly of the endogenous Gag polypeptide. In yet another embodiment, the bulky solubility tag is selected from NusA (SEQ ID NO: 93), beta-lactamase (SEQ ID NO: 94), elongation factor Ts (SEQ ID NO: 95), peptidylprolyl isomerase (SEQ ID NO: 96), SUMO (SEQ ID NO: 97), bdSUMO, maltose binding protein (SEQ ID NO: 98 or SEQ ID NO: 64), and TriggerFactor (SEQ ID NO: 99). The cleavage site may be selected from a TEV, HRV3C, thrombin, enterokinase, factor Xa, or carboxypeptidase cleavage site (e.g., a TEV cleavage site).

Yet another embodiment is a kit comprising (a) a first container containing a composition comprising an endogenous Gag polypeptide conjugate where the endogenous Gag polypeptide conjugate comprises an endogenous Gag polypeptide conjugated to a N-terminal component which prevents, inhibits, or suppresses oligomerization of the endogenous Gag polypeptide; and (b) a second container containing a protease for cleaving the N-terminal component from the endogenous Gag polypeptide. In the kit, the N-terminal component of the endogenous Gag polypeptide conjugate may comprise (i) a cleavage site directly attached to the endogenous Gag polypeptide and (ii) a bulky solubility tag which prevents, inhibits, or suppresses oligomerization of the endogenous Gag polypeptide. In one embodiment, the bulky solubility tag comprises a polypeptide. In another embodiment, the bulky solubility tag increases the solubility of the endogenous Gag polypeptide. In yet another embodiment, the bulky solubility tag sterically blocks assembly of the endogenous Gag polypeptide. In yet another embodiment, the bulky solubility tag is selected from NusA (SEQ ID NO: 93), beta-lactamase (SEQ ID NO: 94), elongation factor Ts (SEQ ID NO: 95), peptidylprolyl isomerase (SEQ ID NO: 96), SUMO (SEQ ID NO: 97), bdSUMO, maltose binding protein (SEQ ID NO: 98), and TriggerFactor (SEQ ID NO: 99). The cleavage site may be selected from a TEV, HRV3C, thrombin, enterokinase, factor Xa, or carboxypeptidase cleavage site (e.g., a TEV cleavage site).

Disclosed herein, in some aspects, is a method of making a capsid, the method comprising: (a) expressing an endogenous Gag polypeptide in a host cell or a cell-free expression system; (b) isolating the endogenous Gag polypeptide; and (c) treating the isolated endogenous Gag polypeptide with an assembly buffer, thereby assembling the isolated endogenous Gag polypeptide into endogenous Gag capsids.

In some embodiments, the method further comprises contacting the endogenous Gag polypeptide with a cargo during step (c), wherein at least a subset of the endogenous Gag capsids comprise the cargo. In some embodiments, the method further comprises treating the isolated endogenous Gag polypeptide with a disassembly buffer prior to step (c), thereby (e.g., disassembling a capsid comprising the endogenous Gag polypeptide) producing or maintaining the endogenous Gag polypeptide in a disassembled state. In some embodiments, the isolated endogenous Gag polypeptide is at least partially present in a capsid form prior to the treating with the disassembly buffer. In some embodiments, at least about 5% of the endogenous Gag polypeptide that is present in capsid form prior to the treating with the disassembly buffer is in the disassembled state after the treating with the disassembly buffer. In some embodiments, at least about 5% of the endogenous Gag polypeptide that is present in the disassembled state is present in capsid form after step (c).

Disclosed herein, in some aspects, is a method of loading endogenous retroviral capsids with a cargo, the method comprising: treating endogenous Gag polypeptide with a disassembly buffer, thereby (e.g., disassembling a capsid comprising the endogenous Gag polypeptide) producing or maintaining the endogenous Gag polypeptide in a disassembled state; contacting the endogenous Gag polypeptide with a cargo; and treating the endogenous Gag polypeptide with an assembly buffer, thereby assembling the endogenous Gag polypeptide into endogenous Gag capsids, wherein at least a subset of the endogenous Gag capsids comprise the cargo.

In some embodiments, the endogenous Gag polypeptide is a native endogenous Gag polypeptide. In some embodiments, the endogenous Gag polypeptide is an engineered endogenous Gag polypeptide. In some embodiments, the endogenous Gag polypeptide is a PNMA family protein. In some embodiments, the endogenous Gag polypeptide is a mammalian PNMA family protein. In some embodiments, the endogenous Gag polypeptide is a human PNMA family protein. In some embodiments, the endogenous Gag polypeptide comprises an amino acid sequence with at least 90% sequence identity to at least 100 consecutive amino acids of any one of SEQ ID NOs: 1-8, 38, 40, 41, 43, 44, 46-49, 52, 53, 55 and 77-92. In some embodiments, the endogenous Gag polypeptide comprises an amino acid sequence with at least 90% sequence identity to any one of SEQ ID NOs: 1-8, 38, 40, 41, 43, 44, 46-49, 52, 53, 55 and 77-92. In some embodiments, the endogenous Gag polypeptide is PNMA5. In some embodiments, the endogenous Gag polypeptide is PNMA5 and comprises an amino acid sequence with at least 90% sequence identity to at least 100 consecutive amino acids of any one of SEQ ID NOs: 5, 60, and 61. In some embodiments, the endogenous Gag polypeptide is PNMA5 and comprises an amino acid sequence with at least 90% sequence identity to any one of SEQ ID NOs: 5, 60, and 61. In some embodiments, the endogenous Gag polypeptide is PNMA5 and comprises an amino acid sequence with at least 90% sequence identity to at least 100 consecutive amino acids of SEQ ID NO: 60. In some embodiments, the endogenous Gag polypeptide is PNMA5 and comprises an amino acid sequence with at least 90% sequence identity to SEQ ID NO: 60. In some embodiments, the endogenous Gag polypeptide is PNMA2. In some embodiments, the endogenous Gag polypeptide is PNMA2 and comprises an amino acid sequence with at least 90% sequence identity to at least 100 consecutive amino acids of any one of SEQ ID NOs: 1, 7, 8, 38, 40, 43, 46, 48, 49, and 52. In some embodiments, the endogenous Gag polypeptide is PNMA2 and comprises an amino acid sequence with at least 90% sequence identity to any one of SEQ ID NOs: 1, 7, 8, 38, 40, 43, 46, 48, 49, and 52. In some embodiments, the endogenous Gag polypeptide is PNMA2 and comprises an amino acid sequence with at least 90% sequence identity to at least 100 consecutive amino acids of SEQ ID NO: 7. In some embodiments, the endogenous Gag polypeptide is PNMA2 and comprises an amino acid sequence with at least 90% sequence identity to SEQ ID NO: 7. In some embodiments, the endogenous Gag polypeptide comprises a sequence modification relative to any one of SEQ ID NOs: 1-8, 38, 40, 41, 43, 44, 46-49, 52, 53, 55 and 77-92.

In some embodiments, the endogenous Gag polypeptide is an RTL family protein. In some embodiments, the endogenous Gag polypeptide is a mammalian RTL family protein. In some embodiments, the endogenous Gag polypeptide is a human RTL family protein. In some embodiments, the endogenous Gag polypeptide comprises an amino acid sequence with at least 90% sequence identity to at least 100 consecutive amino acids of any one of SEQ ID NOs: 60-62 and 65-74. In some embodiments, the endogenous Gag polypeptide comprises an amino acid sequence with at least 90% sequence identity to any one of SEQ ID NOs: 60-62 and 65-74. In some embodiments, the endogenous Gag polypeptide is RTL10 (BOP). In some embodiments, the endogenous Gag polypeptide is RTL10 and comprises an amino acid sequence with at least 90% sequence identity to at least 100 consecutive amino acids of any one of SEQ ID NOs: 60, 61, or 69. In some embodiments, the endogenous Gag polypeptide is RTL10 and comprises an amino acid sequence with at least 90% sequence identity to any one of SEQ ID NOs: 60, 61, or 69. In some embodiments, the endogenous Gag polypeptide is RTL10 and comprises an amino acid sequence with at least 90% sequence identity to at least 100 consecutive amino acids of SEQ ID NO: 60. In some embodiments, the endogenous Gag polypeptide is RTL10 and comprises an amino acid sequence with at least 90% sequence identity to SEQ ID NO: 60. In some embodiments, the endogenous Gag polypeptide is PEG10. In some embodiments, the endogenous Gag polypeptide is PEG10 and comprises an amino acid sequence with at least 90% sequence identity to at least 100 consecutive amino acids of any one of SEQ ID NOs: 65-74. In some embodiments, the endogenous Gag polypeptide is PEG10 and comprises an amino acid sequence with at least 90% sequence identity to any one of SEQ ID NOs: 65-74. In some embodiments, the endogenous Gag polypeptide is PEG10 and comprises an amino acid sequence with at least 90% sequence identity to at least 100 consecutive amino acids of SEQ ID NO: 65. In some embodiments, the endogenous Gag polypeptide is PEG10 and comprises an amino acid sequence with at least 90% sequence identity to SEQ ID NO: 65. In some embodiments, the endogenous Gag polypeptide comprises a sequence modification relative to any one of SEQ ID NOs: 60-62 and 65-74.

In some embodiments, the sequence modification comprises an amino acid deletion. In some embodiments, the sequence modification comprises an amino acid insertion. In some embodiments, the sequence modification comprises an amino acid substitution. In some embodiments, the sequence modification comprises a cargo binding domain. In some embodiments, the sequence modification comprises nucleic acid binding domain. In some embodiments, the sequence modification comprises a zinc finger domain. In some embodiments, the sequence modification comprises a sub-cellular localization signal, a nuclear localization signal, or an antibody or antigen-binding fragment thereof. In some embodiments, the sequence modification is at an N-terminus of the endogenous Gag polypeptide. In some embodiments, the sequence modification is at a C-terminus of the endogenous Gag polypeptide. In some embodiments, the sequence modification within the endogenous Gag polypeptide. In some embodiments, the sequence modification comprises addition of a cysteine residue that is not present in a native form of the endogenous Gag polypeptide or elimination of a cysteine residue that is present in native form of the endogenous Gag polypeptide.

In some embodiments, the disassembly buffer comprises a reducing agent. In some embodiments, the reducing agent comprises reduced glutathione (GSH), beta mercaptoethanol (β-ME), Dithiothreitol (DTT), or tris(2-carboxyethyl)phosphine (TCEP). In some embodiments, the disassembly buffer comprises the reducing agent at a concentration of about 1 mM to about 20 mM. In some embodiments, the disassembly buffer comprises the reducing agent at a concentration of about 5 mM to about 100 mM. In some embodiments, the disassembly buffer comprises the reducing agent at a concentration of about 5-10 mM. In some embodiments, the disassembly buffer comprises the reducing agent at a concentration of about 10-50 mM. In some embodiments, the disassembly buffer comprises a solubilizing agent. In some embodiments, the solubilizing agent comprises a non-denaturing detergent. In some embodiments, the non-denaturing detergent is CHAPS. In some embodiments, the disassembly buffer comprises about 5-10% CHAPS. In some embodiments, the solubilizing agent comprises urea. In some embodiments, the disassembly buffer comprises about 2-10M urea. In some embodiments, the disassembly buffer comprises a pH of about 7-11. In some embodiments, the disassembly buffer comprises a pH of about 8-10. In some embodiments, the disassembly buffer comprises a pH of about 8. In some embodiments, the disassembly buffer further comprises a divalent cation. In some embodiments, the disassembly buffer further comprises MgCl2. In some embodiments, the disassembly buffer comprises CHAPS, TCEP, and optionally Tris. In some embodiments, the disassembly buffer comprises about 5-10% CHAPS, about 10-20 mM TCEP, and optionally about 5-100 mM Tris. In some embodiments, the disassembly buffer comprises TCEP, MgCl₂, and optionally Tris. In some embodiments, the disassembly buffer comprises urea, DTT, optionally NaCl, optionally NaP, and optionally glycerol. In some embodiments, the disassembly buffer comprises about 4-8M urea, about 10-80 mM DTT, optionally about 50-500 mM NaCl, optionally about 5-50 mM NaP, and optionally about 5-15% glycerol. In some embodiments, the disassembly buffer comprises urea, DTT, optionally NaCl, and optionally MgCl2. In some embodiments, the assembly buffer contains less than 500 mOsm/kg of salt. In some embodiments, the assembly buffer contains about 270-330 mOsm/kg of salt. In some embodiments, the assembly buffer is a physiological buffer. In some embodiments, the assembly buffer is a phosphate buffered saline. In some embodiments, the assembly buffer comprises NaCl, KCl, Na₂HPO₄, and KH₂PO₄. In some embodiments, the assembly buffer comprises about 50 mM to about 750 mM of a monovalent salt. In some embodiments, the assembly buffer comprises about 50 mM to about 750 mM of NaCl. In some embodiments, the assembly buffer comprises glycerol. In some embodiments, the assembly buffer comprises about 5-15% glycerol. In some embodiments, the assembly buffer comprises about 10% glycerol. In some embodiments, the assembly buffer comprises a pH of about 6-9. In some embodiments, the assembly buffer comprises a pH of 7.3-8.5. In some embodiments, the assembly buffer comprises Na₂HPO₄/NaH₂PO₄. In some embodiments, the assembly buffer does not contain a chaotropic agent and/or a reducing agent. In some embodiments, the treating in step (c) comprises incubating in in a dialysis cassette with an about 3,000-10,000 Da molecular cutoff. In some embodiments, the method produces assembled capsids of at least about 50% purity as determined by SDS-PAGE. In some embodiments, the method produces assembled capsids with at least about 50% particle homogeneity as determined by multi-angle dynamic light scattering. In some embodiments, at least about 5% of the isolated endogenous Gag polypeptide assembles to form the capsid. In some embodiments, at least 20% of the isolated endogenous Gag polypeptide assembles to form the capsid. In some embodiments, the isolated endogenous Gag polypeptide that assembles to form the capsid is determined by size exclusion chromatography. In some embodiments, the cargo is a heterologous cargo that is not associated the endogenous Gag polypeptide in nature. In some embodiments, the cargo comprises a nucleic acid. In some embodiments, the cargo comprises an RNA. In some embodiments, the cargo comprises a DNA. In some embodiments, the cargo comprises or encodes a gene editing system or a component thereof. In some embodiments, the cargo comprises or encodes a CRISPR/Cas system or a component thereof. In some embodiments, the cargo comprises a polypeptide. In some embodiments, the cargo comprises a therapeutic agent. In some embodiments, the cargo comprises an antibody or antigen-binding fragment thereof, a peptidomimetic, a nucleotidomimetic, a drug, a diagnostic tool, an imaging tool, a small molecule, or a combination thereof.

In some embodiments, the disassembly buffer comprises a monovalent salt, a reducing agent, and optionally a basic buffering agent. In some embodiments, the disassembly buffer comprises NaCl, DTT, and optionally Tris. In some embodiments, the disassembly buffer comprises about 25-1800 mM NaCl, 1-20 mM DTT, and optionally 20-75 mM Tris. In some embodiments, the disassembly buffer comprises about 500 mM NaCl, about 5 mM DTT, and optionally 50 mM Tris. In some embodiments, the disassembly buffer comprises about 50 mM NaCl, about 10 mM DTT, and optionally 25 mM Tris. In some embodiments, the disassembly buffer comprises at least 800 mOsm/kg of salt. In some embodiments, the disassembly buffer comprises less than 150 mOsm/kg of salt. In some embodiments, the assembly buffer contains less than 500 mOsm/kg of salt. In some embodiments, the assembly buffer contains about 270-330 mOsm/kg of salt. In some embodiments, the assembly buffer comprises a nucleic acid. In some embodiments, the assembly buffer comprises a divalent cation. In some embodiments, the assembly buffer comprises ZnCl₂. In some embodiments, the assembly buffer comprises MgCl2. In some embodiments, the assembly buffer comprises NaCl, ZnCl₂, and optionally Tris. In some embodiments, the assembly buffer comprises about 150 mM NaCl, about 10 μM ZnCl₂, and optionally about 25 mM Tris. In some embodiments, the assembly buffer comprises about 150 mM NaCl, about 1 mM DTT, about 10 μM ZnCl₂, and optionally about 50 mM Tris. In some embodiments, the assembly buffer comprises MES and MgCl2. In some embodiments, the assembly buffer comprises about 50 mM MES and about 40 mM MgCl₂. In some embodiments, the assembly buffer comprises a pH of about 6-9. In some embodiments, the assembly buffer comprises a pH of about 7.5-8. In some embodiments, the assembly buffer comprises a pH of about 5-7. In some embodiments, the assembly buffer comprises a pH of about 6. In some embodiments, the assembly buffer does not contain a chaotropic agent and/or a reducing agent. In some embodiments, the treating in step (c) comprises incubating in in a dialysis cassette with an about 3,000-10,000 Da molecular cutoff. In some embodiments, the method produces assembled capsids of at least about 50% purity as determined by SDS-PAGE. In some embodiments, the method produces assembled capsids with at least about 50% particle homogeneity as determined by multi-angle dynamic light scattering. In some embodiments, at least about 5% of the isolated endogenous Gag polypeptide assembles to form the capsid. In some embodiments, at least 20% of the isolated endogenous Gag polypeptide assembles to form the capsid. In some embodiments, the percentage of the isolated endogenous Gag polypeptide that assembles to form the capsid is determined by size exclusion chromatography. In some embodiments, the cargo is a heterologous cargo that is not associated the endogenous Gag polypeptide in nature. In some embodiments, the cargo comprises a nucleic acid. In some embodiments, the cargo comprises an RNA. In some embodiments, the cargo comprises a DNA. In some embodiments, the cargo comprises or encodes a gene editing system or a component thereof. In some embodiments, the cargo comprises or encodes a CRISPR/Cas system or a component thereof. In some embodiments, the cargo comprises a therapeutic agent. In some embodiments, the cargo comprises a polypeptide. In some embodiments, the cargo comprises an antibody or antigen-binding fragment thereof, a peptidomimetic, a nucleotidomimetic, a drug, a diagnostic tool, an imaging tool, a small molecule, or a combination thereof.

Disclosed herein, in some aspects, is a composition comprising an endogenous Gag polypeptide that is not Arc and an assembly buffer.

In some embodiments, the assembly buffer contains less than 500 mOsm/kg of salt. In some embodiments, the assembly buffer contains about 270-330 mOsm/kg of salt. In some embodiments, the assembly buffer is a physiological buffer. In some embodiments, the assembly buffer is a phosphate buffered saline. In some embodiments, the assembly buffer comprises NaCl, KCl, Na₂HPO₄, and KH₂PO₄. In some embodiments, the assembly buffer comprises about 50 mM to about 750 mM of a monovalent salt. In some embodiments, the assembly buffer comprises about 50 mM to about 750 mM of NaCl. In some embodiments, the assembly buffer comprises glycerol. In some embodiments, the assembly buffer comprises about 5-15% glycerol. In some embodiments, the assembly buffer comprises about 10% glycerol. In some embodiments, the assembly buffer comprises Na₂HPO₄/NaH₂PO₄. In some embodiments, the assembly buffer comprises a pH of about 6-9. In some embodiments, the assembly buffer comprises a pH of 7.3-8.5. In some embodiments, the assembly buffer does not contain a chaotropic agent and/or a reducing agent.

In some embodiments, the assembly buffer comprises a nucleic acid. In some embodiments, the assembly buffer comprises a divalent cation. In some embodiments, the assembly buffer comprises ZnCl₂. In some embodiments, the assembly buffer comprises MgCl₂. In some embodiments, the assembly buffer comprises NaCl, ZnCl₂, and optionally Tris. In some embodiments, the assembly buffer comprises about 150 mM NaCl, about 10 μM ZnCl₂, and optionally about 25 mM Tris. In some embodiments, the assembly buffer comprises about 150 mM NaCl, about 1 mM DTT, about 10 μM ZnCl₂, and optionally about 50 mM Tris. In some embodiments, the assembly buffer comprises MES and MgCl₂. In some embodiments, the assembly buffer comprises about 50 mM MES and about 40 mM MgCl₂. In some embodiments, the assembly buffer comprises a pH of about 6-9. In some embodiments, the assembly buffer comprises a pH of about 7.5-8. In some embodiments, the assembly buffer comprises a pH of about 5-7. In some embodiments, the assembly buffer comprises a pH of about 6. In some embodiments, the assembly buffer does not contain a chaotropic agent and/or a reducing agent.

Disclosed herein in some aspects, is a composition comprising an endogenous Gag polypeptide and a disassembly buffer.

In some embodiments, the disassembly buffer comprises a reducing agent. In some embodiments, the reducing agent comprises reduced glutathione (GSH), beta mercaptoethanol (β-ME), Dithiothreitol (DTT), or tris(2-carboxyethyl)phosphine (TCEP).

In some embodiments, the disassembly buffer comprises the reducing agent at a concentration of about 5 mM to about 100 mM. In some embodiments, wherein the disassembly buffer comprises the reducing agent at a concentration of about 10-50 mM. In some embodiments, the disassembly buffer comprises a solubilizing agent. In some embodiments, the solubilizing agent comprises a non-denaturing detergent. In some embodiments, the non-denaturing detergent is CHAPS. In some embodiments, the disassembly buffer comprises about 5-10% CHAPS. In some embodiments, the solubilizing agent comprises urea. In some embodiments, the disassembly buffer comprises about 2-10M urea. In some embodiments, the disassembly buffer comprises a pH of about 7-11. In some embodiments, the disassembly buffer comprises a pH of about 8-10. In some embodiments, the disassembly buffer comprises a pH of about 8. In some embodiments, the disassembly buffer further comprises a divalent cation. In some embodiments, the disassembly buffer further comprises MgCl2. In some embodiments, the disassembly buffer comprises CHAPS, TCEP, and optionally Tris. In some embodiments, the disassembly buffer comprises about 5-10% CHAPS, about 10-20 mM TCEP, and optionally about 5-100 mM Tris. In some embodiments, the disassembly buffer comprises TCEP, MgCl2, and optionally Tris. In some embodiments, the disassembly buffer comprises urea, DTT, optionally NaCl, optionally NaP, and optionally glycerol. In some embodiments, the disassembly buffer comprises about 4-8M urea, about 10-80 mM DTT, optionally about 50-500 mM NaCl, optionally about 5-50 mM NaP, and optionally about 5-15% glycerol. In some embodiments, the disassembly buffer comprises urea, DTT, optionally NaCl, and optionally MgCl₂.

In some embodiments, the disassembly buffer comprises the reducing agent at a concentration of about 5-10 mM. In some embodiments, the disassembly buffer comprises a pH of about 7-11. In some embodiments, the disassembly buffer comprises a pH of about 8-10. In some embodiments, the disassembly buffer comprises a pH of about 8. In some embodiments, the disassembly buffer comprises a monovalent salt, a reducing agent, and optionally a basic buffering agent. In some embodiments, the disassembly buffer comprises NaCl, DTT, and optionally Tris. In some embodiments, the disassembly buffer comprises about 25-1800 mM NaCl, 1-20 mM DTT, and optionally 20-75 mM Tris. In some embodiments, the disassembly buffer comprises about 500 mM NaCl, about 5 mM DTT, and optionally 50 mM Tris. In some embodiments, the disassembly buffer comprises about 50 mM NaCl, about 10 mM DTT, and optionally 25 mM Tris. In some embodiments, the disassembly buffer comprises at least 800 mOsm/kg of salt. In some embodiments, the disassembly buffer comprises less than 150 mOsm/kg of salt.

Disclosed herein, in some aspects, is a composition comprising a plurality of capsids comprising an endogenous Gag polypeptide that is not Arc, wherein the plurality of capsids is at least 50% pure as determined by SDS-PAGE.

Disclosed herein, in some aspects, is a composition comprising a plurality of capsids comprising an endogenous Gag polypeptide that is not Arc, wherein the composition comprises at least 50% particle homogeneity as determined by multi-angle dynamic light scattering.

In some embodiments, the endogenous Gag polypeptide is a native endogenous Gag polypeptide. In some embodiments, the endogenous Gag polypeptide is an engineered endogenous Gag polypeptide. In some embodiments, the endogenous Gag polypeptide is a PNMA family protein. In some embodiments, the endogenous Gag polypeptide is a mammalian PNMA family protein. In some embodiments, the endogenous Gag polypeptide is a human PNMA family protein. In some embodiments, the endogenous Gag polypeptide comprises an amino acid sequence with at least 90% sequence identity to at least 100 consecutive amino acids of any one of SEQ ID NOs: 1-8, 38, 40, 41, 43, 44, 46-49, 52, 53, 55 and 77-92. In some embodiments, the endogenous Gag polypeptide comprises an amino acid sequence with at least 90% sequence identity to any one of SEQ ID NOs: 1-8, 38, 40, 41, 43, 44, 46-49, 52, 53, 55 and 77-92. In some embodiments, the endogenous Gag polypeptide is PNMA5. In some embodiments, the endogenous Gag polypeptide is PNMA5 and comprises an amino acid sequence with at least 90% sequence identity to at least 100 consecutive amino acids of any one of SEQ ID NOs: 5, 77, and 78. In some embodiments, the endogenous Gag polypeptide is PNMA5 and comprises an amino acid sequence with at least 90% sequence identity to any one of SEQ ID NOs: 5, 77, and 78. In some embodiments, the endogenous Gag polypeptide is PNMA5 and comprises an amino acid sequence with at least 90% sequence identity to at least 100 consecutive amino acids of SEQ ID NO: 77. In some embodiments, the endogenous Gag polypeptide is PNMA5 and comprises an amino acid sequence with at least 90% sequence identity to SEQ ID NO: 77. In some embodiments, the endogenous Gag polypeptide is PNMA2. In some embodiments, the endogenous Gag polypeptide is PNMA2 and comprises an amino acid sequence with at least 90% sequence identity to at least 100 consecutive amino acids of any one of SEQ ID NOs: 1, 7, 8, 38, 40, 43, 46, 48, 49, and 52. In some embodiments, the endogenous Gag polypeptide is PNMA2 and comprises an amino acid sequence with at least 90% sequence identity to any one of SEQ ID NOs: 1, 7, 8, 38, 40, 43, 46, 48, 49, and 52. In some embodiments, the endogenous Gag polypeptide is PNMA2 and comprises an amino acid sequence with at least 90% sequence identity to at least 100 consecutive amino acids of SEQ ID NO: 7. In some embodiments, the endogenous Gag polypeptide is PNMA2 and comprises an amino acid sequence with at least 90% sequence identity to SEQ ID NO: 7. In some embodiments, the endogenous Gag polypeptide comprises a sequence modification relative to any one of SEQ ID NOs: 1-8, 38, 40, 41, 43, 44, 46-49, 52, 53, 55 and 77-92.

In some embodiments, the endogenous Gag polypeptide is An RTL family protein. In some embodiments, the endogenous Gag polypeptide is a mammalian RTL family protein. In some embodiments, the endogenous Gag polypeptide is a human RTL family protein. In some embodiments, the endogenous Gag polypeptide comprises an amino acid sequence with at least 90% sequence identity to at least 100 consecutive amino acids of any one of SEQ ID NOs: 60-62 and 65-74. In some embodiments, the endogenous Gag polypeptide comprises an amino acid sequence with at least 90% sequence identity to any one of SEQ ID NOs: 60-62 and 65-74. In some embodiments, the endogenous Gag polypeptide is RTL10 (BOP). In some embodiments, the endogenous Gag polypeptide is RTL10 and comprises an amino acid sequence with at least 90% sequence identity to at least 100 consecutive amino acids of any one of SEQ ID NOs: 60, 61, or 69. In some embodiments, the endogenous Gag polypeptide is RTL10 and comprises an amino acid sequence with at least 90% sequence identity to any one of SEQ ID NOs: 60, 61, or 69. In some embodiments, the endogenous Gag polypeptide is RTL10 and comprises an amino acid sequence with at least 90% sequence identity to at least 100 consecutive amino acids of SEQ ID NO: 60. In some embodiments, the endogenous Gag polypeptide is RTL10 and comprises an amino acid sequence with at least 90% sequence identity to SEQ ID NO: 60. In some embodiments, the endogenous Gag polypeptide is PEG10. In some embodiments, the endogenous Gag polypeptide is PEG10 and comprises an amino acid sequence with at least 90% sequence identity to at least 100 consecutive amino acids of any one of SEQ ID NOs: 65-74. In some embodiments, the endogenous Gag polypeptide is PEG10 and comprises an amino acid sequence with at least 90% sequence identity to any one of SEQ ID NOs: 65-74. In some embodiments, the endogenous Gag polypeptide is PEG10 and comprises an amino acid sequence with at least 90% sequence identity to at least 100 consecutive amino acids of SEQ ID NO: 65. In some embodiments, the endogenous Gag polypeptide is PEG10 and comprises an amino acid sequence with at least 90% sequence identity to SEQ ID NO: 65. In some embodiments, the endogenous Gag polypeptide comprises a sequence modification relative to any one of SEQ ID NOs: 60-62 and 65-74.

In some embodiments, the sequence modification comprises an amino acid deletion. In some embodiments, the sequence modification comprises an amino acid insertion. In some embodiments, the sequence modification comprises an amino acid substitution. In some embodiments, the sequence modification comprises a cargo binding domain. In some embodiments, the sequence modification comprises nucleic acid binding domain. In some embodiments, the sequence modification comprises a zinc finger domain. In some embodiments, the sequence modification comprises a sub-cellular localization signal, a nuclear localization signal, or an antibody or antigen-binding fragment thereof. In some embodiments, the sequence modification is at an N-terminus of the endogenous Gag polypeptide. In some embodiments, the sequence modification is at a C-terminus of the endogenous Gag polypeptide. In some embodiments, the sequence modification within the endogenous Gag polypeptide. In some embodiments, the sequence modification comprises addition of a cysteine residue that is not present in a native form of the endogenous Gag polypeptide or elimination of a cysteine residue that is present in native form of the endogenous Gag polypeptide. In some embodiments, the plurality of capsids is at least 95% pure as determined by SDS-PAGE. In some embodiments, the endogenous Gag polypeptide is capable of disassembling into a non-capsid state with an efficiency of at least 1% and reassembling into a capsid state with an efficiency of at least 1%. In some embodiments, the efficiency of the disassembling and the efficiency of the reassembling are as determined by size exclusion chromatography. In some embodiments, the size exclusion chromatography comprises quantifying a capsid peak and a monomer peak. In some embodiments, the endogenous Gag polypeptide is capable of disassembling into a non-capsid state with an efficiency of at least 20%. In some embodiments, the endogenous Gag polypeptide is capable of reassembling into the capsid state with an efficiency of at least 20%. In some embodiments, the composition further comprises a heterologous cargo that is not associated the endogenous Gag polypeptide in nature. In some embodiments, the heterologous cargo comprises a nucleic acid. In some embodiments, the heterologous cargo comprises an RNA. In some embodiments, the heterologous cargo comprises a DNA. In some embodiments, the heterologous cargo comprises or encodes a gene editing system or a component thereof. In some embodiments, the heterologous cargo comprises or encodes a CRISPR/Cas system or a component thereof. In some embodiments, the heterologous cargo comprises a polypeptide. In some embodiments, the heterologous cargo comprises a therapeutic agent. In some embodiments, the heterologous cargo comprises an antibody or antigen-binding fragment thereof, a peptidomimetic, a nucleotidomimetic, a drug, a diagnostic tool, an imaging tool, a small molecule, or a combination thereof. In some embodiments, the composition further comprises a delivery component. In some embodiments, the delivery component comprises a lipid, optionally wherein the lipid is a cationic lipid. In some embodiments, the delivery component comprises a polypeptide, optionally wherein the polypeptide is a cationic polypeptide. In some embodiments, the delivery component comprises polymer, optionally wherein the polymer is a cationic polymer. In some embodiments, the delivery component comprises a cell-penetrating peptide. In some embodiments, the delivery component comprises a fusogenic protein. In some embodiments, the delivery component comprises an endogenous retroviral envelope protein. In some embodiments, the delivery component comprises a liposome.

Disclosed herein, in some aspects is a nucleic acid encoding the endogenous Gag polypeptide of any one of the preceding embodiments. Disclosed herein, in some aspects is a vector comprising the nucleic acid. Disclosed herein, in some aspects is a cell comprising the nucleic acid.

Disclosed herein, in some aspects is a method of delivering a heterologous cargo to a cell, comprising contacting the cell with the composition of any one of the preceding embodiments.

In some embodiments, the cell is a mammalian cell. In some embodiments, the cell is a human cell. In some embodiments, the cell is contacted with the capsid at a concentration of at least about 0.001 μg/mL. In some embodiments, the contacting is in vivo. In some embodiments, the contacting is in vitro or ex vivo.

In some embodiments, the endogenous Gag polypeptide is not PNMA2.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings below.

FIG. 1 depicts a size exclusion chromatography trace demonstrating separation of assembled PNMA2 capsids from disassembled monomers. The vertical axis is mAU of 280.

FIG. 2 provides a schematic of an illustrative PNMA5 polypeptide construct that comprises a 6×His tag (SEQ ID NO: 27), TEV cleavage site, linkers, and PNMA5 sequence. FIG. 2 discloses “GSGSGS” as SEQ ID NO: 58 and “6×His” as SEQ ID NO: 27.

FIG. 3 provides an illustrative image of protein gel stained with Coomassie R-250 showing purified recombinant PNMA5.

FIG. 4 shows multi-angle dynamic light scattering (MADLS) profiles of purified PNMA5 before and after disassembly using conditions disclosed herein.

FIG. 5A provides an illustrative electron microscopy image showing purified PNMA5 capsids.

FIG. 5B provides an illustrative electron microscopy image showing purified PNMA5 capsids after disassembly using conditions disclosed herein.

FIG. 5C provides an illustrative electron microscopy image showing purified PNMA5 capsids after disassembly followed by reassembly with RNA cargo using conditions disclosed herein.

FIG. 6 is a schematic of an illustrative experiment in which PNMA5 is disassembled then reassembled with RNA cargo. FIG. 6 discloses “6×His” as SEQ ID NO: 27, and “GSGSGS” as SEQ ID NO: 58.

FIG. 7 shows results of a gel-shift assay demonstrating association of a Cre RNA with reassembled PNMA5 capsids.

FIG. 8 is a diagram showing an experiment in which PNMA2 capsids were disassembled and re-assembled with positively charged GFP present.

FIG. 9A is a size exclusion chromatogram of PNMA2 capsids that were mixed with GFP after capsid assembly.

FIG. 9B is a size exclusion chromatogram of PNMA2 capsids that were assembled with positively charged GFP present.

FIG. 10 depicts a size exclusion chromatography trace demonstrating separation of assembled PNMA2 capsids from disassembled monomers. The vertical axis is mAU of 280.

FIG. 11 provides a schematic of an illustrative RTL10 polypeptide construct that comprises a 6×His tag (SEQ ID NO: 27), TEV cleavage site, linkers, and RTL10 sequence. FIG. 11 discloses “GSGSGS” as SEQ ID NO: 58 and “6×His” as SEQ ID NO: 27.

FIG. 12 provides an illustrative image of a protein gel stained with Coomassie R-250 showing purified recombinant RTL10.

FIG. 13 shows an illustrative electron microscopy image showing purified RTL10 capsids.

FIG. 14 provides multi-angle dynamic light scattering (MADLS) profiles of purified RTL10 before and after disassembly using conditions disclosed herein.

FIG. 15A provides an illustrative electron microscopy image showing purified RTL10 capsids.

FIG. 15B provides an illustrative electron microscopy image showing purified RTL10 capsids after disassembly using conditions disclosed herein.

FIG. 15C provides an illustrative electron microscopy image showing purified RTL10 capsids after disassembly followed by reassembly with RNA cargo using conditions disclosed herein.

FIG. 16 is a schematic of an illustrative experiment in which RTL10 is disassembled then reassembled with RNA cargo. FIG. 16 discloses “6×His” as SEQ ID NO: 27, and “GSGSGS” as SEQ ID NO: 58.

FIG. 17 shows results of a gel-shift assay demonstrating association of RNA with reassembled RTL10 capsids.

FIG. 18 provides a schematic of an illustrative PEG10 polypeptide construct that comprises an N-terminal maltose binding protein (MBP), TEV cleavage site, and linkers, PEG10, and a C-terminal 6×His tag (SEQ ID NO: 27). FIG. 18 discloses “GSGSGS” as SEQ ID NO: 58 and “6×His” as SEQ ID NO: 27.

FIG. 19 provides an illustrative image of protein gel stained with Coomassie R-250 showing purified recombinant PEG10.

FIG. 20 is a size exclusion chromatogram of purified PEG10.

FIG. 21 provides multi-angle dynamic light scattering (MADLS) profiles of purified PEG10 before and after assembly using conditions disclosed herein.

FIG. 22 shows a representative electron microscopy image of PEG10 capsids assembled in the presence of RNA.

FIG. 23 shows results of a gel-shift assay demonstrating association of RNA with reassembled PEG10 capsids.

FIG. 24 is a schematic summarizing purification and assembly of PEG10 capsids.

FIG. 24 discloses “6×His” as SEQ ID NO: 27, and “GSGSGS” as SEQ ID NO: 58.

FIG. 25 is a diagram showing an experiment in which PNMA2 capsids were disassembled and re-assembled with positively charged GFP present.

FIG. 26A is a size exclusion chromatogram of PNMA2 capsids that were mixed with GFP after assembly.

FIG. 26B is a size exclusion chromatogram of PNMA2 capsids that were assembled with positively charged GFP present.

### DETAILED DESCRIPTION OF THE INVENTION

Administering diagnostic or therapeutic agents to a site of interest with precision has presented an ongoing challenge. Available methods of delivering nucleic acids to cells suffer from a number of limitations. For example, AAV viral vectors often used for gene therapy are immunogenic, have a limited payload capacity, suffer from poor bio-distribution, can only be administered by direct injection, and pose a risk of disrupting host genes by integration. Some studies have suggested that a significant proportion (e.g., ≥50%) of the population have pre-existing immunity to viral vectors such as AAV, which could severely limit their effectiveness in therapeutic applications. The utility of existing non-viral methods is also restricted by a number of shortcomings. Liposomes can be primarily delivered to the liver. Extracellular vesicles can have a limited payload capacity, limited scalability, and be subject to purification difficulties. Thus, there is a need for new and improved compositions and methods for delivering therapeutic payloads. Capsids (or virus-like particles, VLPs) disclosed herein have the potential to address many of these shortcomings.

Disclosed herein, in certain embodiments, are endogenous retroviral capsid polypeptides, such as endogenous Gag (endo-Gag) polypeptides. In some embodiments, endo-Gag polypeptides of the disclosure, such as RTL and PNMA family polypeptides, assemble into capsids for delivery of a cargo of interest. Endo-Gag polypeptides of the disclosure exhibit surprising and unexpected advantages over existing and alternate capsid-forming polypeptides, such as improved efficiency in capsid assembly, capsid disassembly, and/or capsid reassembly, e.g., reassembly with a heterologous cargo. In additional embodiments, described herein are capsids, e.g., RTL family-based, RTL10-based, PEG10-based, PNMA family-based, PNMA2-based, PNMA5-based, or other endo-Gag-based capsids, for delivery of a cargo of interest. Also disclosed herein are engineered endo-Gag polypeptides, such as RTL or PNMA family polypeptides, with additional modifications of particular advantage and utility.

The sequences referenced herein are provided in Table A at the end of the specification.

The disclosure of International Application No. PCT/US2022/013954, filed Jan. 26, 2022, published as International Publication No. WO 2022/164942, which is hereby incorporated by reference in its entirety, including its disclosure of capsids and components thereof as well as their preparation and uses.

### I. ENDOGENOUS GAG POLYPEPTIDES AND PNMA FAMILY POLYPEPTIDES

Endogenous Gag (endo-Gag) proteins are eukaryotic proteins that have predicted and annotated similarity to viral Gag proteins. As described herein, in some embodiments an endo-Gag protein is capable of assembling into a capsid (VLP) state or form. Endo-Gag proteins of the disclosure can be useful, for example, for packaging and delivering cargos (e.g., heterologous cargos) to cells.

Illustrative endo-Gag polypeptides include PNMA1, PNMA2, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, RTL10 (BOP), LDOC1, PEG10 (RTL2), RTL3, RTL6, RTL8A, RTL8B, and ZNF18.

An endogenous Gag protein disclosed herein can be a member of the retrotransposon Gag-like family (e.g. PEG10 (RTL2), RTL3, RTL6, RTL7 (LDOC), RTL8A, RTL8B, or RTL10 (BOP)).

An endogenous Gag protein disclosed herein can be PEG10 (RTL2). In some embodiments, certain versions of PEG10 can contains two overlapping open reading frames, RF1 and RF2, and can be expressed as two proteins by −1 translational frameshifting (−1 FS): (1) a shorter gag-like polypeptide from RF1, comprising a capsid (CA) domain (e.g., including an N-terminal CA and a C-terminal CA), a nucleocapsid (NC) domain, and CCHC-type zinc finger motif), and (2) a longer, gag/pol-like fusion protein from RF1/RF2 that can additionally comprise a protease (PRO), and a reverse transcriptase-like (RT) domain. Additional isoforms resulting from alternatively spliced transcript variants, and use of upstream non-AUG (CUG) start codon have been reported for PEG10. PEG10 can bind mRNA (e.g., its own mRNA) in the 5′-UTR region, in the region near the boundary between the nucleocapsid (NC) and protease (PRO) coding sequences, and in the beginning of the 3′-UTR region. Illustrative examples of PEG10 amino acid sequences are provided in Table A as SEQ ID NOs: 69-74.

In some embodiments, a PEG10 polypeptide disclosed herein comprises or consists essentially of the shorter gag-like polypeptide from RF1, comprising a capsid (CA) domain (e.g., including an N-terminal CA and a C-terminal CA), a nucleocapsid (NC) domain, and/or a CCHC-type zinc finger motif. In some embodiments, a PEG10 polypeptide disclosed herein does not contain the protease (PRO) or reverse transcriptase-like (RT) domain. In some embodiments, a PEG10 polypeptide disclosed herein comprises or consists essentially of the longer, gag/pol-like fusion protein from RF1/RF2.

In some embodiments, a PEG10 polypeptide disclosed herein comprises a capsid (CA) domain (e.g., including an N-terminal CA and a C-terminal CA), a nucleocapsid (NC) domain, and CCHC-type zinc finger motif.

In some embodiments, a PEG10 polypeptide disclosed herein comprises a capsid (CA) domain and CCHC-type zinc finger motif. In some embodiments, a PEG10 polypeptide disclosed herein comprises a capsid (CA) domain and a nucleocapsid (NC) domain. In some embodiments, a PEG10 polypeptide disclosed herein comprises a nucleocapsid (NC) domain and CCHC-type zinc finger motif.

In some embodiments, a PEG10 polypeptide disclosed herein comprises an N-terminal CA domain, a C-terminal CA domain, a nucleocapsid (NC) domain, and CCHC-type zinc finger motif.

In some embodiments, a PEG10 polypeptide disclosed herein comprises a C-terminal CA domain, a nucleocapsid (NC) domain, and CCHC-type zinc finger motif. In some embodiments, a PEG10 polypeptide disclosed herein comprises an N-terminal CA domain, a nucleocapsid (NC) domain, and CCHC-type zinc finger motif). In some embodiments, a PEG10 polypeptide disclosed herein comprises an N-terminal CA domain, a C-terminal CA domain, and CCHC-type zinc finger motif. In some embodiments, a PEG10 polypeptide disclosed herein comprises an N-terminal CA domain, a C-terminal CA domain, and a nucleocapsid (NC) domain.

In some embodiments, a PEG10 polypeptide disclosed herein comprises an N-terminal CA domain and a C-terminal CA domain. In some embodiments, a PEG10 polypeptide disclosed herein comprises an N-terminal CA domain, and a nucleocapsid (NC) domain. In some embodiments, a PEG10 polypeptide disclosed herein comprises an N-terminal CA domain and CCHC-type zinc finger motif. In some embodiments, a PEG10 polypeptide disclosed herein comprises an a C-terminal CA domain and a nucleocapsid (NC) domain. In some embodiments, a PEG10 polypeptide disclosed herein a C-terminal CA domain and a CCHC-type zinc finger motif. In some embodiments, a PEG10 polypeptide disclosed herein comprises an a nucleocapsid (NC) domain and CCHC-type zinc finger motif).

An endogenous Gag protein disclosed herein can be RTL10 (BOP). RTL10 can comprise a Bcl-2 homology 3 (BH3) domain, a capsid (CA) domain (e.g., including an N-terminal CA and a C-terminal CA domain), and/or a nucleocapsid (NC) domain (e.g., a cryptic NC domain).

In some embodiments, an RTL10 polypeptide disclosed herein comprises a BH3 domain, an N-terminal CA domain, a C-terminal CA domain, and a nucleocapsid (NC) domain (e.g., a cryptic NC domain).

In some embodiments, an RTL10 polypeptide disclosed herein comprises an N-terminal CA domain, a C-terminal CA domain, and a nucleocapsid (NC) domain (e.g., a cryptic NC domain). In some embodiments, an RTL10 polypeptide disclosed herein comprises a BH3 domain, a C-terminal CA domain, and a nucleocapsid (NC) domain (e.g., a cryptic NC domain). In some embodiments, an RTL10 polypeptide disclosed herein comprises a BH3 domain, an N-terminal CA domain, and a nucleocapsid (NC) domain (e.g., a cryptic NC domain). In some embodiments, an RTL10 polypeptide disclosed herein comprises a BH3 domain, an N-terminal CA domain, and a C-terminal CA domain.

In some embodiments, an RTL10 polypeptide disclosed herein comprises a C-terminal CA domain and a nucleocapsid (NC) domain (e.g., a cryptic NC domain). In some embodiments, an RTL10 polypeptide disclosed herein comprises a BH3 domain and a nucleocapsid (NC) domain (e.g., a cryptic NC domain). In some embodiments, an RTL10 polypeptide disclosed herein comprises a BH3 domain, and an N-terminal CA domain. In some embodiments, an RTL10 polypeptide disclosed herein comprises an N-terminal CA domain, and a C-terminal CA domain. In some embodiments, an RTL10 polypeptide disclosed herein comprises an N-terminal CA domain a nucleocapsid (NC) domain (e.g., a cryptic NC domain). In some embodiments, an RTL10 polypeptide disclosed herein comprises a BH3 domain and a C-terminal CA domain.

In some embodiments, an RTL10 polypeptide comprises a BH3 domain, e.g, LAQLGDYMS (SEQ ID NO: 76). In some embodiments, an RTL10 polypeptide lacks a BH3 domain. In some embodiments, an RTL10 polypeptide lacks a functional BH3 domain, for example, contains a variant of the BH3 domain sequence that reduces or abolishes one or more functions of the BH3 domain, such as reducing or abolishing binding to an interaction partner (e.g., VDAC binding, Bcl-2 family member binding), or reducing or abolishing induction of apoptosis. Illustrative modifications to reduce function of the BH3 domain can comprise mutation (e.g., substitution) of L118 and/or D123 residues, e.g., L118A and/or D123A substitutions.

Illustrative examples of RTL10 amino acid sequences are provided in Table A as SEQ ID NOs: 69-74.

An endogenous Gag protein disclosed herein can be a member of the Paraneoplastic Ma (PNMA) family. The Paraneoplastic Ma (PNMA) family of endo-Gag proteins comprises 15 members, PNMA1, PNMA2, PNMA3, PNMA4 (MOAP1), PNMA5, PNMA6A, PNMA6B/6D, PNMA6E, PNMA6F, PNMA7A (ZCCHC12), PNMA7B (ZCCHC18), PNMA8A, PNMA8B, PNMA8C, and CCDC8. PNMA family members share sequence homology to the Gag protein of Long Terminal Repeat (LTR) retrotransposons. PNMA proteins are discussed in Pang et al., (2018) PNMA family: Protein interaction network and cell signalling pathways implicated in cancer and apoptosis. Cellular signalling, 45, 54-62, which is incorporated herein by reference for such disclosure.

PNMA polypeptides comprise several domains. Most PNMA family members share high sequence homology at the N-terminal conserved domain (NCD), and the central conserved domain (CCD). In some family members, a unique protein sequence or domain (UPD) is situated between NCD and CCD domains. A region rich in Lysine and Arginine basic residues, designated KRs is also found in the protein sequences of most members of PNMA family. Many PNMA family members share sequence homology at a poly-glutamic acid rich region (PolyE) near the C-terminus. Beyond the polyglutamic acid rich region is the C-terminus with varying length and low sequence homology (variable C-terminal sequence, VCS) that can be identified in the protein sequences among the PNMA family members. Homologues of PNMA proteins exist in other mammalian species, for example, chimpanzee, monkey, rat, and mouse. In some family members, electrostatic interaction between the KRs and PolyE contribute to protein conformation.

PNMA2 is one member of the PNMA family. An illustrative PNMA2 is human PNMA2, which can comprise about 364 amino acids, e.g., as provided in SEQ ID NO: 1. PNMA2 comprises a central conserved domain (CCD), for example, at residues 202-204 of SEQ ID NO: 1, and a poly-glutamic acid rich region (PolyE) near the C-terminus, for example, at residues 333-338 of SEQ ID NO: 1. PNMA2 can form heterodimers with PNMA1 and PNMA4 (MOAP1), for example to modulate (e.g., inhibit) apoptotic signaling. PNMA2 can also comprise cysteine residues that have the potential to for disulfide bonds, for example, at positions 10, 136, 233, and 310 of SEQ ID NO: 1.

PNMA5 is another member of the PNMA family. An illustrative PNMA5 is human PNMA5, which can comprise about 448 amino acids, e.g., as provided in SEQ ID NO: 5.

Full sequences of illustrative PNMA family members are provided in Table A as SEQ ID NOs: 1-6 and 80-82.

Additional examples of endo-Gag proteins are disclosed in Campillos et al., (2006) Computational characterization of multiple Gag-like human proteins, Trends Genet 22 (11): 585-9, which is incorporated herein by reference for such disclosure. Arc (activity-regulated cytoskeleton-associated protein) is another example of an endo-Gag protein. Arc regulates the endocytic trafficking of α-amino-3-hydroxy-5-methylisoxazole-4-propionic acid (AMPA) type glutamate receptors. Arc activities have been linked to synaptic strength and neuronal plasticity. Phenotypes of loss of Arc in experimental murine model included defective formation of long-term memory and reduced neuronal activity and plasticity.

In some embodiments, a nucleic acid sequence or amino acid sequence disclosed herein comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to a nucleic acid sequence or an amino acid sequence provided herein.

In some embodiments, an endo-Gag, RTL, or PNMA polypeptide disclosed herein essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to at least 50 consecutive amino acids of any one of SEQ ID NOs: 1-8, 38, 40, 41, 43, 44, 46-49, 52, 53, 55, 60-62, 65-74, and 77-92.

In some embodiments, an endo-Gag, RTL, or PNMA polypeptide disclosed herein (e.g., a recombinant or engineered endo-Gag, RTL, or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to at least 100 consecutive amino acids of any one of SEQ ID NOs: 1-8, 38, 40, 41, 43, 44, 46-49, 52, 53, 55, 60-62, 65-74, and 77-92.

In some embodiments, an endo-Gag, RTL, or PNMA polypeptide disclosed herein (e.g., a recombinant or engineered endo-Gag, RTL, or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to at least 200 consecutive amino acids of any one of SEQ ID NOs: 1-8, 38, 40, 41, 43, 44, 46-49, 52, 53, 55, 60-62, 65-74, and 77-92.

In some embodiments, an endo-Gag, RTL, or PNMA polypeptide disclosed herein (e.g., a recombinant or engineered endo-Gag, RTL, or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to at least 300 consecutive amino acids of any one of SEQ ID NOs: 1-8, 38, 40, 41, 43, 44, 46-49, 52, 53, 55, 60-62, 65-74, and 77-92.

In some embodiments, an endo-Gag, RTL, or PNMA polypeptide disclosed herein essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to any one of SEQ ID NOs: 1-8, 38, 40, 41, 43, 44, 46-49, 52, 53, 55, 60-62, 65-74, and 77-92.

In some embodiments, an endo-Gag, RTL, or PNMA polypeptide disclosed herein (e.g., a recombinant or engineered PEG10, RTL10, endo-Gag or PNMA polypeptide) comprises one or more amino acid substitutions, deletions or insertions, from the polypeptide having the amino acid sequence of any one of SEQ ID NOs: 1-8, 38, 40, 41, 43, 44, 46-49, 52, 53, 55, 60-62, 65-74, and 77-92.

In some embodiments, an endo-Gag, RTL, or PNMA polypeptide disclosed herein (e.g., a recombinant or engineered PEG10, RTL10, endo-Gag or PNMA polypeptide) comprises from 1, 2, 3, 4, 5, 6, 7, 8, 9, 10 or more up to about 100, 90, 80, 70, 60, 50, 45, 40, 35, 30, 25, 20, 15 amino acid insertions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 amino acid insertions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid insertions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-4, 3-5, 3-6, 3-7, 3-8, 3-9, 3-10, 3-15, 3-20, 3-30, 3-40, 4-5, 4-6, 4-7, 4-8, 4-9, 4-10, 4-15, 4-20, 4-30, 5-6, 5-7, 5-8, 5-9, 5-10, 5-15, 5-20, 5-30, 5-40, 10-15, 15-20, or 20-25 amino acid insertions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises 1, 2, 3, 4, 5, 6, 7, 8, 9, 10, 11, 12, 13, 14, 15, 16, 17, 18, 19, or 20 amino acid insertions relative to any one of SEQ ID NOs: 1-8, 38, 40, 41, 43, 44, 46-49, 52, 53, 55, 60-62, 65-74, and 77-92.

The one or more amino acid insertions can be at the N-terminus, the C-terminus, within the amino acid sequence, or a combination thereof. The amino acid insertions can be contiguous, non-contiguous, or a combination thereof.

In some embodiments, an endo-Gag, RTL, or PNMA polypeptide disclosed herein (e.g., a recombinant or engineered endo-Gag or PNMA polypeptide) comprises from 1, 2, 3, 4, 5, 6, 7, 8, 9, 10 or more up to about 100, 90, 80, 70, 60, 50, 45, 40, 35, 30, 25, 20, 15 amino acid deletions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 amino acid deletions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid deletions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-4, 3-5, 3-6, 3-7, 3-8, 3-9, 3-10, 3-15, 3-20, 3-30, 3-40, 4-5, 4-6, 4-7, 4-8, 4-9, 4-10, 4-15, 4-20, 4-30, 5-6, 5-7, 5-8, 5-9, 5-10, 5-15, 5-20, 5-30, 5-40, 10-15, 15-20, or 20-25 amino acid deletions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises 1, 2, 3, 4, 5, 6, 7, 8, 9, 10, 11, 12, 13, 14, 15, 16, 17, 18, 19, or 20 amino acid deletions relative to any one of SEQ ID NOs: 1-8, 38, 40, 41, 43, 44, 46-49, 52, 53, 55, 60-62, 65-74, and 77-92.

The one or more amino acid deletions can be at the N-terminus, the C-terminus, within the amino acid sequence, or a combination thereof. The amino acid deletions can be contiguous, non-contiguous, or a combination thereof.

In some embodiments, an endo-Gag, RTL, or PNMA polypeptide disclosed herein (e.g., a recombinant or engineered PEG10, RTL10, endo-Gag or PNMA polypeptide) comprises from 1, 2, 3, 4, 5, 6, 7, 8, 9, 10 or more up to about 100, 90, 80, 70, 60, 50, 45, 40, 35, 30, 25, 20, 15 amino acid substitutions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least or at least 50 amino acid substitutions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, or at most 50 amino acid substitutions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises 1-2, 1-3, 1-4, 1-5, 1-6, 1-7, 1-8, 1-9, 1-10, 1-15, 1-20, 1-30, 1-40, 2-3, 2-4, 2-5, 2-6, 2-7, 2-8, 2-9, 2-10, 2-15, 2-20, 2-30, 2-40, 3-4, 3-5, 3-6, 3-7, 3-8, 3-9, 3-10, 3-15, 3-20, 3-30, 3-40, 4-5, 4-6, 4-7, 4-8, 4-9, 4-10, 4-15, 4-20, 4-30, 5-6, 5-7, 5-8, 5-9, 5-10, 5-15, 5-20, 5-30, 5-40, 10-15, 15-20, or 20-25 amino acid substitutions relative to any one of SEQ ID NOs: 1-8, 38, 40, 41, 43, 44, 46-49, 52, 53, 55, 60-62, 65-74, and 77-92.

In some embodiments, the endo-Gag, RTL, or PNMA polypeptide comprises 1, 2, 3, 4, 5, 6, 7, 8, 9, 10, 11, 12, 13, 14, 15, 16, 17, 18, 19, or 20 amino acid substitutions relative to any one of SEQ ID NOs: 1-8, 38, 40, 41, 43, 44, 46-49, 52, 53, 55, 60-62, 65-74, and 77-92.

An amino acid substitution can be a conservative or a non-conservative substitution. The one or more amino acid substitutions can be at the N-terminus, the C-terminus, within the amino acid sequence, or a combination thereof. The amino acid substitutions can be contiguous, non-contiguous, or a combination thereof.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 1.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 2.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 3.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 4.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 5.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 6.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 7.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 8.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 38.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 40.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 43.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 46.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 48.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 49.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 52.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 55.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 77.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 78.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 80.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 81.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 82.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 83.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 84.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 85.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 86.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 87.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 88.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 89.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 90.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 91.

In some embodiments, an endo-Gag or PNMA polypeptide disclosed herein (e.g., a recombinant or engineered endo-Gag or PNMA polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 92.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 60.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 61.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 65.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 66.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 67.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 68.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 69.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 70.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 71.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 72.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 73.

In some embodiments, an endo-Gag or RTL polypeptide disclosed herein (e.g., a recombinant or engineered endo-Gag or RTL polypeptide) comprises, consists essentially of, or consists of an amino acid sequence with at least 70%, at least 75%, at least 80%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.5%, or substantially 100% sequence identity or similarity to SEQ ID NO: 74.

Various methods and software programs are used to determine the homology (e.g., identity or similarity) between two or sequences. For example, the degree of sequence identity or similarity between two sequences can be determined by comparing the two sequences using computer programs commonly employed for this purpose, such as global or local alignment algorithms. Non-limiting examples include NCBI BLAST, BLASTp, BLASTn, BLASTx, tBLASTn, Clustal W, MAFFT, Clustal Omega, AlignMe, Praline, GAP, BESTFIT, Needle (EMBOSS), Stretcher (EMBOSS), GGEARCH2SEQ, Water (EMBOSS), Matcher (EMBOSS), LALIGN, SSEARCH2SEQ, or another suitable method or algorithm, e.g., using default parameters. A Needleman and Wunsch global alignment algorithm can be used to align two sequences over their entire length, maximizing the number of matches and minimizes the number of gaps.

In some embodiments, an endo-Gag polypeptide or PNMA polypeptide disclosed herein is not Arc or does not comprise an amino acid sequence from Arc. In some embodiments, an endo-Gag polypeptide or PNMA polypeptide disclosed herein is not PNMA6 or does not comprise an amino acid sequence from PNMA6. In some embodiments, an endo-Gag polypeptide or PNMA polypeptide disclosed herein is not PNMA6A or does not comprise an amino acid sequence from PNMA6A. In some embodiments, an endo-Gag polypeptide or PNMA polypeptide disclosed herein is not PNMA6B or does not comprise an amino acid sequence from PNMA6B. In some embodiments, an endo-Gag polypeptide disclosed herein is not a protein from the parancoplastic Ma (PNMA) family or does not comprise an amino acid sequence of a protein from the PNMA family. In some embodiments, an endo-Gag polypeptide or PNMA polypeptide disclosed herein is not a protein from the retrotransposon Gag-like family or does not comprise an amino acid sequence from a protein from the retrotransposon Gag-like family. In some embodiments, an endo-Gag polypeptide or PNMA polypeptide disclosed herein is not PEG10 (RTL2) or does not comprise an amino acid sequence from PEG10 (RTL2). In some embodiments, an endo-Gag polypeptide or PNMA polypeptide disclosed herein is not RTL1 or does not comprise an amino acid sequence from RTL1. In some embodiments, an endo-Gag polypeptide or PNMA polypeptide disclosed herein is not RTL3 or does not comprise an amino acid sequence from RTL3. In some embodiments, an endo-Gag polypeptide or PNMA polypeptide disclosed herein is not RTL6 or does not comprise an amino acid sequence from RTL6. In some embodiments, an endo-Gag polypeptide or PNMA polypeptide disclosed herein is not RTL8A or does not comprise an amino acid sequence from RTL8A. In some embodiments, an endo-Gag polypeptide or PNMA polypeptide disclosed herein is not RTL8B or does not comprise an amino acid sequence from RTL8B. In some embodiments, an endo-Gag polypeptide or PNMA polypeptide disclosed herein is not BOP (RTL10) or does not comprise an amino acid sequence from BOP (RTL10). In some embodiments, an endo-Gag polypeptide or PNMA polypeptide disclosed herein is not LDOC1 (RTL7) or does not comprise an amino acid sequence from LDOC1 (RTL7). In some embodiments, an endo-Gag polypeptide or PNMA polypeptide disclosed herein is not MOAP1 (PNMA4) or does not comprise an amino acid sequence from MOAP1 (PNMA4). In some embodiments, an endo-Gag polypeptide or PNMA polypeptide disclosed herein is not ZNF18 or does not comprise an amino acid sequence from ZNF18. In some embodiments, an endo-Gag polypeptide or PNMA polypeptide disclosed herein is not Asprv1 or does not comprise an amino acid sequence from Asprv1. In some embodiments, an endo-Gag polypeptide or PNMA polypeptide disclosed herein is not CCDC8 or does not comprise an amino acid sequence from CCDC8. In some embodiments, an endo-Gag polypeptide or PNMA polypeptide disclosed herein is not PGBD1 or does not comprise an amino acid sequence from PGBD1. In some embodiments, an endo-Gag polypeptide or PNMA polypeptide disclosed herein is not PNMA1 or does not comprise an amino acid sequence from PNMA1. In some embodiments, an endo-Gag polypeptide or PNMA polypeptide disclosed herein is not PNMA3 or does not comprise an amino acid sequence from PNMA3. In some embodiments, an endo-Gag polypeptide or PNMA polypeptide disclosed herein is not PNMA5 or does not comprise an amino acid sequence from PNMA5. In some embodiments, an endo-Gag polypeptide or PNMA polypeptide disclosed herein is not PNMA6B/6D or does not comprise an amino acid sequence from PNMA6B/6D. In some embodiments, an endo-Gag polypeptide or PNMA polypeptide disclosed herein is not PNMA6E or does not comprise an amino acid sequence from PNMA6E. In some embodiments, an endo-Gag polypeptide or PNMA polypeptide disclosed herein is not PNMA6F or does not comprise an amino acid sequence from PNMA6F. In some embodiments, an endo-Gag polypeptide or PNMA polypeptide disclosed herein is not PNMA7A (ZCCHC12) or does not comprise an amino acid sequence from PNMA7A (ZCCHC12). In some embodiments, an endo-Gag polypeptide or PNMA polypeptide disclosed herein is not PNMA7B (ZCCHC18) or does not comprise an amino acid sequence from PNMA7B (ZCCHC18). In some embodiments, an endo-Gag polypeptide or PNMA polypeptide disclosed herein is not PNMA8A or does not comprise an amino acid sequence from PNMA8A. In some embodiments, an endo-Gag polypeptide or PNMA polypeptide disclosed herein is not PNMA8B or does not comprise an amino acid sequence from PNMA8B. In some embodiments, an endo-Gag polypeptide or PNMA polypeptide disclosed herein is not PNMA8C or does not comprise an amino acid sequence from PNMA8C. In some embodiments, an endo-Gag polypeptide or PNMA polypeptide disclosed herein is not CCDC8 or does not comprise an amino acid sequence from CCDC8. In some embodiments, an endo-Gag polypeptide or PNMA polypeptide disclosed herein is not SCAN or does not comprise an amino acid sequence from SCAN. In some embodiments, an endo-Gag polypeptide or PNMA polypeptide disclosed herein is not ZCCHC12 or does not comprise an amino acid sequence from ZCCHC12. In some embodiments, an endo-Gag polypeptide or PNMA polypeptide disclosed herein is not ZCCHC18 or does not comprise an amino acid sequence from ZCCHC18. In some embodiments, an endo-Gag polypeptide or PNMA polypeptide disclosed herein is not ZNF274 or does not comprise an amino acid sequence from ZNF274. In some embodiments, an endo-Gag polypeptide or PNMA polypeptide disclosed herein is not a SUSHI family member or does not comprise an amino acid sequence from a SUSHI family member. In some embodiments, an endo-Gag polypeptide or PNMA polypeptide disclosed herein is not a SCAN family member or does not comprise an amino acid sequence from a SCAN family member. In some embodiments, an endo-Gag polypeptide or PNMA polypeptide disclosed herein is not PNMA2 or does not comprise an amino acid sequence from PNMA2.

In some instances, the endo Gag polypeptide (e.g., RTL family polypeptide, such as RTL10 or PEG10) comprises a full-length endo Gag polypeptide. In other instances, the endo Gag polypeptide comprises a fragment of the endo Gag, such as a truncated endo Gag (e.g., RTL family, RTL10, or PEG10) polypeptide, that participates in the formation of a capsid. In further instances, the endo Gag polypeptide is a recombinant endo Gag polypeptide.

In some instances, the PNMA family polypeptide (e.g., PNMA2 or PNMA5) comprises a full-length PNMA polypeptide. In other instances, the PNMA family polypeptide comprises a fragment of the PNMA, such as a truncated PNMA polypeptide, that participates in the formation of a capsid. In further instances, the PNMA polypeptide is a recombinant PNMA family polypeptide.

In some instances, the endo-Gag polypeptide comprises a full-length endo-Gag protein. In other instances, the endo-Gag polypeptide comprises a fragment of an endo-Gag protein, such as a truncated endo-Gag polypeptide, that can participate in the formation of a capsid. In further instances, the endo-Gag polypeptide is a recombinant endo-Gag polypeptide.

In some instances, the PNMA family or endo-Gag polypeptide comprises from N-terminus to C-terminus the following domains: NCD, UPD, CCD, KRs, PolyE, and VCS.

In some embodiments, one or more non-essential regions which are not involved in capsid formation are removed from a PNMA family or endo-Gag protein (e.g., RTL family, RTL10, or PEG10) to generate an engineered PNMA or endo-Gag polypeptide. In such instances, one or more non-essential regions, e.g., an N-terminal region (e.g., up to 10 amino acids, up to 15 amino acids, up to 20 amino acids, up to 25 amino acids, up to 30 amino acids, up to 40 amino acids, or up to 50 amino acids), a C-terminal region (e.g., up to 10 amino acids, up to 15 amino acids, up to 20 amino acids, up to 25 amino acids, up to 30 amino acids, up to 40 amino acids, or up to 50 amino acids), or a combination thereof, are deleted from a PNMA or endo-Gag protein (e.g., a native or wild type RTL family, RTL10, PEG10, PNMA or endo-Gag protein) to generate an engineered PNMA or endo-Gag polypeptide.

In some embodiments, at least 5, at least 10, at least 15, at least 20, at least 25, at least 30, at least 35, at least 40, at least 45, or at least 50 amino acids are removed from an N-terminus of an RTL family, a PNMA or endo-Gag protein (e.g., a native or wild type RTL10, PEG10, PNMA or endo-Gag protein) to generate an engineered PNMA or endo-Gag polypeptide. In some embodiments, at most 5, at most 10, at most 15, at most 20, at most 25, at most 30, at most 35, at most 40, at most 45, or at most 50 amino acids are removed from an N-terminus of a RTL family, PNMA or endo-Gag protein (e.g., a native or wild type RTL, PNMA or endo-Gag protein) to generate an engineered PNMA or endo-Gag polypeptide. In some embodiments, about 5, about 10, about 15, about 20, about 25, about 30, about 35, about 40, about 45, or about 50, amino acids are removed from an N-terminus of a RTL, PNMA or endo-Gag protein (e.g., a native or wild type RTL, PNMA or endo-Gag protein) to generate an engineered RTL, PNMA or endo-Gag polypeptide. In some embodiments, about 1 to about 50, about 1 to about 40, about 1 to about 35, about 1 to about 30, about 1 to about 27, about 1 to about 25, about 1 to about 23, about 1 to about 20, about 1 to about 15, about 1 to about 10, about 1 to about 5, about 5 to 50, about 5 to 40, about 5 to 35, about 5 to 30, about 5 to 27, about 5 to 25, about 5 to 23, about 5 to 20, about 5 to 15, about 5 to 10, about 10 to 50, about 10 to 40, about 10 to 35, about 10 to 30, about 10 to 27, about 10 to 25, about 10 to 23, about 10 to 20, about 10 to 15, about 15 to 50, about 15 to 40, about 15 to 35, about 15 to 30, about 15 to 27, about 15 to 25, about 15 to 23, about 15 to 20, about 20 to 50, about 20 to 40, about 20 to 35, about 20 to 30, about 20 to 27, about 20 to 25, or about 20 to 23 amino acids are removed from an N-terminus of a RTL, PNMA or endo-Gag protein (e.g., a native or wild type RTL, PNMA or endo-Gag protein) to generate an engineered RTL, PNMA or endo-Gag polypeptide.

In some embodiments, at least 5, at least 10, at least 15, at least 20, at least 25, at least 30, at least 35, at least 40, at least 45, or at least 50 amino acids are removed from a C-terminus of an RTL family, a PNMA or endo-Gag protein (e.g., a native or wild type RTL, PNMA or endo-Gag protein, such as a native or wild type PEG10 or RTL10) to generate an engineered RTL, PNMA or endo-Gag polypeptide. In some embodiments, at most 5, at most 10, at most 15, at most 20, at most 25, at most 30, at most 35, at most 40, at most 45, or at most 50 amino acids are removed from a C-terminus of a RTL, PNMA or endo-Gag protein (e.g., a native or wild type RTL, PNMA or endo-Gag protein) to generate an engineered RTL, PNMA or endo-Gag polypeptide. In some embodiments, about 5, about 10, about 15, about 20, about 25, about 30, about 35, about 40, about 45, or about 50, amino acids are removed from a C-terminus of a RTL, RTL, PNMA or endo-Gag protein (e.g., a native or wild type RTL, PNMA or endo-Gag protein) to generate an engineered RTL, PNMA or endo-Gag polypeptide. In some embodiments, about 1 to about 50, about 1 to about 40, about 1 to about 35, about 1 to about 30, about 1 to about 27, about 1 to about 25, about 1 to about 23, about 1 to about 20, about 1 to about 15, about 1 to about 10, about 1 to about 5, about 5 to 50, about 5 to 40, about 5 to 35, about 5 to 30, about 5 to 27, about 5 to 25, about 5 to 23, about 5 to 20, about 5 to 15, about 5 to 10, about 10 to 50, about 10 to 40, about 10 to 35, about 10 to 30, about 10 to 27, about 10 to 25, about 10 to 23, about 10 to 20, about 10 to 15, about 15 to 50, about 15 to 40, about 15 to 35, about 15 to 30, about 15 to 27, about 15 to 25, about 15 to 23, about 15 to 20, about 20 to 50, about 20 to 40, about 20 to 35, about 20 to 30, about 20 to 27, about 20 to 25, or about 20 to 23 amino acids are removed from a C-terminus of a RTL, PNMA or endo-Gag protein (e.g., a native or wild type RTL, PNMA or endo-Gag protein) to generate an engineered RTL, PNMA or endo-Gag polypeptide.

In some embodiments, at least 15 amino acids are removed from a native or wild type PNMA family polypeptide (e.g., PNMA2 or PNMA5) to generate an engineered PNMA polypeptide. In some embodiments, at most 25 amino acids are removed from a C-terminus of a native or wild type PNMA to generate an engineered PNMA polypeptide. In some embodiments, about 25 amino acids are removed from a native or wild type PNMA to generate an engineered PNMA polypeptide. In some embodiments, about 10 to 30 amino acids are removed from a native or wild type PNMA to generate an engineered PNMA polypeptide. In some embodiments, about 20 to 27 amino acids are removed from a native or wild type PNMA to generate an engineered PNMA polypeptide. In some embodiments, about 24 to 26 amino acids are removed from a native or wild type PNMA to generate an engineered PNMA polypeptide.

In some cases, only the essential regions or substantially only the regions involved in capsid assembly/forming and cargo binding remain in an RTL family, a PNMA or endo-Gag polypeptide. In some embodiments, only the essential regions or substantially only the regions involved in capsid assembly/forming remain in a PEG10, RTL10, PNMA2 or PNMA5 polypeptide. In some embodiments essential regions or regions involved in capsid assembly/forming comprise a capsid (CA) domain, an N-terminal CA domain, a C-terminal CA domain, a nucleocapsid (NC) domain, or a combination thereof. In some embodiments essential regions or regions involved in cargo binding comprise a nucleocapsid (NC) domain, a CCHC type zinc finger motif, N-terminal conserved domain (NCD), central conserved domain (CCD), unique protein sequence or domain (UPD), KRs, PolyE, variable C-terminal sequence, (VCS), or a combination thereof.

In some embodiments, an RTL family, PEG10, RTL10, PNMA, PNMA2, PNMA5, or endo-Gag polypeptide comprises truncations or modifications of domains involved in capsid forming, nucleic acid binding, or delivery.

In some instances, the PNMA or endo-Gag polypeptide comprises from N-terminus to C-terminus the following domains: UPD, CCD, KRs, PolyE, and VCS. In some instances, the PNMA or endo-Gag polypeptide comprises from N-terminus to C-terminus the following domains: NCD, CCD, KRs, PolyE, and VCS. In some instances, the PNMA or endo-Gag polypeptide comprises from N-terminus to C-terminus the following domains: NCD, UPD, KRs, PolyE, and VCS. In some instances, the PNMA or endo-Gag polypeptide comprises from N-terminus to C-terminus the following domains: NCD, UPD, CCD, PolyE, and VCS. In some instances, the PNMA or endo-Gag polypeptide comprises from N-terminus to C-terminus the following domains: NCD, UPD, CCD, KRs, and VCS. In some instances, the PNMA or endo-Gag polypeptide comprises from N-terminus to C-terminus the following domains: NCD, UPD, CCD, KRs, and PolyE.

In some instances, each of the domains is either directly or indirectly fused to the respective two flanking domains. In some instances, a domain of an RTL, PNMA, or endo Gag polypeptide (e.g., PEG10 or RTL10) is either directly or indirectly fused to two flanking domains (e.g., domains on the C-terminal and N-terminal sides). In some instances, the domains are arranged in an order that does not impede capsid assembly and cargo binding. In some embodiments a RTL family, PNMA, or endo-Gag polypeptide (e.g., PEG10 or RTL10) disclosed herein comprises a tag, for example, a maltose binding protein (MBP tag), or an epitope tag or an affinity tag, such as a polyhistidine tag. In some embodiments a RTL family, PNMA, or endo-Gag polypeptide disclosed herein lacks a tag, for example, does not contain an epitope tag, does not contain an affinity tag, or does not contain a polyhistidine tag. In some embodiments a tag is initially present but is removed, e.g., by enzymatic digestion. In some embodiments a RTL family, PNMA, or endo-Gag polypeptide disclosed herein comprises an N-terminal methionine. In some embodiments a RTL, PNMA, or endo-Gag polypeptide disclosed herein lacks an N-terminal methionine. In some embodiments an N-terminal methionine is removed, co-translationally and/or via enzymatic digestion.

In some instances, the RTL or PNMA family or endo-Gag polypeptide (e.g., PEG10 or RTL10) is an engineered PNMA or endo-Gag polypeptide. As used herein, an engineered polypeptide is a recombinant polypeptide that is not identical in sequence to a full length, wild-type polypeptide. An engineered RTL, PNMA, or endo-Gag polypeptide (e.g., PEG10 or RTL10) can be functionally distinct from a native RTL, PNMA, or endo-Gag polypeptide, for example, comprising an additional function than the native polypeptide or lacking a function that the native polypeptide has. In some instances, the RTL, PNMA, or endo-Gag polypeptide (e.g., PEG10 or RTL10) is a native RTL, PNMA or endo-Gag polypeptide, for example, comprising, consisting essentially of, or consisting of a wild type amino acid sequence.

In some instances, a PNMA or endo-Gag polypeptide (e.g., an RTL family, such as PEG10 or RTL10, or PNMA2, or PNMA5) is a human endo-Gag or PNMA polypeptide. In some instances, an endo-Gag or PNMA is a non-human endo-Gag or PNMA polypeptide. In additional instances, the endo-Gag or PNMA polypeptide comprises one or more domains of a human endo-Gag or PNMA polypeptide, in which at least one of the domains participates in the formation of a capsid. In additional instances, the endo-Gag or PNMA polypeptide comprises one or more domains of a non-human endo-Gag or PNMA polypeptide, in which at least one of the domains participates in the formation of a capsid.

In some instances, an engineered RTL, PNMA or endo-Gag polypeptide comprises a fragment of a RTL, PNMA or endo-Gag polypeptide from a first species and at least an additional fragment from a RTL, PNMA or endo-Gag polypeptide of a second species.

In some embodiments, an illustrative mammalian RTL, PNMA or endo-Gag protein for expression as a recombinant or engineered RTL, PNMA or endo-Gag polypeptide is from the species Homo sapiens. Additional illustrative species of primate RTL, PNMA or endo-Gag proteins for expression as a recombinant or engineered RTL, PNMA or endo-Gag polypeptide include: Gorilla, Pongo abelii, Pan paniscus, Macaca nemestrina, Chlorocebus sabaeus, Papio anubis, Rhinopithecus roxellana, Macaca fascicularis, Nomascus leucogenys, Callithrix jacchus, Aotus nancymaae, Cebus capucinus imitator, Saimiri boliviensis boliviensis, Otolemur garnettii, Macaca mulatta, and Macaca fascicularis.

Illustrative species of rodent RTL, PNMA or endo-Gag proteins for expression as a recombinant or engineered RTL, PNMA or endo-Gag polypeptide includes: Fukomys damarensis, Microcebus murinus, Heterocephalus glaber, Propithecus coquereli, Marmota marmota marmota, Galeopterus variegatus, Cavia porcellus, Dipodomys ordii, Octodon degus, Castor canadensis Nannospalax galili, Carlito syrichta, Chinchilla lanigera, Mus musculus, Ictidomys Tridecemlineatus, Rattus norvegicus, Microtus ochrogaster, Otolemur garnettii, Meriones unguiculatus, Cricetulus griseus, Rattus norvegicus, Neotoma lepida, Jaculus jaculus, Mustela putorius furo, Mesocricetus auratus, Tupaia chinensis, Cricetulus griseus, Chrysochloris asiatica, Elephantulus edwardii, Erinaceus europaeus, Ochotona princeps, Sorex araneus, Monodelphis domestica, Echinops telfairi, and Condylura cristata.

### A. Cargo Binding Domain

In some instances, the RTL family, PNMA or endo-Gag polypeptide (e.g., PEG10 or RTL10) comprises a cargo binding domain. In some embodiments, an RTL family, PNMA or endo-Gag polypeptide (e.g., PEG10 or RTL10) is engineered to comprise a cargo binding domain, for example, through insertion of a cargo binding domain, or modification of a cargo binding domain that is endogenous to the RTL, PNMA or endo-Gag polypeptide. In some embodiments, the cargo binding domain is endogenous to the RTL, PNMA or endo-Gag polypeptide.

A cargo binding domain can bind a cargo covalently or non-covalently, e.g., via a linker disclosed herein. A linker can be a peptide linker, for example, a peptide linker that binds the cargo binding domain to a peptide or polypeptide cargo. A linker can be a non-peptide linker disclosed herein that binds to the cargo via a covalent bond or a non-covalent bond as disclosed herein. A Cargo binding domain can be joined to an RTL, PNMA or endo-Gag polypeptide covalently or non-covalently, e.g., via a linker disclosed herein.

In some embodiments a cargo binding domain binds a heterologous cargo, for example, a cargo that is not native to capsids formed from the RTL, PNMA or endo-Gag polypeptide in nature.

In some cases, the cargo binding domain comprises a nucleic acid binding domain, an RNA binding domain, a DNA binding domain, a protein binding domain, a peptide binding domain, an antibody binding domain, a small molecule binding domain, or a peptidomimetic/nucleotidomimetic binding domain. Illustrative cargo binding domains include, but are not limited to, synthetic nucleic acid (e.g., RNA and/or DNA) binding domains, zinc finger domains, arginine-rich domains, domains from GPCRs, antibodies or binding fragments thereof, lipoproteins, integrins, tyrosine kinases, DNA-binding proteins, RNA-binding proteins, nucleases, ligases, proteases, integrases, isomerases, phosphatases, GTPases, aromatases, esterases, adaptor proteins, G-proteins, GEFs, cytokines, interleukins, interleukin receptors, interferons, interferon receptors, caspases, transcription factors, neurotrophic factors and their receptors, growth factors and their receptors, signal recognition particle and receptor components, extracellular matrix proteins, integral components of membrane, ribosomal proteins, translation elongation factors, translation initiation factors, GPI-anchored proteins, tissue factors, dystrophin, utrophin, dystrobrevin, any fusions, combinations, subunits, derivatives, or domains thereof. In some embodiments, a cargo binding domain comprises a domain from an endogenous Gag polypeptide, RTL family polypeptide, or a PNMA family polypeptide that is heterologous with respect to the remainder of the protein or the part of the protein that induces capsid formation. For example, a PEG10, RTL10, PNMA2 or PNMA5 polypeptide can be engineered to comprise a cargo-binding domain that is from or derived from a heterologous endo-Gag polypeptide, such as a heterologous PNMA protein, e.g., PNMA3 or another endo-Gag protein. A zinc finger domain in an endo Gag polypeptide disclosed herein can be a nucleic acid binding domain with specificity of a 3′ UTR of the cognate gene.

In some embodiments, a cargo biding domain or a nucleic acid binding domain is from or derived from a viral protein, for example, TAT protein of HIV. In some embodiments, a cargo biding domain is inverted, for example, compared to a wild type version of the cargo binding domain.

A Cargo binding domain can be a synthetic cargo binding domain, for example, a synthetic nucleic acid binding domain. In some embodiments, a synthetic cargo binding domain is designed such that it comprises an extension of a C-terminal alpha helix of a RTL or PNMA polypeptide or endo-Gag polypeptide. In some embodiments, a synthetic cargo binding domain is designed such that the cargo binding domain is oriented to the interior of an assembled RTL, PNMA or endo-Gag capsid.

A nucleic acid binding domain can be from or derived from an RNA binding protein. A nucleic acid binding domain can be from or derived from a DNA binding protein.

In some embodiments, a cargo binding domain is lysine-rich, for example, comprising at least 5%, at least 10%, at least 15%, at least 20%, at least 25%, at least 30%, at least 35%, or at least 40% lysine residues. In some embodiments, a cargo binding domain is arginine-rich, for example, comprising at least 5%, at least 10%, at least 15%, at least 20%, at least 25%, at least 30%, at least 35%, or at least 40% arginine residues. In some embodiments, a cargo binding domain comprises one or more Lys/Arg (RK) repeats, for example, at least 1, at least 2, at least 3, at least 4, at least 5, at most 2, at most 3, at most 4, at most 5, about 1, about 2, about 3, about 4, or about 5 Lys/Arg repeats.

Illustrative, non-limiting examples of cargo binding domains that are nucleic acid-binding domains are the amino acid sequences SEQ ID NO: 39, SEQ ID NO: 42, and SEQ ID NO: 45, and amino acid sequences that comprise at least 70%, at least 80%, at least 85%, at least 90%, or at least 95% sequence identity to any one of SEQ ID NOs: 39, 72, and 45.

In some cases, the cargo binding domain binds to an RNA, for example, an mRNA, hairpin RNA, guide RNA, shRNA, siRNA, an mRNA, a tRNA, an rRNA, a snRNA, a microRNA, or a non-coding RNA. In some cases, the cargo binding domain binds to a DNA, for example, a ssDNA, dsDNA, or oligonucleotide. In some embodiments, a cargo binding domain binds to RNA and DNA. In some embodiments, a cargo binding domain preferentially binds RNA over DNA. In some embodiments, a cargo binding domain preferentially binds DNA over RNA. In some embodiments, a cargo binding domain preferentially binds ssDNA. In some embodiments, a cargo binding domain specifically or preferentially binds a particular nucleic acid structural motif, for example, a hairpin, such as a bulge hairpin. In some embodiments, a cargo binding domain non-specifically binds nucleic acids, RNA, and/or DNA, such as ssDNA.

In some embodiments, the PNMA family polypeptide is an engineered PNMA polypeptide with at least an RNA binding domain inserted and/or modified to bind to a heterologous cargo that is not native to capsids formed from the PNMA polypeptide in nature. In some instances, the PNMA polypeptide comprises a full-length PNMA polypeptide with at least an RNA binding domain inserted and/or modified to bind to a heterologous cargo that is not native to capsids formed from the PNMA protein in nature. In additional instances, the engineered PNMA polypeptide comprises one or more domains of a PNMA polypeptide, in which at least one of the domains participates in the formation of a capsid and in which an RNA binding domain is inserted and/or modified to bind to a heterologous cargo that the native PNMA protein does not bind to.

In some embodiments, an endo-Gag disclosed herein is an engineered Endo-Gag polypeptide with at least an RNA binding domain inserted and/or modified to bind to a heterologous cargo that is not native to capsids formed from the endo-Gag protein in nature. In some instances, the endo-Gag polypeptide comprises a full-length endo-Gag polypeptide with at least its RNA binding domain modified to bind to a heterologous cargo that is not native to capsids formed from the endo-Gag protein in nature. In other instances, the endo-Gag polypeptide comprises an engineered endo-Gag fragment comprising modification(s) in at least its RNA binding domain to bind to a heterologous cargo that a native endo-Gag protein does not bind to. In additional instances, the endo-Gag polypeptide comprises one or more domains of an engineered endo-Gag polypeptide, in which at least one of the domains participates in the formation of a capsid and in which the RNA binding domain is modified to bind to a heterologous cargo that is not native to capsids formed from the endo-Gag protein in nature.

In some embodiments, a cargo binding domain is at a C-terminus of an endo-Gag polypeptide or RTL or PNMA polypeptide disclosed herein. In some embodiments, a cargo binding domain is at an N-terminus of an endo-Gag polypeptide or RTL or PNMA polypeptide disclosed herein. In some embodiments, a cargo binding domain is within the sequence of an endo-Gag polypeptide or RTL or PNMA polypeptide disclosed herein.

In some embodiments, a cargo binding domain can be inserted adjacent to a domain of an RTL, PNMA or endo-Gag polypeptide. For example, in some embodiments, a cargo binding domain can be inserted adjacent to (e.g., N-terminal or C-terminal to) one or more of an N-terminal CA domain, C terminal CA domain, NCD, CCD, UPD, KRs, PolyE, or VCS domain.

In some instances, the PNMA or endo-Gag polypeptide comprises from N-terminus to C-terminus the following domains: cargo binding domain, and KRs. In some instances, the PNMA or endo-Gag polypeptide comprises from N-terminus to C-terminus the following domains: KRs, and cargo binding domain.

In some instances, the PNMA or endo-Gag polypeptide comprises from N-terminus to C-terminus the following domains: cargo binding domain, KRs, and PolyE. In some instances, the PNMA or endo-Gag polypeptide comprises from N-terminus to C-terminus the following domains: KRs, cargo binding domain, and PolyE. In some instances, the PNMA or endo-Gag polypeptide comprises from N-terminus to C-terminus the following domains: KRs, PolyE, and cargo binding domain. In some instances, each of the domains is either directly or indirectly fused to the respective two flanking domains.

In some instances, the PNMA or endo-Gag polypeptide comprises from N-terminus to C-terminus the following domains: cargo binding domain, NCD, UPD, CCD, KRs, PolyE, and VCS. In some instances, the PNMA or endo-Gag polypeptide comprises from N-terminus to C-terminus the following domains: NCD, cargo binding domain, UPD, CCD, KRs, PolyE, and VCS. In some instances, the PNMA or endo-Gag polypeptide comprises from N-terminus to C-terminus the following domains: NCD, UPD, cargo binding domain, CCD, KRs, PolyE, and VCS. In some instances, the PNMA or endo-Gag polypeptide comprises from N-terminus to C-terminus the following domains: NCD, UPD, CCD, cargo binding domain, KRs, PolyE, and VCS. In some instances, the PNMA or endo-Gag polypeptide comprises from N-terminus to C-terminus the following domains: NCD, UPD, CCD, KRs, cargo binding domain, PolyE, and VCS. In some instances, the PNMA or endo-Gag polypeptide comprises from N-terminus to C-terminus the following domains: NCD, UPD, CCD, KRs, PolyE, cargo binding domain, and VCS. In some instances, the PNMA or endo-Gag polypeptide comprises from N-terminus to C-terminus the following domains: NCD, UPD, CCD, KRs, PolyE, VCS, and cargo binding domain. In some instances, each of the domains is either directly or indirectly fused to the respective two flanking domains.

In certain embodiments, a domain of a RTL, PNMA or endo-Gag polypeptide is replaced, partially replaced, or acts as an insertion site for a cargo binding domain. For example, in some embodiments, one or more of an N-terminal CA domain, C terminal CA domain, NCD, CCD, UPD, KRs, PolyE, or VCS domain can be replaced, partially replaced, or act as an insertion site for a cargo binding domain.

In some embodiments, the cargo binding domain comprises a sequence that binds to an nucleic acid secondary structure. In some embodiments, the cargo binding domain comprises a sequence that binds to a hairpin loop element. A hairpin may be capable of forming more than one loop. For example, a hairpin capable of forming two intramolecular duplexes and two loops is referred to herein as a “double hairpin”. In some embodiments, the cargo binding domain comprises a sequence that binds to an aptamer sequence. For example, a cargo binding domain can comprise a binding domain of MS2, PP7, Qβ, F2, GA, fr, JP501, M12, R17, BZ13, JP34, JP500, KU1, M11, MX1, TW18, VK, SP, FI, ID2, NL95, TW19, AP205, φCb5, φCb8r, φCb12r, φCb23r, 7s, or PRR1.

In some embodiments, a cargo binding domain sequence comprises folate (e.g, to bind folate receptor or a fragment thereof on a cargo), transferrin (e.g, to bind transferrin or a fragment thereof on a cargo), antibody CC52 (e.g, to bind rat CC531 on a cargo), anti-HER2 (e.g, to bind HER2 or a fragment thereof on a cargo), anti-GD2 (e.g, to bind GD2 or a fragment thereof on a cargo), anti-EGFR (e.g, to bind EGFR or a fragment thereof on a cargo), anti-VEGF or anti-VEGFR (e.g, to bind VEGF, VEGFR or a fragment thereof on a cargo), anti-CD19 (e.g, to bind CD19 or a fragment thereof on a cargo), cyclic arginine-glycine-aspartic acid-tyrosine-cysteine peptide (e.g, to bind avβ3 or a fragment thereof on a cargo), PR b peptide (e.g, to bind alpha-5 beta-1 integrin or a fragment thereof on a cargo), AG86 (e.g, to bind alpha 6 beta 4 integrin or a fragment thereof on a cargo), P6.1 peptide (e.g, to bind X HER2 receptor X or a fragment thereof on a cargo), affinity peptide LN (e.g, to bind aminopeptidase N or a fragment thereof on a cargo), a synthetic somatostatin analogue (e.g, to bind SSTR2 or a fragment thereof on a cargo), anti-CD20 (e.g, to bind CD20 or a fragment thereof on a cargo), or vice versa (e.g., the cargo binding domain comprises the domain indicated as present on the cargo, and the cargo comprises the domain indicated for the cargo binding domain.

In some embodiments, a cargo binding domain sequence comprises lambdaN (e.g, to bind BoxB or a fragment thereof on a cargo), L7A peptide (e.g, to bind C/D box or a fragment thereof on a cargo), an M52 domain (e.g, to bind MS2 RNA or a fragment thereof on a cargo), TAT peptide (e.g, to bind TAR RNA or a fragment thereof on a cargo), BC2 nanobody (e.g, to bind BC2 peptide or a fragment thereof on a cargo), Sun tag antibody (e.g, to bind sun tag or a fragment thereof on a cargo), a SUMO domain (e.g, to bind SIM or a fragment thereof on a cargo), 4WW (e.g, to bind PPXY or a fragment thereof on a cargo), or vice versa (e.g., the cargo binding domain comprises the domain indicated as present on the cargo, and the cargo comprises the domain indicated for the cargo binding domain.

### B. Exogenous Polypeptide Sequence

In some embodiments, an engineered RTL, PNMA or endo-Gag polypeptide (e.g., PEG10 or RTL10) is modified to comprise an exogenous polypeptide sequence, e.g., in addition to the cargo-binding domain. The exogenous polypeptide sequence can be fused to the RTL, PNMA or endo-Gag polypeptide. The exogenous polypeptide sequence can be covalently or non-covalently attached to the RTL, PNMA or endo-Gag polypeptide, e.g., via a linker disclosed herein.

In some embodiments, the exogenous polypeptide sequence can comprise a sub-cellular localization signal, for example, a nuclear localization signal (NLS), or a sequence that targets the polypeptide to a membrane (e.g., an Arginine-rich domain). In some embodiments, the exogenous polypeptide sequence comprises a domain that binds to a cell surface molecule, for example, an antigen, a polypeptide, a receptor, a lipid (e.g., phospholipid), a lipoprotein, a glycoprotein, or the like. In some embodiments, an exogenous polypeptide recognizes and binds to receptors displayed on the surface of targeted cells. Upon reaching a cell of interest, the cargo is optionally further delivered to an intracellular target. For example, a therapeutic RNA can be translated to a protein if it comes into contact with a ribosome in the cytoplasm of the cell.

In some embodiments, the cell surface molecule is a cell surface protein. In some instances, the cell surface molecule is an antigen expressed by a cancerous cell. In some instances, the cell surface molecule is a neoepitope. In some instances, the cell surface molecule comprises one or more mutations compared to a wild-type protein. Illustrative cancer antigens that can be bound by an exogenous polypeptide sequence include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAPIB, SPAP1C), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Ra, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, anti-transferrin receptor, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) and the like.

In some instances, the cell surface molecule bound by an exogenous polypeptide sequence comprises a cluster of differentiation (CD) cell surface marker, for example, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), or the like.

In some cases, the exogenous polypeptide sequence is or is derived from a protein (e.g., a human protein), an antibody or binding fragment thereof, a viral protein, a Gag-like protein (e.g., a human Gag-like protein), or a de novo engineered protein designed to bind to a target receptor of interest. In some instances, the antibody or binding fragment thereof comprises a humanized antibody or binding fragments thereof, a murine antibody or binding fragment thereof, a chimeric antibody or binding fragment thereof, a monoclonal antibody or binding fragment thereof, a multi-specific antibody or binding fragment thereof, a bispecific antibody or biding fragment thereof, a monovalent Fab′, a divalent Fab2, F (ab)′3 fragments, a single-chain variable fragment (scFv), a bis-scFv, an (scFv) 2, a diabody, a minibody, a nanobody, a triabody, a tetrabody, a disulfide stabilized Fv protein (dsFv), a single-domain antibody (sdAb), an Ig NAR, a camelid antibody or binding fragment thereof (e.g., VHH domain), or a chemically modified derivative thereof. In some instances, the exogenous polypeptide sequence guides the delivery of a capsid formed by the engineered RTL, PNMA polypeptide to a target site of interest.

An antibody or an antigen-binding fragment thereof disclosed herein (e.g., as an exogenous polypeptide sequence or a cargo) can comprise complementarity determining regions (CDRs). In some embodiments, the CDRs determine or substantially determine binding specificity and/or affinity of the antibody or antigen-binding fragment. For example, the CDRs can be grafted onto a different suitable framework, or the framework region can be altered (e.g., via amino acid substitutions, deletions, and/or insertions), and the antigen-binding fragment or domain can retain binding for the target, and the extracellular binding domain remains functional despite the alterations outside of the CDRs. CDRs can be identified by various methods, including but not limited to the Kabat method, the Chothia method, the IMGT method, the AHO method, and the Paratome method. One antigen binding site of an antibody with heavy and light chains or variable regions therefrom comprises six CDRs, three in the hypervariable regions of the light chain variable region, and three in the hypervariable regions of the heavy chain variable region. The CDRs in the light chain are designated L1, L2, and L3, while the CDRs in the heavy chain are designated H1, H2, and H3. CDRs can also be designated LCDR1, LCDR2, LCDR3, HCDR1, HCDR2, and HCDR3, respectively. Certain antibodies or antigen-binding domains contain less than six CDRs. For example, certain antibodies lack a light chain, and can be referred to as heavy chain only antibodies (HCAbs). HCAbs have three CDRs in a variable region referred to as VHH. A single domain antibody, or nanobody, can be generated from such a VHH region of a heavy chain only antibody.

Non-liming examples of exogenous polypeptide sequences include those that encode zinc finger domains, arginine-rich domains, domains from GPCRs, antibodies or binding fragments thereof, lipoproteins, integrins, tyrosine kinases, DNA-binding proteins, RNA-binding proteins, nucleases, ligases, proteases, integrases, isomerases, phosphatases, GTPases, aromatases, esterases, adaptor proteins, G-proteins, GEFs, cytokines, interleukins, interleukin receptors, interferons, interferon receptors, caspases, transcription factors, neurotrophic factors and their receptors, growth factors and their receptors, signal recognition particle and receptor components, extracellular matrix proteins, integral components of membrane, ribosomal proteins, translation elongation factors, translation initiation factors, GPI-anchored proteins, tissue factors, dystrophin, utrophin, dystrobrevin, cell penetrating peptides, fusogenic proteins, viral envelope proteins, endogenous retroviral envelope proteins, any fusions, combinations, subunits, derivatives, or domains thereof.

An exogenous polypeptide sequence can be or can comprise a cell penetrating peptide (CPP). Cell-penetrating peptides (CPPs) can facilitate uptake of macromolecules through cellular membranes and enhance the delivery of CPP-modified molecules to the inside of a cell. A cell-penetrating peptide can comprise or can be a cationic CPP (e.g., TAT, R8, DPV3, DPV6, Penetratin, R9-TAT), an amphipathic CPP (e.g., pVEC, ARF, MPG, MAP, transportan), or a hydrophobic CPP (e.g., Bip4, C105Y, Melttin, gH625). A CPP can be a protein-derived CPP, a synthetic CPP, or a chimeric CPP. CPPs can be comprise amphipathic helical peptides, such as transportan and MAP, where lysine residues are major contributors to the positive charge, and Arg-rich peptides, such as TATp, Antennapedia or penetratin. Other CPPs can include: the minimal protein transduction domain of Antennapedia, a Drosophilia homeoprotein, called penetratin, which is a 16-mer peptide (residues 43-58) present in the third helix of the homeodomain; a 27-amino acid-long chimeric CPP, containing the peptide sequence from the amino terminus of the neuropeptide galanin bound via the Lys residue, mastoparan, a wasp venom peptide; VP22, a major structural component of HSV-1 facilitating intracellular transport, and transportan (18-mer) amphipathic model peptide that translocates plasma membranes of mast cells and endothelial cells by both energy-dependent and—independent mechanisms. In some embodiments, a lipid moiety is modified with CPP(s), for intracellular.

In some embodiments, the exogenous polypeptide sequence comprises a non-native cysteine residue, for example, a cysteine residue that is not present in a native form of the RTL or PNMA polypeptide or endo-Gag polypeptide (e.g., RTL10 or PEG10). The exogenous cysteine residue can be used, for example, for conjugation to a binging partner via suitable chemical reactions, such as Maleimide chemistry. In some embodiments, the exogenous polypeptide sequence comprises a non-native cysteine residue and lacks one or more native cysteine residues, for example, the one or more native cysteine residues are deleted or substituted for non-cysteine residues. The non-native cysteine residue or an exogenous polypeptide sequence comprising the non-native cysteine residue can be fused directly or via a linker to a C-terminus, N-terminus, or within the RTL or PNMA polypeptide or endo Gag polypeptide, e.g., within or between domains of the RTL or PNMA polypeptide or endo Gag polypeptide as disclosed herein.

In some instances, the exogenous polypeptide sequence is fused directly, indirectly via a linker, or chemically to one or more of: a cargo binding domain, N-terminal CA domain, C terminal CA domain, NCD, UPD, CCD, KRs, PolyE, or VCS, if present. In some instances, the exogenous polypeptide sequence is fused directly, indirectly via a linker, or chemically to a C-terminus of the RTL, PNMA or endo-Gag polypeptide. In some instances, the exogenous polypeptide sequence is fused directly, indirectly via a linker, or chemically to an N-terminus of the RTL, PNMA or endo-Gag polypeptide.

In some embodiments, an exogenous polypeptide sequence can be inserted adjacent to a domain of a PNMA or endo-Gag polypeptide. For example, in some embodiments, an exogenous polypeptide sequence can be inserted adjacent to (e.g., N-terminal or C-terminal to) one or more of an NCD, CCD, UPD, KRs, PolyE, or VCS domain.

In some instances, the PNMA or endo-Gag polypeptide comprises from N-terminus to C-terminus the following domains: exogenous polypeptide sequence, and PolyE. In some instances, the PNMA or endo-Gag polypeptide comprises from N-terminus to C-terminus the following domains: PolyE, and exogenous polypeptide sequence.

In some instances, the PNMA or endo-Gag polypeptide comprises from N-terminus to C-terminus the following domains: exogenous polypeptide sequence, and KRs. In some instances, the PNMA or endo-Gag polypeptide comprises from N-terminus to C-terminus the following domains: KRs, and exogenous polypeptide sequence.

In some instances, the PNMA or endo-Gag polypeptide comprises from N-terminus to C-terminus the following domains: exogenous polypeptide sequence, KRs, and PolyE. In some instances, the PNMA or endo-Gag polypeptide comprises from N-terminus to C-terminus the following domains: KRs, exogenous polypeptide sequence, and PolyE. In some instances, the PNMA or endo-Gag polypeptide comprises from N-terminus to C-terminus the following domains: KRs, PolyE, and exogenous polypeptide sequence. In some instances, each of the domains is either directly or indirectly fused to the respective two flanking domains.

In some instances, the PNMA or endo-Gag polypeptide comprises from N-terminus to C-terminus the following domains: exogenous polypeptide sequence, NCD, UPD, CCD, KRs, PolyE, and VCS. In some instances, the PNMA or endo-Gag polypeptide comprises from N-terminus to C-terminus the following domains: NCD, exogenous polypeptide sequence, UPD, CCD, KRs, PolyE, and VCS. In some instances, the PNMA or endo-Gag polypeptide comprises from N-terminus to C-terminus the following domains: NCD, UPD, exogenous polypeptide sequence, CCD, KRs, PolyE, and VCS. In some instances, the PNMA or endo-Gag polypeptide comprises from N-terminus to C-terminus the following domains: NCD, UPD, CCD, exogenous polypeptide sequence, KRs, PolyE, and VCS. In some instances, the PNMA or endo-Gag polypeptide comprises from N-terminus to C-terminus the following domains: NCD, UPD, CCD, KRs, exogenous polypeptide sequence, PolyE, and VCS. In some instances, the PNMA or endo-Gag polypeptide comprises from N-terminus to C-terminus the following domains: NCD, UPD, CCD, KRs, PolyE, exogenous polypeptide sequence, and VCS. In some instances, the PNMA or endo-Gag polypeptide comprises from N-terminus to C-terminus the following domains: NCD, UPD, CCD, KRs, PolyE, VCS, and exogenous polypeptide sequence. In some instances, each of the domains is either directly or indirectly fused to the respective two flanking domains.

In certain embodiments, a domain of a RTL, PNMA or endo-Gag polypeptide is replaced, partially replaced, or acts as an insertion site for an exogenous polypeptide sequence. For example, in some embodiments, one or more of an N-terminal CA domain, C terminal CA domain, NCD, CCD, UPD, KRs, PolyE, or VCS domain can be replaced, partially replaced, or act as an insertion site for an exogenous polypeptide sequence.

### C. Linker

In certain embodiments, a polypeptide or capsid of the disclosure comprises a linker, for example, a peptide or non-peptide linker that joins two components covalently or non-covalently.

A linker can join a first component (e.g., domain, polypeptide, cargo binding domain, cargo, delivery component, or exogenous polypeptide sequence) to a second component (e.g., domain, polypeptide, cargo binding domain, cargo, delivery component, or exogenous polypeptide sequence).

A linker can join a cargo binding domain to a cargo (e.g., covalently or non-covalently). A linker can join a first cargo binding domain to a second cargo binding domain. A linker can join a cargo binding domain to a different domain. A linker can join a cargo binding domain to a polypeptide (e.g., a RTL or PNMA polypeptide or endo-Gag polypeptide). A linker can join a cargo binding domain to an exogenous polypeptide sequence.

A linker can join a cargo to a cargo binding domain. A linker can join a first cargo to a second cargo. A linker can join a cargo to a domain. A linker can join a cargo to a polypeptide (e.g., a RTL or PNMA polypeptide or endo-Gag polypeptide). A linker can join a cargo to an exogenous polypeptide sequence.

A linker can join an exogenous polypeptide sequence to a polypeptide (e.g., a RTL or PNMA polypeptide or endo-Gag polypeptide). A linker can join a first exogenous polypeptide sequence to a second exogenous polypeptide sequence. A linker can join an exogenous polypeptide sequence to a domain. A linker can join an exogenous polypeptide sequence to a cargo binding domain. A linker can join an exogenous polypeptide sequence to a cargo.

A linker can join a first domain to a second domain (e.g., a cargo binding domain). A linker can join a domain to a polypeptide (e.g., a RTL or PNMA polypeptide or endo-Gag polypeptide). A linker can join a domain to a cargo. A linker can join a domain to an exogenous polypeptide sequence.

A linker can join a first polypeptide (e.g., a RTL or PNMA polypeptide or end-Gag polypeptide) to a second polypeptide. A linker can join a polypeptide (e.g., a RTL or PNMA polypeptide or endo-Gag polypeptide) to a domain. A linker can join a polypeptide (e.g., a RTL or PNMA polypeptide or endo-Gag polypeptide) to a cargo binding domain. A linker can join a polypeptide (e.g., a RTL or PNMA polypeptide or end-Gag polypeptide) to a cargo. A linker can join a polypeptide (e.g., a RTL or PNMA polypeptide or end-Gag polypeptide) to an exogenous polypeptide sequence.

A linker can join any two components of a polypeptide, capsid, complex, or composition disclosed herein. For example, a linker can join a RTL or PNMA polypeptide to a cargo, an endo-Gag polypeptide to a cargo, a RTL or PNMA polypeptide to a cargo-binding domain, an endo-Gag polypeptide to a cargo-binding domain, a RTL or PNMA polypeptide to an exogenous polypeptide sequence, an endo Gag polypeptide to an exogenous polypeptide sequence, a RTL or PNMA polypeptide to a delivery component, an endo-Gag polypeptide to a delivery component, or any other two domains disclosed herein. A linker can join two domains within a RTL or PNMA polypeptide, two domains within a cargo, to domains within a cargo-binding domain, two domains within an exogenous polypeptide sequence, two domains within a delivery component, and the like. A polypeptide, capsid, complex, or composition can comprise multiple linkers. For example, a cargo, cargo-binding domain, or exogenous polypeptide sequence that comprises an antigen-binding fragment can comprise two variable regions joined by a linker, such as an scFv. A polypeptide or domain that comprises one or more linkers can be joined to another polypeptide or domains via another linker that is the same or different, for example, a cargo, cargo binding domain, or exogenous polypeptide sequence that is an scFv can comprise a linker between a VH and VL domain, and the scFv can in turn be joined to a RTL, PNMA or endo-Gag polypeptide by a second linker.

In some embodiments, the linker is a peptide linker. In some instances, the linker is a rigid linker. In other instances, the linker is a flexible linker. In some cases, the linker is a non-cleavable linker. In other cases, the linker is a cleavable linker. In additional cases, the linker comprises a linear structure, or a non-linear structure (e.g., a cyclic structure).

A flexible peptide linker can have a sequence containing stretches of glycine and serine residues. The small size of the glycine and serine residues provides flexibility and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid peptide linker can have, for example, an alpha helix-structure. An alpha-helical rigid linker can act as a spacer between certain protein domains. A peptide linker can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. In some cases, a linker sequence can be, for example at least about 2, at least about 3, at least about 4, at least about 5, at least about 6, at least about 7, at least about 8, at least about 9, at least about 10, at least about 15, at least about 20, at least about 30, at least about 40, or at least about 50 amino acids in length. In some cases, a linker sequence can be, for example at most about 2, at most about 3, at most about 4, at most about 5, at most about 6, at most about 7, at most about 8, at most about 9, at most about 10, at most about 15, at most about 20, at most about 30, at most about 40, at most about 50, at most about 60, at most about 70, at most about 80, or at most about 100 amino acids in length. In some cases, a linker is 5-20 amino acids in length. In some cases, a linker is 10-20 amino acids in length. In some cases, a linker is 4-8 amino acids in length.

A linker can be a non-cleavable linker. A non-cleavable linker of the present disclosure can include a chemical linker that is stable. Examples of non-cleavable linkers that can be used in proteins of the present disclosure to link domains and/or polypeptides can include a thioether linker, an alkyl linker, a polymeric linker. A linker may be an SMCC linker or a PEG linker. In some embodiments, the linker may be a PEG linker. A non-cleavable linker can also include a non-proteolytically cleavable peptide linker. A non-proteolytically cleavable peptide can be inert to proteases present in a given sample, tissue, or organism. For example, a peptide can be inert or substantially inert to all or most human protease cleavage sequences, and thereby can comprise a high degree of stability within humans and human samples. Such a peptide can also comprise a secondary structure which renders a protease cleavage site inert or inaccessible to a protease. A non-cleavable linker of the present disclosure can comprise a half-life for cleavage of at least 1 hour, at least 2 hours, at least 4 hours, at least 8 hours, at least 12 hours, at least 16 hours, at least 1 day, at least 2 days, at least 3 days, at least 1 week, at least 2 weeks, or at least 1 month in the presence of human proteases at 25 °C in pH 7 buffer.

In certain embodiments, non-cleavable linkers comprise short peptides of varying lengths. Exemplary non-cleavable linkers include (EAAAK) n (SEQ ID NO: 9), or (EAAAR) n (SEQ ID NO: 10), where n is from 1 to 5, and up to 30 residues of glutamic acid-proline or lysine-proline repeats. In some embodiments, the non-cleavable linker comprises (GS) n (SEQ ID NO: 100), (SG) n (SEQ ID NO: 101), (GGGGS) n (SEQ ID NO: 11) or (GGGS) n (SEQ ID NO: 12), wherein n is 1 to 10 (e.g., 1, 2, 3, 4, 5, 6, 7, 8, 9, or 10); KESGSVSSEQLAQFRSLD (SEQ ID NO: 13); or EGKSSGSGSESKST (SEQ ID NO: 14). In some embodiments, the non-cleavable linker comprises a poly-Gly/Ala polymer.

In certain embodiments, the linker is a cleavable linker, e.g., an extracellular cleavable linker or an intracellular cleavable linker. In some instances, the linker is designed for cleavage in the presence of particular conditions or in a particular environment (e.g., under physiological conditions). For example, the design of a linker for cleavage by specific conditions, such as by a specific enzyme, allows the targeting of cellular uptake to a specific location.

In some embodiments, the linker is a pH-sensitive linker. In one instance, the linker is cleaved under basic pH conditions. In other instance, the linker is cleaved under acidic pH conditions.

In some embodiments, the linker is cleaved in vivo by endogenous enzymes (e.g., proteases) such as serine proteases including but not limited to thrombin, metalloproteases, furin, cathepsin B, necrotic enzymes (e.g., calpains), and the like. For example, a cleavable linker can be used to covalently link a cargo to a cargo binding domain, and linker can be cleaved in vivo to release the cargo. Exemplary cleavable linkers include, but are not limited to, GGAANLVRGG (SEQ ID NO: 15); SGRIGFLRTA (SEQ ID NO: 16); SGRSA (SEQ ID NO: 17); GFLG (SEQ ID NO: 18); ALAL (SEQ ID NO: 19); FK; PIC(Et)F-F (SEQ ID NO: 20), where C(Et) indicates S-ethylcysteine; PR(S/T)(L/I)(S/T); DEVD (SEQ ID NO: 22); GWEHDG (SEQ ID NO: 23); RPLALWRS (SEQ ID NO: 24); or a combination thereof.

In certain embodiments, a cleavable linker comprises a 2A linker, which can be processed into separate polypeptides co-translationally or after translation. Inclusion of a 2A linker can increase the likelihood that an appropriate ratio of components are produced (e.g., a 1:1, 1:2, 1:3, 1:4, or 1:5 ratio of two components). In some cases, inclusion of a 2A linker can increase the likelihood that equal or close to equal levels of two components are produced (e.g., a capsid subunit and a cargo).

A linker can be a non-peptide linker. A linker can be a chemical linker. A linker can be a chemical bond, for example, a covalent bond or a non-covalent bond. A linker of the disclosure can include a chemical linker. For example, two a first component (e.g., domain, polypeptide, cargo binding domain, cargo, or exogenous polypeptide sequence) can be joined to a second component (e.g., domain, polypeptide, cargo binding domain, cargo, or exogenous polypeptide sequence) by a chemical linker. Each chemical linker of the disclosure can be alkylene, alkenylene, alkynylene, heteroalkylene, cycloalkylene, heterocycloalkylene, arylene, or heteroarylene, any of which is optionally substituted. In some embodiments, a chemical linker of the disclosure can be an ester, ether, amide, thioether, or polyethyleneglycol (PEG). In some embodiments, a linker can reverse the order of the amino acids sequence in a compound where two amino acid sequences are linked, for example, so that the amino acid sequences linked by the linked are head-to-head, rather than head-to-tail. Non-limiting examples of such linkers include diesters of dicarboxylic acids, such as oxalyl diester, malonyl diester, succinyl diester, glutaryl diester, adipyl diester, pimetyl diester, fumaryl diester, maleyl diester, phthalyl diester, isophthalyl diester, and terephthalyl diester. Non-limiting examples of such linkers include diamides of dicarboxylic acids, such as oxalyl diamide, malonyl diamide, succinyl diamide, glutaryl diamide, adipyl diamide, pimetyl diamide, fumaryl diamide, maleyl diamide, phthalyl diamide, isophthalyl diamide, and terephthalyl diamide. Non-limiting examples of such linkers include diamides of diamino linkers, such as ethylene diamine, 1,2-di(methylamino)ethane, 1,3-diaminopropane, 1,3-di(methylamino)propane, 1,4-di(methylamino)butane, 1,5-di(methylamino)pentane, 1,6-di(methylamino)hexane, and pipyrizine.

Non-limiting examples of optional substituents include hydroxyl groups, sulfhydryl groups, halogens, amino groups, nitro groups, nitroso groups, cyano groups, azido groups, sulfoxide groups, sulfone groups, sulfonamide groups, carboxyl groups, carboxaldehyde groups, imine groups, alkyl groups, halo-alkyl groups, alkenyl groups, halo-alkenyl groups, alkynyl groups, halo-alkynyl groups, alkoxy groups, aryl groups, aryloxy groups, aralkyl groups, arylalkoxy groups, heterocyclyl groups, acyl groups, acyloxy groups, carbamate groups, amide groups, ureido groups, epoxy groups, and ester groups.

In some embodiments, a non-peptide linker or a chemical linker is a cleavable linker, such as a self-immolative linker.

In some embodiments, a cleavable linker comprises a chemical trigger that controls cleavage (and, e.g., release of a cargo). A chemical trigger that can be used in a linker of the disclosure can be a dipeptide trigger, cathepsin-cleavable trigger, acid-cleavable trigger, GSH-cleavable trigger, Fe (II)-cleavable trigger, enzyme-cleavable trigger, photo-responsive-cleavable trigger, bioorthogonal cleavable trigger, glycosidase cleavable triggers, phosphatase cleavable trigger, Sulfatase cleavable trigger, Hydrazone trigger, Carbonate trigger, Silyl ether trigger, Disulfide trigger, 1,2,4-Trioxolane trigger, Dipeptide trigger, Triglycyl (CX) trigger, cBu-Cit trigger, β-Glucuronide trigger, β-Galactoside trigger, Pyrophosphate trigger, Arylsulfate trigger, Heptamethine cyanine fluorophore trigger, O-Nitrobenzyl trigger, PC4AP trigger, or a dsProc trigger.

In some embodiments, a linker comprises a Maleimide attachment, Bis(vinylsulfonyl)piperazine attachment, N-methyl-N-phenylvinylsulfonamide attachment, Pt(II)-based attachment, carbamate attachment, carbonate attachment, quaternary ammonium attachment.

In some embodiments, a linker comprises a bond generated by carbonyl condensation, a Staudinger reaction, modified Staudinger ligation, traceless Staudinger ligation, Inverse-electron demand Diels-Alter cycloadditions, Copper-catalyzed azide-alkyne cycloaddition (CuAAC), strain-promoted azide-alkyne cycloaddition (SPAAC), Huisgen cycloaddition, click chemistry, or the like.

In some embodiments, a linker comprises a bond generated between cysteine and a cysteine reactive group, such as malemide or iodoacetamide. For example, cysteine-based conjugation can be used to link two components, such as a cargo binding domain to a cargo. One or more reactive cysteine residue(s) can be introduced at selected positions, for example, via insertion or substitution at a C-terminus of a RTL, PNMA or endo-Gag polypeptide, an N-terminus of a RTL, PNMA or endo-Gag polypeptide, or within the RTL, PNMA or endo-Gag polypeptide (e.g., in a domain disclosed herein or between to domains). The cysteine residue(s) introduced can be joined to another part of a polypeptide via a linker or spacer disclosed herein. The cysteine residue can be introduced such that it does not interfere with structure or function of the RTL, PNMA or endo-Gag polypeptide, e.g., the ability to assemble into a capsid state or form, disassemble into a non-capsid state or form, and/or re-assemble into a capsid state or form. Cysteine residues can optionally be deleted or substituted for non-reactive or less-reactive residues elsewhere to reduce or eliminate reactivity or conjugation at undesirable sites.

In some embodiments, a linker comprises a covalent attachment of two components. In some embodiments, a linker comprises a non-covalent attachment of two components. Two components present in a polypeptide, complex, or capsid can be non-covalently coupled, for example, by ionic bonds, hydrogen bonds, interactions mediated by oligomerization or dimerization domains, etc.

### II. CAPSIDS

In some embodiments, disclosed herein is a capsid. In some instances, the capsid comprises an endo-Gag polypeptide, for example, a RTL or PNMA family polypeptide, such as a PEG10, RTL10 (BOP), PNMA2 or PNMA5 polypeptide. Illustrative endo-Gag polypeptides include PNMA1, PNMA2, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, BOP, LDOC1, PEG10, RTL3, RTL6, RTL8A, RTL8B, and ZNF18. In some instances, the polypeptide is a functional fragment, e.g., that is capable of forming a subunit of a capsid. RTL, PNMA or end-Gag polypeptides of the disclosure can self-assemble to form capsids, also referred to as Virus-like particles (VLPs).

In some embodiments, the capsid comprises a PNMA5-based capsid. In some instances, the PNMA5-based capsid comprises a plurality of recombinant PNMA5 polypeptides of the disclosure. In some instances, the PNMA5-based capsid comprises a plurality of engineered PNMA5 polypeptides of the disclosure. In some embodiments, the PNMA5 polypeptides are recombinant and engineered.

In some embodiments, the capsid comprises a PNMA2-based capsid. In some instances, the PNMA2-based capsid comprises a plurality of recombinant PNMA2 polypeptides of the disclosure. In some instances, the PNMA2-based capsid comprises a plurality of engineered PNMA2 polypeptides of the disclosure. In some embodiments, the PNMA2 polypeptides are recombinant and engineered.

In some embodiments, the capsid comprises an RTL10-based capsid. In some instances, the RTL10-based capsid comprises a plurality of recombinant RTL10 polypeptides of the disclosure. In some instances, the RTL10-based capsid comprises a plurality of engineered RTL10 polypeptides of the disclosure. In some embodiments, the RTL10 polypeptides are recombinant and engineered.

In some embodiments, the capsid comprises a PEG10-based capsid. In some instances, the PEG10-based capsid comprises a plurality of recombinant PEG10 polypeptides of the disclosure. In some instances, the PEG10-based capsid comprises a plurality of engineered PEG10 polypeptides of the disclosure. In some embodiments, the PEG10 polypeptides are recombinant and engineered.

In some embodiments, the capsid comprises an endo-Gag-based capsid. In some instances, the endo-Gag capsid comprises a plurality of recombinant endo-Gag polypeptides of the disclosure. In some instances, the endo-Gag capsid comprises a plurality of engineered endo-Gag polypeptides of the disclosure. In some embodiments, the endo-Gag polypeptides are recombinant and engineered.

In certain embodiments, the assembly of RTL, PNMA and/or endo-Gag-based capsids occurs ex vivo or in vitro. In some instances, the RTL, PNMA and/or endo-Gag-based capsid is assembled in vivo.

In some embodiments, the capsid comprises a disulfide bond. The RTL, PNMA or endo-Gag polypeptides that assemble to form the capsids can comprise one or more cysteine residues that form one or more disulfide bonds. Disulfide bonds can contribute to, for example, assembly, re-assembly, and/or stability of capsids disclosed herein. In some embodiments, disulfide bonds can be reduced in a target site, such as an intracellular (e.g., cytoplasmic) environment, facilitating capsid disassembly and cargo delivery.

In some embodiments, a first RTL, PNMA or endo-Gag polypeptide subunit of a capsid forms an intermolecular disulfide bond with a second RTL, PNMA or endo-Gag polypeptide. In some embodiments, a RTL, PNMA or endo-Gag polypeptide subunit of a capsid forms an intramolecular disulfide bond. In some embodiments, a RTL, PNMA or endo-Gag polypeptide subunit of a capsid forms an intramolecular disulfide bond and an intermolecular disulfide bond. In some embodiments, disulfide bonds contribute to the formation of RTL, PNMA or endo-Gag polypeptide dimers, trimers, tetramers, and/or or other higher-order multimers.

In some embodiments, a RTL, PNMA polypeptide or endo-Gag polypeptide comprises at least one, at least two, at least three, or at least four cysteines that form disulfide bonds, e.g., upon assembly to form a capsid. In some embodiments, a RTL, PNMA polypeptide or endo-Gag polypeptide comprises one cysteine that forms a disulfide bond. In some embodiments, a RTL, PNMA polypeptide or endo-Gag polypeptide comprises two cysteines that form disulfide bonds. In some embodiments, a RTL, PNMA polypeptide or endo-Gag polypeptide comprises three cysteines that form disulfide bonds. In some embodiments, a RTL, PNMA polypeptide or endo-Gag polypeptide comprises four cysteines that form disulfide bonds.

In some embodiments, a PNMA polypeptide or endo-Gag polypeptide comprises a cysteine residue, e.g., a disulfide-forming cysteine residue at a position corresponding to, for example, any one or more of positions 10, 136, 233, and 310 of SEQ ID NO: 1. In some embodiments, a PNMA (e.g., PNMA2 or PNMA5) polypeptide or endo-Gag polypeptide comprises a cysteine residue (e.g., a disulfide-forming cysteine residue) at a position corresponding to residue 10 of SEQ ID NO: 1. In some embodiments, a PNMA (e.g., PNMA2 or PNMA5) polypeptide or endo-Gag polypeptide comprises a cysteine residue (e.g., a disulfide-forming cysteine residue) at a position corresponding to residue 136 of SEQ ID NO: 1. In some embodiments, a PNMA (e.g., PNMA2 or PNMA5) polypeptide or endo-Gag polypeptide comprises a cysteine residue (e.g., a disulfide-forming cysteine residue) at a position corresponding to residue 233 of SEQ ID NO: 1. In some embodiments, a PNMA (e.g., PNMA2 or PNMA5) polypeptide or endo-Gag polypeptide comprises a cysteine residue (e.g., a disulfide-forming cysteine residue) at a position corresponding to residue 310 of SEQ ID NO: 1.

In some embodiments, the capsid has an average diameter of at least 1 nm, or more. In some instances, the capsid has an average diameter of at least 2 nm, at least 3 nm, at least 4 nm, at least 5 nm, at least 10 nm, at least 11 nm, at least 12 nm, at least 13 nm, at least 14 nm, at least 15 nm, at least 16 nm, at least 17 nm, at least 18 nm, at least 19 nm, at least 20 nm, at least 25 nm, at least 30 nm, at least 40 nm, at least 50 nm, at least 60 nm, at least 70 nm, at least 80 nm, at least 90 nm, at least 100 nm, at least 150 nm, at least 200 nm, at least 300 nm, at least 400 nm, at least 500 nm, at least 600 nm, or more. In some instances, the capsid has an average diameter of at least 5 nm, or more. In some instances, the capsid has an average diameter of at least 7 nm, or more. In some cases, the capsid has an average diameter of at least 10 nm, or more. In some instances, the capsid has an average diameter of at least 12 nm, or more. In some instances, the capsid has an average diameter of at least 15 nm, or more. In some instances, the capsid has an average diameter of at least 17 nm, or more. In some instances, the capsid has an average diameter of at least 20 nm, or more. In some cases, the capsid has an average diameter of at least 30 nm, or more. In some cases, the capsid has an average diameter of at least 40 nm, or more. In some cases, the capsid has an average diameter of at least 50 nm, or more. In some cases, the capsid has an average diameter of at least 80 nm, or more. In some cases, the capsid has an average diameter of at least 100 nm, or more. In some cases, the capsid has an average diameter of at least 200 nm, or more. In some cases, the capsid has an average diameter of at least 300 nm, or more. In some cases, the capsid has an average diameter of at least 400 nm, or more. In some cases, the capsid has an average diameter of at least 500 nm, or more. In some cases, the capsid has an average diameter of at least 600 nm, or more.

In some embodiments, the capsid has an average diameter of at most 100 nm, or less. In some instances, the capsid has an average diameter of at most 1 nm, at most 5 nm, at most 10 nm, at most 20 nm, at most 30 nm, at most 40 nm, at most 50 nm, at most 60 nm, at most 70 nm, at most 80 nm, at most 90 nm, at most 95 nm, at most 100 nm, at most 105 nm, at most 110 nm, at most 115 nm, at most 120 nm, at most 125 nm, at most 130 nm, at most 140 nm, at most 150 nm, at most 200 nm, at most 300 nm, at most 400 nm, at most 500 nm, at most 600 nm, or less. In some cases, the capsid has an average diameter of at most 50 nm, or less. In some cases, the capsid has an average diameter of at most 80 nm, or less.

In some cases, the capsid has an average diameter of at most 90 nm, or less. In some cases, the capsid has an average diameter of at most 95 nm, or less. In some cases, the capsid has an average diameter of at most 100 nm, or less. In some cases, the capsid has an average diameter of at most 110 nm, or less. In some cases, the capsid has an average diameter of at most 120 nm, or less. In some cases, the capsid has an average diameter of at most 150 nm, or less. In some cases, the capsid has an average diameter of at most 200 nm, or less. In some cases, the capsid has an average diameter of at most 300 nm, or less. In some cases, the capsid has an average diameter of at most 400 nm, or less. In some cases, the capsid has an average diameter of at most 500 nm, or less. In some cases, the capsid has an average diameter of at most 600 nm, or less.

In some embodiments, the capsid has an average diameter of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 120 nm, about 150 nm, about 200 nm, about 300 nm, about 400 nm, about 500 nm, or about 600 nm. In some instances, the capsid has an average diameter of about 20 nm. In some cases, the capsid has an average diameter of about 30 nm. In some cases, the capsid has an average diameter of about 40 nm. In some cases, the capsid has an average diameter of about 50 nm. In some cases, the capsid has an average diameter of about 60 nm. In some cases, the capsid has an average diameter of about 70 nm. In some cases, the capsid has an average diameter of about 80 nm. In some cases, the capsid has an average diameter of about 100 nm. In some cases, the capsid has an average diameter of about 200 nm.

In some embodiments, the capsid has an average diameter of from about 1 nm to about 600 nm. In some instances, the capsid has an average diameter of from about 5 nm to about 500 nm, from about 5 nm to about 400 nm, from about 5 nm to about 300 nm, from about 5 nm to about 200 nm, from about 5 nm to about 100 nm, from about 5 nm to about 50 nm, from about 5 nm to about 30 nm, 10 nm to about 500 nm, from about 10 nm to about 400 nm, from about 10 nm to about 300 nm, from about 10 nm to about 200 nm, from about 10 nm to about 150 nm, from about 10 nm to about 120 nm, from about 10 nm to about 100 nm, from about 10 nm to about 90 nm, from about 10 nm to about 50 nm, from about 10 nm to about 30 nm, 15 nm to about 500 nm, from about 15 nm to about 400 nm, from about 15 nm to about 300 nm, from about 15 nm to about 200 nm, from about 15 nm to about 150 nm, from about 15 nm to about 120 nm, from about 15 nm to about 100 nm, from about 15 nm to about 90 nm, from about 15 nm to about 50 nm, from about 20 nm to about 500 nm, from about 20 nm to about 400 nm, from about 20 nm to about 300 nm, from about 20 nm to about 200 nm, from about 20 nm to about 150 nm, from about 20 nm to about 120 nm, from about 20 nm to about 100 nm, from about 20 nm to about 90 nm, from about 20 nm to about 50 nm, from about 30 nm to about 500 nm, from about 30 nm to about 400 nm, from about 30 nm to about 300 nm, from about 30 nm to about 200 nm, from about 30 nm to about 100 nm, from about 30 nm to about 50 nm, from about 50 nm to about 300 nm, from about 50 nm to about 200 nm, or from about 50 nm to about 100 nm.

Capsids in a composition of the disclosure can be, for example, at least about 5%, at least about 10%, at least about 20%, at least about 30%, at least about 40%, at least about 50%, at least about 60%, at least about 70%, at least about 80%, at least about 90%, at least 92%, at least 94%, at least 96%, at least 98%, or at least 99% pure. Purity can be determined, for example, by SDS-PAGE.

Capsids in a composition of the disclosure can exhibit, for example, at least about 5%, at least about 10%, at least about 20%, at least about 30%, at least about 40%, at least about 50%, at least about 60%, at least about 70%, at least about 80%, or at least about 90% particle homogeneity. Particle homogeneity can be determined, for example, by multi-angle dynamic light scattering (MADLS), e.g., using a particle diameter size range disclosed herein.

In some instances, the RTL (e.g., PEG10 or RTL10), PNMA and/or endo-Gag-based capsid is stable at room temperature. In some cases, the RTL (e.g., PEG10 or RTL10), PNMA and/or endo-Gag-based capsid is empty. In other cases, the RTL (e.g., PEG10 or RTL10), PNMA and/or endo-Gag-based capsid is loaded (for example, loaded with a heterologous cargo disclosed herein). In some embodiments, the heterologous cargo is in an interior of at least 1%, at least 5%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of a plurality of capsids. In some embodiments, the heterologous cargo is in an interior of at least 50% of a plurality of the capsids. In some embodiments, the heterologous cargo is on an exterior of at least 1%, at least 3%, at least 5%, at least 7%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of a plurality of capsids.

In some embodiments, at least 1%, at least 5%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of the heterologous cargo is in an interior of a plurality of capsids. In some embodiments, at most 1%, at most 3%, at most 5%, at most 7%, at most 10%, at most 20%, at most 30%, at most 40%, at most 50%, at most 60%, at most 70%, at most 80%, at most 90%, at most 91%, at most 92%, at most 93%, at most 94%, at most 95%, at most 96%, at most 97%, at most 98%, at most 99%, or at most 99.5% of the heterologous cargo is on an exterior of a plurality of capsids. In some embodiments, at least 1%, at least 3%, at least 5%, at least 7%, at least 10%, at least 20%, at least 30%, at least 40%, at least 50%, at least 60%, at least 70%, at least 80%, at least 90%, at least 91%, at least 92%, at least 93%, at least 94%, at least 95%, at least 96%, at least 97%, at least 98%, at least 99%, or at least 99.5% of the heterologous cargo is on an exterior of a plurality of capsids.

In some instances, the RTL (e.g., PEG10 or RTL10), PNMA and/or endo-Gag-based capsid is stable at a temperature from about 2 °C to about 37 °C. In some instances, the RTL (e.g., PEG10 or RTL10), PNMA and/or endo-Gag-based capsid is stable at a temperature from about 2 °C to about 40 °C, 2 °C to about 37 °C, 2 °C to about 25 °C, 2 °C to about 20 °C, 4 °C to about 40 °C, 4 °C to about 37 °C, 4 °C to about 25 °C, 4 °C to about 20 °C, 2 °C to about 8 °C, about 2 °C to about 4 °C, about 20 °C to about 37 °C, about 25 °C to about 37 °C, about 20 °C to about 30 °C, about 25 °C to about 30 °C, or about 30 °C to about 37 °C. In some cases, the RTL (e.g., PEG10 or RTL10), PNMA and/or endo-Gag-based capsid is empty. In other cases, the RTL (e.g., PEG10 or RTL10), PNMA and/or endo-Gag-based capsid is loaded (for example, loaded with a heterologous cargo and/or a therapeutic agent, e.g., a DNA or an RNA).

In some instances, the RTL (e.g., PEG10 or RTL10), PNMA and/or endo-Gag-based capsid is stable for at least about 1 day, at least about 2 days, at least about 4 days, at least about 5 days, at least about 7 days, at least about 14 days, at least about 28 days, at least about 30 days, at least about 60 days, at least about 2 months, at least about 3 months, at least about 4 months, at least about 5 months, at least about 6 months, at least about 12 months, at least about 18 months, at least about 24 months, at least about 3 years, at least about 5 years, or longer. In some case, the RTL (e.g., PEG10 or RTL10), PNMA and/or endo-Gag-based capsid exhibits low or minimal degradation, e.g., less than 10%, less than 5%, less than 4%, less than 3%, less than 2%, less than 1%, or less than 0.5% based on the total population of the RTL (e.g., PEG10 or RTL10), PNMA and/or endo-Gag-based capsids that are degraded. In some cases, the RTL (e.g., PEG10 or RTL10), PNMA and/or endo-Gag-based capsid is empty. In other cases, the RTL (e.g., PEG10 or RTL10), PNMA and/or endo-Gag-based capsid is loaded (for example, loaded with a therapeutic agent, e.g., a DNA or an RNA).

In some embodiments, a capsid comprises a first endogenous retroviral capsid polypeptide and a second endogenous retroviral capsid polypeptide; wherein the amino acid sequence of the first endogenous retroviral capsid polypeptide is not identical to the amino acid sequence of the second endogenous retroviral capsid polypeptide.

In some embodiments, the first endogenous retroviral capsid polypeptide is an endo Gag polypeptide or comprises an amino acid sequence of an endo Gag polypeptide. In some embodiments, the first endogenous retroviral capsid polypeptide is a native endo Gag polypeptide or comprises an amino acid sequence of a native endo Gag polypeptide. In some embodiments, the first endogenous retroviral capsid polypeptide is an engineered endo Gag polypeptide or comprises an amino acid sequence of an engineered endo Gag polypeptide. In some embodiments, the first endogenous retroviral capsid polypeptide is an engineered endo Gag polypeptide that comprises an amino acid deletion relative to a corresponding native endo Gag polypeptide. In some embodiments, the first endogenous retroviral capsid polypeptide is an engineered endo Gag polypeptide that comprises an amino acid substitution relative to a corresponding native endo Gag polypeptide. In some embodiments, the first endogenous retroviral capsid polypeptide is an engineered endo Gag polypeptide that comprises an amino acid insertion relative to a corresponding native endo Gag polypeptide. In some embodiments, the first endogenous retroviral capsid polypeptide is an engineered endo Gag polypeptide that comprises a non-native cysteine that is not present in a corresponding native endo Gag polypeptide.

In some embodiments, the first endogenous retroviral capsid polypeptide is a RTL or PNMA polypeptide (e.g., PEG10, RTL10, PNMA2 or PNMA5) or comprises an amino acid sequence of a RTL or PNMA polypeptide. In some embodiments, the first endogenous retroviral capsid polypeptide is a native RTL or PNMA polypeptide or comprises an amino acid sequence of a native RTL or PNMA polypeptide. In some embodiments, the first endogenous retroviral capsid polypeptide is an engineered RTL or PNMA polypeptide or comprises an amino acid sequence of an engineered RTL or PNMA polypeptide. In some embodiments, the first endogenous retroviral capsid polypeptide is an engineered RTL or PNMA polypeptide that comprises an amino acid deletion relative to a corresponding native RTL or PNMA polypeptide. In some embodiments, the first endogenous retroviral capsid polypeptide is an engineered RTL or PNMA polypeptide that comprises an amino acid substitution relative to a corresponding native RTL or PNMA polypeptide. In some embodiments, the first endogenous retroviral capsid polypeptide is an engineered RTL or PNMA polypeptide that comprises an amino acid insertion relative to a corresponding native RTL or PNMA polypeptide. In some embodiments, the first endogenous retroviral capsid polypeptide is an engineered RTL or PNMA polypeptide that comprises a non-native cysteine that is not present in a corresponding native RTL or PNMA polypeptide.

In some embodiments, the first endogenous retroviral capsid polypeptide is not a RTL or PNMA polypeptide or does not contain an amino acid sequence of a RTL or PNMA polypeptide. In some embodiments, the first endogenous retroviral capsid polypeptide is not a native RTL or PNMA polypeptide or does not contain an amino acid sequence of a native RTL or PNMA polypeptide. In some embodiments, the first endogenous retroviral capsid polypeptide is not an engineered RTL or PNMA polypeptide or does not contain an amino acid sequence of an engineered RTL or PNMA polypeptide.

In some embodiments, the first endogenous retroviral capsid polypeptide is not a PNMA2 polypeptide or does not contain an amino acid sequence of a PNMA2 polypeptide. In some embodiments, the first endogenous retroviral capsid polypeptide is not a native PNMA2 polypeptide or does not contain an amino acid sequence of a native PNMA2 polypeptide. In some embodiments, the first endogenous retroviral capsid polypeptide is not an engineered PNMA2 polypeptide or does not contain an amino acid sequence of an engineered PNMA2 polypeptide.

In some embodiments, the first endogenous retroviral capsid polypeptide is not a PNMA5 polypeptide or does not contain an amino acid sequence of a PNMA5polypeptide. In some embodiments, the first endogenous retroviral capsid polypeptide is not a native PNMA5 polypeptide or does not contain an amino acid sequence of a native PNMA5 polypeptide. In some embodiments, the first endogenous retroviral capsid polypeptide is not an engineered PNMA5 polypeptide or does not contain an amino acid sequence of an engineered PNMA5 polypeptide.

In some embodiments, the first endogenous retroviral capsid polypeptide is not an RTL10 polypeptide or does not contain an amino acid sequence of an RTL10 polypeptide. In some embodiments, the first endogenous retroviral capsid polypeptide is not a native RTL10 polypeptide or does not contain an amino acid sequence of a native RTL10 polypeptide. In some embodiments, the first endogenous retroviral capsid polypeptide is not an engineered RTL10 polypeptide or does not contain an amino acid sequence of an engineered RTL10 polypeptide.

In some embodiments, the first endogenous retroviral capsid polypeptide is not a PEG10 polypeptide or does not contain an amino acid sequence of a PEG10 polypeptide. In some embodiments, the first endogenous retroviral capsid polypeptide is not a native PEG10 polypeptide or does not contain an amino acid sequence of a native PEG10 polypeptide. In some embodiments, the first endogenous retroviral capsid polypeptide is not an engineered PEG10 polypeptide or does not contain an amino acid sequence of an engineered PEG10 polypeptide.

In some embodiments, the first endogenous retroviral capsid polypeptide comprises an exogenous polypeptide sequence disclosed herein. In some embodiments, the first endogenous retroviral capsid polypeptide comprises a cargo binding domain disclosed herein (e.g., an RNA, DNA, or protein-binding domain). In some embodiments, the first endogenous retroviral capsid polypeptide comprises a domain that binds to a cell surface molecule. In some embodiments, the first endogenous retroviral capsid polypeptide comprises an antibody or antigen-binding fragment thereof.

In some embodiments, the second endogenous retroviral capsid polypeptide is an endo Gag polypeptide or comprises an amino acid sequence of an endo Gag polypeptide. In some embodiments, the second endogenous retroviral capsid polypeptide is a native endo Gag polypeptide or comprises an amino acid sequence of a native endo Gag polypeptide. In some embodiments, the second endogenous retroviral capsid polypeptide is an engineered endo Gag polypeptide or comprises an amino acid sequence of an engineered endo Gag polypeptide. In some embodiments, the second endogenous retroviral capsid polypeptide is an engineered endo Gag polypeptide that comprises an amino acid deletion relative to a corresponding native endo Gag polypeptide. In some embodiments, the second endogenous retroviral capsid polypeptide is an engineered endo Gag polypeptide that comprises an amino acid substitution relative to a corresponding native endo Gag polypeptide. In some embodiments, the second endogenous retroviral capsid polypeptide is an engineered endo Gag polypeptide that comprises an amino acid insertion relative to a corresponding native endo Gag polypeptide. In some embodiments, the second endogenous retroviral capsid polypeptide is an engineered endo Gag polypeptide that comprises a non-native cysteine that is not present in a corresponding native endo Gag polypeptide.

In some embodiments, the second endogenous retroviral capsid polypeptide is a PNMA (e.g., PNMA2 or PNMA5) polypeptide or comprises an amino acid sequence of a PNMA (e.g., PNMA2 or PNMA5) polypeptide. In some embodiments, the second endogenous retroviral capsid polypeptide is a native PNMA (e.g., PNMA2 or PNMA5) polypeptide or comprises an amino acid sequence of a native PNMA polypeptide. In some embodiments, the second endogenous retroviral capsid polypeptide is an engineered PNMA polypeptide or comprises an amino acid sequence of an engineered PNMA polypeptide. In some embodiments, the second endogenous retroviral capsid polypeptide is an engineered PNMA polypeptide that comprises an amino acid deletion relative to a corresponding native PNMA polypeptide. In some embodiments, the second endogenous retroviral capsid polypeptide is an engineered PNMA polypeptide that comprises an amino acid substitution relative to a corresponding native PNMA polypeptide. In some embodiments, the second endogenous retroviral capsid polypeptide is an engineered PNMA polypeptide that comprises an amino acid insertion relative to a corresponding native PNMA2 polypeptide. In some embodiments, the second endogenous retroviral capsid polypeptide is an engineered PNMA polypeptide that comprises a non-native cysteine that is not present in a corresponding native PNMA polypeptide.

In some embodiments, the second endogenous retroviral capsid polypeptide is an RTL (e.g., RTL10 or PEG10) polypeptide or comprises an amino acid sequence of an RTL polypeptide. In some embodiments, the second endogenous retroviral capsid polypeptide is a native RTL (e.g., RTL10 or PEG10) polypeptide or comprises an amino acid sequence of a native RTL polypeptide. In some embodiments, the second endogenous retroviral capsid polypeptide is an engineered RTL (e.g., RTL10 or PEG10) polypeptide or comprises an amino acid sequence of an engineered RTL polypeptide. In some embodiments, the second endogenous retroviral capsid polypeptide is an engineered RTL polypeptide that comprises an amino acid deletion relative to a corresponding native RTL polypeptide. In some embodiments, the second endogenous retroviral capsid polypeptide is an engineered RTL polypeptide that comprises an amino acid substitution relative to a corresponding native RTL polypeptide. In some embodiments, the second endogenous retroviral capsid polypeptide is an engineered RTL polypeptide that comprises an amino acid insertion relative to a corresponding native RTL polypeptide. In some embodiments, the second endogenous retroviral capsid polypeptide is an engineered RTL polypeptide that comprises a non-native cysteine that is not present in a corresponding native RTL polypeptide.

In some embodiments, the second endogenous retroviral capsid polypeptide comprises an exogenous polypeptide sequence disclosed herein. In some embodiments, the second endogenous retroviral capsid polypeptide comprises a cargo binding domain disclosed herein (e.g., an RNA, DNA, or protein-binding domain). In some embodiments, the second endogenous retroviral capsid polypeptide comprises a domain that binds to a cell surface molecule. In some embodiments, the second endogenous retroviral capsid polypeptide comprises an antibody or antigen-binding fragment thereof.

In some embodiments, the first endogenous retroviral capsid polypeptide and the second endogenous retroviral capsid polypeptide are present in the capsid at a ratio of about 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits. In some instances, the ratio based on the mass (e.g., number of ng) of the capsid forming subunits present.

In some embodiments, the PNMA-based capsid or endo-Gag-based capsid comprises a plurality of recombinant or engineered PNMA polypeptides and a plurality of non-PNMA proteins. In some instances, the ratio of the plurality of recombinant or engineered PNMA polypeptides to the plurality of non-PNMA proteins is 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits.

In some embodiments, the PNMA2-based capsid or endo-Gag-based capsid comprises a plurality of recombinant or engineered PNMA2 polypeptides and a plurality of non-PNMA2 proteins. Exemplary species of non-PNMA2 proteins include but are not limited to, PNMA1, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, RTL10 (BOP), LDOC1, PEG10, RTL3, RTL6, RTL8A, RTL8B, ZNF18, Copia, ASPRV1, a protein or a combination of proteins chosen from the SCAN domain family, and a protein or a combination of proteins chosen from the retrotransposon Gag-like family.

In some instances, the ratio of the plurality of recombinant or engineered PNMA2 polypeptides to the plurality of non-PNMA2 proteins is 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits.

In some embodiments, the PNMA5-based capsid or endo-Gag-based capsid comprises a plurality of recombinant or engineered PNMA5 polypeptides and a plurality of non-PNMA5 proteins. Exemplary species of non-PNMA5 proteins include but are not limited to, PNMA1, PNMA2, PNMA3, PNMA4 (MOAP1), PNMA6A, PNMA6B/6D, PNMA6E, PNMA6F, PNMA7A, PNMA7B, PNMA8A, PNMA8B, PNMA8C, CCDC8, Arc, RTL10 (BOP), LDOC1, PEG10, RTL3, RTL6, RTL8A, RTL8B, ZNF18, Copia, ASPRV1, a protein or a combination of proteins chosen from the SCAN domain family, and a protein or a combination of proteins chosen from the retrotransposon Gag-like family.

In some instances, the ratio of the plurality of recombinant or engineered PNMA5 polypeptides to the plurality of non-PNMA5 proteins is 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits.

In some embodiments, the RTL10-based capsid or endo-Gag-based capsid comprises a plurality of recombinant or engineered RTL10 polypeptides and a plurality of non-RTL10 proteins. Exemplary species of non-RTL10 proteins include but are not limited to, PNMA1, PNMA2, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, LDOC1, PEG10, RTL3, RTL6, RTL8A, RTL8B, ZNF18, Copia, ASPRV1, a protein or a combination of proteins chosen from the SCAN domain family, and a protein or a combination of proteins chosen from the retrotransposon Gag-like family.

In some instances, the ratio of the plurality of recombinant or engineered RTL10 polypeptides to the plurality of non-RTL10 proteins is 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits.

In some embodiments, the PEG10-based capsid or endo-Gag-based capsid comprises a plurality of recombinant or engineered PEG10 polypeptides and a plurality of non-PEG10 proteins. Exemplary species of non-PEG10 proteins include but are not limited to, PNMA1, PNMA2, PNMA3, PNMA4 (MOAP1), PNMA5, PNMA6A, PNMA6B/6D, PNMA6E, PNMA6F, PNMA7A, PNMA7B, PNMA8A, PNMA8B, PNMA8C, CCDC8, Arc, RTL10 (BOP), LDOC1, RTL3, RTL6, RTL8A, RTL8B, ZNF18, Copia, ASPRV1, a protein or a combination of proteins chosen from the SCAN domain family, and a protein or a combination of proteins chosen from the retrotransposon Gag-like family.

In some instances, the ratio of the plurality of recombinant or engineered PEG10 polypeptides to the plurality of non-PEG10 proteins is 1:1, 2:1, 3:1, 4:1, 5:1, 6:1, 7:1, 8:1, 9:1, 10:1, 20:1, 50:1, 100:1, 1:2, 1:3, 1:4, 1:5, 1:6, 1:7, 1:8, 1:9, 1:10, 1:20, or 1:50. In some instances, the ratio is the comparison in molar concentration. In some instances, the ratio is the comparison in the number of capsid forming subunits.

### A. Delivery Component

In some embodiments, a delivery component is combined with an RTL (e.g., PEG10 or RTL10) based or PNMA-based capsid or endo-Gag-based capsid for a targeted delivery to a site of interest. In some instances, the delivery component comprises a carrier, e.g., an extracellular vesicle such as a micelle, a liposome, or a microvesicle; or a viral envelope.

In some instances, the delivery component serves as a primary delivery vehicle for a RTL-based or PNMA-based capsid or endo-Gag-based capsid which does not comprise its own delivery component. In such cases, the delivery component directs the RTL-based or PNMA-based capsid or endo-Gag-based capsid to a target site of interest and optionally facilitates intracellular uptake.

In some embodiments, the delivery component enhances target specificity and/or sensitivity of a RTL-based or PNMA-based capsid. In such cases, the delivery component enhances the specificity and/or affinity of the RTL-based or PNMA-based capsid or endo-Gag-based capsid to the target site. In some embodiments, the delivery components enhance the specificity and/or affinity by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 100-fold, at least 200-fold, at least 500-fold, or more. In additional cases, the delivery components enhance the specificity and/or affinity by about 2-fold, 3-fold, 4-fold, 5-fold, 6-fold, 7-fold, 8-fold, 9-fold, 10-fold, 20-fold, 30-fold, 50-fold, 100-fold, 200-fold, 500-fold, or more. In further cases, the delivery components enhance the specificity and/or affinity by at least 10%, at least 20%, at least 30%, at least 40%, at least 50%, at least 60%, at least 70%, at least 80%, at least 90%, at least 100%, at least 200%, at least 500%, or more.

In some embodiments, the capsid has a low or reduced off-target effect. An off-target effect can be, for example, an effect that occurs when a cargo is delivered to an unintended tissue or cell type, or an effect that occurs when a cargo binds to an unintended interaction partner. In some cases, the off-target effect is less than 10%, less than 5%, less than 4%, less than 3%, less than 2%, less than 1%, or less than 0.5%. In some cases, the capsid does not have an off-target effect.

In some cases, the delivery component(s) enhance the specificity and/or affinity by at least 10%, at least 20%, at least 30%, at least 40%, at least 50%, at least 60%, at least 70%, at least 80%, at least 90%, at least 100%, at least 200%, at least 500%, or more. In further cases, the delivery components enhance the specificity and/or affinity by about 10%, 20%, 30%, 40%, 50%, 60%, 70%, 80%, 90%, 100%, 200%, 500%, or more. Further still, the delivery component optionally reduces an off-target effect by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 100-fold, at least 200-fold, at least 500-fold, or more. Further still, the delivery component optionally reduces off-target effect by at least 10%, at least 20%, at least 30%, at least 40%, at least 50%, at least 60%, at least 70%, at least 80%, at least 90%, at least 100%, at least 200%, at least 500%, or more.

In additional instances, the delivery component serves as a first vehicle that transports an RTL-based or PNMA-based capsid to a general target region (e.g., a tumor microenvironment) and the RTL-based or PNMA-based or endo-Gag-based capsid comprises a second delivery component (for example, an exogenous polypeptide sequence as disclosed herein) that directs the RTL-based or PNMA-based capsid or endo-Gag-based capsid to a more specific target site and optionally facilitates intracellular uptake. In some embodiments, the delivery component (e.g., delivery component that serves as the first vehicle) minimizes off-target effect by at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 50-fold, at least 100-fold, at least 200-fold, at least 500-fold, or more. In some embodiments, the delivery component minimizes off-target effect by at least 10%, at least 20%, at least 30%, at least 40%, at least 50%, at least 60%, at least 70%, at least 80%, at least 90%, at least 100%, at least 200%, at least 500%, or more.

In further instances, the delivery component serves as a first vehicle that transports an RTL-based or PNMA-based capsid to a target site of interest, and the RTL-based or PNMA-based or endo-Gag-based capsid comprises a second delivery component that facilitates intracellular uptake, for example, an exogenous polypeptide sequence as disclosed herein.

In some embodiments, the delivery component comprises an extracellular vesicle. In some instances, the extracellular vesicle comprises a microvesicle, a liposome, or a micelle. In some instances, the extracellular vesicle has an average diameter of from about 10 nm to about 2000 nm, from about 10 nm to about 1000 nm, from about 10 nm to about 800 nm, from about 20 nm to about 600 nm, from about 30 nm to about 500 nm, from about 50 nm to about 200 nm, or from about 80 nm to about 100 nm.

In some embodiments, the delivery component comprises a microvesicle. Also known as circulating microvesicles, microvesicles are membrane-bound vesicles that comprise phospholipids. In some instances, the microvesicle has an average diameter of from about 50 nm to about 1000 nm, from about 100 nm to about 800 nm, from about 200 nm to about 500 nm, or from about 50 nm to about 400 nm.

In some instances, the microvesicle is originated from cell membrane inversion, exocytosis, shedding, blebbing, or budding. In some instances, the microvesicles are generated from differentiated cells. In other instances, the microvesicles are generated from undifferentiated cells, e.g., by blast cells, progenitor cells, or stem cells.

In some embodiments, the delivery component comprises a microparticle. In some embodiments, the delivery component comprises a nanoparticle. In some embodiments, the delivery component comprises a lipid nanoparticle.

In some embodiments, the delivery component comprises a liposome. In some instances, the liposome comprises a plurality of lipopeptides, which are presented on the surface of the liposome, for targeted delivery to a site or region of interest. In some cases, the liposomes fuse with the target cell, whereby the contents of the liposome are then emptied into the target cell. In some cases, a liposome is endocytosed by cells that are phagocytic. Endocytosis is then followed by intralysosomal degradation of liposomal lipids and release of the encapsulated agents.

Illustrative liposomes suitable for incorporation include, and are not limited to, multilamellar vesicles (MLV), oligolamellar vesicles (OLV), unilamellar vesicles (UV), small unilamellar vesicles (SUV), medium-sized unilamellar vesicles (MUV), large unilamellar vesicles (LUV), giant unilamellar vesicles (GUV), multivesicular vesicles (MVV), single or oligolamellar vesicles made by reverse-phase evaporation method (REV), multilamellar vesicles made by the reverse-phase evaporation method (MLV-REV), stable plurilamellar vesicles (SPLV), frozen and thawed MLV (FATMLV), vesicles prepared by extrusion methods (VET), vesicles prepared by French press (FPV), vesicles prepared by fusion (FUV), dehydration-rehydration vesicles (DRV), and bubblesomes (BSV). In some instances, a liposome comprises Amphipol (A8-35). Techniques for preparing liposomes are described in, for example, COLLOIDAL DRUG DELIVERY SYSTEMS, vol. 66 (J. Kreuter ed., Marcel Dekker, Inc. (1994)), which is incorporated herein by reference for such disclosure.

Depending on the method of preparation, liposomes are unilamellar or multilamellar, and vary in size with diameters ranging from about 20 nm to greater than about 1000 nm.

In some instances, liposomes provided herein also comprise carrier lipids. In some embodiments the carrier lipids are phospholipids. Carrier lipids capable of forming liposomes include, but are not limited to, dipalmitoylphosphatidylcholine (DPPC), phosphatidylcholine (PC; lecithin), phosphatidic acid (PA), phosphatidylglycerol (PG), phosphatidylethanolamine (PE), or phosphatidylserine (PS). Other suitable phospholipids further include distearoylphosphatidylcholine (DSPC), dimyristoylphosphatidylcholine (DMPC), dipalmitoylphosphatidyglycerol (DPPG), distearoylphosphatidyglycerol (DSPG), dimyristoylphosphatidylglycerol (DMPG), dipalmitoylphosphatidic acid (DPPA); dimyristoylphosphatidic acid (DMPA), distearoylphosphatidic acid (DSPA), dipalmitoylphosphatidylserine (DPPS), dimyristoylphosphatidylserine (DMPS), distearoylphosphatidylserine (DSPS), dipalmitoylphosphatidyethanolamine (DPPE), dimyristoylphosphatidylethanolamine (DMPE), distearoylphosphatidylethanolamine (DSPE) and the like, or combinations thereof. In some embodiments, the liposomes further comprise a sterol (e.g., cholesterol) which modulates liposome formation. The carrier lipids are optionally any non-phosphate polar lipids. In some embodiments, a liposome comprises an electroneutral lipid.

In some embodiments, a liposome or a delivery component comprises a cationic lipid. Cationic lipids have a head group with permanent positive charges. Non-limiting examples of cationic lipids include 1,2-di-O-octadecenyl-3-trimethylammonium-propane (DOTMA), 1,2-dioleoyl-3-trimethylammonium-propane (DOTAP), Dimethyldioctadecylammonium bromide (DDAB), and 2,3-dioleyloxy-N-[2-(sperminecarboxamido)ethyl]-N,N-dimethyl-1-propanaminium trifluoroacetate (DOSPA), and commercially available transfection reagents.

In some embodiments, the delivery component comprises a micelle. In some instances, the micelle has an average diameter from about 2 nm to about 250 nm, from about 20 nm to about 200 nm, from about 20 nm to about 100 nm, or from about 50 to about 100 nm.

In some instances, the micelle is a polymeric micelle, characterized by a core shell structure, in which the hydrophobic core is surrounded by a hydrophilic shell. In some cases, the hydrophilic shell further comprises a hydrophilic polymer or copolymer and a pH sensitive component.

Exemplary hydrophilic polymers or copolymers include, but are not limited to, poly(N-substituted acrylamides), poly(N-acryloyl pyrrolidine), poly(N-acryloyl piperidine), poly(N-acryl-L-amino acid amides), poly(ethyl oxazoline), methylcellulose, hydroxypropyl acrylate, hydroxyalkyl cellulose derivatives and poly(vinyl alcohol), poly(N-isopropylacrylamide), poly(N-vinyl-2-pyrrolidone), polyethyleneglycol derivatives, and combinations thereof.

The delivery component may comprise a pH-sensitive moiety, which can include, but is not limited to, an alkylacrylic acid such as methacrylic acid, ethylacrylic acid, propyl acrylic acid and butyl acrylic acid, or an amino acid such as glutamic acid.

In some instances, a hydrophobic moiety constitutes the core of the micelle and includes, for example, a single alkyl chain, such as octadecyl acrylate or a double chain alkyl compound such as phosphatidylethanolamine or dioctadecylamine. In some cases, the hydrophobic moiety is optionally a water insoluble polymer such as a poly (lactic acid) or a poly(e-caprolactone).

Polymeric micelles exhibiting pH-sensitive properties are also contemplated and are formed, e.g., by using pH-sensitive polymers including, but not limited to, copolymers from methacrylic acid, methacrylic acid esters and acrylic acid esters, polyvinyl acetate phthalate, hydroxypropyl methyl cellulose phthalate, cellulose acetate phthalate, or cellulose acetate trimellitate.

A Delivery component can comprise a cationic moiety, for example, a cationic lipid, a cationic peptide, or a cationic polymer. A cationic moiety (e.g., lipid, peptide, or moiety) can associate with a capsid (e.g., a negative spike portion thereof) via electrostatic interactions. The presence of a cationic lipid, polymer, or peptide on a capsid can enhance binding to the negatively charged surface of a cell, facilitating increased capsid uptake.

In some embodiments, the delivery component comprises a cationic peptide, e.g., a cationic cell-penetrating peptide disclosed herein.

In some embodiments, the delivery component comprises a cationic polymer. Non-limiting examples of cationic polymers include cationic peptides and their derivatives (e.g., polylysine, polyornithine), linear or branched synthetic polymers (e.g., polybrene, polyethyleneimine), polysaccharide-based delivery molecules (e.g., cyclodextrin, chitosan), natural polymers (e.g., histone, collagen), and activated and non-activated dendrimers. Cationic reagents can adhere to the cell membrane through electrostatic interactions and promote cellular uptake via endocytosis.

In some embodiments, the delivery component comprises a viral envelope or a component thereof. For example, viral envelopes comprise glycoproteins, phospholipids, and additional proteins obtained from a host, any of which a delivery component can comprise. In some instances, the viral envelope or component thereof is permissive to a wide range of target cells. In other instances, the viral envelope or component thereof is non-permissive and is specific to a target cell of interest. In some cases, the viral envelope or component thereof comprises a cell-specific binding protein and optionally a fusogenic molecule that aids in the fusion of the cargo into a target cell. In some cases, the viral envelope or component thereof comprises an endogenous viral envelope. In other cases, the viral envelope is a modified envelope, comprising one or more foreign proteins.

In some instances, the viral envelope or component thereof is derived from a DNA virus. Illustrative enveloped DNA viruses include viruses from the family of Herpesviridae, Poxviridae, and Hepadnavirdae. In other instances, the viral envelope or component thereof is derived from an RNA virus. Illustrative enveloped RNA viruses include viruses from the family of Bunyaviridae, Coronaviridae, Filoviridae, Flaviviridae, Orthomyxoviridae, Paramyxoviridae, Rhabdoviridae, and Togaviridae. In additional instances, the viral envelope or component thereof is derived from a virus from the family of Retroviridae.

In some embodiments, the viral envelope or component thereof is from an oncolytic virus, such as an oncolytic DNA virus from the family of Herpesviridae (for example, HSV1) or Poxviridae (for example, Vaccinia virus and myxoma virus); or an oncolytic RNA virus from the family of Rhabdoviridae (for example, VSV) or Paramyxoviridae (for example MV and NDV).

A component from a virus can be used for pseudotyping a capsid disclosed herein. Env is a retroviral gene that encodes the protein that forms the viral envelope. The expression of the env gene allows retroviruses to target and attach to specific cell types, and to infiltrate the target cell membrane. In some embodiments, a delivery component comprises a protein encoded by a retroviral Env gene.

In some instances, the delivery component comprises a fusogenic protein, for example, a FAST protein or an engineered variant thereof.

In some instances, the delivery component, viral envelope, or component thereof further comprises a foreign or engineered protein that binds to an antigen or a cell surface molecule. Illustrative antigens and cell surface molecules for targeting include, but are not limited to, P-glycoprotein, Her2/Neu, erythropoietin (EPO), epidermal growth factor receptor (EGFR), vascular endothelial growth factor receptor (VEGF-R), cadherin, carcinoembryonic antigen (CEA), CD4. CD8, CD19. CD20, CD33, CD34, CD45, CD117 (c-kit), CD133, HLA-A. HLA-B, HLA-C, chemokine receptor 5 (CCRS), stem cell marker ABCG2 transporter, ovarian cancer antigen CA125, immunoglobulins, integrins, prostate specific antigen (PSA), prostate stem cell antigen (PSCA), dendritic cell-specific intercellular adhesion molecule 3-grabbing nonintegrin (DC-SIGN), thyroglobulin, granulocyte-macrophage colony stimulating factor (GM-CSF), myogenic differentiation promoting factor-1 (MyoD-1), Leu-7 (CD57), LeuM-1, cell proliferation-associated human nuclear antigen defined by the monoclonal antibody Ki-67 (Ki-67), viral envelope proteins, HIV gp120, or transferrin receptor.

### III. CARGOS

In some embodiments, a composition or capsid disclosed herein comprises a cargo. In some embodiments, the cargo is a heterologous cargo that is not native to the endo Gag or PNMA polypeptide (e.g., is not native to the PNMA2 or PNMA5 polypeptide). In some embodiments, the cargo is a therapeutic agent. In some embodiments, the cargo is a nucleic acid molecule, a small molecule, a protein, a peptide, an antibody or binding fragment thereof, a peptidomimetic, or a nucleotidomimetic. In some instances, the cargo is a therapeutic cargo, comprising e.g., one or more drugs. In some instances, the cargo comprises a diagnostic tool or component thereof, for profiling, e.g., one or more markers (such as markers associates with one or more disease phenotypes). In additional instances, the cargo comprises an imaging tool or component thereof.

In some embodiments, the cargo is a peptidomimetic. A peptidomimetic is a small protein-like polymer designed to mimic a peptide. In some instances, the peptidomimetic comprises D-peptides. In other instances, the peptidomimetic comprises L-peptides. Exemplary peptidomimetics include peptoids and β-peptides.

In some embodiments, the cargo is a nucleotidomimetic.

### A. Nucleic Acids

In some instances, the cargo is a nucleic acid molecule. Examples of nucleic acid molecules include DNA, RNA, and mixtures of DNA and RNA. In some embodiments, the nucleic acid molecule comprises a hybrid of DNA and RNA.

In some instances, the nucleic acid molecule is a DNA polymer. In some cases, the DNA is a single stranded DNA polymer. In other cases, the DNA is a double stranded DNA polymer. In additional cases, the DNA is a hybrid of single and double stranded DNA polymers.

In some embodiments, the nucleic acid molecule is an RNA polymer, e.g., a single stranded RNA polymer, a double stranded RNA polymer, or a hybrid of single and double stranded RNA polymers. In some instances, the RNA comprises and/or encodes an antisense oligoribonucleotide, a siRNA, an mRNA, a tRNA, an rRNA, a snRNA, a shRNA, microRNA, or a non-coding RNA.

In some embodiments, the nucleic acid molecule is an antisense oligonucleotide, optionally comprising DNA, RNA, or a hybrid of DNA and RNA. In some instances, the nucleic acid molecule comprises and/or encodes an mRNA molecule. In some embodiments, the nucleic acid molecule comprises and/or encodes an RNAi molecule. In some cases, the RNAi molecule is a microRNA (miRNA) molecule. In other cases, the RNAi molecule is a siRNA molecule. The miRNA and/or siRNA are optionally double-stranded or as a hairpin, and further optionally encapsulated as precursor molecules.

In some embodiments, the nucleic acid molecule is for use in a nucleic acid-based therapy. In some instances, the nucleic acid molecule is for regulating gene expression (e.g., modulating mRNA translation or degradation), modulating RNA splicing, or RNA interference. In some cases, the nucleic acid molecule comprises and/or encodes an antisense oligonucleotide, microRNA molecule, siRNA molecule, mRNA molecule, for use in regulation of gene expression, modulating RNA splicing, or RNA interference.

In some instances, the nucleic acid molecule is for use in gene editing. Exemplary gene editing systems include, but are not limited to, CRISPR-Cas systems, zinc finger nuclease (ZFN) systems, and transcription activator-like effector nuclease (TALEN) systems. In some cases, the nucleic acid molecule comprises and/or encodes a component involved in the CRISPR-Cas systems, the ZFN systems, or the TALEN systems.

In some cases, the nucleic acid molecule is for use in antigen production for therapeutic and/or prophylactic vaccine production. For example, the nucleic acid molecule encodes an antigen that is expressed and elicits a desirable immune response (e.g., a pro-inflammatory immune response, an anti-inflammatory immune response, a tolerogenic immune response, an B cell response, an antibody response, a T cell response, a CD4+ T cell response, a CD8+ T cell response, a Th1 immune response, a Th2 immune response, a Th17 immune response, a Treg immune response, an M1 macrophage response, an M2 macrophage response, or a combination thereof).

In some cases, the nucleic acid molecule comprises a nucleic acid enzyme. Nucleic acid enzymes are RNA molecules (e.g., ribozymes) or DNA molecules (e.g., deoxyribozymes) that have catalytic activities. In some instances, the nucleic acid molecule is a ribozyme. In other instances, the nucleic acid molecule is a deoxyribozyme. In some cases, the nucleic acid molecule is a MNAzyme, which functions as a biosensor and/or a molecular switch (see, e.g., Mokany, et al., (2010) MNAzymes, a versatile new class of nucleic acid enzymes that can function as biosensors and molecular switches, JACS 132 (2): 1051-1059).

In some instances, exemplary targets of the nucleic acid molecule include, but are not limited to, UL123 (human cytomegalovirus), APOB, AR (androgen receptor) gene, KRAS, PCSK9, CFTR, and SMN (e.g., SMN2).

In some embodiments, the nucleic acid molecule is at least 5 nucleotides or more in length. In some instances, the nucleic acid molecule is at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 35, at least 40, at least 50, at least 60, at least 70, at least 80, at least 90, at least 100, at least 150, at least 200, at least 250, at least 300, at least 400, at least 500, at least 1000, at least 1500, at least 2000, at least 3000, at least 4000, at least 5000, at least 6000, at least 7000, at least 8000, or at least 9000 nucleotides or more in length. In some instances, the nucleic acid molecule is at least 10 nucleotides or more in length. In some instances, the nucleic acid molecule is at least 15 nucleotides or more in length. In some instances, the nucleic acid molecule is at least 20 nucleotides or more in length. In some instances, the nucleic acid molecule is at least 30 nucleotides or more in length. In some instances, the nucleic acid molecule is at least 40 nucleotides or more in length. In some instances, the nucleic acid molecule is at least 50 nucleotides or more in length. In some instances, the nucleic acid molecule is at least 100 nucleotides or more in length. In some instances, the nucleic acid molecule is at least 200 nucleotides or more in length. In some instances, the nucleic acid molecule is at least 300 nucleotides or more in length. In some instances, the nucleic acid molecule is at least 500 nucleotides or more in length. In some instances, the nucleic acid molecule is at least 1000 nucleotides or more in length. In some instances, the nucleic acid molecule is at least 2000 nucleotides or more in length. In some instances, the nucleic acid molecule is at least 3000 nucleotides or more in length. In some instances, the nucleic acid molecule is at least 4000 nucleotides or more in length. In some instances, the nucleic acid molecule is at least 5000 nucleotides or more in length. In some instances, the nucleic acid molecule is at least 8000 nucleotides or more in length.

In some embodiments, the nucleic acid molecule is at most 10,000 nucleotides or less in length. In some instances, the nucleic acid molecule is at most 15, at most 20, at most 25, at most 30, at most 35, at most 40, at most 50, at most 60, at most 70, at most 80, at most 90, at most 100, at most 150, at most 200, at most 250, at most 300, at most 400, at most 500, at most 1000, at most 1500, at most 2000, at most 3000, at most 4000, at most 5000, at most 6000, at most 7000, at most 8000, at most 9000, or at most 10,000 nucleotides or less in length. In some instances, the nucleic acid molecule is at most 15 nucleotides or less in length. In some instances, the nucleic acid molecule is at most 20 nucleotides or less in length. In some instances, the nucleic acid molecule is at most 25 nucleotides or less in length. In some instances, the nucleic acid molecule is at most 30 nucleotides or less in length. In some instances, the nucleic acid molecule is at most 40 nucleotides or less in length. In some instances, the nucleic acid molecule is at most 50 nucleotides or less in length. In some instances, the nucleic acid molecule is at most 100 nucleotides or less in length. In some instances, the nucleic acid molecule is at most 200 nucleotides or less in length. In some instances, the nucleic acid molecule is at most 300 nucleotides or less in length. In some instances, the nucleic acid molecule is at most 500 nucleotides or less in length. In some instances, the nucleic acid molecule is at most 1000 nucleotides or less in length. In some instances, the nucleic acid molecule is at most 2000 nucleotides or less in length. In some instances, the nucleic acid molecule is at most 3000 nucleotides or less in length. In some instances, the nucleic acid molecule is at most 4000 nucleotides or less in length. In some instances, the nucleic acid molecule is at most 5000 nucleotides or less in length. In some instances, the nucleic acid molecule is at most 8000 nucleotides or less in length. In some instances, the nucleic acid molecule is at most 9000 nucleotides or less in length.

In some embodiments, the nucleic acid molecule is about 100 nucleotides in length. In some instances, the nucleic acid molecule is about 6, about 7, about 8, about 9, about 10, about 15, about 20, about 25, about 30, about 35, about 40, about 50, about 60, about 70, about 80, about 90, about 100, about 150, about 200, about 250, about 300, about 400, about 500, about 1000, about 1500, about 2000, about 3000, about 4000, about 5000, about 6000, about 7000, about 8000, about 9000, or about 10000 nucleotides in length. In some instances, the nucleic acid molecule is about 10 nucleotides in length. In some instances, the nucleic acid molecule is about 15 nucleotides in length. In some instances, the nucleic acid molecule is about 20 nucleotides in length. In some instances, the nucleic acid molecule is about 25 nucleotides in length. In some instances, the nucleic acid molecule is about 30 nucleotides in length. In some instances, the nucleic acid molecule is about 40 nucleotides in length. In some instances, the nucleic acid molecule is about 50 nucleotides in length. In some instances, the nucleic acid molecule is about 100 nucleotides in length. In some instances, the nucleic acid molecule is about 150 nucleotides in length. In some instances, the nucleic acid molecule is about 200 nucleotides in length. In some instances, the nucleic acid molecule is about 300 nucleotides in length. In some instances, the nucleic acid molecule is about 500 nucleotides in length. In some instances, the nucleic acid molecule is about 1000 nucleotides in length. In some instances, the nucleic acid molecule is about 2000 nucleotides in length. In some instances, the nucleic acid molecule is about 3000 nucleotides in length. In some instances, the nucleic acid molecule is about 4000 nucleotides in length. In some instances, the nucleic acid molecule is about 5000 nucleotides in length. In some instances, the nucleic acid molecule is about 8000 nucleotides in length. In some instances, the nucleic acid molecule is about 9000 nucleotides in length.

In some embodiments, the nucleic acid molecule is from about 5 to about 10,000 nucleotides in length. In some instances, the nucleic acid molecule is from about 5 to about 9000 nucleotides in length, from about 5 to about 8000 nucleotides in length, from about 5 to about 7000 nucleotides in length, from about 5 to about 6000 nucleotides in length, from about 5 to about 5000 nucleotides in length, from about 5 to about 4000 nucleotides in length, from about 5 to about 3000 nucleotides in length, from about 5 to about 2000 nucleotides in length, from about 5 to about 1000 nucleotides in length, from about 5 to about 500 nucleotides in length, from about 5 to about 100 nucleotides in length, from about 5 to about 50 nucleotides in length, from about 5 to about 40 nucleotides in length, from about 5 to about 30 nucleotides in length, from about 5 to about 25 nucleotides in length, from about 5 to about 20 nucleotides in length, from about 10 to about 10,000 nucleotides in length. In some instances, the nucleic acid molecule is from about 10 to about 9000 nucleotides in length, from about 10 to about 8000 nucleotides in length, from about 10 to about 7000 nucleotides in length, from about 10 to about 6000 nucleotides in length, from about 10 to about 5000 nucleotides in length, from about 10 to about 4000 nucleotides in length, from about 10 to about 3000 nucleotides in length, from about 10 to about 2000 nucleotides in length, from about 10 to about 1000 nucleotides in length, from about 10 to about 500 nucleotides in length, from about 10 to about 100 nucleotides in length, from about 10 to about 50 nucleotides in length, from about 10 to about 40 nucleotides in length, from about 10 to about 30 nucleotides in length, from about 10 to about 25 nucleotides in length, from about 10 to about 20 nucleotides in length, from about 50 to about 10,000 nucleotides in length. In some instances, the nucleic acid molecule is from about 50 to about 9000 nucleotides in length, from about 50 to about 8000 nucleotides in length, from about 50 to about 7000 nucleotides in length, from about 50 to about 6000 nucleotides in length, from about 50 to about 5000 nucleotides in length, from about 50 to about 4000 nucleotides in length, from about 50 to about 3000 nucleotides in length, from about 50 to about 2000 nucleotides in length, from about 50 to about 1000 nucleotides in length, from about 50 to about 500 nucleotides in length, from about 50 to about 100 nucleotides in length, from about 50 to about 50 nucleotides in length, from about 50 to about 40 nucleotides in length, from about 50 to about 30 nucleotides in length, from about 50 to about 25 nucleotides in length, from about 50 to about 20 nucleotides in length, from about 100 to about 10,000 nucleotides in length. In some instances, the nucleic acid molecule is from about 100 to about 9000 nucleotides in length, from about 100 to about 8000 nucleotides in length, from about 100 to about 7000 nucleotides in length, from about 100 to about 6000 nucleotides in length, from about 100 to about 5000 nucleotides in length, from about 100 to about 4000 nucleotides in length, from about 100 to about 3000 nucleotides in length, from about 100 to about 2000 nucleotides in length, from about 100 to about 1000 nucleotides in length, from about 100 to about 500 nucleotides in length, from about 100 to about 100 nucleotides in length, from about 100 to about 50 nucleotides in length, from about 100 to about 40 nucleotides in length, from about 100 to about 30 nucleotides in length, from about 100 to about 25 nucleotides in length, from about 100 to about 20 nucleotides in length, from about 500 to about 10,000 nucleotides in length. In some instances, the nucleic acid molecule is from about 500 to about 9000 nucleotides in length, from about 500 to about 8000 nucleotides in length, from about 500 to about 7000 nucleotides in length, from about 500 to about 6000 nucleotides in length, from about 500 to about 5000 nucleotides in length, from about 500 to about 4000 nucleotides in length, from about 500 to about 3000 nucleotides in length, from about 500 to about 2000 nucleotides in length, from about 500 to about 1000 nucleotides in length, from about 500 to about 500 nucleotides in length, from about 500 to about 100 nucleotides in length, from about 500 to about 50 nucleotides in length, from about 500 to about 40 nucleotides in length, from about 500 to about 30 nucleotides in length, from about 500 to about 25 nucleotides in length, or from about 500 to about 20 nucleotides in length.

In some embodiments, the nucleic acid molecule comprises natural, synthetic, or artificial nucleotide analogues or bases. In some cases, the nucleic acid molecule comprises combinations of DNA, RNA and/or nucleotide analogues. In some instances, the synthetic or artificial nucleotide analogues or bases comprise modifications at one or more of ribose moiety, phosphate moiety, nucleoside moiety, or a combination thereof.

In some embodiments, a nucleotide analogue or artificial nucleotide base described above comprises a nucleic acid with a modification at a 2′ hydroxyl group of the ribose moiety. In some instances, the modification includes an H, OR, R, halo, SH, SR, NH2, NHR, NR2, or CN, wherein R is an alkyl moiety. Exemplary alkyl moiety includes, but is not limited to, halogens, sulfurs, thiols, thioethers, thioesters, amines (primary, secondary, or tertiary), amides, ethers, esters, alcohols and oxygen. In some instances, the alkyl moiety further comprises a modification. In some instances, the modification comprises an azo group, a keto group, an aldehyde group, a carboxyl group, a nitro group, a nitroso, group, a nitrile group, a heterocycle (e.g., imidazole, hydrazino or hydroxylamino) group, an isocyanate or cyanate group, or a sulfur containing group (e.g., sulfoxide, sulfone, sulfide, or disulfide). In some instances, the alkyl moiety further comprises a hetero substitution. In some instances, the carbon of the heterocyclic group is substituted by a nitrogen, oxygen or sulfur. In some instances, the heterocyclic substitution includes but is not limited to, morpholino, imidazole, and pyrrolidino.

In some instances, the modification at the 2′ hydroxyl group is a 2′-O-methyl modification or a 2′-O-methoxyethyl(2′-O-MOE) modification. In some cases, the 2′-O-methyl modification adds a methyl group to the 2′ hydroxyl group of the ribose moiety whereas the 2′ O-methoxyethyl modification adds a methoxyethyl group to the 2′ hydroxyl group of the ribose moiety.

In some instances, the modification at the 2′ hydroxyl group is a 2′-O-aminopropyl modification in which an extended amine group comprising a propyl linker binds the amine group to the 2′ oxygen. In some instances, this modification neutralizes the phosphate-derived overall negative charge of the oligonucleotide molecule by introducing one positive charge from the amine group per sugar and thereby improves cellular uptake properties due to its zwitterionic properties.

In some instances, the modification at the 2′ hydroxyl group is a locked or bridged ribose modification (e.g., locked nucleic acid or LNA) in which the oxygen molecule bound at the 2′ carbon is linked to the 4′ carbon by a methylene group, thus forming a 2′-C,4′-C-oxy-methylene-linked bicyclic ribonucleotide monomer.

In some embodiments, additional modifications at the 2′ hydroxyl group include 2′-deoxy, T-deoxy-2′-fluoro, 2′-O-aminopropyl(2′-O-AP), 2′-O-dimethylaminoethyl(2′-O-DMAOE), 2′-O-dimethylaminopropyl(2′-O-DMAP), T-O-dimethylaminoethyloxyethyl(2′-O-DMAEOE), or 2′-O—N-methylacetamido (2′-O-NMA).

In some embodiments, a nucleotide analogue comprises a modified base such as, but not limited to, N1-methylpseudouridine, 5-propynyluridine, 5-propynylcytidine, 6-methyladenine, 6-methylguanine, N, N,-dimethyladenine, 2-propyladenine, 2propylguanine, 2-aminoadenine, 1-methylinosine, 3-methyluridine, 5-methylcytidine, 5-methyluridine and other nucleotides having a modification at the 5 position, 5-(2-amino) propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides (such as 7-deaza-adenosine, 6-azouridine, 6-azocytidine, or 6-azothymidine), 5-methyl-2-thiouridine, other thio bases (such as 2-thiouridine, 4-thiouridine, and 2-thiocytidine), dihydrouridine, pseudouridine, queuosine, archaeosine, naphthyl and substituted naphthyl groups, any O-and N-alkylated purines and pyrimidines (such as N6-methyladenosine, 5-methylcarbonylmethyluridine, uridine 5-oxyacetic acid, pyridine-4-one, or pyridine-2-one), phenyl and modified phenyl groups such as aminophenol or 2,4, 6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyi nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties, in some cases are or are based on, mannoses, arabinoses, glucopyranoses, galactopyranoses, 4′-thioribose, and other sugars, heterocycles, or carbocycles. The term nucleotide also includes universal bases. By way of example, universal bases include but are not limited to 3-nitropyrrole, 5-nitroindole, or nebularine.

In some embodiments, a nucleotide analogue further comprises a morpholino, a peptide nucleic acid (PNA), a methylphosphonate nucleotide, a thiolphosphonate nucleotide, a 2′-fluoro N3-P5′-phosphoramidite, or a 1′, 5′-anhydrohexitol nucleic acid (HNA). Morpholino or phosphorodiamidate morpholino oligo (PMO) comprises synthetic molecules whose structure mimics natural nucleic acid structure but deviates from the normal sugar and phosphate structures. In some instances, the five-member ribose ring is substituted with a six-member morpholino ring containing four carbons, one nitrogen, and one oxygen. In some cases, the ribose monomers are linked by a phosphordiamidate group instead of a phosphate group. In such cases, the backbone alterations remove all positive and negative charges making morpholinos neutral molecules capable of crossing cellular membranes without the aid of cellular delivery agents such as those used by charged oligonucleotides.

In some embodiments, peptide nucleic acid (PNA) does not contain sugar ring or phosphate linkage and the bases are attached and appropriately spaced by oligoglycine-like molecules, therefore, eliminating a backbone charge.

In some embodiments, one or more modifications optionally occur at the internucleotide linkage. In some instances, modified internucleotide linkage includes, but is not limited to, phosphorothioates; phosphorodithioates; methylphosphonates; 5′-alkylenephosphonates; 5′-methylphosphonate; 3′-alkylene phosphonates; borontrifluoridates; borano phosphate esters and selenophosphates of 3′-5′ linkage or 2′-5′ linkage; phosphotriesters; thionoalkylphosphotriesters; hydrogen phosphonate linkages; alkyl phosphonates; alkylphosphonothioates; arylphosphonothioates; phosphoroselenoates; phosphorodiselenoates; phosphinates; phosphoramidates; 3′-alkylphosphoramidates; aminoalkylphosphoramidates; thionophosphoramidates; phosphoropiperazidates; phosphoroanilothioates; phosphoroanilidates; ketones; sulfones; sulfonamides; carbonates; carbamates; methylenehydrazos; methylenedimethylhydrazos; formacetals; thioformacetals; oximes; methyleneiminos; methylenemethyliminos; thioamidates; linkages with riboacetyl groups; aminoethyl glycine; silyl or siloxane linkages; alkyl or cycloalkyl linkages with or without heteroatoms of, for example, 1 to 10 carbons that are saturated or unsaturated and/or substituted and/or contain heteroatoms; linkages with morpholino structures, amides, or polyamides wherein the bases are attached to the aza nitrogens of the backbone directly or indirectly; and combinations thereof.

In some embodiments, one or more modifications comprise a modified phosphate backbone in which the modification generates a neutral or uncharged backbone. In some instances, the phosphate backbone is modified by alkylation to generate an uncharged or neutral phosphate backbone. As used herein, alkylation includes methylation, ethylation, and propylation. In some cases, an alkyl group, as used herein in the context of alkylation, refers to a linear or branched saturated hydrocarbon group containing from 1 to 6 carbon atoms. In some instances, exemplary alkyl groups include, but are not limited to, methyl, ethyl, n-propyl, isopropyl, n-butyl, isobutyl, sec-butyl, tert-butyl, n-pentyl, isopentyl, neopentyl, hexyl, isohexyl, 1, 1-dimethylbutyl, 2,2-dimethylbutyl, 3.3-dimethylbutyl, and 2-ethylbutyl groups. In some cases, a modified phosphate is a phosphate group as described in U.S. Pat. No. 9,481,905.

In some embodiments, additional modified phosphate backbones comprise methylphosphonate, ethylphosphonate, methylthiophosphonate, or methoxyphosphonate. In some cases, the modified phosphate is methylphosphonate. In some cases, the modified phosphate is ethylphosphonate. In some cases, the modified phosphate is methylthiophosphonate. In some cases, the modified phosphate is methoxyphosphonate.

In some embodiments, one or more modifications further optionally include modifications of the ribose moiety, phosphate backbone and the nucleoside, or modifications of the nucleotide analogues at the 3′ or the 5′ terminus. For example, the 3′ terminus optionally include a 3′ cationic group, or by inverting the nucleoside at the 3′-terminus with a 3′-3′ linkage. In another alternative, the 3′-terminus is optionally conjugated with an aminoalkyl group, e.g., a 3′ C5-aminoalkyl dT. In an additional alternative, the 3′-terminus is optionally conjugated with an abasic site, e.g., with an apurinic or apyrimidinic site. In some instances, the 5′-terminus is conjugated with an aminoalkyl group, e.g., a 5′-O-alkylamino substituent. In some cases, the 5′-terminus is conjugated with an abasic site, e.g., with an apurinic or apyrimidinic site.

In some embodiments, exemplary nucleic acid cargos include, but are not limited to, Fomivirsen, Mipomersen, AZD5312 (AstraZeneca), Nusinersen, and SB010 (Sterna Biologicals).

In some embodiments, a nucleic acid cargo comprises a recruitment domain. For example, an nucleic acid sequence (e.g., RNA) can comprise a secondary structure to bind to a cargo binding domain (e.g., an endogenous or engineered cargo binding domain) of a PNMA family polypeptide disclosed herein. The recruitment domain can be or comprise, for example, a hairpin loop element. The recruitment domain can be or comprise, for example, an aptamer sequence. The recruitment domain can be or comprise, for example, two or more aptamer sequences specific to the same or different cargo binding domains. In some embodiments, the recruitment domain comprises an aptamer that binds to MS2, PP7, Qβ, F2, GA, fr, JP501, M12, R17, BZ13, JP34, JP500, KU1, M11, MX1, TW18, VK, SP, FI, ID2, NL95, TW19, AP205, φCb5, φCb8r, φCb12r, φCb23r, 7s, or PRR1.

### B. Gene Editing Systems

In some embodiments, the cargo comprises or encodes a gene editing system or a component thereof. Non-limiting examples of gene editing tools and techniques include CRISPR, TALEN, zinc finger nuclease (ZFN), meganuclease, Mega-TAL, and transposon-based systems.

In some embodiments, the cargo comprises or encodes a CRISPR-associated polypeptide (Cas), zinc finger nuclease (ZFN), zinc finger associate gene regulation polypeptide, transcription activator-like effector nuclease (TALEN), transcription activator-like effector associated gene regulation polypeptides, meganuclease, natural master transcription factors, epigenetic modifying enzymes, recombinase, flippase, transposase, RNA-binding proteins (RBP), an Argonaute protein, any derivative thereof, any variant thereof, or any fragment thereof.

In some embodiments, the cargo comprises or encodes a CRISPR system or a component thereof. A CRISPR system can be utilized to facilitate insertion of a recombinant nucleic acid encoding a regulatable membrane protein or a component thereof into a cell genome. For example, a CRISPR system can introduce a double stranded break at a target site in a genome or a random site of a genome.

In some embodiments, the cargo comprises or encodes a Cas protein. Non-limiting examples of Cas proteins that can be used in the CRISPR systems include Cas1, Cas1B, Cas2, Cas3, Cas4, Cas5, Cas6, Cas7, Cas8, Cas9 (also known as Csn1 or Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Cpf1, c2c1, c2c3, Cas9HiFi, homologues thereof, and modified versions thereof. An unmodified CRISPR enzyme can have DNA cleavage activity, such as Cas9. A CRISPR enzyme can direct cleavage of one or both strands at a target sequence, such as within a target sequence and/or within a complement of a target sequence. For example, a CRISPR enzyme can direct cleavage of one or both strands within or within about 1, 2, 3, 4, 5, 6, 7, 8, 9, 10, 15, 20, 25, 50, 100, 200, 500, or more base pairs from the first or last nucleotide of a target sequence. A Cas protein can be a high fidelity Cas protein. Alternatives to S. pyogenes Cas9 may include RNA-guided endonucleases from the Cpf1 family that display cleavage activity in mammalian cells.

In some embodiments, the cargo comprises or encodes a guide RNA (gRNA), e.g., that complexes with a Cas protein.

In some embodiments, the cargo comprises or encodes a nucleotide sequence to be inserted in to a genome. In some embodiments, the cargo comprises or encodes a repair template, e.g., for homology-directed repair.

In some embodiments, the cargo comprises or encodes a dual nickase CRISPR system or a component thereof. A dual nickase approach may be used to introduce a double stranded break. Cas proteins can be mutated at certain amino acids within either nuclease domains, thereby deleting activity of one nuclease domain and generating a nickase Cas protein capable of generating a single strand break. A nickase along with two distinct guide RNAs targeting opposite strands may be utilized to generate a DSB within a target site (often referred to as a “double nick” or “dual nickase” CRISPR system).

In some embodiments, the cargo comprises or encodes a transposon based system or a component thereof. A transposon-based system can be utilized for insertion of a recombinant nucleic acid encoding a regulatable membrane protein of the disclosure or a component thereof into a genome. A transposon can comprise a recombinant nucleic acid that can be inserted into a DNA sequence. A class I transposon can be transcribed into an RNA intermediate, then reverse transcribed and inserted into a DNA sequence. A class II transposon can comprise a DNA sequence that is excised from one DNA sequence and/or inserted into another DNA sequence. A class II transposon system can comprise (i) a transposon vector that contains a sequence (e.g., comprising a transgene) flanked by inverted terminal repeats, and (ii) a source for the transposase enzyme.

In some embodiments, the cargo comprises or encodes a TALEN system or a component thereof. TALENs can refer to engineered transcription activator-like effector nucleases that generally contain a central domain of DNA-binding tandem repeats and a cleavage domain. TALENs can be produced by fusing a TAL effector DNA binding domain to a DNA cleavage domain. In some cases, a DNA-binding tandem repeat comprises 33-35 amino acids in length and contains two hypervariable amino acid residues at positions 12 and 13 that can recognize at least one specific DNA base pair. A transcription activator-like effector (TALE) protein can be fused to a nuclease such as a wild-type or mutated Fok1 endonuclease or the catalytic domain of Fok1.

In some embodiments, the cargo comprises or encodes a zinc finger nuclease (ZFN) or a variant, fragment, or derivative thereof. ZFN can refer to a fusion between a cleavage domain, such as a cleavage domain of Fokl, and at least one zinc finger motif (e.g., at least 2, at least 3, at least 4, or at least 5 zinc finger motifs) which can bind polynucleotides such as DNA and RNA.

In some embodiments, the cargo comprises or encodes a meganuclease. Meganucleases generally refer to rare-cutting endonucleases or homing endonucleases that can be highly sequence specific. Meganucleases can recognize DNA target sites ranging from at least 12 base pairs in length, e.g., from 12 to 40 base pairs, 12 to 50 base pairs, or 12 to 60 base pairs in length. Meganucleases can be modular DNA-binding nucleases such as any fusion protein comprising at least one catalytic domain of an endonuclease and at least one DNA binding domain or protein specifying a nucleic acid target sequence. The DNA-binding domain can contain at least one motif that recognizes single- or double-stranded DNA. A nuclease-active meganuclease can generate a double-stranded break. The meganuclease can be monomeric or dimeric. In some embodiments, the meganuclease is naturally-occurring (found in nature) or wild-type, and in other instances, the meganuclease is non-natural, artificial, engineered, synthetic, rationally designed, or man-made. In some embodiments, the meganuclease of the present disclosure includes an I-CreI meganuclease, I-CeuI meganuclease, I-Msol meganuclease, I-SceI meganuclease, variants thereof, derivatives thereof, and fragments thereof.

### C. Small Molecules

In some embodiments, the cargo is a small molecule. In some instances, the small molecule is an inhibitor (e.g., a pan inhibitor or a selective inhibitor). In other instances, the small molecule is an activator. In additional cases, the small molecule is an agonist, antagonist, a partial agonist, a mixed agonist/antagonist, or a competitive antagonist.

In some embodiments, the small molecule is a drug that falls under the class of analgesics, antianxiety drugs, antiarrhythmics, antibacterials, antibiotics, anticoagulants and thrombolytics, anticonvulsants, antidepressants, antidiarrheals, antiemetics, antifungals, antihistamines, antihypertensives, anti-inflammatories, antineoplastics, antipsychotics, antipyretics, antivirals, barbiturates, beta-blockers, bronchodilators, common cold treatments, corticosteroids, cough suppressants, cytotoxics, decongestants, diuretics, expectorant, hormones, hypoglycemics, immunosuppressives, laxatives, muscle relaxants, sex hormones, sleeping drugs, or tranquilizers.

In some embodiments, the small molecule is an inhibitor, e.g., an inhibitor of a kinase pathway such as the Tyrosine kinase pathway or a Serine/Threonine kinase pathway. In some cases, the small molecule is a dual protein kinase inhibitor. In some cases, the small molecule is a lipid kinase inhibitor.

In some cases, the small molecule is a neuraminidase inhibitor.

In some cases, the small molecule is a carbonic anhydrase inhibitor.

In some embodiments, exemplary targets of the small molecule include, but are not limited to, vascular endothelial growth factor receptor 1 (VEGFR1), vascular endothelial growth factor receptor 2 (VEGFR2), vascular endothelial growth factor receptor 3 (VEGFR3), fibroblast growth factor receptor 1 (FGFR1), fibroblast growth factor receptor 2 (FGFR2), fibroblast growth factor receptor 3 (FGFR3), fibroblast growth factor receptor 4 (FGFR4), cyclin-dependent kinase 4 (CDK4), cyclin-dependent kinase 6 (CDK6), a receptor tyrosine kinase, a phosphoinositide 3-kinase (PI3K) isoform (e.g., PI3Kδ, also known as p110δ), Janus kinase 1 (JAK1), Janus kinase 3 (JAK3), a receptor from the family of platelet-derived growth factor receptors (PDFG-R), and carbonic anhydrase (e.g., carbonic anhydrase I).

In some embodiments, the small molecule targets a viral protein, e.g., a viral envelope protein. In some embodiments, the small molecule decreases viral adsorption to a host cell. In some embodiments, the small molecule decreases viral entry into a host cell. In some embodiments, the small molecule decreases viral replication in a host or a host cell. In some embodiments, the small molecule decreases viral assembly.

In some embodiments, exemplary small molecule cargos include, but are not limited to, lenvatinib, palbociclib, regorafenib, idelalisib, tofacitinib, nintedanib, zanamivir, ethoxzolamide, and artemisinin.

### D. Proteins and Peptides

In some embodiments, the cargo is a protein. In some instances, the protein is a full-length protein. In other instances, the protein is a fragment, e.g., a functional fragment. In some cases, the protein is a naturally occurring protein. In additional cases, the protein is a de novo engineered protein. In further cases, the protein is a fusion protein. In further cases, the protein is a recombinant protein. Exemplary proteins include, but are not limited to, Fc fusion proteins, anticoagulants, blood factors, bone morphogenetic proteins, enzymes, growth factors, hormones, interferons, interleukins, and thrombolytics.

In some instances, the protein is for use in an enzyme replacement therapy.

In some cases, the protein is for use in antigen production for therapeutic and/or prophylactic vaccine production. For example, the protein comprises an antigen that elicits a desirable immune response (e.g., a pro-inflammatory immune response, an anti-inflammatory immune response, a tolerogenic immune response, an B cell response, an antibody response, a T cell response, a CD4+ T cell response, a CD8+ T cell response, a Th1 immune response, a Th2 immune response, a Th17 immune response, a Treg immune response, an M1 macrophage response, an M2 macrophage response, or a combination thereof).

In some instances, exemplary protein cargos include, but are not limited to, romiplostim, liraglutide, a human growth hormone (rHGH), human insulin (BHI), follicle-stimulating hormone (FSH), Factor VIII, erythropoietin (EPO), granulocyte colony-stimulating factor (G-CSF), alpha-galactosidase A, alpha-L-iduronidase, N-acetylgalactosamine-4-sulfatase, dornase alfa, tissue plasminogen activator (TPA), glucocerebrosidase, interferon-beta-1a, insulin-like growth factor 1 (IGF-1), or rasburicase.

In some embodiments, the cargo is a peptide. In some instances, the peptide is a naturally occurring peptide. In other instances, the peptide is an artificial engineered peptide or a recombinant peptide. In some cases, the peptide targets a G-protein coupled receptor, an ion channel, a microbe, an anti-microbial target, a catalytic or other Ig-family of receptors, an intracellular target, a membrane-anchored target, or an extracellular target.

In some cases, the peptide comprises at least 2 amino acids. In some cases, the peptide comprises at least 3, at least 4, at least 5, at least 6, at least 7, at least 8, at least 9, at least 10, at least 15, at least 20, at least 25, at least 30, at least 35, at least 40, at least 45, at least 50, at least 60, at least 70, at least 80, at least 90, or at least 100 amino acids. In some cases, the peptide comprises at least 10 amino acids. In some cases, the peptide comprises at least 15 amino acids. In some cases, the peptide comprises at least 20 amino acids. In some cases, the peptide comprises at least 30 amino acids. In some cases, the peptide comprises at least 40 amino acids. In some cases, the peptide comprises at least 50 amino acids. In some cases, the peptide comprises at least 60 amino acids. In some cases, the peptide comprises at least 70 amino acids. In some cases, the peptide comprises at least 80 amino acids. In some cases, the peptide comprises at least 90 amino acids. In some cases, the peptide comprises at least 100 amino acids.

In some cases, the peptide comprises at most 3 amino acids. In some cases, the peptide comprises at most 4, at most 5, at most 6, at most 7, at most 8, at most 9, at most 10, at most 15, at most 20, at most 25, at most 30, at most 35, at most 40, at most 45, at most 50, at most 60, at most 70, at most 80, at most 90, or at most 100 amino acids. In some cases, the peptide comprises at most 10 amino acids. In some cases, the peptide comprises at most 15 amino acids. In some cases, the peptide comprises at most 20 amino acids. In some cases, the peptide comprises at most 30 amino acids. In some cases, the peptide comprises at most 40 amino acids. In some cases, the peptide comprises at most 50 amino acids. In some cases, the peptide comprises at most 60 amino acids. In some cases, the peptide comprises at most 70 amino acids. In some cases, the peptide comprises at most 80 amino acids. In some cases, the peptide comprises at most 90 amino acids. In some cases, the peptide comprises at most 100 amino acids.

In some cases, the peptide comprises from about 1 to about 10 kDa. In some cases, the peptide comprises from about 1 to about 9 kDa, about 1 to about 6 kDa, about 1 to about 5 kDa, about 1 to about 4 kDa, about 1 to about 3 kDa, about 2 to about 8 kDa, about 2 to about 6 kDa, about 2 to about 4 kDa, about 1.2 to about 2.8 kDa, about 1.5 to about 2.5 kDa, or about 1.5 to about 2 kDa.

In some embodiments, the peptide is a cyclic peptide. In some instances, the cyclic peptide is a macrocyclic peptide. In other instances, the cyclic peptide is a constrained peptide. The cyclic peptides are assembled with varied linkages, such as for example, head-to-tail, head-to-side-chain, side-chain-to-tail, and side-chain-to-side-chain linkages. In some instances, a cyclic peptide (e.g., a macrocyclic or a constrained peptide) has a molecular weight from about 500 Dalton to about 2000 Dalton. In other instances, a cyclic peptide (e.g., a macrocyclic or a constrained peptide) ranges from about 10 amino acids to about 100 amino acids, from about 10 amino acids to about 70 amino acids, or from about 10 amino acids to about 50 amino acids.

In some cases, the peptide is for use in antigen production for therapeutic and/or prophylactic vaccine production. For example, the peptide comprises an antigen that elicits a desirable immune response (e.g., a pro-inflammatory immune response, an anti-inflammatory immune response, a tolerogenic immune response, an B cell response, an antibody response, a T cell response, a CD4+ T cell response, a CD8+ T cell response, a Th1 immune response, a Th2 immune response, a Th17 immune response, a Treg immune response, an M1 macrophage response, an M2 macrophage response, or a combination thereof).

In some embodiments, the peptide comprises natural amino acids, unnatural amino acids, or a combination thereof. In some instances, an amino acid residue refers to a molecule containing both an amino group and a carboxyl group. Suitable amino acids include, without limitation, both the D- and L-isomers of the naturally occurring amino acids, as well as non-naturally occurring amino acids prepared by organic synthesis or other metabolic routes. The term amino acid, as used herein, includes, without limitation, α-amino acids, natural amino acids, non-natural amino acids, and amino acid analogs.

In some instances, α-amino acid refers to a molecule containing both an amino group and a carboxyl group bound to a carbon which is designated the α-carbon.

In some instances, β-amino acid refers to a molecule containing both an amino group and a carboxyl group in a β configuration.

In some embodiments, an amino acid analog is a racemic mixture. In some instances, the D isomer of the amino acid analog is used. In some cases, the L isomer of the amino acid analog is used. In some instances, the amino acid analog comprises chiral centers that are in the R or S configuration.

In some embodiments, exemplary peptide cargos include, but are not limited to, peginesatide, insulin, adrenocorticotropic hormone (ACTH), calcitonin, oxytocin, vasopressin, octreolide, and leuprorelin.

In some embodiments, exemplary peptide cargos include, but are not limited to, Telavancin, Dalbavancin, Oritavancin, Anidulafungin, Lanreotide, Pasireotide, Romidepsin, Linaclotide, and Peginesatide.

### E. Antibodies

In some embodiments, the cargo is an antibody or a binding fragment thereof. In some instances, the antibody or binding fragment thereof comprises a humanized antibody or binding fragment thereof, murine antibody or binding fragment thereof, chimeric antibody or binding fragment thereof, monoclonal antibody or binding fragment thereof, bispecific antibody or biding fragment thereof, monovalent Fab′, divalent Fab2, F(ab)′3 fragments, single-chain variable fragment (scFv), bis-scFv, (scFv)2, diabody, minibody, nanobody, triabody, tetrabody, disulfide stabilized Fv protein (dsFv), single-domain antibody (sdAb), Ig NAR, camelid antibody or binding fragment thereof, or a chemically modified derivative thereof.

In some instances, the antibody or binding fragment thereof recognizes a cell surface protein. In some instances, the cell surface protein is an antigen expressed by a cancerous cell. In some instances, the cell surface protein is a neoepitope. In some instances, the cell surface protein comprises one or more mutations compared to a wild-type protein. Exemplary cancer antigens include, but are not limited to, alpha fetoprotein, ASLG659, B7-H3, BAFF-R, Brevican, CA125 (MUC16), CA15-3, CA19-9, carcinoembryonic antigen (CEA), CA242, CRIPTO (CR, CR1, CRGF, CRIPTO, TDGF1, teratocarcinoma-derived growth factor), CTLA-4, CXCR5, E16 (LAT1, SLC7A5), FcRH2 (IFGP4, IRTA4, SPAP1A (SH2 domain containing phosphatase anchor protein 1a), SPAP1B, SPAPIC), epidermal growth factor, ETBR, Fc receptor-like protein 1 (FCRH1), GEDA, HLA-DOB (Beta subunit of MHC class II molecule (Ia antigen), human chorionic gonadotropin, ICOS, IL-2 receptor, IL20Ra, Immunoglobulin superfamily receptor translocation associated 2 (IRTA2), L6, Lewis Y, Lewis X, MAGE-1, MAGE-2, MAGE-3, MAGE 4, MART1, mesothelin, MDP, MPF (SMR, MSLN), MCP1 (CCL2), macrophage inhibitory factor (MIF), MPG, MSG783, mucin, MUC1-KLH, Napi3b (SLC34A2), nectin-4, Neu oncogene product, NCA, placental alkaline phosphatase, prostate specific membrane antigen (PMSA), prostatic acid phosphatase, PSCA hlg, anti-transferrin receptor, p97, Purinergic receptor P2X ligand-gated ion channel 5 (P2X5), LY64 (Lymphocyte antigen 64 (RP105), gp100, P21, six transmembrane epithelial antigen of prostate (STEAP1), STEAP2, Sema 5b, tumor-associated glycoprotein 72 (TAG-72), TrpM4 (BR22450, FLJ20041, TRPM4, TRPM4B, transient receptor potential cation channel, subfamily M, member 4) and the like.

In some instances, the cell surface protein comprises clusters of differentiation (CD) cell surface markers. Exemplary CD cell surface markers include, but are not limited to, CD1, CD2, CD3, CD4, CD5, CD6, CD7, CD8, CD9, CD10, CD11a, CD11b, CD11c, CD11d, CDw12, CD13, CD14, CD15, CD15s, CD16, CDw17, CD18, CD19, CD20, CD21, CD22, CD23, CD24, CD25, CD26, CD27, CD28, CD29, CD30, CD31, CD32, CD33, CD34, CD35, CD36, CD37, CD38, CD39, CD40, CD41, CD42, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD61, CD62E, CD62L (L-selectin), CD62P, CD63, CD64, CD65, CD66a, CD66b, CD66c, CD66d, CD66e, CD71, CD79 (e.g., CD79a, CD79b), CD90, CD95 (Fas), CD103, CD104, CD125 (IL5RA), CD134 (OX40), CD137 (4-1BB), CD152 (CTLA-4), CD221, CD274, CD279 (PD-1), CD319 (SLAMF7), CD326 (EpCAM), and the like.

In some embodiments, exemplary antibodies or binding fragments thereof include, but are not limited to, zalutumumab (HuMax-EFGr, Genmab), abagovomab (Menarini), abituzumab (Merck), adecatumumab (MT201), alacizumab pegol, alemtuzumab (Campath®, MabCampath, or Campath-1H; Leukosite), AlloMune (BioTransplant), amatuximab (Morphotek, Inc.), anti-VEGF (Genetech), anatumomab mafenatox, apolizumab (hu1D10), ascrinvacumab (Pfizer Inc.), atezolizumab (MPDL3280A; Genentech/Roche), B43.13 (OvaRex, AltaRex Corporation), basiliximab (Simulect®, Novartis), belimumab (Benlysta®, GlaxoSmithKline), bevacizumab (Avastin®, Genentech), blinatumomab (Blincyto, AMG103; Amgen), BEC2 (ImGlone Systems Inc.), carlumab (Janssen Biotech), catumaxomab (Removab, Trion Pharma), CEAcide (Immunomedics), Cetuximab (Erbitux®, ImClone), citatuzumab bogatox (VB6-845), cixutumumab (IMC-A12, ImClone Systems Inc.), conatumumab (AMG 655, Amgen), dacetuzumab (SGN-40, huS2C6; Seattle Genetics, Inc.), daratumumab (Darzalex®, Janssen Biotech), detumomab, drozitumab (Genentech), durvalumab (MedImmune), dusigitumab (MedImmune), edrecolomab (MAb17-1A, Panorex, Glaxo Wellcome), elotuzumab (Empliciti™, Bristol-Myers Squibb), emibetuzumab (Eli Lilly), enavatuzumab (Facet Biotech Corp.), enfortumab vedotin (Seattle Genetics, Inc.), enoblituzumab (MGA271, MacroGenics, Inc.), ensituxumab (Neogenix Oncology, Inc.), epratuzumab (LymphoCide, Immunomedics, Inc.), ertumaxomab (Rexomun®, Trion Pharma), etaracizumab (Abegrin, MedImmune), farletuzumab (MORAb-003, Morphotek, Inc), FBTA05 (Lymphomun, Trion Pharma), ficlatuzumab (AVEO Pharmaceuticals), figitumumab (CP-751871, Pfizer), flanvotumab (ImClone Systems), fresolimumab (GC1008, Aanofi-Aventis), futuximab, glaximab, ganitumab (Amgen), girentuximab (Rencarex®, Wilex AG), IMAB362 (Claudiximab, Ganymed Pharmaceuticals AG), imalumab (Baxalta), IMC-1C11 (ImClone Systems), IMC-C225 (Imclone Systems Inc.), imgatuzumab (Genentech/Roche), intetumumab (Centocor, Inc.), ipilimumab (Yervoy®, Bristol-Myers Squibb), iratumumab (Medarex, Inc.), isatuximab (SAR650984, Sanofi-Aventis), labetuzumab (CEA-CIDE, Immunomedics), lexatumumab (ETR2-ST01, Cambridge Antibody Technology), lintuzumab (SGN-33, Seattle Genetics), lucatumumab (Novartis), lumiliximab, mapatumumab (HGS-ETR1, Human Genome Sciences), matuzumab (EMD 72000, Merck), milatuzumab (hLL1, Immunomedics, Inc.), mitumomab (BEC-2, ImClone Systems), narnatumab (ImClone Systems), necitumumab (Portrazza™, Eli Lilly), nesvacumab (Regeneron Pharmaceuticals), nimotuzumab (h-R3, BIOMAb EGFR, TheraCIM, Theraloc, or CIMAher; Biotech Pharmaceutical Co.), nivolumab (Opdivo®, Bristol-Myers Squibb), obinutuzumab (Gazyva or Gazyvaro; Hoffmann-La Roche), ocaratuzumab (AME-133v, LY2469298; Mentrik Biotech, LLC), ofatumumab (Arzerra®, Genmab), onartuzumab (Genentech), Ontuxizumab (Morphotek, Inc.), oregovomab (OvaRex®, AltaRex Corp.), otlertuzumab (Emergent BioSolutions), panitumumab (ABX-EGF, Amgen), pankomab (Glycotope GMBH), parsatuzumab (Genentech), patritumab, pembrolizumab (Keytruda®, Merck), pemtumomab (Theragyn, Antisoma), pertuzumab (Perjeta, Genentech), pidilizumab (CT-011, Medivation), polatuzumab vedotin (Genentech/Roche), pritumumab, racotumomab (Vaxira®, Recombio), ramucirumab (Cyramza®, ImClone Systems Inc.), rituximab (Rituxan®, Genentech), robatumumab (Schering-Plough), Seribantumab (Sanofi/Merrimack Pharmaceuticals, Inc.), sibrotuzumab, siltuximab (Sylvant™, Janssen Biotech), Smart MI95 (Protein Design Labs, Inc.), Smart ID10 (Protein Design Labs, Inc.), tabalumab (LY2127399, Eli Lilly), taplitumomab paptox, tenatumomab, teprotumumab (Roche), tetulomab, TGN1412 (CD28-SuperMAB or TAB08), tigatuzumab (CD-1008, Daiichi Sankyo), tositumomab, trastuzumab (Herceptin®), tremelimumab (CP-672,206; Pfizer), tucotuzumab celmoleukin (EMD Pharmaceuticals), ublituximab, urelumab (BMS-663513, Bristol-Myers Squibb), volociximab (M200, Biogen Idec), and zatuximab.

In some instances, the antibody or binding fragments thereof is an antibody-drug conjugate (ADC). In some cases, the payload of the ADC comprises, for example, but is not limited to, an auristatin derivative, maytansine, a maytansinoid, a taxane, a calicheamicin, cemadotin, a duocarmycin, a pyrrolobenzodiazepine (PDB), or a tubulysin. In some instances, the payload comprises monomethyl auristatin E (MMAE) or monomethyl auristatin F (MMAF). In some instances, the payload comprises DM2 (mertansine) or DM4. In some instances, the payload comprises a pyrrolobenzodiazepine dimer.

### IV. VECTORS AND EXPRESSION SYSTEMS

RTL or PNMA family (e.g., RTL10 or PEG10) and endo-Gag polypeptides of the disclosure can be encoded by nucleic acids, for example, for expression in a host cell or using a cell-free expression system. In certain embodiments, the RTL or PNMA polypeptides, endo-Gag polypeptides, engineered RTL or PNMA and engineered endo-Gag polypeptides described herein are encoded by vectors, e.g., plasmid vectors. In some embodiments, vectors include any suitable vector derived from either a eukaryotic or prokaryotic source. In some cases, vectors are obtained from bacteria (e.g. E. coli), insects, yeast (e.g. Pichia pastoris), algae, or mammalian sources.

Illustrative bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, and pTAC-MAT-2.

Illustrative insect vectors include pFastBac1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Illustrative algae vectors include pChlamy-4 vector or MCS vector.

Examples of mammalian vectors include transient expression vectors or stable expression vectors. Mammalian transient expression vectors include p3×FLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3×FLAG-CMV 7.1, pFLAG-CMV 20, p3×FLAG-Myc-CMV 24, pCMV-FLAG-MAT1, pCMV-FLAG-MAT2, pBICEP-CMV 3, or pBICEP-CMV 4. Mammalian stable expression vector include pFLAG-CMV 3, p3×FLAG-CMV 9, p3×FLAG-CMV 13, pFLAG-Myc-CMV 21, p3×FLAG-Myc-CMV 25, pFLAG-CMV 4, p3×FLAG-CMV 10, p3×FLAG-CMV 14, pFLAG-Myc-CMV 22, p3×FLAG-Myc-CMV 26, pBICEP-CMV 1, or pBICEP-CMV 2.

In certain embodiments, the RTL or PNMA polypeptides, endo-Gag polypeptides, engineered RTL or PNMA and engineered endo-Gag polypeptides described herein and/or heterologous cargos are expressed using a cell-free expression system. In some instances, a cell-free system is a mixture of cytoplasmic and/or nuclear components from a cell, or isolated transcription and/or translation machinery, and is used for in vitro RNA and/or protein expression. In some cases, a cell-free system utilizes either prokaryotic cell components or eukaryotic cell components, or transcription and/or translation machinery derived therefrom. In some embodiments, nucleic acid synthesis is obtained in a cell-free system based on for example Drosophila cell, Xenopus egg, or HeLa cells (ATCC® CCL-2™). Exemplary cell-free systems include, but are not limited to, E. coli S30 Extract system, E. coli T7 S30 system, or PURExpress®.

In certain embodiments, the RTL (e.g., PEG10 or RTL10) or PNMA polypeptides, endo-Gag polypeptides, engineered RTL or PNMA and engineered endo-Gag polypeptides described herein, and/or heterologous cargos are expressed by a host cell. In some embodiments, a host cell includes any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., a yeast cell), an animal cell, or a plant cell. In some cases, a prokaryotic cell is a bacterial cell. Examples of bacterial cell include gram-positive bacteria or gram-negative bacteria. In some embodiments the gram-negative bacteria are anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes-Verrucomicrobia/Chlamydiae (PVC group), Proteobacteria, Spirochaetes or Synergistetes. In some embodiments, bacteria is Acidobacteria, Chloroflexi, Chrysiogenetes, Cyanobacteria, Deferribacteres, Dictyoglomi, Thermodesulfobacteria or Thermotogae. In some embodiments, a bacterial cell is Escherichia coli, Clostridium botulinum, or Coli bacilli.

Exemplary prokaryotic host cells include, but are not limited to, BL21, Mach1™, DH10B™, TOP10, DH5α, DH10Bac™, OmniMax™, MegaX™, DH12S™, INV110, TOP10F′, INVαF, TOP10/P3, ccdB Survival, PIR1, PIR2, Stbl2™, Stbl3™, or Stbl4™

In some instances, animal cells include a cell from a vertebrate or from an invertebrate. In some cases, an animal cell includes a cell from a marine invertebrate, fish, insects, amphibian, reptile, mammal, or human. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomyces (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a human, primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293 FT cell line, 293F cells, 293 H cells, CHO DG44 cells, CHO-S cells, CHO-K1 cells, Expi293F™ cells, Flp-In™ T-REX™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REX™ Jurkat cell line, Per.C6 cells, T-REX™-293 cell line, T-REX™-CHO cell line, and T-REX™-HeLa cell line.

In some instances, a mammalian host cell is a primary cell. In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High Five™ cells, and expresSF+® cells. Exemplary insect cell lines include, but are not limited to, strains from Chlamydomonas reinhardtii 137c, or Synechococcus elongatus PPC 7942. In some instances, plant cells include cells from algae.

### V. METHODS

Disclosed herein, in certain embodiments, are methods of preparing a capsid, e.g., a capsid which encapsulates a heterologous cargo. In some embodiments, RTL or PNMA family (e.g., RTL10, PEG10, PNMA2, PNMA5, and/or other RTL or PNMA polypeptides) and/or endo-Gag polypeptides of the disclosure exhibit favorable properties for capsid assembly, disassembly, and/or reassembly.

The presence of RTL or PNMA or endo-Gag polypeptides in a capsid form or non-capsid form can be determined using, for example, size exclusion chromatography, multi-angle dynamic light scattering (MADLS), electron microscopy (EM, e.g., scanning or transmission EM), or a combination thereof. Capsid assembly, disassembly, and re-assembly efficiency can be calculated, for example, by comparing the quantity of protein present in assembled capsids to the total quantity of protein, or to the quantity of protein present in particles of smaller size and/or larger size than the capsids. In some embodiments, when calculating reassembly, the amount of protein loss during disassembly and reassembly can be measured and accounted for in the calculations.

In some embodiments, measuring capsid assembly efficiency comprises quantifying the amount of purified protein that is present in a capsid state (e.g., as capsid particles), and quantifying the amount of purified protein that is present in non-capsid states (e.g., non-assembled and/or partially-assembled states). For example, size exclusion chromatography can be used to separate monomers and oligomers from assembled capsids, and to quantify the amount of protein that is present in a capsid state versus un-assembled or partially-assembled non-capsid states.

In some embodiments, measuring capsid disassembly efficiency comprises quantifying the amount of capsid-forming protein that remains in solution after disassembling from a capsid state to a non-capsid state (e.g., oligomers, capsomers, and/or monomers). Capsomers can comprise partially assembled capsid subunits, for example, comprising at least 2, at least 3, at least 4, at least 5, at least 6, at least 7, at least 8, at least 9, at least 10, at least 11, at least 12, at least 13, at least 14, at least 15, at most 2, at most 3, at most 4, at most 5, at most 6, at most 7, at most 8, at most 9, at most 10, at most 11, at most 12, at most 13, at most 14, at most 15, about 2, about 3, about 4, about 5, about 6, about 7, about 8, about 9, about 10, about 11, about 12, about 13, about 14, or about 15 endo Gag polypeptide monomers.

In some embodiments, the efficiency of disassembly can be determined by quantifying the percent of total protein that is a monomer peak as measured by MADLS or SEC. In some embodiments, capsids are treated with a disassembly buffer, and optionally centrifuged to get precipitate (e.g., aggregates) and/or remaining capsids out of solution. The loss of capsid structures confirmed by electron microscopy (e.g., TEM) and/or dynamic light scattering (e.g., MADLS). The efficiency of disassembly can be determined by MADLS (e.g., via a capsid peak and a monomer peak) or size exclusion chromatography (SEC). The amount of protein that remains in solution can be quantified. The amount of monomer in solution can be quantified. Disassembly efficiency can be calculated. For example, a recovery of 90 ng of protein in solution from 100 ng of starting capsid material indicates a disassembly efficiency of 90%.

In some embodiments, measuring capsid reassembly efficiency comprises quantifying an amount of disassembled protein in solution that reassembles into a capsid state. In some embodiments, an amount of input protein in a non-capsid state in solution is quantified (e.g., after treating with a disassembly buffer, optionally centrifugation to remove precipitate and/or capsids, and confirming a lack of capsid structures via TEM and/or MADLS). In some embodiments, disassembled protein is treated with a reassembly buffer disclosed herein, e.g., that comprises a physiological buffer, low salt, high salt, an acidic buffer plus divalent cation, does not contain a chaotropic agent, does not contain a reducing agent, and/or another assembly or reassembly buffer disclosed herein. Aggregates can be removed by centrifugation, e.g., at 15,000×g for 5 min at +4 °C. The efficiency of reassembly can be determined by comparing the amounts of endo-Gag polypeptides in the monomer and capsid peaks as assayed by MADLS or SEC. Capsid formation can be confirmed by TEM and/or MADLS. In some embodiments, size exclusion chromatography performed to quantify the amount of protein in a capsid state, and this can be compared to an amount of input protein. Reassembly efficiency is calculated. For example, 81 ng of protein detected in a capsid state by size exclusion chromatography after starting with 90 ng of protein in a non-capsid state in solution indicates a 90% reassembly efficiency. In some embodiments, reassembly efficiency is determined by comparing the amount of endo-Gag polypeptides in reassembled capsids without further purification to the input amount of endo-Gag monomer polypeptides. In some embodiments, reassembly efficiency is determined by comparing the amount of endo-Gag polypeptides in reassembled capsids with further purification (e.g., via SEC or IEC) to the input amount of endo-Gag monomer polypeptides

In some embodiments, MADLS is used to determine particle concentration and verify whether it is within the expected range for the amount of protein in solution. For example, for PNMA2, which has a molecular weight of 41509.37, the expected number of particles can be calculated from the amount of PNMA2 polypeptide based on an assumption of 60 PNMA2 monomers per capsid: 1 g Capsids=2.42×10¹⁷ Capsids. In another embodiment, based on the endo Gag polypeptide molecular weight, the expected number of particles can be calculated from the amount of endo Gag polypeptide based on an assumption of x (e.g., 60) monomers per capsid.

In some embodiments, an observed concentration of a capsid disclosed herein is within about ±5%, within about ±10%, within about ±20%, within about ±30%, within about ±40%, within about ±50%, within about ±60%, within about ±70%, within about ±80%, within about ±90%, within about ±2-fold, within about ±5-fold, or within about ±10-foldof an expected particle concentration.

In some embodiments, further purification is performed (e.g., via SEC or ion-exchange chromatography) if capsid reassembly is less than 5%, less than 10%, less than 20%, less than 30%, less than 40%, less than 50%, less than 60%, less than 70%, less than less than 80%, less than 85%, less than 86%, less than 87%, less than 88%, less than 89%, less than 90%, less than 91%, less than 92%, less than 93%, less than 94%, less than 95%, less than 96%, less than 97%, less than 98%, less than 99%, or less than 99.5% efficient.

In some embodiments, a RTL or PNMA family or endo-Gag polypeptide disclosed herein exhibits favorable assembly properties. In some embodiments, upon isolation, a RTL or PNMA or endo-Gag polypeptide of the disclosure (e.g., PEG10, RTL10, PNMA2, PNMA5, or another PNMA polypeptide or another RTL or endo-Gag polypeptide) assembles to form capsids with an efficiency of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99%. In some embodiments, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99% of the isolated RTL or PNMA or endo-Gag polypeptide assembles to form the capsid.

In some embodiments, an RTL or PNMA or endo-Gag polypeptide disclosed herein exhibits favorable disassembly properties. In some embodiments, after incubation in a disassembly buffer, RTL or PNMA or endo-Gag capsids (e.g., comprising PEG10, RTL10, PNMA2, PNMA5, or another RTL, endo-Gag, or PNMA polypeptide) disassemble (e.g., to monomers, or smaller subunits than the capsids) with an efficiency of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99%. In some embodiments, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99% of the RTL or PNMA or endo-Gag polypeptide present in capsids disassembles to a non-capsid state.

In some embodiments, an RTL, PNMA or endo-Gag polypeptide disclosed herein exhibits favorable reassembly properties. In some embodiments, after disassembly, upon incubation in a suitable reassembly buffer, an RTL or PNMA (e.g., comprising RTL10, PEG10, PNMA2, PNMA5, or another RTL or PNMA polypeptide) or endo-Gag polypeptide of the disclosure reassembles to form capsids with an efficiency of at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99%. In some embodiments, at least about 0.1%, at least about 0.5%, at least about 1%, at least about 2%, at least about 3%, at least about 4%, at least about 5%, at least about 6%, at least about 7%, at least about 8%, at least about 9%, at least about 10%, at least about 11%, at least about 12%, at least about 13%, at least about 14%, at least about 15%, at least about 20%, at least about 25%, at least about 30%, at least about 40%, at least about 50%, at least about 60%, at least about 70%, at least about 80%, at least about 90%, at least about 95%, at least about 97%, or at least about 99% of the RTL or PNMA or endo-Gag polypeptide reassembles to form the capsid, e.g., as a percentage of the RTL or PNMA or endo-Gag protein that is present after disassembly, as a percentage of the RTL or PNMA or endo-Gag protein that was originally present in capsids, or as a percentage of the RTL or PNMA or endo-Gag protein that is present upon initial protein isolation.

In some embodiments, a method disclosed herein comprises incubating a plurality of RTL or PNMA (e.g., RTL10, PEG10, PNMA2, PNMA5, or another RTL or PNMA family member) or endo-Gag polypeptides, engineered RTL, PNMA or endo-Gag polypeptides, and/or recombinant RTL, PNMA or endo-Gag polypeptides with a heterologous cargo in a solution for a time sufficient to generate a loaded RTL-based or PNMA-based capsid or endo-Gag-based capsid. The solution can be or can comprise, for example, an assembly buffer or reassembly buffer disclosed herein. In some embodiments the heterologous cargo is incubated with the RTL, PNMA or endo-Gag polypeptide in a disassembly buffer disclosed herein.

In some embodiments, the time sufficient to generate a loaded RTL-based or PNMA-based capsid or endo-Gag-based capsid is at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more. In some embodiments, the time sufficient to generate a loaded RTL-based or PNMA-based capsid or endo-Gag-based capsid is at most about 5 minutes, at most about 10 minutes, at most about 20 minutes, at most about 30 minutes, at most about 1 hour, at most about 2 hours, at most about 4 hours, at most about 6 hours, at most about 8 hours, at most about 10 hours, at most about 12 hours, at most about 16 hours, at most about 20 hours, at most about 24 hours, or at most about 48 hours.

In some cases, the RTL-based or PNMA-based capsid or endo-Gag-based capsid is assembled, disassembled, or reassembled at a temperature from about 2 °C to about 37 °C. In some instances, the RTL-based or PNMA-based capsid or endo-Gag-based capsid is prepared at a temperature from about 2 °C to about 8 °C, about 2 °C to about 4 °C, about 20 °C to about 37 °C, about 25 °C to about 37 °C, about 20 °C to about 30 °C, about 25 °C to about 30 °C, about 30 °C to about 37 °C, from about 2 °C to about 40 °C, 2 °C to about 25 °C, 2 °C to about 20 °C, 4 °C to about 40 °C, 4 °C to about 37 °C, 4 °C to about 25 °C, or 4 °C to about 20 °C. In some cases, the RTL-based or PNMA-based capsid or endo-Gag-based capsid is assembled, disassembled, or reassembled at room temperature.

In some instances, the method comprises mixing a solution comprising a plurality of an engineered and/or recombinant PNMA family polypeptide (e.g., PNMA1, PNMA2, PNMA3, PNMA4 (MOAP1), PNMA5, PNMA6A, PNMA6B/6D, PNMA6E, PNMA6F, PNMA7A (ZCCHC12), PNMA7B (ZCCHC18), PNMA8A, PNMA8B, PNMA8C, or CCDC8) with a plurality of a non-PNMA family capsid forming subunit (e.g., another endogenous Gag polypeptide disclosed herein) prior to, at the same time as, or after incubating with the cargo. In some cases, the plurality of non-PNMA family capsid forming subunits are mixed with the plurality of engineered and/or recombinant PNMA polypeptides at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some instances, the method comprises mixing a solution comprising a plurality of a first engineered and/or recombinant PNMA polypeptide (e.g., PNMA1, PNMA2, PNMA3, PNMA4 (MOAP1), PNMA5, PNMA6A, PNMA6B/6D, PNMA6E, PNMA6F, PNMA7A (ZCCHC12), PNMA7B (ZCCHC18), PNMA8A, PNMA8B, PNMA8C, or CCDC8) with a plurality of a second engineered and/or recombinant PNMA polypeptide (e.g., PNMA1, PNMA2, PNMA3, PNMA4 (MOAP1), PNMA5, PNMA6A, PNMA6B/6D, PNMA6E, PNMA6F, PNMA7A (ZCCHC12), PNMA7B (ZCCHC18), PNMA8A, PNMA8B, PNMA8C, or CCDC8) prior to, at the same time as, or after incubating with the cargo. In some cases, the first engineered and/or recombinant PNMA polypeptide is mixed with the second engineered and/or recombinant PNMA polypeptide at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some instances, the method comprises mixing a solution comprising a plurality of an engineered and/or recombinant PNMA2 polypeptide with a plurality of a non-PNMA2 capsid forming subunit (e.g., another PNMA or endogenous Gag polypeptide disclosed herein) prior to, at the same time as, or after incubating with the cargo. In some cases, the plurality of non-PNMA2 capsid forming subunits are mixed with the plurality of engineered and/or recombinant PNMA2 polypeptides at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some instances, the method comprises mixing a solution comprising a plurality of an engineered and/or recombinant PNMA5 polypeptide with a plurality of a non-PNMA5 capsid forming subunit (e.g., another PNMA or endogenous Gag polypeptide disclosed herein) prior to, at the same time as, or after incubating with the cargo. In some cases, the plurality of non-PNMA5 capsid forming subunits are mixed with the plurality of engineered and/or recombinant PNMA5 polypeptides at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some instances, the method comprises mixing a solution comprising a plurality of an engineered and/or recombinant RTL family polypeptide with a plurality of a non-RTL family capsid forming subunit (e.g., another endogenous Gag polypeptide disclosed herein) prior to, at the same time as, or after incubating with the cargo. In some cases, the plurality of non-RTL family capsid forming subunits are mixed with the plurality of engineered and/or recombinant RTL polypeptides at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some instances, the method comprises mixing a solution comprising a plurality of an engineered and/or recombinant RTL10 polypeptide with a plurality of a non-RTL10 capsid forming subunit (e.g., another endogenous Gag polypeptide disclosed herein) prior to, at the same time as, or after incubating with the cargo. In some cases, the plurality of non-RTL10 capsid forming subunits are mixed with the plurality of engineered and/or recombinant RTL10 polypeptides at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

In some instances, the method comprises mixing a solution comprising a plurality of an engineered and/or recombinant PEG10 polypeptide with a plurality of a non-PEG10 capsid forming subunit (e.g., another endogenous Gag polypeptide disclosed herein) prior to, at the same time as, or after incubating with the cargo. In some cases, the plurality of non-PEG10 capsid forming subunits are mixed with the plurality of engineered and/or recombinant PEG10 polypeptides at a ratio of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1.

Various prospective assembly buffers, disassembly buffers, and reassembly buffers can be tested on endo Gag proteins disclosed herein to identify suitable buffers for the particular endo Gag polypeptide. For example, assembly efficiency, disassembly efficiency, reassembly efficiency, cargo loading, or a combination thereof can be assessed, e.g., using techniques disclosed herein.

### A. Assembly Buffer

In some embodiments, an assembly buffer comprises a physiological buffer. In some embodiments, an assembly buffer is or comprises a balanced salt solution. In some embodiments, an assembly buffer is or comprises a saline, e.g., a buffered saline. In some embodiments, an assembly buffer is or comprises phosphate buffered saline (PBS). In some embodiments, an assembly buffer is or comprises HBSS. In some embodiments, an assembly buffer is or comprises ringer's solution. In some embodiments, an assembly buffer comprises sodium phosphate.

In some embodiments, an assembly buffer comprises one or more osmolytes, for example, glycerol, ethylene glycol, mannitol, glycine, glycine betaine, trehalose, sorbitol, or a combination thereof.

In some embodiments, an assembly buffer does not contain or substantially lacks a chaotropic agent. In some embodiments, an assembly buffer does not contain or substantially lacks a reducing agent.

In some embodiments, a RTL (e.g., RTL10 or PEG10), PNMA or endo-Gag polypeptide of the disclosure exhibits favorable assembly properties in buffers with low or physiological levels of salt. In some embodiments, an assembly buffer comprises low salt or low ionic strength, e.g., with less than about 250 mM of a salt, such as NaCl. In some embodiments, an assembly buffer comprises high salt or high ionic strength. In some embodiments, an assembly buffer is not a balanced salt solution.

In some embodiments, an assembly buffer comprises less than 10000, less than 9000, less than 8000, less than 7000, less than 6000, less than 5000, less than 4000, less than 3000, less than 2000, less than 1000, less than 900, less than 800, less than 700, less than 600, less than 500, less than 400, less than 300, or less than 200 mOsm/kg of salt.

In some embodiments, an assembly buffer comprises at least 8000, at least 7000, at least 6000, at least 5000, at least 4000, at least 3000, at least 2000, at least 1000, at least 900, at least 800, at least 700, at least 600, at least 500, at least 400, at least 300, or at least 200 mOsm/kg of salt.

In some embodiments, an assembly buffer comprises about 150-8000, about 150-5000, about 150-4000, about 150-3000, about 150-2000, about 150-1000, about 150-750, about 150-600, about 150-500, about 150-400, about 150-300, about 150-250, about 150-200, about 200-8000, about 200-5000, about 200-4000, about 200-3000, about 200-2000, about 200-1000, about 200-750, about 200-600, about 200-500, about 200-400, about 200-300, about 200-250, about 250-8000, about 250-5000, about 250-4000, about 250-3000, about 250-2000, about 250-1000, about 250-750, about 250-600, about 250-500, about 250-400, about 250-300, about 270-8000, about 270-5000, about 270-4000, about 270-3000, about 270-2000, about 270-1000, about 270-750, about 270-600, about 270-500, about 270-400, about 270-300, about 300-8000, about 300-5000, about 300-4000, about 300-3000, about 300-2000, about 300-1000, about 300-750, about 300-600, about 300-500, about 300-400, about 500-8000, about 500-5000, about 500-4000, about 500-3000, about 500-2000, about 500-1000, about 500-750, about 500-600, about 1000-8000, about 1000-5000, about 1000-4000, about 1000-3000, about 1000-2000, about 1000-8000, about 1000-5000, about 1000-4000, about 1000-3000, about 1000-2000, about 2000-8000, about 2000-5000, about 2000-4000, about 2000-3000, about 2000-2000, about 2000-8000, about 2000-5000, about 2000-4000, or about 2000-3000 mOsm/kg of salt.

In some embodiments, an assembly buffer comprises glycerol. The glycerol can be present at a concentration of, for example, at least 1%, at least 5%, at least 10%, at least 15%, at least 20%, at least 25%, at least 30%, at least 35%, at least 40%, at least 45%, or at least 50% (e.g., w/w, v/v, or w/v). The glycerol can be present at a concentration of, for example, at most 5%, at most 10%, at most 15%, at most 20%, at most 25%, at most 30%, at most 35%, at most 40%, at most 45%, or at most 50% (e.g., w/w, v/v, or w/v). The glycerol can be present at a concentration of, for example, about 5%, about 10%, about 15%, about 20%, about 25%, about 30%, about 35%, about 40%, about 45%, or about 50% (e.g., w/w, v/v, or w/v). The glycerol can be present at a concentration of, for example, 1-50%, 5-50%, 10-50%, 15-50%, 20-50%, 25-50%, 30-50%, 1-30%, 5-30%, 10-30%, 15-30%, 20-30%, 25-30%, 1-20%, 5-20%, 10-20%, 15-20%, 1-15%, 5-15%, 10-15%, 1-10%, or 5-10% (e.g., w/w, v/v, or w/v).

In some embodiments, an assembly buffer comprises a monovalent salt. The monovalent salt can be, for example, NaCl, KCl, LiCl, NaBr, or NaF.

The monovalent salt can be at a concentration of, for example, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 250 mM, at least 300 mM, at least 350 mM, at least 400 mM, at least 450 mM, at least 500 mM, at least 600 mM, at least 700 mM, at least 800 mM, at least 900 mM, at least 1000 mM, at least 1250 mM, at least 1500 mM, at least 2000 mM, at least 3000 mM, at least 4000 mM, or at least 5000 mM.

In some embodiments, the monovalent salt is at a concentration of at most 50 mM, at most 60 mM, at most 70 mM, at most 80 mM, at most 90 mM, at most 100 mM, at most 150 mM, at most 200 mM, at most 250 mM, at most 300 mM, at most 350 mM, at most 400 mM, at most 450 mM, at most 500 mM, at most 600 mM, at most 700 mM, at most 800 mM, at most 900 mM, at most 1000 mM, at most 1250 mM, at most 1500 mM, at most 2000 mM, at most 3000 mM, at most 4000 mM, or at most 5000 mM.

In some embodiments, the monovalent salt is at a concentration of about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1000 mM, about 1250 mM, about 1500 mM, about 2000 mM, about 3000 mM, about 4000 mM, or about 5000 mM.

In some embodiments, the monovalent salt is at a concentration of about 50-5000 mM, about 100-5000 mM, about 150-5000 mM, about 250-5000 mM, about 500-5000 mM, about 1000-5000 mM, about 50-2000 mM, about 100-2000 mM, about 150-2000 mM, about 250-2000 mM, about 500-2000 mM, about 1000-2000 mM, about 50-1500 mM, about 100-1500 mM, about 150-1500 mM, about 250-1500 mM, about 500-1500 mM, about 1000-1500 mM, about 50-1000 mM, about 100-1000 mM, about 150-1000 mM, about 250-1000 mM, about 500-1000 mM, about 50-750 mM, about 100-750 mM, about 150-750 mM, about 250-750 mM, about 500-750 mM, about 50-500 mM, about 100-500 mM, about 150-500 mM, about 250-500 mM, about 50-250 mM, about 100-250 mM, about 150-250 mM, or about 50-100 mM.

In some embodiments, an assembly buffer comprises Na₂HPO₄. In some embodiments, an assembly buffer comprises KH₂PO₄.

In some embodiments, an assembly buffer comprises a divalent salt or divalent cation e.g., from CaCl₂, MgCl₂, or ZnCl₂. In some embodiments, an assembly buffer comprises CaCl₂. In some embodiments, an assembly buffer comprises MgCl₂. The divalent cation can be present at a concentration of, for example, at least 1 μM, at least 10 μM, at least 25 μM, at least 50 μM, at least 100 μM, at least 500 μM, at least 1 mM, at least 10 mM, at least 20 mM, at least 30 mM, at least 40 mM, at least 50 mM, at least 75 mM, at least 100 mM, or at least 250 mM.

The divalent cation can be present at a concentration of, for example, at most 25 μM, at most 50 μM, at most 100 μM, at most 500 μM, at most 1 mM, at most 10 mM, at most 20 mM, at most 30 mM, at most 40 mM, at most 50 mM, at most 75 mM, at most 100 mM, or at most 250 mM.

The divalent cation can be present at a concentration of, for example, about 1 μM, about 10 μM, about 25 μM, about 50 μM, about 100 μM, about 500 μM, about 1 mM, about 10 mM, about 20 mM, about 30 mM, about 40 mM, about 50 mM, about 75 mM, about 100 mM, or about 250 mM.

The divalent cation can be present at a concentration of, for example, about 1 μM-250 mM, about 10 μM-250 mM, about 50 μM-250 mM, about 100 μM-250 mM, about 1 mM-250 mM, about 10 mM-250 mM, about 25 mM-250 mM, about 50-250 mM, about 100-250 mM, about 1 μM-100 mM, about 10 μM-100 mM, about 50 μM-100 mM, about 100 μM-100 mM, about 1 mM-100 mM, about 10 mM-100 mM, about 25 mM-100 mM, about 50-100 mM, about 1 μM-50 mM, about 10 μM-50 mM, about 50 μM-50 mM, about 100 μM-50 mM, about 1 mM-50 mM, about 10 mM-50 mM, about 25 mM-50 mM, about 1 μM-10 mM, about 10 μM-10 mM, about 50 μM-10 mM, about 100 μM-10 mM, about 1 mM-10 mM, about 10 mM-10 mM, about 25 mM-10 mM, about 50-10 mM, about 1 μM-1 mM, about 10 μM-1 mM, about 50 μM-1 mM, about 100 μM-1 mM, about 1 μM-100 μM, about 10 μM-100 μM, or about 50 μM-100 μM.

In some embodiments, an assembly buffer comprises a buffering agent, e.g., an acidic buffer. In some embodiments, an assembly buffer comprises Tris. In some embodiments, an assembly buffer comprises imidazole. In some embodiments, an assembly buffer comprises DTT.

Non-limiting examples of buffering agents that can be used include MES, Tris, NaP, CHES, and CAPS.

The buffering agent can be at a concentration of, for example, at least 1 mM, 10 mM, at least 20 mM, at least 25 mM, at least 30 mM, at least 40 mM, at least 50 mM, at least 75 mM, at least 100 mM, at least 150 mM, at least 200 mM, at least 250 mM, or at least 500 mM.

The buffering agent can be at a concentration of, for example, at most 10 mM, at most 25 mM, of at most 50 mM, at most 100 mM, at most 150 mM, at most 200 mM, at most 250 mM, or at most 500 mM.

The buffering agent can be at a concentration of, for example, about 10 mM, about 25 mM, of about 50 mM, about 100 mM, about 150 mM, about 200 mM, about 250 mM, or about 500 mM.

In some embodiments, the buffering agent is at a concentration of about 1-500 mM, about 1-250 mM, about 1-150 mM, about 1-100 mM, about 1-75 mM, about 1-50 mM, about 1-25 mM, about 1-10 mM, about 5-500 mM, about 5-250 mM, about 5-150 mM, about 5-100 mM, about 5-75 mM, about 5-50 mM, about 5-25 mM, about 5-10 mM, about 10-500 mM, about 10-250 mM, about 10-150 mM, about 10-100 mM, about 10-75 mM, about 10-50 mM, about 10-25 mM, about 20-500 mM, about 20-250 mM, about 20-150 mM, about 20-100 mM, about 20-75 mM, about 20-50 mM, about 20-25 mM, about 50-500 mM, about 50-250 mM, about 50-150 mM, or about 50-100 mM.

In some embodiments, an assembly buffer comprises NaCl, KCl, Na₂HPO₄, and KH₂PO₄.

In some embodiments, an assembly buffer comprises DTT, e.g., at a low concentration.

The assembly buffer may contain a heterologous cargo (e.g., nucleic acid). In some embodiments, the heterologous cargo (e.g., RNA) can promote capsid assembly.

In some embodiments, an assembly buffer comprises a monovalent salt, a divalent cation, and optionally a buffering agent. In some embodiments, an assembly buffer comprises NaCl, ZnCl₂, and optionally Tris. In some embodiments, an assembly buffer comprises about 150 mM NaCl, about 10 μM ZnCl₂, and optionally about 25 mM Tris. In some embodiments, an assembly buffer comprises about 150 mM NaCl, about 1 mM DTT, about 10 μM ZnCl₂, and optionally about 50 mM Tris.

In some embodiments, an assembly buffer comprises 2-(N-morpholino)-ethanesulfonic acid (MES) and MgCl₂. In some embodiments, an assembly buffer comprises about 50 mM MES and about 40 mM MgCl₂.

Illustrative, non-limiting examples of assembly buffers include: (1) 137 mM NaCl, 2.7 mM KCl, 10 mM Na₂HPO₄, 1.8 mM KH₂PO₄; (2) 137 mM NaCl, 2.7 mM KCl, 8 mM Na₂HPO₄, 1.5 mM KH₂PO₄, 0.9 mM CaCl₂, 0.5 mM MgCl₂); (3) 75 mM NaCl, 50 mM Tris, 10% glycerol, pH 8.0; and (4) 20 mM Na₂HPO₄/NaH₂PO₄, 0.5 M NaCl, 10% glycerol, 40 mM imidazole, 1 mM DTT, pH 7.4. In some embodiments the assembly buffer does not contain a chaotropic agent and/or a reducing agent. The assembly buffer can contain a heterologous cargo.

Illustrative, non-limiting examples of assembly buffers that can be used for PEG10 include: (1) a low salt buffer with 150 mM NaCl, 25 mM Tris, 10 μM ZnCl₂, pH 7.5; and (2) a low salt buffer with 150 mM NaCl, 1 mM DTT, 10 μM ZnCl₂, 50 mM Tris, pH 8.0.

An illustrative, non-limiting example of an assembly buffer that can be used for RTL10 is an acidic buffer plus divalent cation: 50 mM MES (2-(N-morpholino) ethanesulfonic acid), 40 mM MgCl₂, pH 6.0.

In some embodiments, capsid assembly is conducted at a temperature of at least-20 °C, at least −10 °C, at least 0 °C, at least 2 °C, at least 4 °C, at least 6 °C, at least 8 °C, at least 10 °C, at least 12 °C, at least 14 °C, at least 16 °C, at least 18 °C, at least 20 °C, at least 22 °C, at least 25 °C, at least 30 °C, or at least 37 °C. In some embodiments, capsid assembly is conducted at a temperature of at most 2 °C, at most 4 °C, at most 6 °C, at most 8 °C, at most 10 °C, at most 12 °C, at most 14 °C, at most 16 °C, at most 18 °C, at most 20 °C, at most 22 °C, at most 25 °C, at most 30 °C, at most 37 °C, at most 40 °C or at most 45 °C. In some embodiments, capsid assembly is conducted at a temperature of about −20 °C, about −10 °C, about 0 °C, about 2 °C, about 4 °C, about 6 °C, about 8 °C, about 10 °C, about 12 °C, about 14 °C, about 16 °C, about 18 °C, about 20 °C, about 22 °C, about 25 °C, about 30 °C, or about 37 °C. In some embodiments, capsid assembly is conducted at a temperature of about 2 °C to about 40 °C, 2 °C. to about 37 °C, 2 °C to about 25 °C, 2 °C to about 20 °C, 4 °C to about 40 °C, 4 °C to about 37 °C, 4 °C to about 25 °C, 4 °C to about 20 °C, 2 °C to about 8 °C, about 2 °C to about 4 °C, about 20 °C to about 37 °C, about 25 °C to about 37 °C, about 20 °C to about 30 °C, about 25 °C to about 30 °C, or about 30 °C to about 37 °C.

In some embodiments, capsid assembly comprises incubating in an assembly buffer for at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more.

In some embodiments, an assembly buffer comprises a pH of about 3, about 4, about 5, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 10, or about 11. In some embodiments, an assembly buffer comprises a pH of at least about 3, at least about 4, at least about 5, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8. In some embodiments, an assembly buffer comprises a pH of at most about 6, at most about 6.5, at most about 7, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 10, or at most about 11. In some embodiments, an assembly buffer comprises a pH of about 5-10, 5-9, 5-8, 5-7.8, 5-7.6, 5-7.5, 5-7.4, 5-7, 5-6.5, 5-6, 8-8.5, 6-9, 6-8, 6-7.8, 6-7.6, 6-7.5, 6-7.4, 6-7, 6-6.5, 7-9, 7-8.5, 7-8, 7-7.8, 7-7.6, 7-7.5, 7.5-8, 8-10, 8-9.5, 8-9, or 8-8.5.

An assembly buffer can comprise a component disclosed herein at a concentration of at least 1 μm, at least 10 μM, at least 100 μM, at least 1 nM, at least 10 nM, at least 100 nM, at least 1 μM, at least 10 μM, at least 100 μM, at least 1 mM, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 250 mM, at least 300 mM, at least 350 mM, at least 400 mM, at least 450 mM, at least 500 mM, at least 1M, at least 2M, or at least 5M.

An assembly buffer can comprise a component disclosed herein at a concentration of at most 1 μm, at most 10 μM, at most 100 μM, at most 1 nM, at most 10 nM, at most 100 nM, at most 1 μM, at most 10 μM, at most 100 μM, at most 1 mM, at most 10 mM, at most 50 mM, at most 60 mM, at most 70 mM, at most 80 mM, at most 90 mM, at most 100 mM, at most 150 mM, at most 200 mM, at most 250 mM, at most 300 mM, at most 350 mM, at most 400 mM, at most 450 mM, at most 500 mM, at most 1M, at most 2M, or at most 5M.

An assembly buffer can comprise a component disclosed herein at a concentration of about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1000 mM, about 1250 mM, about 1500 mM, about 2000 mM, about 3000 mM, about 4000 mM, or about 5000 mM.

### B. Disassembly Buffer

A disassembly buffer can comprise one or more reducing agents, e.g., reduced glutathione (GSH), beta mercaptoethanol (β-ME), Dithiothreitol (DTT), tris(2-carboxyethyl)phosphine (TCEP), or a combination thereof.

The reducing agent can be present at a concentration of at least 0.1 mM, at least 0.5 mM, at least 1 mM, at least 5 mM, at least 10 mM, at least 20 mM, at least 30 mM, at least 40 mM, at least 50 mM, at least 60 mM, at least 70 mM, at least 80 mM, at least 90 mM, at least 100 mM, at least 150 mM, or at least 200 mM.

The reducing agent can be present at a concentration of at most 5 mM, at most 10 mM, at most 20 mM, at most 30 mM, at most 40 mM, at most 50 mM, at most 60 mM, at most 70 mM, at most 80 mM, at most 90 mM, at most 100 mM, at most 150 mM, at most 200 mM, at most 300 mM, or at most 500 mM.

The reducing agent can be present at a concentration of about 1 mM, about 5 mM, about 10 mM, about 12 mM, about 20 mM, about 30 mM, about 40 mM, about 50 mM, about 60 mM, about 66.67 mM, about 70 mM, about 80 mM, about 90 mM, about 100 mM, about 150 mM, about 200 mM, about 300 mM, or about 500 mM.

The reducing agent can be present at a concentration of about 0.1-200 mM, about 1-200 mM, about 5-200 mM, about 10-200 mM, about 15-200 mM, about 20-200 mM, about 30-200 mM, about 50-200 mM, about 75-200 mM, about 0.1-100 mM, about 1-100 mM, about 5-100 mM, about 10-100 mM, about 15-100 mM, about 20-100 mM, about 30-100 mM, about 50-100 mM, about 75-100 mM, about 0.1-75 mM, about 1-75 mM, about 5-75 mM, about 10-75 mM, about 15-75 mM, about 20-75 mM, about 30-75 mM, about 50-75 mM, about 0.1-50 mM, about 1-50 mM, about 5-50 mM, about 10-50 mM, about 15-50 mM, about 20-50 mM, about 30-50 mM, about 0.1-30 mM, about 1-30 mM, about 5-30 mM, about 10-30 mM, about 15-30 mM, about 20-30 mM, about 0.1-20 mM, about 1-20 mM, about 5-20 mM, about 10-20 mM, about 15-20 mM, about 0.1-15 mM, about 1-15 mM, about 5-15 mM, about 10-15 mM, about 0.1-10 mM, about 1-10 mM, about 5-10 mM, about 0.1-5 mM, or about 1-5 mM.

In some embodiments, an RTL or endo-Gag polypeptide (e.g., RTL10 or PEG10) disclosed herein exhibits favorable disassembly properties in buffers with low levels of salt. The disassembly buffer can be a low salt buffer or a buffer with a low ionic strength, e.g., with less than about 75 mM of a salt, such as NaCl. For example, in some embodiments RTL 10 can exhibit favorable disassembly properties in buffers with low levels of salt or low ionic strength. In some embodiments a disassembly buffer comprises a reducing agent and a low level of salt.

In some embodiments, a disassembly buffer comprises less than 10000, less than 9000, less than 8000, less than 7000, less than 6000, less than 5000, less than 4000, less than 3000, less than 2000, less than 1000, less than 900, less than 800, less than 700, less than 600, less than 500, less than 400, less than 300, less than 200, less than 150, or less than 100 mOsm/kg of salt. In some embodiments, a disassembly buffer comprises less than 150 mOsm/kg of salt.

In some embodiments, a disassembly buffer comprises high salt or high ionic strength. For example, in some embodiments PEG10 exhibits favorable disassembly properties in buffers with high salt or high ionic strength, e.g., with at least 500 mM of a salt, such as NaCl. In some embodiments a disassembly buffer comprises a reducing agent and a high level of salt.

In some embodiments, a disassembly buffer comprises at least 8000, at least 7000, at least 6000, at least 5000, at least 4000, at least 3000, at least 2000, at least 1000, at least 900, at least 800, at least 700, at least 600, at least 500, at least 400, at least 300, or at least 200 mOsm/kg of salt. In some embodiments, a disassembly buffer comprises at least 800 mOsm/g of salt.

In some embodiments, a disassembly buffer comprises about 75-8000, about 75-5000, about 75-4000, about 75-3000, about 75-2000, about 75-1000, about 75-750, about 75-600, about 75-500, about 75-400, about 75-300, about 75-250, about 75-200, about 150-8000, about 150-5000, about 150-4000, about 150-3000, about 150-2000, about 150-1000, about 150-750, about 150-600, about 150-500, about 150-400, about 150-300, about 150-250, about 150-200, about 200-8000, about 200-5000, about 200-4000, about 200-3000, about 200-2000, about 200-1000, about 200-750, about 200-600, about 200-500, about 200-400, about 200-300, about 200-250, about 250-8000, about 250-5000, about 250-4000, about 250-3000, about 250-2000, about 250-1000, about 250-750, about 250-600, about 250-500, about 250-400, about 250-300, about 270-8000, about 270-5000, about 270-4000, about 270-3000, about 270-2000, about 270-1000, about 270-750, about 270-600, about 270-500, about 270-400, about 270-300, about 300-8000, about 300-5000, about 300-4000, about 300-3000, about 300-2000, about 300-1000, about 300-750, about 300-600, about 300-500, about 300-400, about 500-8000, about 500-5000, about 500-4000, about 500-3000, about 500-2000, about 500-1000, about 500-750, about 500-600, about 1000-8000, about 1000-5000, about 1000-4000, about 1000-3000, about 1000-2000, about 1000-8000, about 1000-5000, about 1000-4000, about 1000-3000, about 1000-2000, about 2000-8000, about 2000-5000, about 2000-4000, about 2000-3000, about 2000-2000, about 2000-8000, about 2000-5000, about 2000-4000, or about 2000-3000 mOsm/kg of salt. In some embodiments, a disassembly buffer comprises about 500-1500 mOsm/kg of salt.

In some embodiments, a disassembly buffer comprises a monovalent salt, a reducing agent, and optionally a basic buffering agent. In some embodiments, a disassembly buffer comprises NaCl, DTT, and optionally Tris. In some embodiments, a disassembly buffer comprises about 25-1800 mM NaCl, 1-20 mM DTT, and optionally 20-75 mM Tris. In some embodiments, a disassembly buffer comprises about 500 mM NaCl, about 5 mM DTT, and optionally 50 mM Tris. In some embodiments, a disassembly buffer comprises about 50 mM NaCl, about 10 mM DTT, and optionally 25 mM Tris.

An illustrative, non-limiting example of a disassembly buffer that can be used for PEG10 is 500 mM NaCl, 5 mM DTT, 50 mM Tris, pH 8.0.

An illustrative, non-limiting example of a disassembly buffer that can be used for RTL10 is 50 mM NaCl, 10 mM DTT, 25 mM Tris, pH 8.0.

A disassembly buffer can comprise one or more solubilizing agents, for example, a detergent, including a non-denaturing detergent, such as CHAPS, nP-40, n-OG, DDM, Triton X-100, LDAO, Tween 20, or a combination thereof. A disassembly buffer can comprise one or more solubilizing agents that are non-detergent sulfobetaines, for example, NDSB-195, NDSB-201, NDSB-211, NDSB-221, NDSB-256, NDSB-256-4T, or a combination thereof.

A disassembly buffer can comprise one or more chaotropic agents, for example, a disassembly buffer can comprise n-butanol, ethanol, guanidinium chloride, lithium perchlorate, lithium acetate, magnesium chloride, phenol, 2-propanol, sodium dodecyl sulfate, thiourea, urea, MgCl2, CaCl2, NaI, or a combination thereof.

In some embodiments, the disassembly buffer comprises a solubilizing agent or chaotropic agent at a concentration of at least 1 mM, at least 10 mM, at least 20 mM, at least 30 mM, at least 40 mM, at least 50 mM, at least 100 mM, at least 150 mM, at least 200 mM, at least 250 mM, at least 300 mM, at least 350 mM, at least 400 mM, at least 450 mM, at least 500 mM, at least 600 mM, at least 700 mM, at least 800 mM, at least 900 mM, at least 1M, at least 1.5M, at least 2M, at least 2.5M, at least 3M, at least 4M, at least 5M, at least 6M, at least 7M, at least 8M, or at least 9M.

In some embodiments, the disassembly buffer comprises a solubilizing agent or chaotropic agent at a concentration of at most 1 mM, at most 10 mM, at most 20 mM, at most 30 mM, at most 40 mM, at most 50 mM, at most 100 mM, at most 150 mM, at most 200 mM, at most 250 mM, at most 300 mM, at most 350 mM, at most 400 mM, at most 450 mM, at most 500 mM, at most 600 mM, at most 700 mM, at most 800 mM, at most 900 mM, at most 1M, at most 1.5M, at most 2M, at most 2.5M, at most 3M, at most 4M, at most 5M, at most 6M, at most 7M, at most 8M, at most 9M, or at most 10M.

In some embodiments, the disassembly buffer comprises a solubilizing agent or chaotropic agent at a concentration of about 1 mM, about 10 mM, about 20 mM, about 30 mM, about 40 mM, about 50 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1M, about 1.5M, about 2M, about 2.5M, about 3M, about 4M, about 5M, about 6M, about 7M, about 8M, about 9M, or about 10M.

In some embodiments, the disassembly buffer comprises a solubilizing agent or chaotropic agent at a concentration of about 1 mM-10M, about 1 mM-8M, about 1 mM-6M, about 1 mM-4M, about 1 mM-2M, about 1 mM-1M, about 1 mM-500 mM, about 1 mM-250 mM, about 1 mM-100 mM, about 1 mM-50 mM, about 1 mM-25 mM, about 1 mM-15 mM, about 1 mM-10 mM, about 1 mM-5 mM, about 5 mM-10M, about 5 mM-8M, about 5 mM-6M, about 5 mM-4M, about 5 mM-2M, about 5 mM-1M, about 5 mM-500 mM, about 5 mM-250 mM, about 5 mM-100 mM, about 5 mM-50 mM, about 5 mM-25 mM, about 5 mM-15 mM, about 5 mM-10 mM, about 10 mM-10M, about 10 mM-8M, about 10 mM-6M, about 10 mM-4M, about 10 mM-2M, about 10 mM-1M, about 10 mM-500 mM, about 10 mM-250 mM, about 10 mM-100 mM, about 10 mM-50 mM, about 10 mM-25 mM, about 10 mM-15 mM, about 25 mM-10M, about 25 mM-8M, about 25 mM-6M, about 25 mM-4M, about 25 mM-2M, about 25 mM-1M, about 25 mM-500 mM, about 25 mM-250 mM, about 25 mM-100 mM, about 25 mM-50 mM, about 50 mM-10M, about 50 mM-8M, about 50 mM-6M, about 50 mM-4M, about 50 mM-2M, about 50 mM-1M, about 50 mM-500 mM, about 50 mM-250 mM, about 50 mM-100 mM, about 100 mM-10M, about 100 mM-8M, about 100 mM-6M, about 100 mM-4M, about 100 mM-2M, about 100 mM-1M, about 100 mM-500 mM, about 100 mM-250 mM, about 250 mM-10M, about 250 mM-8M, about 250 mM-6M, about 250 mM-4M, about 250 mM-2M, about 250 mM-1M, about 250 mM-500 mM, about 500 mM-10M, about 500 mM-8M, about 500 mM-6M, about 500 mM-4M, about 500 mM-2M, about 500 mM-1M, about 1M-10M, about 1M-8M, about 1M-6M, about 1M-4M, about 1M-2M, about 2M-10M, about 2M-8M, about 2M-6M, about 2M-4M, about 4M-10M, about 4M-8M, or about 4M-6M.

In some embodiments, the disassembly buffer comprises the solubilizing agent (e.g., CHAPS) at a concentration of about 0.01-20%, about 0.1-20%, about 1-20%, about 5-20%, about 10-20%, about 0.01-15%, about 0.1-15%, about 1-15%, about 5-15%, about 10-15%, about 0.01-10%, about 0.1-10%, about 1-10%, about 5-10%, about 0.01-5%, about 0.1-5%, about 1-5%, about 0.01-1%, or about 0.1-1% (e.g., w/w, v/v, or w/v).

In some embodiments, the disassembly buffer comprises the solubilizing agent (e.g., CHAPS) at a concentration of about 5-10%.

The disassembly buffer can comprise a solubilizing agent and a reducing agent. The disassembly buffer can comprise a chaotropic agent and a reducing agent.

A disassembly buffer can comprise a mono valent salt (e.g., NaCl, KCl, LiCl, NaBr, or NaF).

The monovalent salt can be at a concentration of, for example, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 250 mM, at least 300 mM, at least 350 mM, at least 400 mM, at least 450 mM, at least 500 mM, at least 600 mM, at least 700 mM, at least 800 mM, at least 900 mM, at least 1000 mM, at least 1250 mM, at least 1500 mM, at least 2000 mM, at least 3000 mM, at least 4000 mM, or at least 5000 mM.

In some embodiments, the monovalent salt is at a concentration of at most 50 mM, at most 60 mM, at most 70 mM, at most 80 mM, at most 90 mM, at most 100 mM, at most 150 mM, at most 200 mM, at most 250 mM, at most 300 mM, at most 350 mM, at most 400 mM, at most 450 mM, at most 500 mM, at most 600 mM, at most 700 mM, at most 800 mM, at most 900 mM, at most 1000 mM, at most 1250 mM, at most 1500 mM, at most 2000 mM, at most 3000 mM, at most 4000 mM, or at most 5000 mM.

In some embodiments, the monovalent salt is at a concentration of about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1000 mM, about 1250 mM, about 1500 mM, about 2000 mM, about 3000 mM, about 4000 mM, or about 5000 mM.

In some embodiments, the monovalent salt is at a concentration of about 50-5000 mM, about 100-5000 mM, about 150-5000 mM, about 250-5000 mM, about 500-5000 mM, about 1000-5000 mM, about 50-2000 mM, about 100-2000 mM, about 150-2000 mM, about 250-2000 mM, about 500-2000 mM, about 1000-2000 mM, about 50-1500 mM, about 100-1500 mM, about 150-1500 mM, about 250-1500 mM, about 500-1500 mM, about 1000-1500 mM, about 50-1000 mM, about 100-1000 mM, about 150-1000 mM, about 250-1000 mM, about 500-1000 mM, about 50-750 mM, about 100-750 mM, about 150-750 mM, about 250-750 mM, about 500-750 mM, about 50-500 mM, about 100-500 mM, about 150-500 mM, about 250-500 mM, about 50-250 mM, about 100-250 mM, about 150-250 mM, or about 50-100 mM. The monovalent salt can be present at a concentration of about 50 mM to about 500 mM, e.g., at 50 mM or 500 mM.

In some embodiments, a disassembly buffer comprises a divalent salt or divalent cation e.g., from CaCl₂), MgCl₂, or ZnCl₂. In some embodiments, a disassembly buffer comprises CaCl₂). In some embodiments, a disassembly buffer comprises MgCl₂.

A disassembly buffer can comprise Tris. A disassembly buffer can comprise NaCl. A disassembly buffer can comprise NaP. A disassembly buffer can comprise glycerol. In some embodiments, a disassembly buffer comprises MgCl₂. A disassembly buffer can comprise urea. A disassembly buffer can comprise DTT.

A disassembly buffer can comprise urea and DTT. A disassembly buffer can comprise CHAPS, TCEP, and optionally Tris. A disassembly buffer can comprise about 5-10% CHAPS, about 10-20 mM TCEP, and optionally about 5-100 mM Tris. A disassembly buffer can comprise TCEP, MgCl2, and optionally Tris. A disassembly buffer can comprise urea, DTT, optionally NaCl, optionally NaP, and optionally glycerol. A disassembly buffer can comprise about 4-8M urea, about 10-80 mM DTT, optionally about 50-500 mM NaCl, optionally about 5-50 mM NaP, and optionally about 5-15% glycerol. A disassembly buffer can comprise urea, DTT, optionally NaCl, and optionally MgCl2.

Illustrative, non-limiting examples of disassembly buffers that can be used include: (1) 5-10% CHAPS, 10-20 mM TCEP, 50-100 mM Tris, pH 8.0; (2) 10 mM TCEP, 10 mM MgCl2, 50 mM Tris, pH 8.0; (3) 4M Urea, 50 mM DTT, 500 mM NaCl, 20 mM NaP, 10% glycerol, pH 7.4; (4) 4-6M Urea, 30-50 mM DTT, 500 mM NaCl, 10% glycerol; (5) 8M Urea, 66.67 mM DTT, 50 mM NaCl, 13.33 mM MgCl₂; (6) a buffer comprising about 12 mM GSH (reduced glutathione); (7) 10% CHAPS, 20 mM TCEP, 100 mM Tris pH 8.0; and (8) 5.33M Urea, 66.67 mM DTT, 500 mM NaCl, 20 mM NaP, 10% glycerol pH 7.4.

A disassembly buffer can comprise a component disclosed herein at a concentration of at least 1 μm, at least 10 μM, at least 100 μM, at least 1 nM, at least 10 nM, at least 100 nM, at least 1 μM, at least 10 μM, at least 100 μM, at least 1 mM, at least 10 mM, at least 100 mM, at least 250 mM, at least 500 mM, at least 1M, at least 2M, or at least 5M.

A disassembly buffer can comprise a component disclosed herein at a concentration of at most 1 μm, at most 10 μM, at most 100 μM, at most 1 nM, at most 10 nM, at most 100 nM, at most 1 μM, at most 10 μM, at most 100 μM, at most 1 mM, at most 10 mM, at most 100 mM, at most 250 mM, at most 500 mM, at most 1M, at most 2M, or at most 5M.

In some embodiments, capsid disassembly is conducted at a temperature of at least −20 °C, at least −10 °C, at least 0 °C, at least 2 °C, at least 4 °C, at least 6 °C, at least 8 °C, at least 10 °C, at least 12 °C, at least 14 °C, at least 16 °C, at least 18 °C, at least 20 °C, at least 22 °C, at least 25 °C, at least 30 °C, or at least 37 °C. In some embodiments, capsid disassembly is conducted at a temperature of at most 2 °C, at most 4 °C, at most 6 °C, at most 8 °C, at most 10 °C, at most 12 °C, at most 14 °C, at most 16 °C, at most 18 °C, at most 20 °C, at most 22 °C, at most 25 °C, at most 30 °C, at most 37 °C, at most 40 °C or at most 45 °C. In some embodiments, capsid disassembly is conducted at a temperature of about −20 °C, about −10 °C, about 0 °C, about 2 °C, about 4 °C, about 6 °C, about 8 °C, about 10 °C, about 12 °C, about 14 °C, about 16 °C, about 18 °C, about 20 °C, about 22 °C, about 25 °C, about 30 °C, or about 37 °C. In some embodiments, capsid disassembly is conducted at a temperature of about 2 °C to about 40 °C, 2 °C to about 37 °C, 2 °C. to about 25 °C, 2 °C to about 20 °C, 4 °C to about 40 °C, 4 °C to about 37 °C, 4 °C to about 25 °C, 4 °C to about 20 °C, 2 °C to about 8 °C, about 2 °C to about 4 °C, about 20 °C to about 37 °C, about 25 °C to about 37 °C, about 20 °C to about 30 °C, about 25 °C to about 30 °C, or about 30 °C to about 37 °C.

In some embodiments, capsid disassembly comprises incubating in a disassembly buffer for at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more.

A disassembly buffer can comprise a buffering agent to maintain a desirable pH, e.g., a basic pH, such as a pH of about 8 or about 8-10. Non-limiting examples of buffering agents that can be used include Tris, NaP, CHES, and CAPS.

The buffering agent can be at a concentration of, for example, at least 1 mM, 10 mM, at least 20 mM, at least 25 mM, at least 30 mM, at least 40 mM, at least 50 mM, at least 75 mM, at least 100 mM, at least 150 mM, at least 200 mM, at least 250 mM, or at least 500 mM.

The buffering agent can be at a concentration of, for example, at most 10 mM, at most 25 mM, of at most 50 mM, at most 100 mM, at most 150 mM, at most 200 mM, at most 250 mM, or at most 500 mM.

The buffering agent can be at a concentration of, for example, about 10 mM, about 25 mM, of about 50 mM, about 100 mM, about 150 mM, about 200 mM, about 250 mM, or about 500 mM.

In some embodiments, the buffering agent is at a concentration of about 1-500 mM, about 1-250 mM, about 1-150 mM, about 1-100 mM, about 1-75 mM, about 1-50 mM, about 1-25 mM, about 1-10 mM, about 5-500 mM, about 5-250 mM, about 5-150 mM, about 5-100 mM, about 5-75 mM, about 5-50 mM, about 5-25 mM, about 5-10 mM, about 10-500 mM, about 10-250 mM, about 10-150 mM, about 10-100 mM, about 10-75 mM, about 10-50 mM, about 10-25 mM, about 20-500 mM, about 20-250 mM, about 20-150 mM, about 20-100 mM, about 20-75 mM, about 20-50 mM, about 20-25 mM, about 50-500 mM, about 50-250 mM, about 50-150 mM, or about 50-100 mM.

In some embodiments, a disassembly buffer comprises a pH of about 3, about 4, about 5, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 10, or about 11. In some embodiments, a disassembly buffer comprises a pH of at least about 3, at least about 4, at least about 5, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, at least about 8, at least about 8.5, or at least about 9. In some embodiments, a disassembly buffer comprises a pH of at most about 7, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 10, or at most about 11. In some embodiments, a disassembly buffer comprises a pH of about 5-11, about 5-10, about 5-9, about 5-8, about 5-7.8, about 5-7.6, about 5-7.5, about 5-7.4, about 6-11, about 6-10, about 6-9, about 6-8, about 6-7.8, about 6-7.6, about 6-7.5, about 6-7.4, about 7-11, about 7-10, about 7-9, about 7-8, about 7-7.8, about 7-7.6, about 7-7.5, about 8-11, or about 8-10. In some embodiments, a disassembly buffer comprises a pH of about 7-10. In some embodiments, a disassembly buffer comprises a pH of about 8-10. In some embodiments, a disassembly buffer comprises a pH of about 8.

In some embodiments, a PNMA or endo-Gag polypeptide (e.g., PNMA2 or PNMA5) disclosed herein exhibits favorable disassembly properties in buffers with low levels of salt. The disassembly buffer can be a low salt buffer or a buffer with a low ionic strength. In some embodiments a disassembly buffer comprises a reducing agent and a low level of salt.

In some embodiments, a disassembly buffer comprises less than 10000, less than 9000, less than 8000, less than 7000, less than 6000, less than 5000, less than 4000, less than 3000, less than 2000, less than 1000, less than 900, less than 800, less than 700, less than 600, less than 500, less than 400, less than 300, less than 200, less than 150, or less than 100 mOsm/kg of salt.

In some embodiments, a disassembly buffer comprises high salt or high ionic strength. In some embodiments a disassembly buffer comprises a reducing agent and a high level of salt.

In some embodiments, a disassembly buffer comprises at least 8000, at least 7000, at least 6000, at least 5000, at least 4000, at least 3000, at least 2000, at least 1000, at least 900, at least 800, at least 700, at least 600, at least 500, at least 400, at least 300, or at least 200 mOsm/kg of salt.

In some embodiments, a disassembly buffer comprises about 75-8000, about 75-5000, about 75-4000, about 75-3000, about 75-2000, about 75-1000, about 75-750, about 75-600, about 75-500, about 75-400, about 75-300, about 75-250, about 75-200, about 150-8000, about 150-5000, about 150-4000, about 150-3000, about 150-2000, about 150-1000, about 150-750, about 150-600, about 150-500, about 150-400, about 150-300, about 150-250, about 150-200, about 200-8000, about 200-5000, about 200-4000, about 200-3000, about 200-2000, about 200-1000, about 200-750, about 200-600, about 200-500, about 200-400, about 200-300, about 200-250, about 250-8000, about 250-5000, about 250-4000, about 250-3000, about 250-2000, about 250-1000, about 250-750, about 250-600, about 250-500, about 250-400, about 250-300, about 270-8000, about 270-5000, about 270-4000, about 270-3000, about 270-2000, about 270-1000, about 270-750, about 270-600, about 270-500, about 270-400, about 270-300, about 300-8000, about 300-5000, about 300-4000, about 300-3000, about 300-2000, about 300-1000, about 300-750, about 300-600, about 300-500, about 300-400, about 500-8000, about 500-5000, about 500-4000, about 500-3000, about 500-2000, about 500-1000, about 500-750, about 500-600, about 1000-8000, about 1000-5000, about 1000-4000, about 1000-3000, about 1000-2000, about 1000-8000, about 1000-5000, about 1000-4000, about 1000-3000, about 1000-2000, about 2000-8000, about 2000-5000, about 2000-4000, about 2000-3000, about 2000-2000, about 2000-8000, about 2000-5000, about 2000-4000, or about 2000-3000 mOsm/kg of salt. In some embodiments, a disassembly buffer comprises about 500-1500 mOsm/kg of salt.

### C. Reassembly Buffer

In some embodiments, a reassembly buffer comprises one or more monovalent salts, for example, NaCl, KCl, LiCl, NaBr, NaF, or a combination thereof. In some embodiments, a reassembly buffer comprises one or more osmolytes, for example, glycerol, ethylene glycol, mannitol, glycine, glycine betaine, trehalose, sorbitol, or a combination thereof.

In some embodiments, a reassembly buffer does not contain or substantially lacks a chaotropic agent. In some embodiments, a reassembly buffer does not contain or substantially lacks a reducing agent.

A reassembly buffer can comprise a component disclosed herein at a concentration of at least 1 μm, at least 10 μM, at least 100 μM, at least 1 nM, at least 10 nM, at least 100 nM, at least 1 μM, at least 10 μM, at least 100 μM, at least 1 mM, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 250 mM, at least 300 mM, at least 350 mM, at least 400 mM, at least 450 mM, at least 500 mM, at least 1M, at least 2M, or at least 5M.

A reassembly buffer can comprise a component disclosed herein at a concentration of at most 1 μm, at most 10 μM, at most 100 μM, at most 1 nM, at most 10 nM, at most 100 nM, at most 1 μM, at most 10 μM, at most 100 μM, at most 1 mM, at most 10 mM, at most 50 mM, at most 60 mM, at most 70 mM, at most 80 mM, at most 90 mM, at most 100 mM, at most 150 mM, at most 200 mM, at most 250 mM, at most 300 mM, at most 350 mM, at most 400 mM, at most 450 mM, at most 500 mM, at most 1M, at most 2M, or at most 5M.

A reassembly buffer can comprise a component disclosed herein at a concentration of about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1000 mM, about 1250 mM, about 1500 mM, about 2000 mM, about 3000 mM, about 4000 mM, or about 5000 mM.

In some embodiments, a reassembly buffer comprises a physiological buffer. In some embodiments, a reassembly buffer is or comprises a balanced salt solution. In some embodiments, a reassembly buffer is or comprises a saline, e.g., a buffered saline. In some embodiments, a reassembly buffer is or comprises phosphate buffered saline (PBS). In some embodiments, a reassembly buffer is or comprises HBSS. In some embodiments, a reassembly buffer is or comprises ringer's solution. In some embodiments, a reassembly buffer comprises sodium phosphate.

In some embodiments, a RTL, PNMA or endo-Gag polypeptide of the disclosure (e.g., RTL10 or PEG10) exhibits favorable reassembly properties in buffers with low or physiological levels of salt. In some embodiments, a reassembly buffer comprises low salt or low ionic strength, e.g., with less than 250 mM of a salt, such as NaCl. In some embodiments, a reassembly buffer comprises high salt or high ionic strength. In some embodiments, a reassembly buffer is not a balanced salt solution.

In some embodiments, a reassembly buffer comprises less than 10000, less than 9000, less than 8000, less than 7000, less than 6000, less than 5000, less than 4000, less than 3000, less than 2000, less than 1000, less than 900, less than 800, less than 700, less than 600, less than 500, less than 400, less than 300, or less than 200 mOsm/kg of salt.

In some embodiments, a reassembly buffer comprises more than 200 mOsm/kg of salt. In some embodiments, a reassembly buffer comprises at least 8000, at least 7000, at least 6000, at least 5000, at least 4000, at least 3000, at least 2000, at least 1000, at least 900, at least 800, at least 700, at least 600, at least 500, at least 400, at least 300, or at least 200 mOsm/kg of salt.

In some embodiments, a reassembly buffer comprises about 75-8000, about 150-5000, about 150-4000, about 150-3000, about 150-2000, about 150-1000, about 150-750, about 150-600, about 150-500, about 150-400, about 150-300, about 150-250, about 150-200, about 200-8000, about 200-5000, about 200-4000, about 200-3000, about 200-2000, about 200-1000, about 200-750, about 200-600, about 200-500, about 200-400, about 200-300, about 200-250, about 250-8000, about 250-5000, about 250-4000, about 250-3000, about 250-2000, about 250-1000, about 250-750, about 250-600, about 250-500, about 250-400, about 250-300, about 270-8000, about 270-5000, about 270-4000, about 270-3000, about 270-2000, about 270-1000, about 270-750, about 270-600, about 270-500, about 270-400, about 270-300, about 300-8000, about 300-5000, about 300-4000, about 300-3000, about 300-2000, about 300-1000, about 300-750, about 300-600, about 300-500, about 300-400, about 500-8000, about 500-5000, about 500-4000, about 500-3000, about 500-2000, about 500-1000, about 500-750, about 500-600, about 1000-8000, about 1000-5000, about 1000-4000, about 1000-3000, about 1000-2000, about 1000-8000, about 1000-5000, about 1000-4000, about 1000-3000, about 1000-2000, about 2000-8000, about 2000-5000, about 2000-4000, about 2000-3000, about 2000-2000, about 2000-8000, about 2000-5000, about 2000-4000, or about 2000-3000 mOsm/kg of salt.

In some embodiments, a reassembly buffer comprises glycerol. The glycerol can be present at a concentration of, for example, at least 1%, at least 5%, at least 10%, at least 15%, at least 20%, at least 25%, at least 30%, at least 35%, at least 40%, at least 45%, or at least 50% (e.g., w/w, v/v, or w/v). The glycerol can be present at a concentration of, for example, at most 5%, at most 10%, at most 15%, at most 20%, at most 25%, at most 30%, at most 35%, at most 40%, at most 45%, or at most 50% (e.g., w/w, v/v, or w/v). The glycerol can be present at a concentration of, for example, about 5%, about 10%, about 15%, about 20%, about 25%, about 30%, about 35%, about 40%, about 45%, or about 50% (e.g., w/w, v/v, or w/v). The glycerol can be present at a concentration of, for example, 1-50%, 5-50%, 10-50%, 15-50%, 20-50%, 25-50%, 30-50%, 1-30%, 5-30%, 10-30%, 15-30%, 20-30%, 25-30%, 1-20%, 5-20%, 10-20%, 15-20%, 1-15%, 5-15%, 10-15%, 1-10%, or 5-10% (e.g., w/w, v/v, or w/v).

In some embodiments, a reassembly buffer comprises a monovalent salt. The monovalent salt can be, for example, NaCl, KCl, LiCl, NaBr, or NaF.

The monovalent salt can be at a concentration of, for example, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 250 mM, at least 300 mM, at least 350 mM, at least 400 mM, at least 450 mM, at least 500 mM, at least 600 mM, at least 700 mM, at least 800 mM, at least 900 mM, at least 1000 mM, at least 1250 mM, at least 1500 mM, at least 2000 mM, at least 3000 mM, at least 4000 mM, or at least 5000 mM.

In some embodiments, the monovalent salt is at a concentration of at most 50 mM, at most 60 mM, at most 70 mM, at most 80 mM, at most 90 mM, at most 100 mM, at most 150 mM, at most 200 mM, at most 250 mM, at most 300 mM, at most 350 mM, at most 400 mM, at most 450 mM, at most 500 mM, at most 600 mM, at most 700 mM, at most 800 mM, at most 900 mM, at most 1000 mM, at most 1250 mM, at most 1500 mM, at most 2000 mM, at most 3000 mM, at most 4000 mM, or at most 5000 mM.

In some embodiments, the monovalent salt is at a concentration of about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1000 mM, about 1250 mM, about 1500 mM, about 2000 mM, about 3000 mM, about 4000 mM, or about 5000 mM.

In some embodiments, the monovalent salt is at a concentration of about 50-5000 mM, about 100-5000 mM, about 150-5000 mM, about 250-5000 mM, about 500-5000 mM, about 1000-5000 mM, about 50-2000 mM, about 100-2000 mM, about 150-2000 mM, about 250-2000 mM, about 500-2000 mM, about 1000-2000 mM, about 50-1500 mM, about 100-1500 mM, about 150-1500 mM, about 250-1500 mM, about 500-1500 mM, about 1000-1500 mM, about 50-1000 mM, about 100-1000 mM, about 150-1000 mM, about 250-1000 mM, about 500-1000 mM, about 50-750 mM, about 100-750 mM, about 150-750 mM, about 250-750 mM, about 500-750 mM, about 50-500 mM, about 100-500 mM, about 150-500 mM, about 250-500 mM, about 50-250 mM, about 100-250 mM, about 150-250 mM, or about 50-100 mM.

In some embodiments, a reassembly buffer comprises Na₂HPO₄. In some embodiments, a reassembly buffer comprises KH₂PO₄.

In some embodiments, a reassembly buffer comprises a divalent salt or divalent cation e.g., from CaCl₂), MgCl₂, or ZnCl₂. In some embodiments, a reassembly buffer comprises CaCl₂). In some embodiments, a reassembly buffer comprises MgCl₂. In some embodiments, a reassembly buffer comprises a buffering agent. In some embodiments, a reassembly buffer comprises Tris. In some embodiments, a reassembly buffer comprises imidazole. In some embodiments, a reassembly buffer comprises DTT.

The divalent cation can be present at a concentration of, for example, at least 1 μM, at least 10 μM, at least 25 μM, at least 50 μM, at least 100 μM, at least 500 μM, at least 1 mM, at least 10 mM, at least 20 mM, at least 30 mM, at least 40 mM, at least 50 mM, at least 75 mM, at least 100 mM, or at least 250 mM.

The divalent cation can be present at a concentration of, for example, at most 25 μM, at most 50 μM, at most 100 μM, at most 500 μM, at most 1 mM, at most 10 mM, at most 20 mM, at most 30 mM, at most 40 mM, at most 50 mM, at most 75 mM, at most 100 mM, or at most 250 mM.

The divalent cation can be present at a concentration of, for example, about 1 μM, about 10 μM, about 25 μM, about 50 μM, about 100 μM, about 500 μM, about 1 mM, about 10 mM, about 20 mM, about 30 mM, about 40 mM, about 50 mM, about 75 mM, about 100 mM, or about 250 mM.

The divalent cation can be present at a concentration of, for example, about 1 μM-250 mM, about 10 μM-250 mM, about 50 μM-250 mM, about 100 μM-250 mM, about 1 mM-250 mM, about 10 mM-250 mM, about 25 mM-250 mM, about 50-250 mM, about 100-250 mM, about 1 μM-100 mM, about 10 μM-100 mM, about 50 μM-100 mM, about 100 μM-100 mM, about 1 mM-100 mM, about 10 mM-100 mM, about 25 mM-100 mM, about 50-100 mM, about 1 μM-50 mM, about 10 μM-50 mM, about 50 μM-50 mM, about 100 μM-50 mM, about 1 mM-50 mM, about 10 mM-50 mM, about 25 mM-50 mM, about 1 μM-10 mM, about 10 μM-10 mM, about 50 μM-10 mM, about 100 μM-10 mM, about 1 mM-10 mM, about 10 mM-10 mM, about 25 mM-10 mM, about 50-10 mM, about 1 μM-1 mM, about 10 μM-1 mM, about 50 μM-1 mM, about 100 μM-1 mM, about 1 μM-100 μM, about 10 μM-100 μM, or about 50 μM-100 μM.

In some embodiments, a reassembly buffer comprises a buffering agent, e.g., an acidic buffer.

Non-limiting examples of buffering agents that can be used include MES, Tris, NaP, CHES, and CAPS.

The buffering agent can be at a concentration of, for example, at least 1 mM, 10 mM, at least 20 mM, at least 25 mM, at least 30 mM, at least 40 mM, at least 50 mM, at least 75 mM, at least 100 mM, at least 150 mM, at least 200 mM, at least 250 mM, or at least 500 mM.

The buffering agent can be at a concentration of, for example, at most 10 mM, at most 25 mM, of at most 50 mM, at most 100 mM, at most 150 mM, at most 200 mM, at most 250 mM, or at most 500 mM.

The buffering agent can be at a concentration of, for example, about 10 mM, about 25 mM, of about 50 mM, about 100 mM, about 150 mM, about 200 mM, about 250 mM, or about 500 mM.

In some embodiments, the buffering agent is at a concentration of about 1-500 mM, about 1-250 mM, about 1-150 mM, about 1-100 mM, about 1-75 mM, about 1-50 mM, about 1-25 mM, about 1-10 mM, about 5-500 mM, about 5-250 mM, about 5-150 mM, about 5-100 mM, about 5-75 mM, about 5-50 mM, about 5-25 mM, about 5-10 mM, about 10-500 mM, about 10-250 mM, about 10-150 mM, about 10-100 mM, about 10-75 mM, about 10-50 mM, about 10-25 mM, about 20-500 mM, about 20-250 mM, about 20-150 mM, about 20-100 mM, about 20-75 mM, about 20-50 mM, about 20-25 mM, about 50-500 mM, about 50-250 mM, about 50-150 mM, or about 50-100 mM.

In some embodiments, a reassembly buffer comprises an acidic buffer (e.g., pH <7, such as a pH of about 6.0) with a divalent cation. For example, in some embodiments RTL10 exhibits favorable reassembly properties in an acidic buffer with a divalent cation (e.g., 50 mM MES, 40 mM MgCl₂, pH 6.0).

In some embodiments, a reassembly buffer comprises a physiological buffer. In some embodiments, a reassembly buffer is or comprises a balanced salt solution. In some embodiments, a reassembly buffer is or comprises a saline, e.g., a buffered saline. In some embodiments, a reassembly buffer is or comprises phosphate buffered saline (PBS). In some embodiments, a reassembly buffer is or comprises HBSS. In some embodiments, an reassembly buffer is or comprises ringer's solution. In some embodiments, an reassembly buffer comprises sodium phosphate.

In some embodiments, a reassembly buffer comprises one or more osmolytes, for example, glycerol, ethylene glycol, mannitol, glycine, glycine betaine, trehalose, sorbitol, or a combination thereof.

In some embodiments, a reassembly buffer comprises glycerol. The glycerol can be present at a concentration of, for example, at least 1%, at least 5%, at least 10%, at least 15%, at least 20%, at least 25%, at least 30%, at least 35%, at least 40%, at least 45%, or at least 50% (e.g., w/w, v/v, or w/v). The glycerol can be present at a concentration of, for example, at most 5%, at most 10%, at most 15%, at most 20%, at most 25%, at most 30%, at most 35%, at most 40%, at most 45%, or at most 50% (e.g., w/w, v/v, or w/v). The glycerol can be present at a concentration of, for example, about 5%, about 10%, about 15%, about 20%, about 25%, about 30%, about 35%, about 40%, about 45%, or about 50% (e.g., w/w, v/v, or w/v). The glycerol can be present at a concentration of, for example, 1-50%, 5-50%, 10-50%, 15-50%, 20-50%, 25-50%, 30-50%, 1-30%, 5-30%, 10-30%, 15-30%, 20-30%, 25-30%, 1-20%, 5-20%, 10-20%, 15-20%, 1-15%, 5-15%, 10-15%, 1-10%, or 5-10% (e.g., w/w, v/v, or w/v).

In some embodiments, a reassembly buffer does not contain or substantially lacks a chaotropic agent. In some embodiments, a reassembly buffer does not contain or substantially lacks a reducing agent. In some embodiments, a reassembly buffer comprises DTT, e.g., at a low concentration.

The reassembly buffer may contain a heterologous cargo (e.g., nucleic acid). In some embodiments, the heterologous cargo (e.g., RNA) can promote capsid reassembly.

In some embodiments, an reassembly buffer comprises a monovalent salt, a divalent cation, and optionally a buffering agent. In some embodiments, an reassembly buffer comprises NaCl, ZnCl₂, and optionally Tris. In some embodiments, an reassembly buffer comprises about 150 mM NaCl, about 10 μM ZnCl₂, and optionally about 25 mM Tris. In some embodiments, an reassembly buffer comprises about 150 mM NaCl, about 1 mM DTT, about 10 μM ZnCl₂, and optionally about 50 mM Tris.

In some embodiments, a reassembly buffer comprises MES and MgCl2. In some embodiments, a reassembly buffer comprises about 50 mM MES and about 40 mM MgCl2.

In some embodiments, a reassembly buffer comprises NaCl, KCl, Na₂HPO₄, and KH₂PO₄.

Illustrative, non-limiting examples of reassembly buffers include: (1) 137 mM NaCl, 2.7 mM KCl, 10 mM Na₂HPO₄, 1.8 mM KH₂PO₄; (2) 137 mM NaCl, 2.7 mM KCl, 8 mM Na₂HPO₄, 1.5 mM KH₂PO₄, 0.9 mM CaCl₂, 0.5 mM MgCl₂); (3) 75 mM NaCl, 50 mM Tris, 10% glycerol, pH 8.0; and (4) 20 mM Na₂HPO₄/NaH₂PO₄, 0.5 M NaCl, 10% glycerol, 40 mM imidazole, 1 mM DTT, pH 7.4. In some embodiments the reassembly buffer does not contain a chaotropic agent and/or a reducing agent. The reassembly buffer can contain a heterologous cargo.

Illustrative, non-limiting examples of reassembly buffers that can be used for PEG10 include: (1) a low salt buffer with 150 mM NaCl, 25 mM Tris, 10 μM ZnCl₂, pH 7.5; and (2) a low salt buffer with 150 mM NaCl, 1 mM DTT, 10 μM ZnCl₂, 50 mM Tris, pH 8.0.

An illustrative, non-limiting example of an reassembly buffer that can be used for RTL10 is an acidic buffer plus divalent cation: 50 mM MES (2-(N-morpholino) ethanesulfonic acid), 40 mM MgCl₂, pH 6.0.

In some embodiments, capsid reassembly is conducted at a temperature of at least-20 °C, at least −10 °C, at least 0 °C, at least 2 °C, at least 4 °C, at least 6 °C, at least 8 °C, at least 10 °C, at least 12 °C, at least 14 °C, at least 16 °C, at least 18 °C, at least 20 °C, at least 22 °C, at least 25 °C, at least 30 °C, or at least 37 °C. In some embodiments, capsid reassembly is conducted at a temperature of at most 2 °C, at most 4 °C, at most 6 °C, at most 8 °C, at most 10 °C, at most 12 °C, at most 14 °C, at most 16 °C, at most 18 °C, at most 20 °C, at most 22 °C, at most 25 °C, at most 30 °C, at most 37 °C, at most 40 °C or at most 45 °C. In some embodiments, capsid reassembly is conducted at a temperature of about −20 °C, about −10 °C, about 0 °C, about 2 °C, about 4 °C, about 6 °C, about 8 °C, about 10 °C, about 12 °C, about 14 °C, about 16 °C, about 18 °C, about 20 °C, about 22 °C, about 25 °C, about 30 °C, or about 37 °C. In some embodiments, capsid reassembly is conducted at a temperature of about 2 °C to about 40 °C, 2 °C to about 37 °C, 2 °C to about 25 °C, 2 °C to about 20 °C, 4 °C to about 40 °C, 4 °C to about 37 °C, 4 °C to about 25 °C, 4 °C to about 20 °C, 2 °C to about 8 °C, about 2 °C to about 4 °C, about 20 °C to about 37 °C, about 25 °C to about 37 °C, about 20 °C to about 30 °C, about 25 °C to about 30 °C, or about 30 °C to about 37 °C.

In some embodiments, capsid reassembly comprises incubating in a reassembly buffer for at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more.

In some embodiments, a reassembly buffer comprises a pH of about 3, about 4, about 5, about 6, about 6.1, about 6.2, about 6.3, about 6.4, about 6.5, about 6.6, about 6.7, about 6.8, about 6.9, about 7, about 7.1, about 7.2, about 7.3, about 7.4, about 7.5, about 7.6, about 7.7, about 7.8, about 7.9, about 8, about 8.1, about 8.2, about 8.3, about 8.4, about 8.5, about 8.6, about 8.7, about 8.8, about 8.9, about 9, about 10, or about 11. In some embodiments, a reassembly buffer comprises a pH of at least about 3, at least about 4, at least about 5, at least about 6, at least about 6.1, at least about 6.2, at least about 6.3, at least about 6.4, at least about 6.5, at least about 6.6, at least about 6.7, at least about 6.8, at least about 6.9, at least about 7, at least about 7.1, at least about 7.2, at least about 7.3, at least about 7.4, at least about 7.5, at least about 7.6, at least about 7.7, at least about 7.8, at least about 7.9, or at least about 8. In some embodiments, a reassembly buffer comprises a pH of at most about 6, at most about 6.5, at most about 7, at most about 7.5, at most about 7.6, at most about 7.7, at most about 7.8, at most about 7.9, at most about 8, at most about 8.1, at most about 8.2, at most about 8.3, at most about 8.4, at most about 8.5, at most about 8.6, at most about 8.7, at most about 8.8, at most about 8.9, at most about 9, at most about 10, or at most about 11. In some embodiments, a reassembly buffer comprises a pH of about 5-10, 5-9, 5-8, 5-7.8, 5-7.6, 5-7.5, 5-7.4, 5-7, 5-6.5, 5-6, 8-8.5, 6-9, 6-8, 6-7.8, 6-7.6, 6-7.5, 6-7.4, 6-7, 6-6.5, 7-9, 7-8.5, 7-8, 7-7.8, 7-7.6, 7-7.5, 7.5-8, 8-10, 8-9.5, 8-9, or 8-8.5.

A reassembly buffer can comprise a component disclosed herein at a concentration of at least 1 μM, at least 10 μM, at least 100 μM, at least 1 nM, at least 10 nM, at least 100 nM, at least 1 μM, at least 10 μM, at least 100 μM, at least 1 mM, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 250 mM, at least 300 mM, at least 350 mM, at least 400 mM, at least 450 mM, at least 500 mM, at least 1M, at least 2M, or at least 5M.

A reassembly buffer can comprise a component disclosed herein at a concentration of at most 1 μM, at most 10 μM, at most 100 μM, at most 1 nM, at most 10 nM, at most 100 nM, at most 1 μM, at most 10 μM, at most 100 μM, at most 1 mM, at most 10 mM, at most 50 mM, at most 60 mM, at most 70 mM, at most 80 mM, at most 90 mM, at most 100 mM, at most 150 mM, at most 200 mM, at most 250 mM, at most 300 mM, at most 350 mM, at most 400 mM, at most 450 mM, at most 500 mM, at most 1M, at most 2M, or at most 5M.

A reassembly buffer can comprise a component disclosed herein at a concentration of about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 600 mM, about 700 mM, about 800 mM, about 900 mM, about 1000 mM, about 1250 mM, about 1500 mM, about 2000 mM, about 3000 mM, about 4000 mM, or about 5000 mM.

### D. Cargo Delivery

In some instances, the RTL-based or PNMA-based capsid (e.g., RTL10, PEG10, PNMA2, PNMA5, or another RTL or PNMA disclosed herein) or endo-Gag-based capsid is formulated for systemic administration. In some instances, the RTL-based or PNMA-based capsid or endo-Gag-based capsid is formulated for local administration. In some instances, the RTL-based or PNMA-based capsid or endo-Gag-based capsid is formulated for parenteral (e.g., intra-arterial, intra-articular, intradermal, intralesional, intramuscular, intraocular, intraosseous infusion, intraperitoneal, intrathecal, intravenous, intravitreal, or subcutaneous) administration. In some instances, the RTL-based or PNMA-based capsid or endo-Gag-based capsid is further formulated for topical administration. In some instances, the RTL-based or PNMA-based capsid or endo-Gag-based capsid is further formulated for oral administration. In some instances, the RTL-based or PNMA-based capsid or endo-Gag-based capsid is further formulated for sublingual administration. In some instances, the RTL-based or PNMA-based capsid or endo-Gag-based capsid is further formulated for aerosol administration.

In certain embodiments, also described herein is a use of a RTL-based or PNMA-based capsid or endo-Gag-based capsid for delivery of a cargo to a site of interest.

In some instances, methods of the disclosure comprise contacting a cell with a RTL-based or PNMA-based capsid or endo-Gag-based capsid. The contacting can be in vitro, ex vivo, or in vivo. In some embodiments, the contacting is in vivo at a site of interest.

In some instances, the method comprises contacting a cell at the site of interest with a RTL-based or PNMA-based capsid (e.g., RTL10 or PEG10-based) or endo-Gag-based capsid for a time sufficient to facilitate cellular uptake of the capsid. The time sufficient to facilitate cellular uptake of the capsid can be at least about 5 minutes, at least about 10 minutes, at least about 20 minutes, at least about 30 minutes, at least about 1 hour, at least about 2 hours, at least about 4 hours, at least about 6 hours, at least about 8 hours, at least about 10 hours, at least about 12 hours, at least about 16 hours, at least about 20 hours, at least about 24 hours, or more. In some embodiments, the time sufficient to facilitate cellular uptake of the capsid is at most about 5 minutes, at most about 10 minutes, at most about 20 minutes, at most about 30 minutes, at most about 1 hour, at most about 2 hours, at most about 4 hours, at most about 6 hours, at most about 8 hours, at most about 10 hours, at most about 12 hours, at most about 16 hours, at most about 20 hours, at most about 24 hours, or at most about 48 hours.

In some embodiments, the method comprises contacting a cell with a RTL-based or PNMA-based capsid or endo-Gag-based capsid at a concentration sufficient to elicit a desired effect on the cell. The concentration sufficient to elicit the desired effect can be at least about 0.001 pg/mL, at least about 0.01 pg/mL, at least about 0.1 pg/mL, at least about 1 pg/mL, at least about 10 pg/mL, at least about 100 pg/mL, at least about 1 ng/mL, at least about 10 ng/ml, at least about 100 ng/ml, at least about 1 μg/mL, at least about 10 μg/mL, at least about 100 μg/mL, at least about 1 mg/mL, at least about 10 mg/mL, or at least about 100 mg/mL.

In some cases, the cell is a muscle cell, a skin cell, a blood cell, or an immune cell (e.g., a T cell or a B cell).

In some instances, the cell is a tumor cell, e.g., a solid tumor cell or a cell from a hematologic malignancy. In some cases, the solid tumor cell is a cell from a bladder cancer, breast cancer, brain cancer, colorectal cancer, kidney cancer, liver cancer, lung cancer, pancreatic cancer, prostate cancer, skin cancer, stomach cancer, or thyroid cancer. In some cases, the cell from a hematologic malignancy is from a B-cell malignancy or a T-cell malignancy. In some cases, the cell is from a leukemia, a lymphoma, a myeloma, chronic lymphocytic leukemia (CLL), small lymphocytic lymphoma (SLL), diffuse large B cell lymphoma (DLBCL), follicular lymphoma, mantle cell lymphoma, Burkitt lymphoma, cutaneous T-cell lymphoma, peripheral T cell lymphoma, multiple myeloma, plasmacytoma, acute lymphoblastic leukemia (ALL), acute myeloid leukemia (AML), or chronic myeloid leukemia (CML).

In some embodiments, the cell is a somatic cell. In some instances, the cell is a blood cell, a skin cell, a connective tissue cell, a bone cell, a muscle cell, or a cell from an organ.

In some embodiments, the cell is an epithelial cell, a connective tissue cell, a muscular cell, or a neuron.

In some instances, the cell is an endodermal cell, a mesodermal cell, or an ectodermal cell. In some instances, the endoderm comprises cells of the respiratory system, the intestine, the liver, the gallbladder, the pancreas, the islets of Langerhans, the thyroid, or the hindgut. In some cases, the mesoderm comprises osteochondroprogenitor cells, muscle cells, cells from the digestive system, renal stem cells, cells from the reproductive system, cells from the circulatory system (such as endothelial cells). Exemplary cells from the ectoderm comprise epithelial cells, cells of the anterior pituitary, cells of the peripheral nervous system, cells of the neuroendocrine system, cells of the eyes, cells of the central nervous system, cells of the ependymal, or cells of the pineal gland. In some cases, cells derived from the central and peripheral nervous system comprise neurons, Schwann cells, satellite glial cells, oligodendrocytes, or astrocytes. In some cases, neurons further comprise interneurons, pyramidal neurons, GABAergic neurons, dopaminergic neurons, serotoninergic neurons, glutamatergic neurons, motor neurons from the spinal cord, or inhibitory spinal neurons.

In some embodiments, the cell is a stem cell or a progenitor cell. In some cases, the cell is a mesenchymal stem or progenitor cell. In other cases, the cell is a hematopoietic stem or progenitor cell.

In some cases, a target protein is overexpressed or is depleted in the cell. In some cases, the target protein is overexpressed in the cell. In additional cases, the target protein is depleted in the cell.

In some cases, a target gene in the cell has one or more mutations. In some cases, the cell comprises an impaired splicing mechanism.

In some instances, the RTL-based or PNMA-based capsid (e.g., PEG10 or RTL10) is administered systemically to a subject in need thereof. In other instances, the RTL-based or PNMA-based capsid or endo-Gag-based capsid is administered locally to a subject in need thereof. In some embodiments, the RTL-based or PNMA-based capsid or endo-Gag-based capsid is administered parenterally, orally, topically, via sublingual, or by aerosol to a subject in need thereof. In some cases, the RTL-based or PNMA-based capsid or endo-Gag-based capsid is administered parenterally to a subject in need thereof. In other cases, the RTL-based or PNMA-based capsid or endo-Gag-based capsid is administered orally to a subject in need thereof. In additional cases, the RTL-based or PNMA-based capsid or endo-Gag-based capsid is administered topically, via sublingual, or by aerosol to a subject in need thereof.

In some embodiments, the RTL-based or PNMA-based capsid (e.g., RTL10-based, PEG10-based, PNMA2-based or PNMA5-based) or endo-Gag-based capsid is for in vitro use. In some instances, the RTL-based or PNMA-based capsid or endo-Gag-based capsid is for ex vivo use. In some cases, the RTL-based or PNMA-based capsid or endo-Gag-based capsid is for in vivo use.

### VI. KITS/ARTICLE OF MANUFACTURE

Disclosed herein, in certain embodiments, are kits and articles of manufacture for use with one or more compositions or methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. In one embodiment, the containers are formed from a variety of materials such as glass or plastic.

For example, the container(s) can include a recombinant or engineered RTL, PNMA or endo-Gag polypeptide described herein. Such kits optionally include an identifying description or label or instructions relating to its use in the methods described herein. For example, a kit can include labels listing contents and/or instructions for use, and package inserts with instructions for use.

### VII. CERTAIN TERMINOLOGIES

Unless defined otherwise, all technical and scientific terms used herein have the same meaning as is commonly understood. It is to be understood that the detailed description is exemplary and explanatory only and not restrictive of any subject matter claimed. In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

Although various features of the disclosure may be described in the context of a single embodiment, the features may also be provided separately or in any suitable combination. Conversely, although the disclosure may be described herein in the context of separate embodiments for clarity, the disclosure may also be implemented in a single embodiment.

Reference in the specification to “some embodiments”, “an embodiment”, “one embodiment” or “other embodiments” means that a particular feature, structure, or characteristic described in connection with the embodiments is included in at least some embodiments, but not necessarily all embodiments, of the inventions.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The section headings used herein are for organizational purposes only and are not to be construed as limiting the subject matter described.

As used herein, the terms “individual(s)”, “subject(s)” and “patient(s)” mean any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

### VIII. EXAMPLES

The following examples are included for illustrative purposes only and are not intended to limit the scope of the invention.

### Example 1: Efficiency of Capsid Assembly, Disassembly, and Reassembly

This example provides illustrative protocols that can be used to calculate the efficiency of assembly, disassembly, and reassembly of capsids disclosed herein that comprise endogenous Gag polypeptides (e.g., native or engineered endogenous Gag polypeptides, such as native or engineered PNMA polypeptides) and the recovery of assembled, disassembled, and reassembled endogenous Gag polypeptides. Also provided are methods to purify assembled and reassembled capsids.

Isolated capsids are disassembled into monomers and other non-capsid forms by incubation in a disassembly buffer. The disassembly buffer can comprise a solubilization/chaotropic agent (e.g. urea or CHAPS) and/or a reducing agent (e.g. GSH, TCEP, β-ME, or DTT).

A disassembly buffer can comprise a monovalent salt (e.g., NaCl, KCl, or NaBr, at a concentration of about 50 mM to about 500 mM, e.g., at 50 mM or 500 mM). A disassembly buffer can comprise a reducing agent (e.g. GSH, TCEP, β-ME, or DTT at a concentration of about 5-70 mM, e.g., 5 mM, 10 mM, 12 mM, 20 mM, 30 mM, 50 mM, or 66.67 mM). A disassembly buffer can be buffered to about pH 7.4 or to a basic pH of about 8-10, for example, with a Tris, NaP, CHES, or CAPS buffer.

Illustrative, non-limiting examples of disassembly buffers that can be used include: (1) 5-10% CHAPS, 10-20 mM TCEP, 50-100 mM Tris, pH 8.0; (2) 10 mM TCEP, 10 mM MgCl₂, 50 mM Tris, pH 8.0; (3) 4M Urea, 50 mM DTT, 500 mM NaCl, 20 mM NaP, 10% glycerol, pH 7.4; (4) 4-6M Urea, 30-50 mM DTT, 500 mM NaCl, 10% glycerol; (5) 8M Urea, 66.67 mM DTT, 50 mM NaCl, 13.33 mM MgCl₂; and (6) a buffer comprising about 12 mM GSH (reduced glutathione).

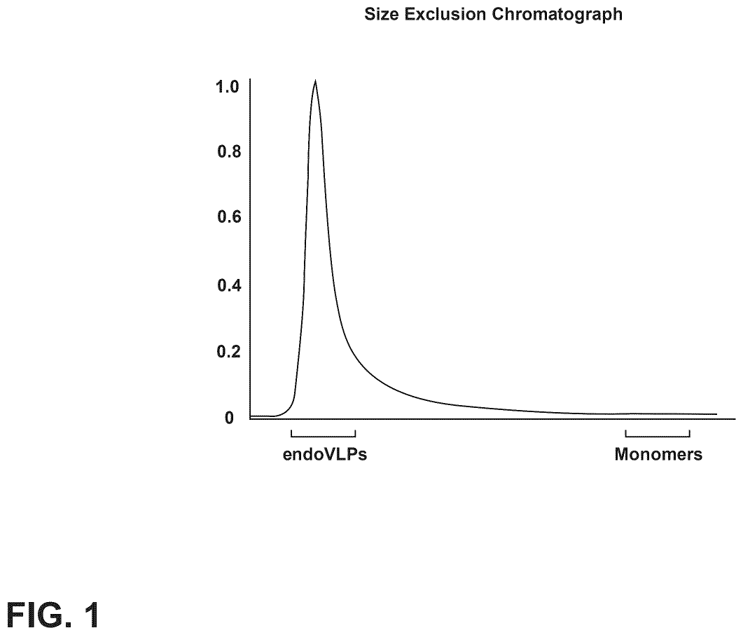

The efficiency of disassembly can be determined by MADLS or size exclusion chromatography (SEC). MADLS shows a monomer peak (e.g., at about 1 nm) and a capsid peak (e.g., at about 30 nm). SEC separates capsids in the void volume from monomers that enter the column matrix and are eluted according to their size. For SEC, samples are loaded onto a Superose 6 Increase 10/300 GL column (GE Healthcare, Cat #29091596) in PBS-500 buffer (1X PBS pH 7.5, 363 mM NaCl). 1 Column Volume of PBS-500 buffer is run through the column and capsids elute in the column Void Volume. Monomers and other non-capsid forms are eluted from the matrix later, after approximately 15 mL of PBS-500 has eluted. Results from an SEC analysis of isolated PNMA2 capsids are shown in FIG. 1. For MADLS or SEC, the efficiency of disassembly is determined by quantifying the percent of total protein in the monomer peak. When the efficiency of disassembly is greater than about 95%, recovery of disassembled endo-Gag polypeptides is determined by comparing the amount of protein in solution after disassembly to the amount of protein in solution before disassembly.

Disassembled endo-Gag monomers are reassembled into capsids by incubation in a reassembly buffer. The reassembly buffer can be a physiological buffer, for example, phosphate a buffered saline (PBS). Illustrative, non-limiting examples of reassembly buffers include: (1) 137 mM NaCl, 2.7 mM KCl, 10 mM Na₂HPO₄, 1.8 mM KH₂PO₄; (2) 137 mM NaCl, 2.7 mM KCl, 8 mM Na₂HPO₄, 1.5 mM KH₂PO₄, 0.9 mM CaCl₂), 0.5 mM MgCl₂); (3) 75 mM NaCl, 50 mM Tris, 10% Glycerol, pH 8.0; and (4) 20 mM Na₂HPO₄/NaH₂PO₄, 0.5 M NaCl, 10% glycerol, 40 mM imidazole, 1 mM DTT, pH 7.4. In some embodiments the reassembly buffer does not contain a chaotropic agent and/or a reducing agent. The reassembly buffer may contain a heterologous cargo.

After reassembly, aggregates are removed by centrifugation at 15,000×g for 5 min at +4 °C. The efficiency of reassembly can be determined by comparing the amounts of endo-Gag polypeptides in the monomer and capsid peaks as assayed by MADLS or SEC. Reassembled capsid compositions where at least 95% of the endo-Gag polypeptides are incorporated into capsids can be used directly for cargo delivery to cells or can be further purified. Further purification can be recommended when reassembly is less than 95% efficient. Reassembled capsids can be purified by SEC or Ion-exchange chromatography (IEC). The recovery of reassembled capsids is determined by comparing the amount of endo-Gag polypeptides in reassembled capsids (with or without further purification) to the input amount of endo-Gag monomer polypeptides.

### Example 2: Design, Vector Construction, Expression, and Purification of PNMA5 Protein

To construct recombinant DNA vectors for PNMA5 expression, full length cDNA open reading frames, excluding the initial methionine, were inserted into a cloning vector and subsequently transferred into an expression vector according to standard methods.

cDNA encoding PNMA5 was inserted into an expression vector derived from pET-41-a(+) (EMD Millipore (Novagen) Cat #70566). The entire cloning site of pET-41-a(+) was removed and replaced with DNA encoding an N-terminal tag having the amino acid sequence of SEQ ID NO: 37, which comprises a 6×His tag (SEQ ID NO: 27), a 6 amino acid spacer (SEQ ID NO: 28), and a TEV cleavage site (SEQ ID NO: 29), in some instances followed by an additional SG linker.

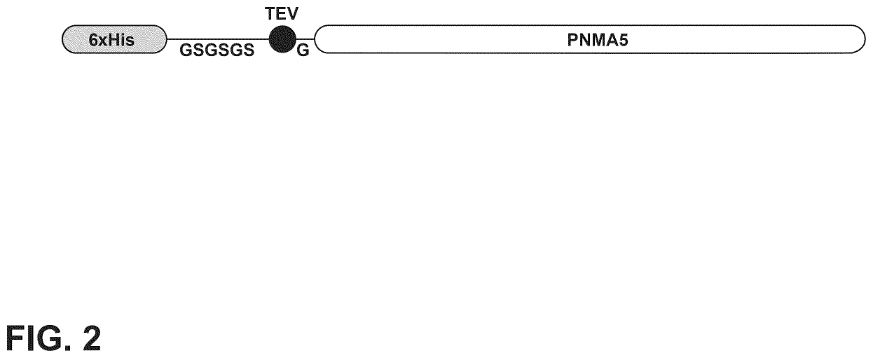

An open reading frame encoding PNMA5 without the starting methionine codon (SEQ ID NO: 77) was inserted after the TEV cleavage site by Gibson assembly. FIG. 2 provides a schematic of the PNMA5 construct, which including the N-terminal tag comprises SEQ ID NO: 79.

PNMA5 was expressed and purified as outlined in Example 2 of International Publication No. WO 2022/164942. The resulting PNMA5 protein was generally more than 95% pure as revealed by SDS-PAGE analysis, with a yield of up to 4 mg per 1 L of bacterial culture.

Purified PNMA5 in the peak fractions was concentrated using a 10 kDa MWCO PES concentrator. The N-terminal 6×His tag (SEQ ID NO: 27) and spacer were then removed by treating with 10% v/v of TEV Protease (10 units/mL). The protein was then diluted into HTA and cleaned by running over HisTrap HP resin to remove 6×His peptides (SEQ ID NO: 27), residual uncleaved PNMA5 containing the 6×His tag (SEQ ID NO: 27), and His-tagged TEV protease.

After expression and Tobacco Etch Virus (TEV) cleavage, the N-terminus of the resulting PNMA5 has a single residual Glycine from TEV cleavage (SEQ ID NO: 78).

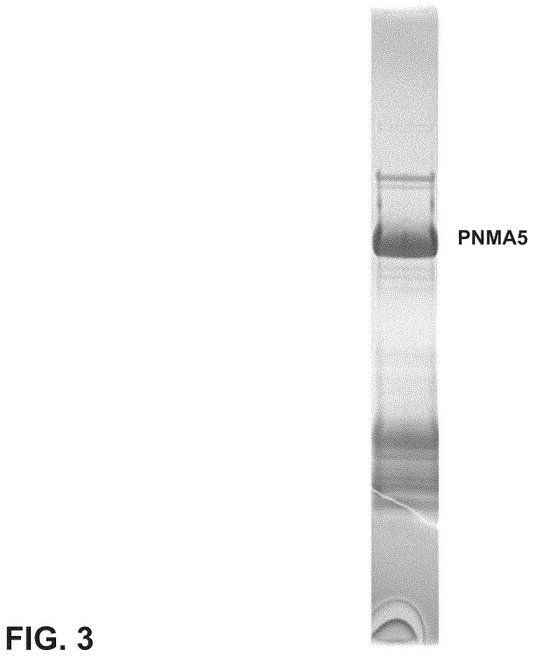

An illustrative image of protein gel stained with Coomassie R-250 showing purified recombinant PNMA5 is provided in FIG. 3.

### Example 3: PNMA5 Capsid Disassembly and Reassembly

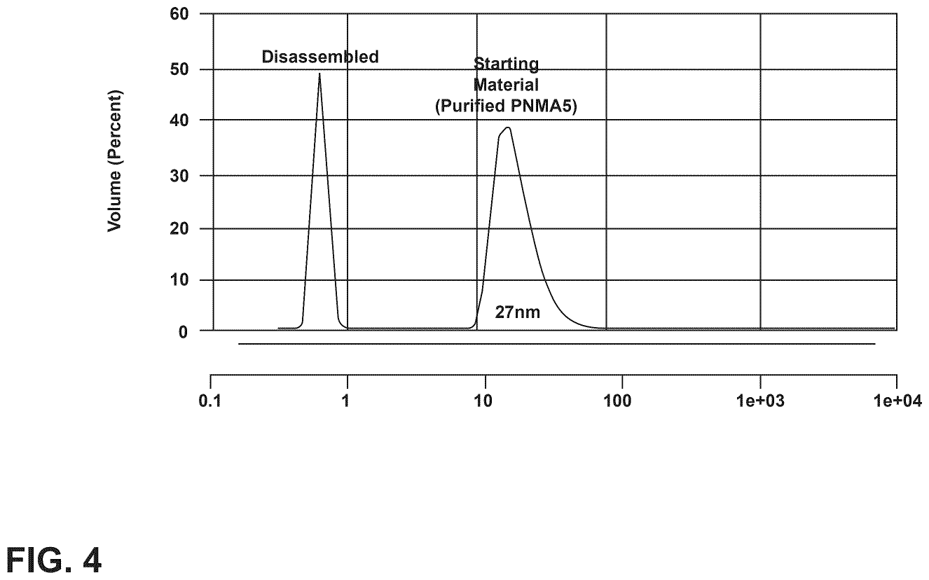

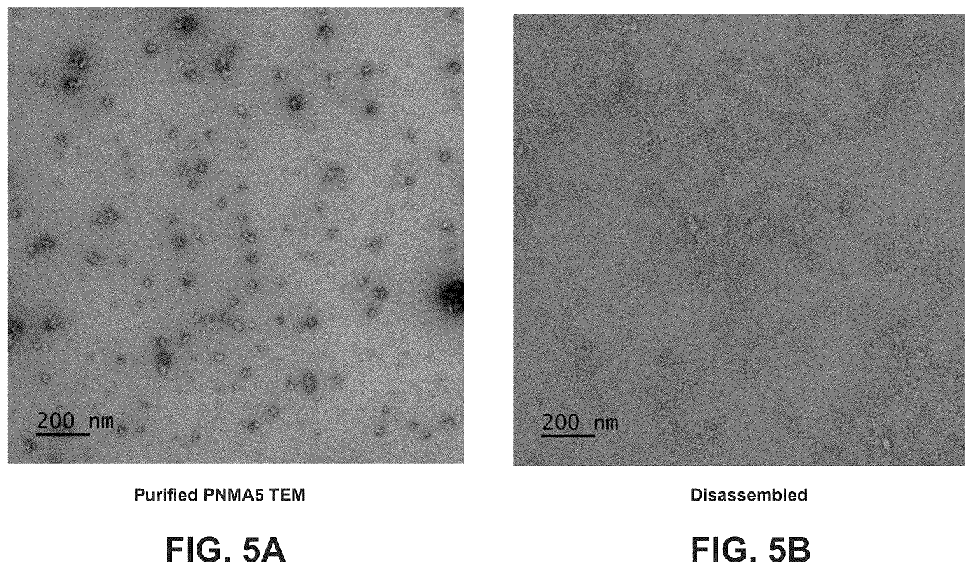

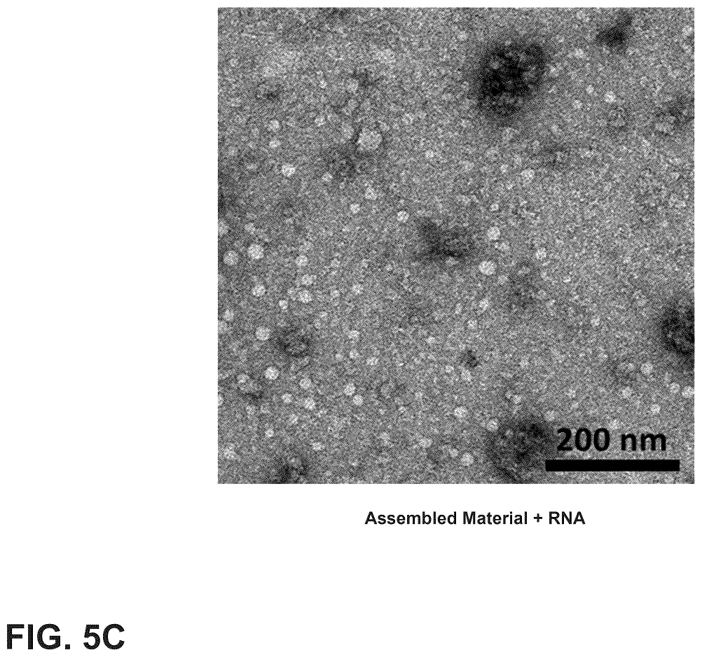

Recently purified PNMA5 (2 mg/mL in HTB) was diluted to 0.5 mg/mL in a disassembly buffer containing 8M Urea, 133.33 mM DTT, 50 mM NaCl, 13.33 mM MgCl₂, to final concentrations of 6M Urea, 100 mM DTT, 10 mM MgCl₂. The solution was incubated on ice for 30 minutes. Following the incubation disassembly was confirmed by MADLS (FIG. 4). Electron microscopy images of PNMA5 capsids before and after disassembly are provided in FIG. 5A and FIG. 5B.

Disassembled PNMA5 was reassembled into capsids by overnight dialysis of the disassembled material supplemented with 6% (w/w) mRNA against reassembly buffer (20 mM Na₂HPO₄/NaH₂PO₄, 0.5M NaCl, 10% glycerol, 40 mM Imidazole, 1 mM DTT, pH 7.4), at 4 °C in a dialysis cassette with a 3,000-10,000 Da molecular cutoff. Capsid assembly was determined by i) MADLS and ii) TEM.

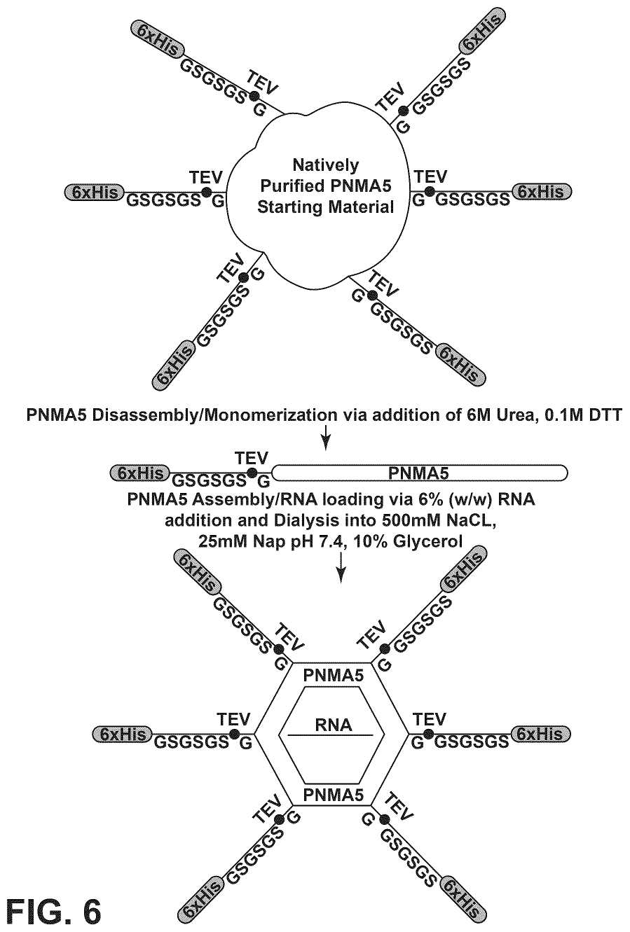

An electron microscopy image of re-assembled PNMA5 capsids is provided in FIG. 5C, and a schematic of the process in FIG. 6.

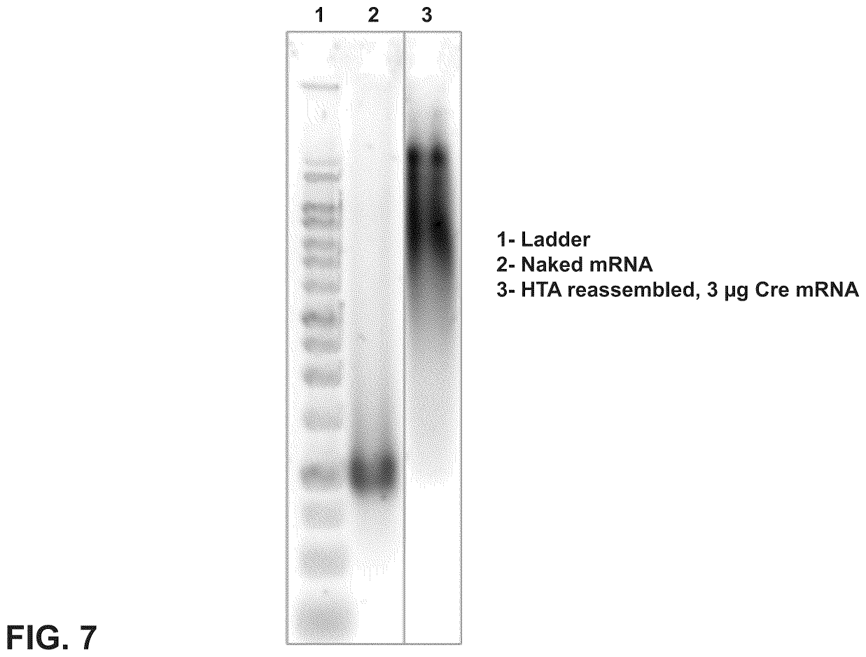

RNA association with the reassembled capsids was evaluated by gel shift analysis. RNA was visualized by staining with SybrGreen II RNA stain. An upshift of RNA was observed for samples with PNMA5 and RNA, indicating that the RNA was packaged in the reassembled PNMA5 capsids (FIG. 7).

### Example 4: Protein Loading of PNMA2 Capsids

This example demonstrates loading of a positively charged illustrative protein into PNMA2 capsids. Wild-type PNMA2 was disassembled according to disassembly protocols disclose herein. (+36) eGFP (SEQ ID NO: 75), a positively supercharged eGFP variant with a net charge of +36, was added to the disassembly mixture, and PNMA2 capsids were assembled as disclosed herein.

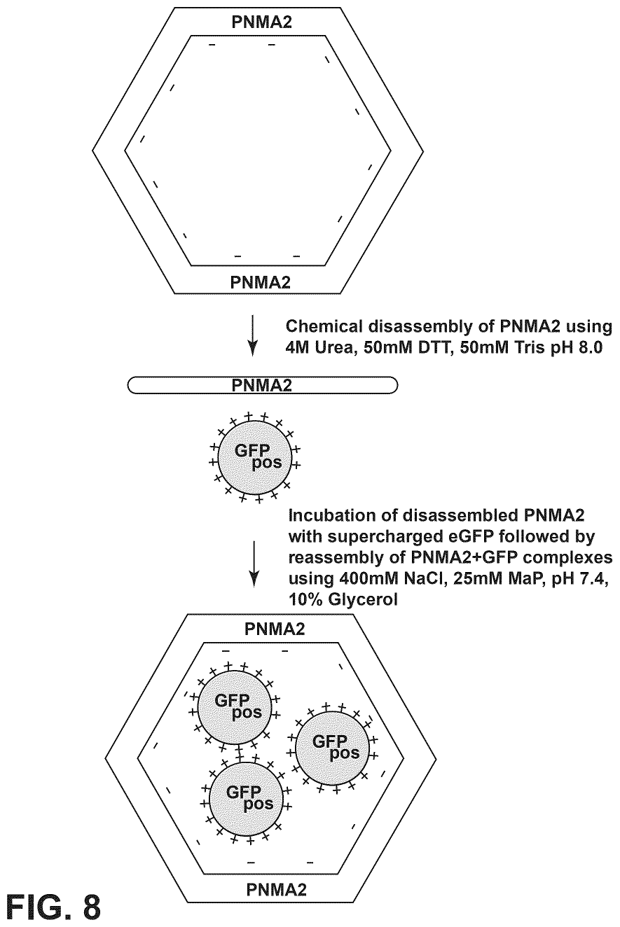

PNMA2 capsids were disassembled and re-assembled with positively charged GFP present (FIG. 8). As a control, PNMA2 capsids were disassembled and re-assembled in the absence of GFP, then GFP was added to the pre-assembled capsids.

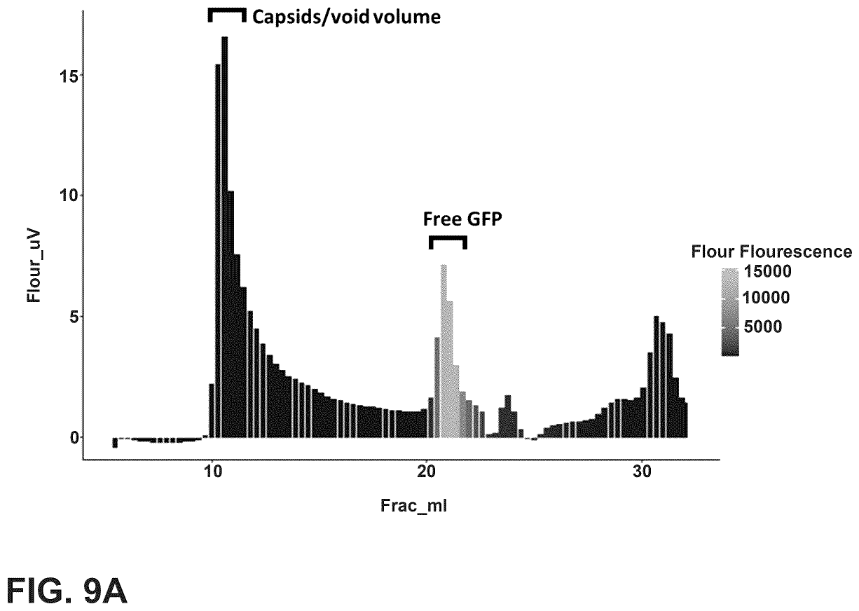

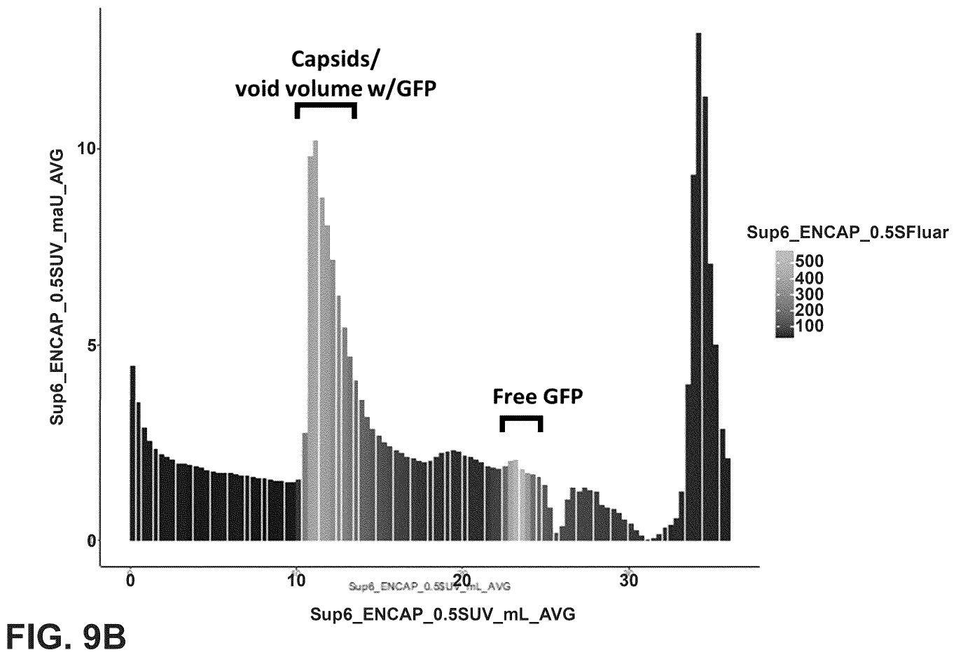

Samples were subjected to size exclusion chromatography. When GFP was added after capsid assembly, capsids eluted in the void volume, while GFP eluted 12 mL later (FIG. 9A). When GFP was present during capsid assembly, GFP fluorescence was detected in the void volume with the capsids (FIG. 9B), while unencapsulated GFP still elutes 12 mL later. The y axes is protein concentration, x axes are mL elution, and the graphs are pseudo-colored for GFP fluorescence.

### Example 5: Expression, Purification, and Capsid Detection of PNMA Family Members

DNA vectors are constructed that encode PNMA family members (e.g., any one or more of SEQ ID NOs: 1-8, 38, 40, 41, 43, 44, 46-49, 52, 53, 55 and 77-92, or a variant or functional fragment thereof disclosed herein). The vectors are generated, for example, as described in Example 1 and/or Example 4 of International Publication No. WO 2022/164942. The vectors can optionally encode an affinity tag and a cleavable linker (e.g., TEV cleavable linker).

Engineered versions of the PNMA family members are also generated (for example, engineered versions of any one or more of SEQ ID NOs: 1-8, 38, and 77-92. The engineered versions include, for example, engineered versions SEQ ID NOs: 1-8, 38, and/or 77-92 comprising a nuclear localization signal (NLS), Arginine rich domain (ARD), zinc finger domain (ZNF), synthetic nucleic acid binding domain (e.g., SEQ ID NO: 39), a nucleic acid binding domain comprising amino acids R381-R403 from human paraneoplastic antigen Ma3 isoform 2 (SEQ ID NO: 42), a nucleic acid binding domain derived from the TAT peptide of HIV-1 (e.g., SEQ ID NO: 45), a moiety to increase cellular uptake or modify intracellular localization upon uptake, such as a single chain antibody fragment (e.g., scFv, HCAb, or VHH targeting a receptor, such as anti-EGFR SEQ ID NO: 50), a receptor ligand, a subcellular localization signal, a cationic peptide (e.g., an R9 peptide, SEQ ID NO: 56), a cell penetrating peptide (e.g., SEQ ID NO: 54), a vectofusion-1 peptide, an introduced cysteine residue, and/or a deletion of one or more amino acids/domains. The modification can be present at the C-terminus, N-terminus, within the PNMA polypeptide, or a combination thereof. The modification can optionally be attached via a linker, such as a flexible linker or other linker disclosed herein.

The vectors are transformed into a suitable expression system, expression is induced, and the resulting PNMA proteins are purified (e.g., as described in Example 2, 6, or 7 of International Publication No. WO 2022/164942).

Purity is assessed by SDS PAGE. The purified PNMA proteins are subjected to transmission electron microscopy (TEM) and multi-angle dynamic light scattering (MADLS) using a Malvern Zetasizer Ultra to evaluate capsid assembly and particle homogeneity.

Capsid structure and integrity are assessed by TEM. EM grids (Carbon Support Film, Square Grid, 400 mesh, 3-4 nm, Copper, CF400-Cu-UL) are prepared by glow discharge. A 5 μL sample of purified capsids is applied to the grid for 30 seconds and then wicked away using filter paper. The grid is then washed twice with MilliQ H₂O. MilliQ H₂O is immediately wicked away using filter paper after each wash. The grid is then washed twice with 5 μL of 1% Uranyl Acetate in H₂O. Uranyl Acetate is immediately wicked away using filter paper after each wash. The grid is then stained using 5 μL of 1% Uranyl Acetate in H₂O for 3 minutes and air dried for 1 minute. Images of capsids are acquired using a FEI Talos L120C TEM equipped with a Gatan 4 k×4 k One View camera. Capsids are also detected and characterized by MADLS.

### Example 6: Capsid Disassembly, Reassembly, and Cargo Loading

Purified PNMA family capsid proteins (e.g., as described in Example 5) are characterized for their ability to be disassembled, reassembled, and package cargo, e.g., using similar experiments to those described in Examples 9 and 11-13 of International Publication No. WO 2022/164942.

The capsid proteins are diluted in disassembly buffers disclosed herein, for example, comprising a solubilization/chaotropic agent (e.g. urea or CHAPS) and/or a reducing agent (e.g. GSH, TCEP, β-ME, or DTT). Illustrative, non-limiting examples of disassembly buffers that can be used include: (1) 5-10% CHAPS, 10-20 mM TCEP, 50-100 mM Tris, pH 8.0; (2) 10 mM TCEP, 10 mM MgCl2, 50 mM Tris, pH 8.0; (3) 4M Urea, 50 mM DTT, 500 mM NaCl, 20 mM NaP, 10% glycerol, pH 7.4; (4) 4-6M Urea, 30-50 mM DTT, 500 mM NaCl, 10% glycerol; (5) 8M Urea, 66.67 mM DTT, 50 mM NaCl, 13.33 mM MgCl₂; and (6) a buffer comprising about 12 mM GSH (reduced glutathione). The solution is incubated on ice for 30 minutes. Following the incubation, disassembly is confirmed by i) MADLS and ii) TEM.

Disassembled capsids are reassembled into capsids by overnight dialysis of the disassembled material against reassembly buffer at +4 °C in a dialysis cassette with a 3,000-10,000 Da molecular weight cutoff. The reassembly buffer can be a physiological buffer, for example, phosphate a buffered saline (PBS). Illustrative, non-limiting examples of reassembly buffers include: (1) 137 mM NaCl, 2.7 mM KCl, 10 mM Na₂HPO₄, 1.8 mM KH₂PO₄; (2) 137 mM NaCl, 2.7 mM KCl, 8 mM Na₂HPO₄, 1.5 mM KH₂PO₄, 0.9 mM CaCl₂), 0.5 mM MgCl₂); (3) 75 mM NaCl, 50 mM Tris, 10% Glycerol, pH 8.0; and (4) 20 mM Na₂HPO₄/NaH₂PO₄, 0.5 M NaCl, 10% glycerol, 40 mM imidazole, 1 mM DTT, pH 7.4. The quality and integrity of the soluble re-assembled capsids is analyzed by i) MADLS and ii) TEM.

The efficiency of capsid formation, disassembly, and reassembly of the capsids are evaluated, e.g., as described in Example 25 of International Publication No. WO 2022/164942 and Example 1, using various disassembly and reassembly conditions disclosed herein. For example, to calculate capsid disassembly and reassembly efficiency, the amount of protein loss is measured, and particle sizes and concentrations were calculated using multi-angle dynamic light scattering, with reassembly confirmed by TEM. An example of a technique for assessing in vitro capsid formation assembly efficiency is provided in Gross et al., (1997) In vitro assembly properties of purified bacterially expressed capsid proteins of human immunodeficiency virus. European Journal of Biochemistry, 249(2), 592-600, which is incorporated herein by reference for such disclosure. For example, the size and/or number of assembled capsid particles per EM mesh grid can be counted as a measure of assembly efficiency.

Additionally or alternatively, the efficiency of disassembly and reassembly can be determined by MADLS or size exclusion chromatography (SEC) as described in Example 1 (e.g., by quantifying the percent of total protein in the monomer peak after disassembly, and by comparing the amounts of endo-Gag polypeptides in the monomer and capsid peaks as assayed by MADLS or SEC after reassembly).

Cargo loading is evaluated for the purified PNMA family capsid proteins. Disassembled capsids are incubated with a cargo (e.g., RNA, DNA, hairpin RNA, ssDNA, protein, or small molecule, at 0.1-10% w/w or w/v) in disassembly buffer for 30 minutes on ice. Disassembled capsid plus cargo is loaded into a 10 kDa MWCO and dialyzed overnight into reassembly Buffer at 4 °C. The following day assembly is confirmed by i) MADLS and ii) TEM. Capsids are assessed for cargo loading, e.g., using a gel shift assay as disclosed herein for DNA or RNA loading, and/or a nuclease protection assay for DNA or RNA loading.

### Example 7: Hybrid PNMA Capsids

Hybrid PNMA capsids are generated with a heterogeneous mixture of (i) a first subunit type (e.g., first wild type or engineered PNMA protein), and (ii) a second subunit type (e.g., a different wild type or engineered PEG10, RTL10, RTL family, PNMA family, Arc, or SCAN family (e.g., ZNF18) protein).

The capsid subunits (e.g., as described in Example 5) are expressed and purified. Following chemical disassembly, the two types of capsid polypeptides are premixed at a desired ratio and reassembled into hybrid capsids. Adjusting relative amounts of the individual polypeptides allows for control of the mass/molar ratio of individual subunits within a given reassembled hybrid capsid.

### Example 8: Capsid Modifications

Chemical attachments: Cargos or other components (e.g., fluorescent dyes, small molecules) are chemically attached to PNMA capsids comprising the PNMA polypeptides described in Example 5. The cargo or component to be attached can be linked to a cysteine, e.g., a cysteine that is appended to the C-terminus, N-terminus, or within the PNMA polypeptide, optionally via a linker, such as a flexible linker disclosed herein. Optionally, one or more other cysteines can be deleted or substituted, e.g., as done for PNMA2 in Example 15 of International Publication No. WO 2022/164942, for targeted attachment at the desired cysteine. Mutants can be generated to test the contribution of cysteines (and, e.g., disulfide bonds) to capsid formation, e.g., as in Examples 8 and 15 of International Publication No. WO 2022/164942. The cargo or other component can be conjugated to the cysteine, e.g., via maleimide chemistry. The conjugated protein is assembled into capsids.

Combination with cationic peptide: Assembled capsids comprising the PNMA polypeptides described in Example 5 are mixed with a cationic peptide (e.g., an R9 peptide, SEQ ID NO: 56, or a vectofusion-1 peptide), incubated prior to contacting cells with the capsids, and the impacts of the cationic peptide on capsid uptake and cargo delivery determined. In some embodiments, the cationic peptide can associate with the capsid (e.g., a negative spike portion thereof) via electrostatic interactions, and the presence of the cationic peptide on the capsid can enhance binding to the negatively charged surface of a cell, facilitating increased capsid uptake.

Combination with cationic lipid: Assembled capsids comprising the PNMA polypeptides described in Example 5 are mixed with a cationic lipid (e.g., jetMESSENGER, or analogous lipid agents) at 0.5% volume to volume and allowed to incubate for 10 minutes at room temperature, and the impacts on capsid cellular uptake and cargo delivery determined, e.g., as described in Example 20 or 22 of International Publication No. WO 2022/164942.

Combination with cationic polymer: Assembled capsids comprising the PNMA polypeptides described in Example 5 are mixed with a cationic polymer (e.g., polybrene at 60 μg/mL), and the impacts on capsid uptake and cargo delivery determined, e.g., as described in Example 22 of International Publication No. WO 2022/164942.

### Example 9: Cellular Internalization of PNMA Capsids

In vitro and in vivo cellular internalization of PNMA capsids generated in Examples 5-8 is evaluated.

Mammalian cells are cultured, capsids added at a concentration of about 0.1 mg/mL, and incubated to allow time for uptake. Cells are washed and capsid uptake determined, e.g., via staining for the capsids and fluorescence microscopy as described in Examples 14 and 16 of International Publication No. WO 2022/164942.

In vivo internalization of PNMA capsids generated in Examples 5-8 is evaluated by injecting C57BL/6 mice, e.g., intramuscularly in the hind quarter with about 10 μg of capsids. Animals are sacrificed and muscle tissue from both the injected side, and the contralateral un-injected side are collected, washed in ice cold PBS, and processed for epifluorescence microcopy to evaluate the presence of capsid in muscle, e.g., as described in Example 14 of International Publication No. WO 2022/164942.

Cre mRNA is tested as an illustrative cargo for delivery by capsids generated in Examples 5-8. GFP/RFP switch reporter cells comprising a GFP-loxP-Stop-loxP-RFP construct are contacted with the capsids, and 24-72 hours later, cells are imaged to visualize RFP expression, e.g., as described in Examples 18, 19, and 21 of International Publication No. WO 2022/164942.

For in vivo delivery, capsids comprising Cre mRNA generated as described in Examples 5-8 are sterile filtered and injected into the hind quarter of Ai14 reporter mice. Seven days later, animals are sacrificed and the muscle tissue of the hind limbs collected and processed for fluorescent imaging to evaluate delivery of functional RNA.

The levels of uptake and cargo delivery is determined for different capsids.

### Example 10: Efficiency of Capsid Assembly, Disassembly, and Reassembly

This example provides illustrative protocols that can be used to calculate the efficiency of assembly, disassembly, and reassembly of capsids disclosed herein that comprise endogenous Gag polypeptides (e.g., native or engineered endogenous Gag polypeptides, such as native or engineered PNMA2, PEG10, or RTL10/BOP polypeptides) and the recovery of assembled, disassembled, and reassembled endogenous Gag polypeptides. Also provided are methods to purify assembled and reassembled capsids.

Isolated capsids are disassembled into monomers and other non-capsid forms, or maintained in a monomeric state or non-capsid form, by incubation in a disassembly buffer.

A disassembly buffer can comprise a mono valent salt (e.g., NaCl, KCl, or NaBr, at a concentration of about 50 mM to about 1.5M, e.g., 50 mM or 500 mM). A disassembly buffer can comprise a reducing agent (e.g. GSH, TCEP, β-ME, or DTT at a concentration of about 5-70 mM, e.g., 5 mM, 10 mM, 12 mM, 20 mM, 30 mM, 50 mM, or 66.67 mM). A disassembly buffer can be buffered to about pH 7.4 or to a basic pH of about 8-10, for example, with a Tris, NaP, CHES, or CAPS buffer.

A disassembly buffer for PEG10 can comprise a high salt buffer, a high ionic strength buffer, and/or a reducing agent (e.g. GSH, TCEP, β-ME, or DTT). The disassembly buffer for PEG10 can comprise about 500 mM to about 1.5M of a monovalent salt, e.g., NaCl. An illustrative, non-limiting example of a disassembly buffer that can be used for PEG10 is 500 mM NaCl, 5 mM DTT, 50 mM Tris, pH 8.0.

A disassembly buffer for RTL10 can comprise a low salt buffer and/or a reducing agent (e.g. GSH, TCEP, β-ME, or DTT). An illustrative, non-limiting example of a disassembly buffer that can be used for RTL10 is 50 mM NaCl, 10 mM DTT, 25 mM Tris, pH 8.0.

A disassembly buffer can comprise a solubilization/chaotropic agent (e.g. urea or CHAPS) and/or a reducing agent (e.g. GSH, TCEP, β-ME, or DTT). Illustrative, non-limiting examples of disassembly buffers that can be used for PNMA2 include: (1) 5-10% CHAPS, 10-20 mM TCEP, 50-100 mM Tris, pH 8.0; (2) 10 mM TCEP, 10 mM MgCl2, 50 mM Tris, pH 8.0; (3) 4M Urea, 50 mM DTT, 500 mM NaCl, 20 mM NaP, 10% glycerol, pH 7.4; (4) 4-6M Urea, 30-50 mM DTT, 500 mM NaCl, 10% glycerol; (5) 8M Urea, 66.67 mM DTT, 50 mM NaCl, 13.33 mM MgCl2; and (6) a buffer comprising about 12 mM GSH (reduced glutathione).

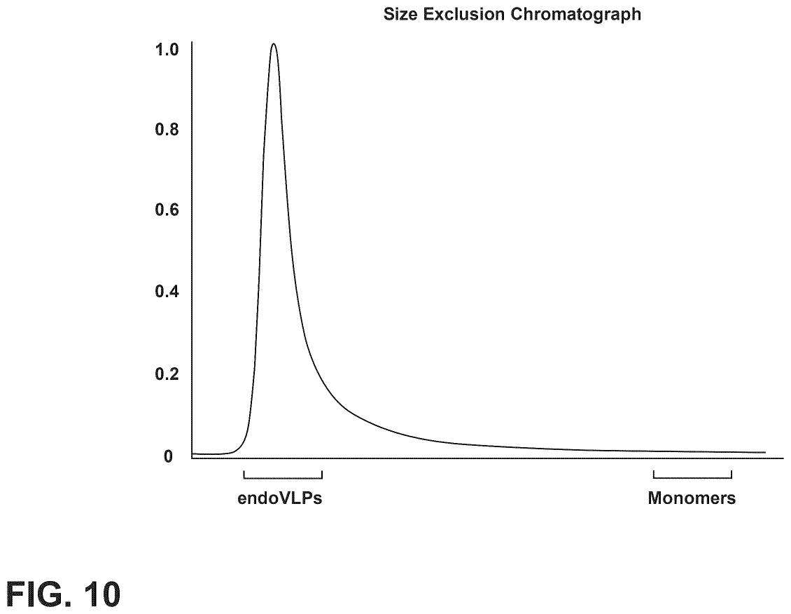

The efficiency of disassembly can be determined by MADLS or size exclusion chromatography (SEC). MADLS shows a monomer peak (e.g., at about 1 nm) and a capsid peak (e.g., at about 30 nm). SEC separates capsids in the void volume from monomers that enter the column matrix and are eluted according to their size. For SEC, samples are loaded onto a Superose 6 Increase 10/300 GL column (GE Healthcare, Cat #29091596) in PBS-500 buffer (1X PBS pH 7.5, 363 mM NaCl). 1 Column Volume of PBS-500 buffer is run through the column and capsids elute in the column Void Volume. Monomers and other non-capsid forms are eluted from the matrix later, after approximately 15 mL of PBS-500 has eluted. Results from an SEC analysis of isolated PNMA2 capsids are shown in FIG. 10. For MADLS or SEC, the efficiency of disassembly is determined by quantifying the percent of total protein in the monomer peak. When the efficiency of disassembly is greater than about 95%, recovery of disassembled endo-Gag polypeptides is determined by comparing the amount of protein in solution after disassembly to the amount of protein in solution before disassembly.

Disassembled endo-Gag monomers are reassembled into capsids by incubation in a reassembly buffer. The reassembly buffer can be a physiological buffer, for example, phosphate a buffered saline (PBS). The reassembly buffer can be a low salt buffer or a buffer with a low ionic strength, e.g., with less than about 200 mM of a salt, such as NaCl. The reassembly buffer can be an acidic buffer with a divalent cation.

Illustrative, non-limiting examples of reassembly buffers that can be used for PEG10 include: (1) a low salt buffer with 150 mM NaCl, 25 mM Tris, 10 μM ZnCl₂, pH 7.5; and (2) a low salt buffer with 150 mM NaCl, 1 mM DTT, 10 μM ZnCl₂, 50 mM Tris, pH 8.0.

An illustrative, non-limiting example of a reassembly buffer that can be used for RTL10 is an acidic buffer plus divalent cation-50 mM MES (2-(N-morpholino) ethanesulfonic acid), 40 mM MgCl₂, pH 6.0.

Illustrative, non-limiting examples of reassembly buffers that can be used for PNMA2 include: (1) 137 mM NaCl, 2.7 mM KCl, 10 mM Na₂HPO₄, 1.8 mM KH₂PO₄; (2) 137 mM NaCl, 2.7 mM KCl, 8 mM Na₂HPO₄, 1.5 mM KH₂PO₄, 0.9 mM CaCl₂), 0.5 mM MgCl₂); (3) 75 mM NaCl, 50 mM Tris, 10% Glycerol, pH 8.0; and (4) 20 mM Na₂HPO₄/NaH₂PO₄, 0.5 M NaCl, 10% glycerol, 40 mM imidazole, 1 mM DTT, pH 7.4.

In some embodiments the reassembly buffer does not contain a chaotropic agent and/or a reducing agent. The reassembly buffer may contain a heterologous cargo (e.g., nucleic acid).

After reassembly, aggregates are removed by centrifugation at 15,000×g for 5 min at +4 °C. The efficiency of reassembly can be determined by comparing the amounts of endo-Gag polypeptides in the monomer and capsid peaks as assayed by MADLS or SEC. Reassembled capsid compositions where at least 95% of the endo-Gag polypeptides are incorporated into capsids can be used directly for cargo delivery to cells or can be further purified. Further purification can be recommended when reassembly is less than 95% efficient. Reassembled capsids can be purified by SEC or Ion-exchange chromatography (IEC). The recovery of reassembled capsids is determined by comparing the amount of endo-Gag polypeptides in reassembled capsids (with or without further purification) to the input amount of endo-Gag monomer polypeptides.

### Example 11: Design, Vector Construction, Expression, and Purification of RTL10 (BOP) Protein

To construct recombinant DNA vectors for RTL10 expression, full length cDNA open reading frames, excluding the initial methionine, were inserted into a cloning vector and subsequently transferred into an expression vector according to standard methods.

cDNA encoding RTL10 was inserted into an expression vector derived from pET-41-a(+) (EMD Millipore (Novagen) Cat #70566). The entire cloning site of pET-41-a(+) was removed and replaced with DNA encoding an N-terminal tag having the amino acid sequence of SEQ ID NO: 37, which comprises a 6×His tag (SEQ ID NO: 27), a 6 amino acid spacer (SEQ ID NO: 28), and a TEV cleavage site (SEQ ID NO: 29), in some instances followed by an additional SG linker.

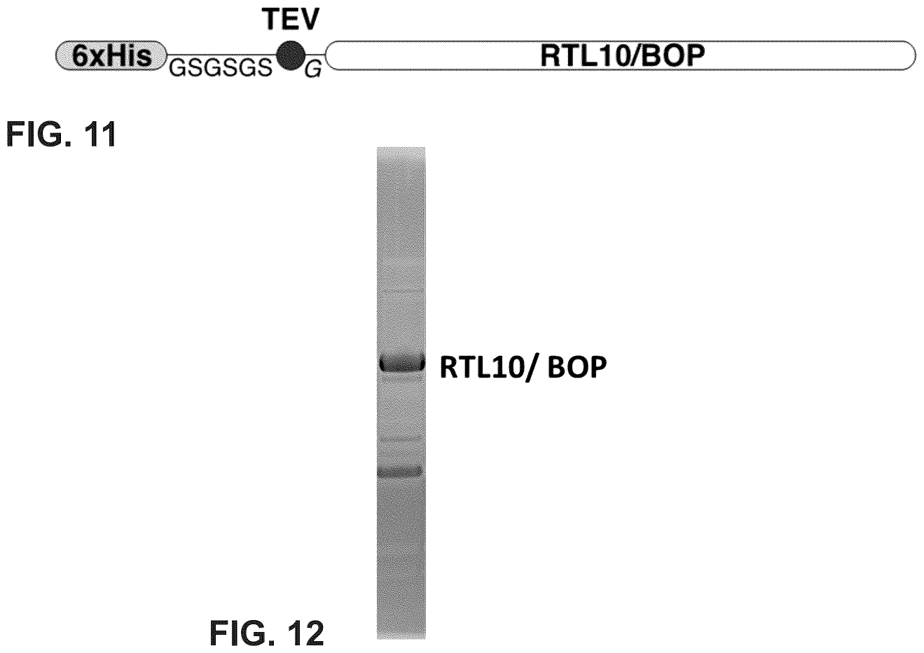

An open reading frame encoding RTL10 without the starting methionine codon (SEQ ID NO: 60) was inserted after the TEV cleavage site by Gibson assembly. FIG. 11 provides a schematic of the RTL10 construct, which including the N-terminal tag comprises SEQ ID NO: 62.

RTL10 was expressed and purified as outlined in Example 2 of International Publication No. WO 2022/164942. The resulting RTL10 protein was generally more than 95% pure as revealed by SDS-PAGE analysis, with a yield of up to 4 mg per 1 L of bacterial culture.

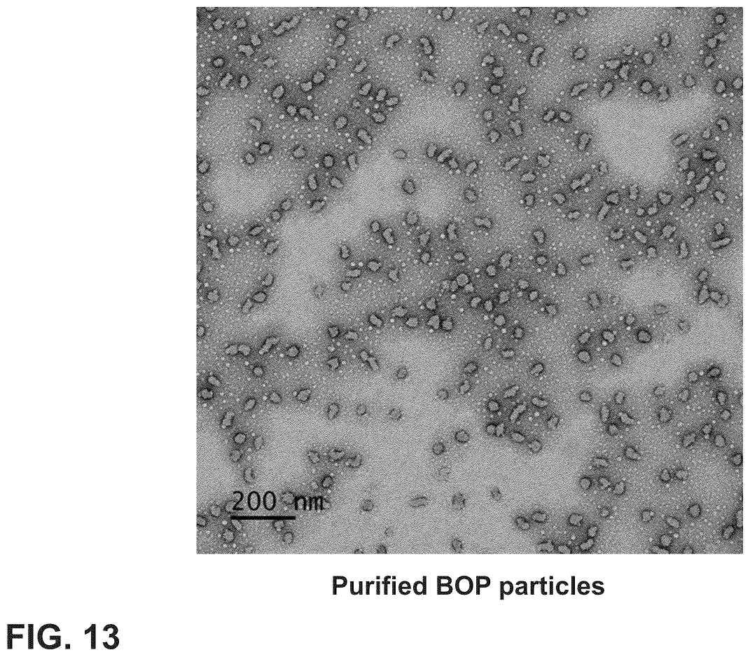

An illustrative image of protein gel stained with Coomassie R-250 showing purified recombinant RTL 10 is provided in FIG. 12, and an illustrative electron microscopy of purified RTL10 particles is provided in FIG. 13.

After expression and Tobacco Etch Virus (TEV) cleavage, the N-terminus of the resulting RTL10 has a single residual Glycine from TEV cleavage (SEQ ID NO: 61).

### Example 12: RTL10 Capsid Disassembly and Reassembly

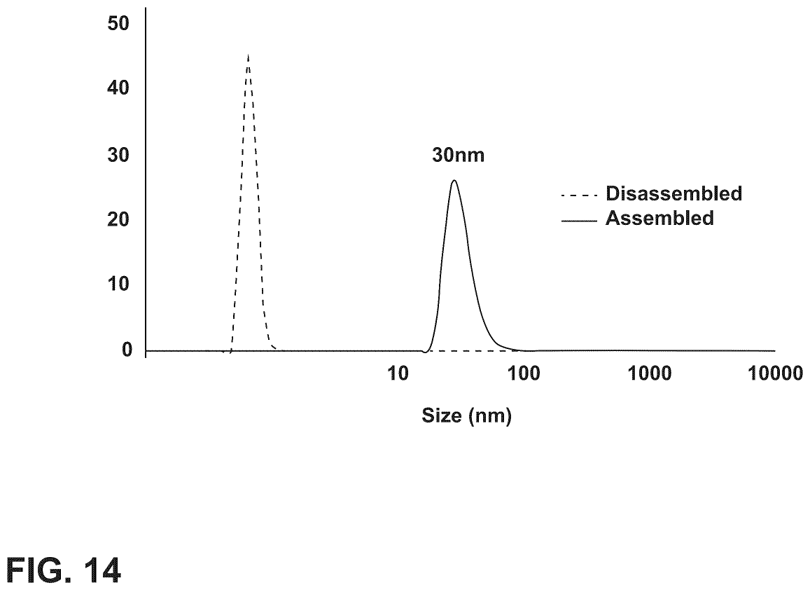

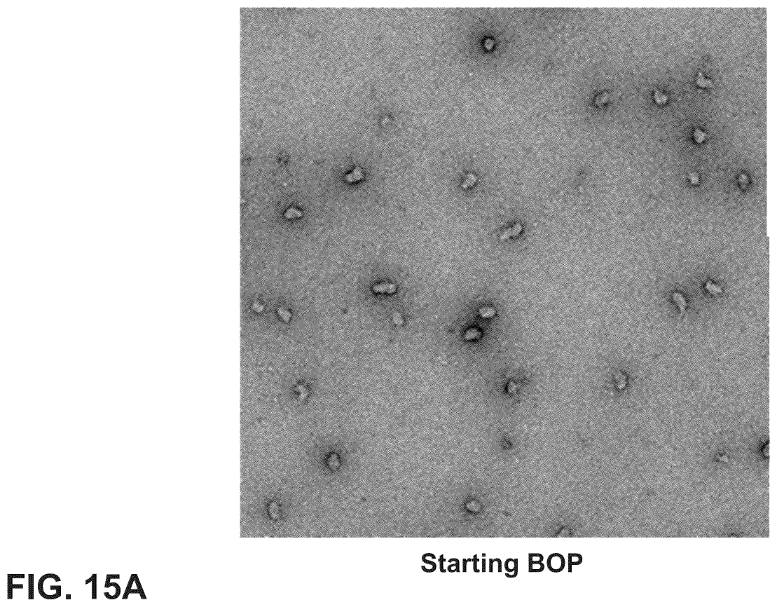

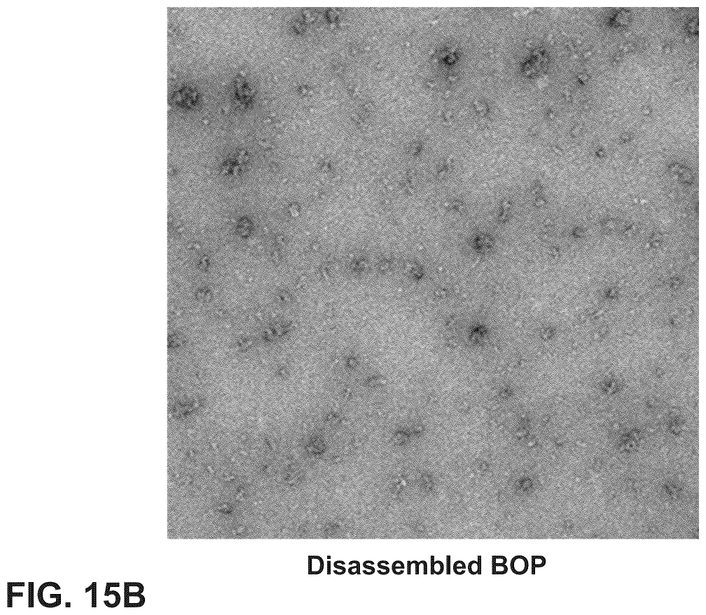

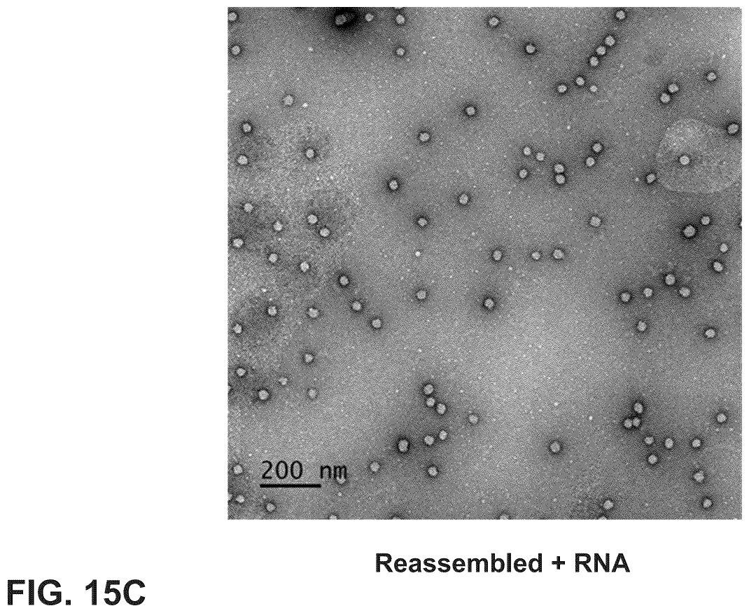

Recently purified BOP protein was up concentrated to 10 mg/mL using a 100 kDa MWCO spin concentrator and rapidly diluted to 0.1 mg/mL into a low salt buffer containing 50 mM NaCl 10 mM DTT 25 mM Tris pH 8.0. The solution was incubated on ice for 30 minutes. Following the incubation disassembly was confirmed by i) MADLS (FIG. 14) and ii) TEM. Representative images of RTL10 before and after disassembly are provided in FIG. 15A and FIG. 15B. Some larger structures were observable after incubation of RTL10 in disassembly buffer. In some embodiments the observed structures represent partial disassembly of the RTL1 capsids into a non-capsid state or capsid subunits (e.g., oligomers, capsomers).

Disassembled BOP/RTL10 was reassembled into capsids by overnight dialysis of the disassembled material supplemented with 4-7% (w/w) mRNA against a reassembly buffer (BOP_RB: 50 mM MES pH 6.0, 40 mM MgCl₂) at 4 °C in a dialysis cassette with a 3,000-10,000 Da molecular cutoff.

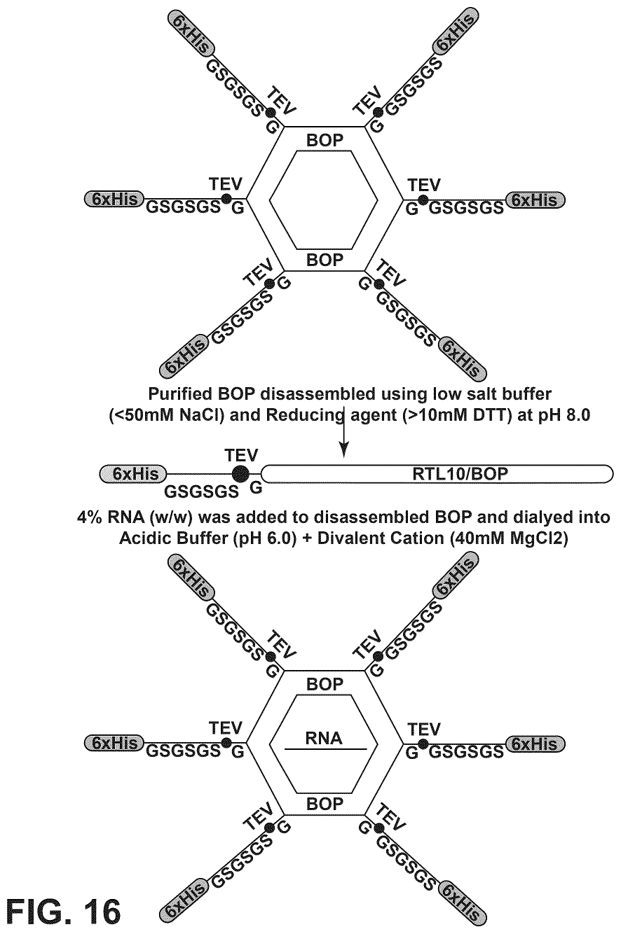

Capsid reassembly was determined by i) MADLS and ii) TEM. An electron microscopy image of re-assembled RTL10 capsids is provided in FIG. 15C, and a schematic of the process in FIG. 16.

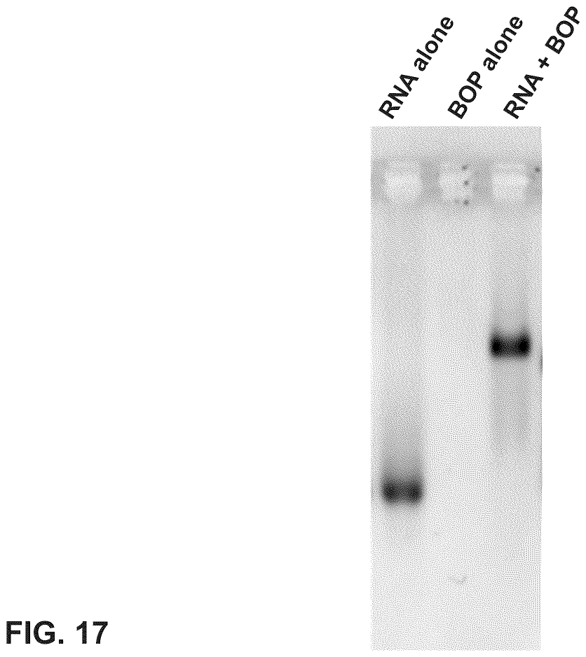

RNA association with the reassembled capsids was evaluated by gel shift analysis. RNA was visualized by staining with SybrGreen II RNA stain. An upshift of RNA was observed for samples with RTL10 and RNA, indicating that the RNA was packaged in the reassembled RTL10 capsids (FIG. 17).

### Example 13: Design, Vector Construction, Expression, and Purification of PEG10 (RTL2) Protein

To construct recombinant DNA vectors for PEG10 expression, full length cDNA open reading frames, excluding the initial methionine, were inserted into a cloning vector and subsequently transferred into an expression vector according to standard methods.

cDNA encoding PEG10 was inserted into an expression vector derived from pMAL-p5x (NEB). The original plasmid sequence encoding a FactorXa cleavage site was replaced with a sequence encoding 3×GS-TEV (SEQ ID NO: 63) downstream of the maltose binding protein (MBP, SEQ ID NO: 64). DNA encoding PEG10 without the initial methionine (SEQ ID NO: 65) was inserted immediately downstream of the previous sequence by Gibson Assembly. In some instances a 6×His tag (SEQ ID NO: 27) was added to the C-terminus of PEG10 prior to the stop codon by Q5 Site directed mutagenesis (NEB cat E0554S). An illustrative sequence of PEG10 lacking the N-terminal methionine, comprising an N-terminal G (e.g., after TEV cleavage), and comprising a C-terminal 6×His tag (SEQ ID NO: 27) is provided in SEQ ID NO: 66, while an equivalent sequence lacking the His tag is provided in SEQ ID NO: 68.

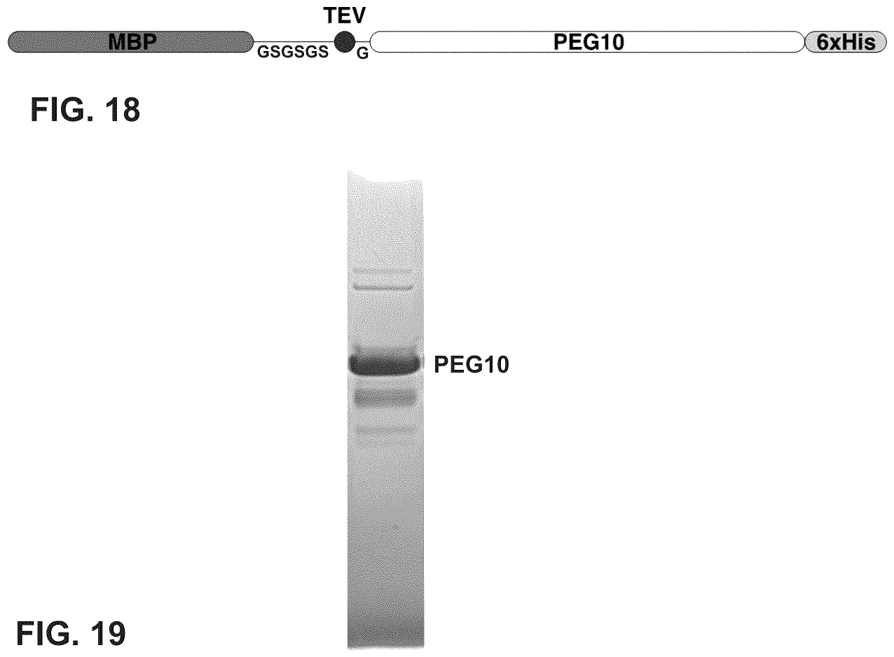

FIG. 18 provides a schematic of the PEG10 that comprises the N-terminal MBP, 3×GS, and AcTEV, and C-terminal 6×His tag (SEQ ID NO: 27) (e.g., as provided in SEQ ID NO: 67).

Expression vector constructs comprising an N terminal MBP tag and C terminal his tag PEG10 open reading frames were transformed into the Rosetta 2 (DE3) E. coli strain (Millipore Sigma, Cat #71400). MBP-PEG10 expression was induced with 0.1 mM IPTG followed by a 16-hour incubation at 16 °C followed by MBP-PEG10 protein purification. Cell pellets were lysed by sonication in 20 mM sodium phosphate pH 7.4, 0.1M NaCl, 40 mM imidazole, 1 mM DTT, 1 mM ZnCl₂, 1 mM ATP, and 10% glycerol. The lysate was treated with excess TURBO DNase (Thermo Fisher Scientific, Cat #AM2238), RNase Cocktail (Thermo Fisher Scientific, Cat #AM2286). The lysate was cleared of cell debris by centrifugation followed by filtration.

MBP-tagged recombinant protein was loaded onto a MBPTrap HP column (Cytiva, Cat #28918778), washed with HisTrap Buffer A (HTA) (20 mM sodium phosphate pH 7.4, 0.5M NaCl, 40 mM imidazole, and 10% glycerol), and eluted with a stepwise elution of HisTrap Buffer A+Maltose (HTAmalt) (20 mM sodium phosphate pH 7.4, 0.5M NaCl, 40 mM imidazole, 10 mM Maltose, and 10% glycerol). The peak of the eluted protein was collected, and the MBP tag was cleaved from the target protein using TEV protease. 10% (v/v) of TEV protease was added to the eluted material and incubated at 30 °C for 1.5 hours. The cleaved C-terminally 6×His-tagged (SEQ ID NO: 27) recombinant protein was loaded onto a HisTrap HP column (Cytiva, Cat #17-5247-01), washed with HisTrap Buffer A (HTA) (20 mM sodium phosphate pH 7.4, 0.5M NaCl, 40 mM imidazole, and 10% glycerol), and eluted with a linear gradient of HisTrap Buffer B (HTB) (20 mM sodium phosphate pH 7.4, 0.5M NaCl, 500 mM imidazole, and 10% glycerol). The resulting PEG10 protein was generally more than 90% pure as revealed by SDS-PAGE analysis, with a yield of up to 6 mg per 1 L of bacterial culture.

An illustrative image of protein gel stained with Coomassie R-250 showing purified recombinant PEG10 is provided in FIG. 19.

### Example 14: PEG10 Capsid Assembly and Cargo Loading

A bulky N-terminal tag prevented oligomerization of PEG-10 while expressing the protein in bacteria. The bulky MBP tag prevented oligomerization during expression, lysis and the initial MBP-based purification.

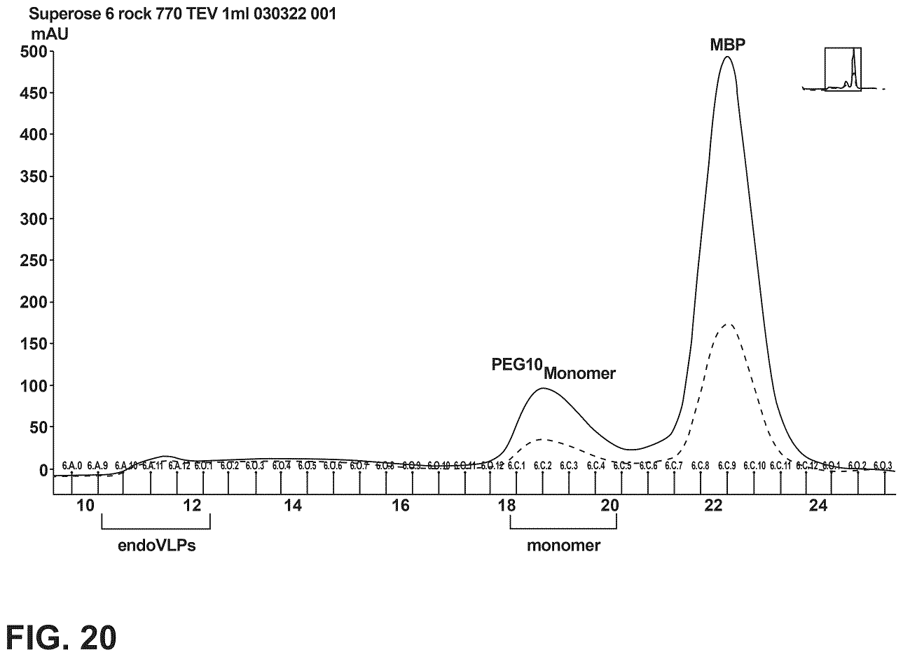

Upon cleavage of the tag, high salt conditions were used to keep the protein in its non-assembled monomeric state. Recently purified MBP-tagged PEG10 was cleaved with 10% (v/v) of TEV protease for 1.5 hours at 30 °C in a pH 8.0 solution containing a monovalent salt concentration of at least 500 mM and no greater than 1.5M NaCl. The solution containing free PEG10 and MBP was then applied onto a HisTrap HP column (GE Healthcare, Cat #17-5247-01), washed with HisTrap buffer A (HTA) (20 mM sodium phosphate pH 7.4, 0.5M NaCl, 40 mM imidazole, and 10% glycerol), and eluted with a linear gradient of HisTrap buffer B (HTB) (20 mM sodium phosphate pH 7.4, 0.5M NaCl, 500 mM imidazole, and 10% glycerol). Purified PEG10 was confirmed to be monomeric by i) SEC chromatography (FIG. 20) and ii) MADLS.

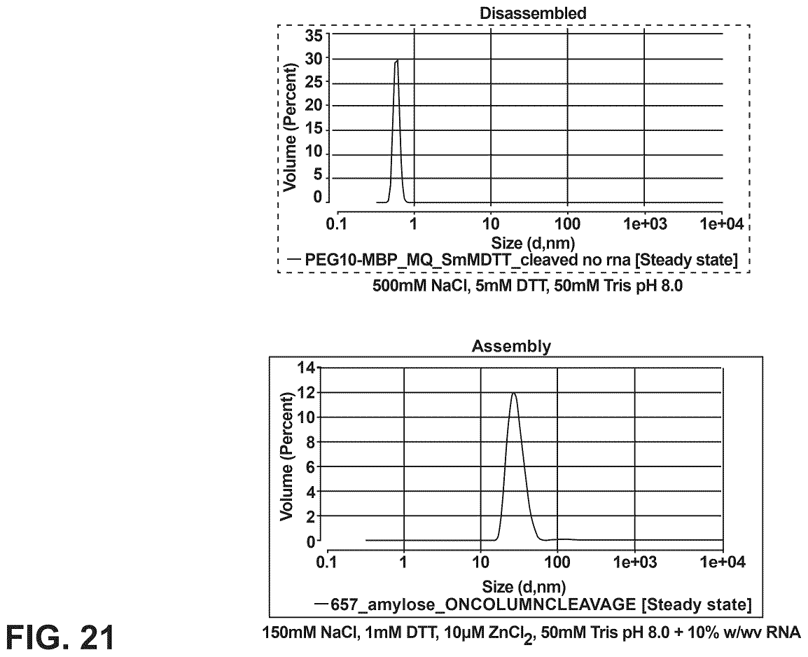

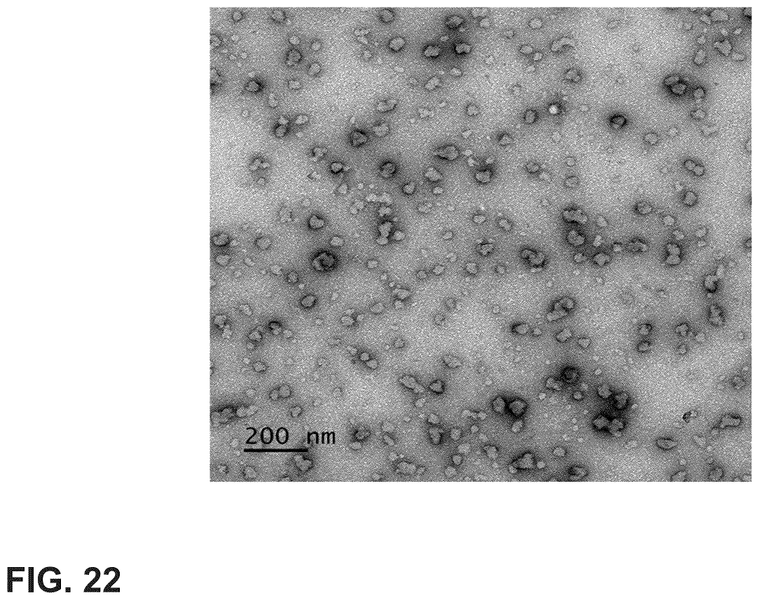

Purified PEG10 monomer was mixed with 10% mRNA (w/w) and dialyzed into TNZ buffer: 150 mM NaCl, 25 mM Tris pH 7.5, 10 μM ZnCl₂, overnight at 4 °C. Capsid assembly was determined by i) MADLS and ii) TEM. FIG. 21 shows a multi-angle dynamic light scattering (MADLS) profile of purified PEG10 before and after capsid assembly. FIG. 22 shows a representative electron microscopy image the assembled PEG10 capsids.

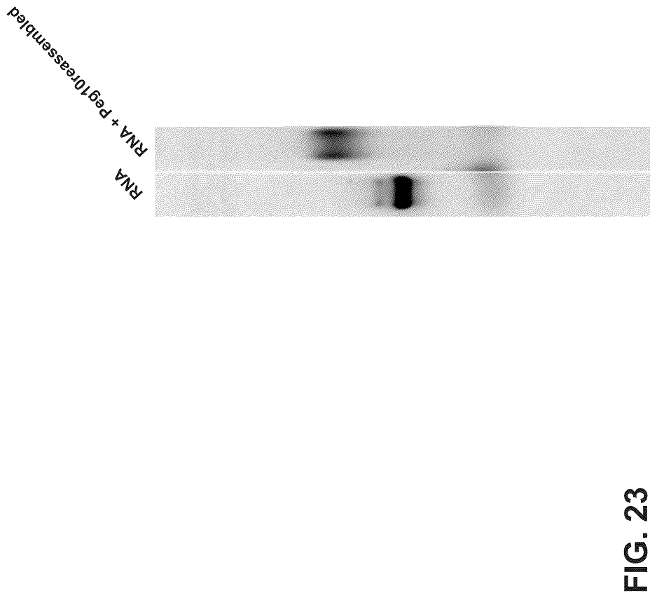

RNA association was evaluated by a 1% native agarose gel EMSA assay using 60V for 1 hour. RNA was visualized by staining with SybrGreen II RNA stain. An upshift of RNA was observed for samples with PEG10 and RNA, indicating that the RNA was packaged in the reassembled PEG10 capsids (FIG. 23).

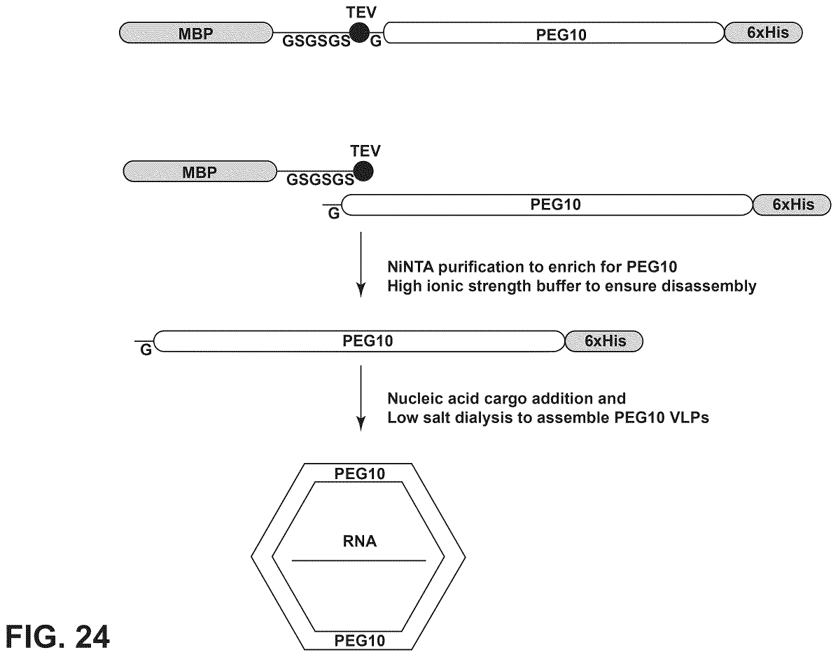

FIG. 24 is a schematic summarizing purification and assembly of PEG10 capsids.

### Example 15: Protein Loading of PNMA2 Capsids

This example demonstrates loading of a positively charged illustrative protein into PNMA2 capsids. Wild-type PNMA2 was disassembled according to disassembly protocols disclose herein. (+36) eGFP, a positively supercharged eGFP variant with a net charge of +36, was added to the disassembly mixture, and PNMA2 capsids were assembled as disclosed herein.

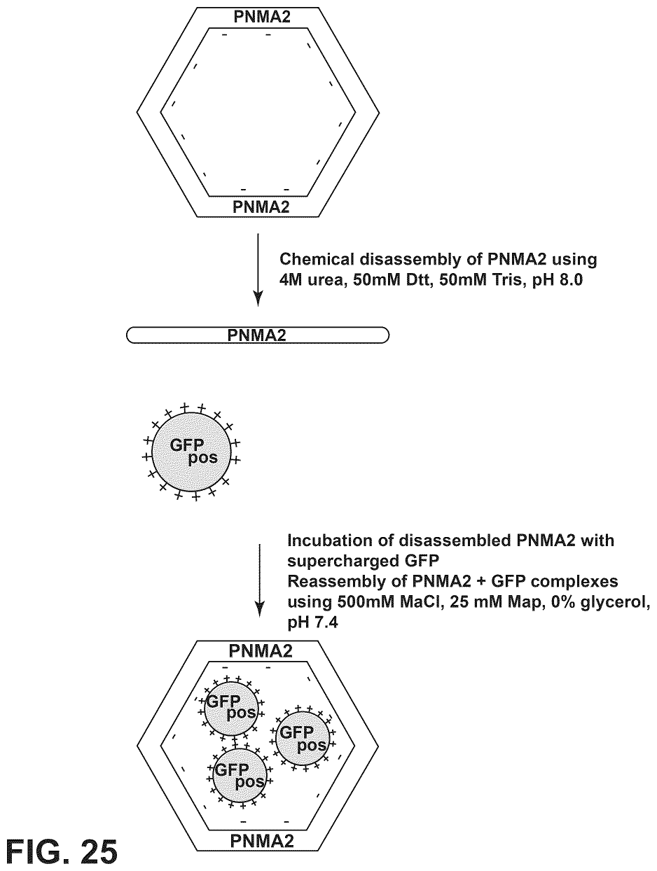

PNMA2 capsids were disassembled and re-assembled with positively charged GFP present (FIG. 25). As a control, PNMA2 capsids were disassembled and re-assembled in the absence of GFP, then GFP was assed to the pre-assembled capsids.

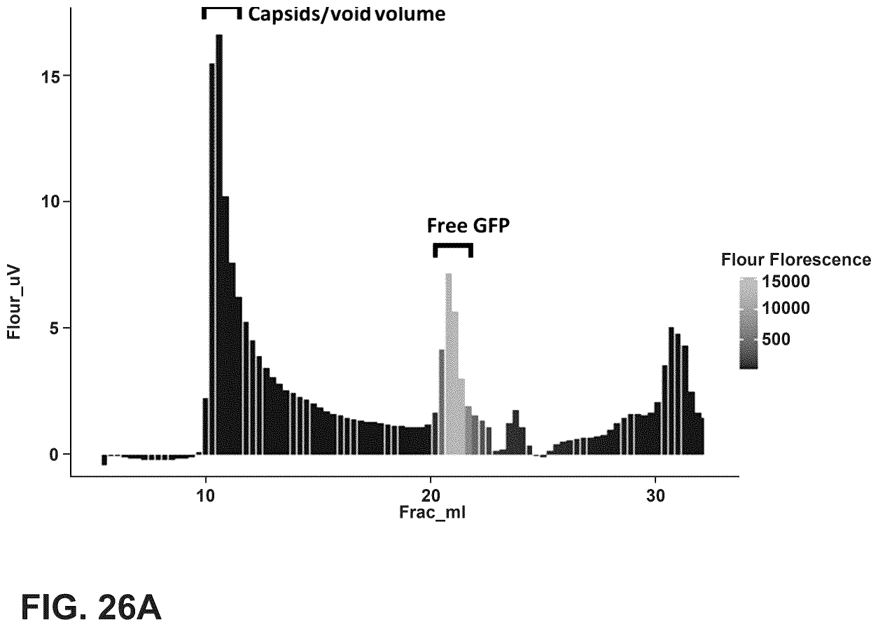

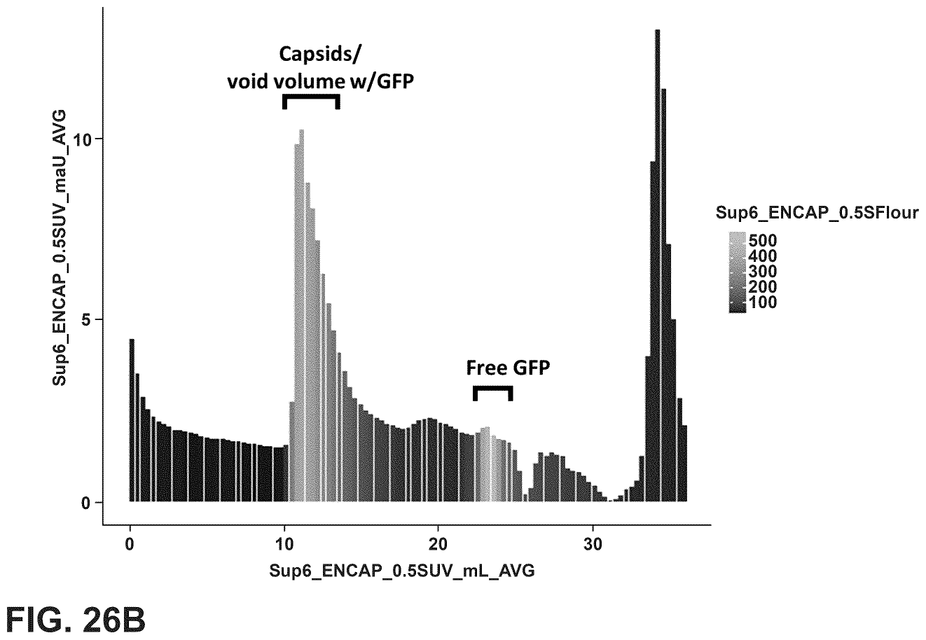

Samples were subjected to size exclusion chromatography. When GFP was added after capsid assembly, capsids eluted in the void volume, while GFP eluted 12 mL later (FIG. 26A). When GFP was present during capsid assembly, GFP fluorescence was detected in the void volume with the capsids (FIG. 26B), while unencapsulated GFP still elutes 12 mL later. The y axes is protein concentration, x axes are mL elution, and the graphs are pseudo-colored for GFP fluorescence.

### Example 16: Expression, Purification, and Capsid Detection of PEG10 and RTL10

DNA vectors are constructed that encode PEG10 and RTL10 (e.g., any one or more of SEQ ID NOs: 60-62 and 65-74, or a variant or functional fragment thereof disclosed herein). The vectors are generated, for example, as described in Example 1 and/or Example 4 of International Publication No. WO 2022/164942. The vectors can optionally encode an affinity tag and a cleavable linker (e.g., TEV cleavable linker).

Engineered versions of PEG10 and RTL10 are also generated (for example, engineered versions of any one or more of SEQ ID NOs: 60-62 and 65-74. The engineered versions include, for example, engineered versions SEQ ID NOs: 60-62 and 65-74 comprising a nuclear localization signal (NLS), Arginine rich domain (ARD), zinc finger domain (ZNF), synthetic nucleic acid binding domain (e.g., SEQ ID NO: 39), a nucleic acid binding domain comprising amino acids R381-R403 from human paraneoplastic antigen Ma3 isoform 2 (SEQ ID NO: 42), a nucleic acid binding domain derived from the TAT peptide of HIV-1 (e.g., SEQ ID NO: 45), a moiety to increase cellular uptake or modify intracellular localization upon uptake, such as a single chain antibody fragment (e.g., scFv, HCAb, or VHH targeting a receptor, such as anti-EGFR SEQ ID NO: 50), a receptor ligand, a subcellular localization signal, a cationic peptide (e.g., an R9 peptide, SEQ ID NO: 56), a cell penetrating peptide (e.g., SEQ ID NO: 54), a vectofusion-1 peptide, an introduced cysteine residue, and/or a deletion of one or more amino acids/domains. The modification can be present at the C-terminus, N-terminus, within the PEG10 or RTL10 polypeptide, or a combination thereof. The modification can optionally be attached via a linker, such as a flexible linker or other linker disclosed herein.

The vectors are transformed into a suitable expression system, expression is induced, and the resulting endo Gag proteins are purified (e.g., as described in Example 2, 6, or 7 of International Publication No. WO 2022/164942 or Example 11 or Example 13). PEG10 capsids can be assembled as described in Example 14.

Purity is assessed by SDS PAGE. The purified endo Gag proteins are subjected to transmission electron microscopy (TEM) and multi-angle dynamic light scattering (MADLS) using a Malvern Zetasizer Ultra to evaluate capsid assembly and particle homogeneity.

Capsid structure and integrity are assessed by TEM. EM grids (Carbon Support Film, Square Grid, 400 mesh, 3-4 nm, Copper, CF400-Cu-UL) are prepared by glow discharge. A 5 μL sample of purified capsids is applied to the grid for 30 seconds and then wicked away using filter paper. The grid is then washed twice with MilliQ H₂O. MilliQ H₂O is immediately wicked away using filter paper after each wash. The grid is then washed twice with 5 μL of 1% Uranyl Acetate in H₂O. Uranyl Acetate is immediately wicked away using filter paper after each wash. The grid is then stained using 5 μL of 1% Uranyl Acetate in H₂O for 3 minutes and air dried for 1 minute. Images of capsids are acquired using a FEI Talos L120C TEM equipped with a Gatan 4 k×4 k One View camera. Capsids are also detected and characterized by MADLS.

### Example 17: Capsid Disassembly, Reassembly, and Cargo Loading

Purified PEG10 and RTL10 capsid proteins (e.g., as described in Examples 11, 13, and 16) are characterized for their ability to be disassembled, reassembled, and package cargo, e.g., using similar experiments to those described in Examples 9 and 11-13 of International Publication No. WO 2022/164942.

The capsid proteins are diluted in disassembly buffers disclosed herein. A disassembly buffer for PEG10 can comprise a high salt buffer, a high ionic strength buffer, and/or a reducing agent (e.g. GSH, TCEP, β-ME, or DTT). The disassembly buffer for PEG10 can comprise about 500 mM to about 1.5M of a monovalent salt, e.g., NaCl. An illustrative disassembly buffer that can be used for PEG10 is 500 mM NaCl, 5 mM DTT, 50 mM Tris, pH 8.0. A disassembly buffer for RTL10 can comprise a low salt buffer and/or a reducing agent (e.g. GSH, TCEP, β-ME, or DTT). An illustrative disassembly buffer that can be used for RTL10 is 50 mM NaCl, 10 mM DTT, 25 mM Tris, pH 8.0. The solution is incubated on ice for 30 minutes. Following the incubation, disassembly is confirmed by i) MADLS and ii) TEM.

Disassembled capsids are reassembled into capsids by overnight dialysis of the disassembled material against reassembly buffer at +4 °C in a dialysis cassette with a 3,000-10,000 Da molecular weight cutoff. Illustrative examples of reassembly buffers that can be used for PEG10 include: (1) a low salt buffer with 150 mM NaCl, 25 mM Tris, 10 μM ZnCl₂, pH 7.5; and (2) a low salt buffer with 150 mM NaCl, 1 mM DTT, 10 μM ZnCl₂, 50 mM Tris, pH 8.0. An illustrative example of a reassembly buffer that can be used for RTL10 is an acidic buffer plus divalent cation-50 mM MES (2-(N-morpholino) ethanesulfonic acid), 40 mM MgCl₂, pH 6.0.

The quality and integrity of the soluble re-assembled capsids is analyzed by i) MADLS and ii) TEM. The efficiency of capsid formation, disassembly, and reassembly of the capsids are evaluated, e.g., as described in Example 25 of International Publication No. WO 2022/164942 and Example 10, using various disassembly and reassembly conditions disclosed herein. For example, to calculate capsid disassembly and reassembly efficiency, the amount of protein loss is measured, and particle sizes and concentrations were calculated using multi-angle dynamic light scattering, with reassembly confirmed by TEM. An example of a technique for assessing in vitro capsid formation assembly efficiency is provided in Gross et al., (1997) In vitro assembly properties of purified bacterially expressed capsid proteins of human immunodeficiency virus. European Journal of Biochemistry, 249 (2), 592-600, which is incorporated herein by reference for such disclosure. For example, the size and/or number of assembled capsid particles per EM mesh grid can be counted as a measure of assembly efficiency.

Additionally or alternatively, the efficiency of disassembly and reassembly can be determined by MADLS or size exclusion chromatography (SEC) as described in Example 10 (e.g., by quantifying the percent of total protein in the monomer peak after disassembly, and by comparing the amounts of endo-Gag polypeptides in the monomer and capsid peaks as assayed by MADLS or SEC after reassembly).

Cargo loading is evaluated for the purified PEG10 and RTL10 capsid proteins. Disassembled capsids are incubated with a cargo (e.g., RNA, DNA, hairpin RNA, ssDNA, protein, or small molecule, at 0.1-10% w/w or w/v) in disassembly buffer for 30 minutes on ice. Disassembled capsid plus cargo is loaded into a 10 kDa MWCO and dialyzed overnight into reassembly Buffer at 4 °C. The following day assembly is confirmed by i) MADLS and ii) TEM. Capsids are assessed for cargo loading, e.g., using a gel shift assay as disclosed herein for DNA or RNA loading, and/or a nuclease protection assay for DNA or RNA loading.

### Example 18: Hybrid Capsids

Hybrid capsids are generated with a heterogeneous mixture of (i) a first endo Gag subunit type (e.g., first wild type or engineered PEG10, RTL10, or PNMA protein), and (ii) a second endo Gag subunit type (e.g., a different wild type or engineered PEG10, RTL10, RTL family, PNMA family, Arc, or SCAN family (e.g., ZNF18) protein).

The capsid subunits (e.g., as described in Examples 11, 13, and 16) are expressed and purified. Following chemical disassembly, the two types of engineered endo Gag polypeptides are premixed at a desired ratio and reassembled into hybrid capsids. Adjusting relative amounts of the individual engineered endo Gag polypeptides allows for control of the mass/molar ratio of individual subunits within a given reassembled hybrid capsid.

### Example 19: Capsid Modifications

Chemical attachments: Cargos or other components (e.g., fluorescent dyes, small molecules) are chemically attached to capsids comprising the PEG10 or RTL10 polypeptides described in Examples 11, 13, and 16. The cargo or component to be attached can be linked to a cysteine, e.g., a cysteine that is appended to the C-terminus, N-terminus, or within the endo Gag polypeptide, optionally via a linker, such as a flexible linker disclosed herein. Optionally, one or more other cysteines can be deleted or substituted, e.g., as done for PNMA2 in Example 15 of International Publication No. WO 2022/164942, for targeted attachment at the desired cysteine. Mutants can be generated to test the contribution of cysteines (and, e.g., disulfide bonds) to capsid formation, e.g., as in Examples 8 and 15 of International Publication No. WO 2022/164942. The cargo or other component can be conjugated to the cysteine, e.g., via maleimide chemistry. The conjugated protein is assembled into capsids.

Combination with cationic peptide: Assembled capsids comprising the PEG10 or RTL10 polypeptides described in Examples 11, 13, and 16 are mixed with a cationic peptide (e.g., an R9 peptide, SEQ ID NO: 56, or a vectofusion-1 peptide), incubated prior to contacting cells with the capsids, and the impacts of the cationic peptide on capsid uptake and cargo delivery determined. In some embodiments, the cationic peptide can associate with the capsid (e.g., a negative spike portion thereof) via electrostatic interactions, and the presence of the cationic peptide on the capsid can enhance binding to the negatively charged surface of a cell, facilitating increased capsid uptake.

Combination with cationic lipid: Assembled capsids comprising the PEG10 or RTL10 polypeptides described in Examples 11, 13, and 16 are mixed with a cationic lipid (e.g., jetMESSENGER, or analogous lipid agents) at 0.5% volume to volume and allowed to incubate for 10 minutes at room temperature, and the impacts on capsid cellular uptake and cargo delivery determined, e.g., as described in Example 20 or 22 of International Publication No. WO 2022/164942.

Combination with cationic polymer: Assembled capsids comprising the PEG10 or RTL10 polypeptides described in Examples 11, 13, and 16 are mixed with a cationic polymer (e.g., polybrene at 60 μg/mL), and the impacts on capsid uptake and cargo delivery determined, e.g., as described in Example 22 of International Publication No. WO 2022/164942.

### Example 20: Cellular Internalization of PEG10 or RTL10 Capsids

In vitro and in vivo cellular internalization of PEG10 or RTL10 capsids generated in Examples 16-19 is evaluated.

Mammalian cells are cultured, capsids added at a concentration of about 0.1 mg/mL, and incubated to allow time for uptake. Cells are washed and capsid uptake determined, e.g., via staining for the capsids and fluorescence microscopy as described in Examples 14 and 16 of International Publication No. WO 2022/164942.

In vivo internalization of PEG10 and RTL10 capsids generated in Examples 16-19 is evaluated by injecting C57BL/6 mice, e.g., intramuscularly in the hind quarter with about 10 μg of capsids. Animals are sacrificed and muscle tissue from both the injected side, and the contralateral un-injected side are collected, washed in ice cold PBS, and processed for epifluorescence microcopy to evaluate the presence of capsid in muscle, e.g., as described in Example 14 of International Publication No. WO 2022/164942.

Cre mRNA is tested as an illustrative cargo for delivery by capsids generated in Examples 16-19. GFP/RFP switch reporter cells comprising a GFP-loxP-Stop-loxP-RFP construct are contacted with the capsids, and 24-72 hours later, cells are imaged to visualize RFP expression, e.g., as described in Example 18, 19, and 21 of International Publication No. WO 2022/164942.

For in vivo delivery, capsids comprising Cre mRNA generated as described in Examples 16-19 are sterile filtered and injected into the hind quarter of Ai14 reporter mice. Seven days later, animals are sacrificed and the muscle tissue of the hind limbs collected and processed for fluorescent imaging to evaluate delivery of functional RNA.

The levels of uptake and cargo delivery is determined for different capsids.

TABLE A
Sequences

| SEQ | | |
|---|---|---|
| ID | | |
| NO: | Description | Sequence |
| 1 | PNMA2 | MALALLEDWCRIMSVDEQKSLMVTGIPADFEEAEIQEVLQETLKSLG |
| | amino acid | RYRLLGKIFRKQENANAVLLELLEDTDVSAIPSEVQGKGGVWKVIFK |
| | sequence | TPNQDTEFLERLNLFLEKEGQTVSGMFRALGQEGVSPATVPCISPELL |
| | | AHLLGQAMAHAPQPLLPMRYRKLRVFSGSAVPAPEEESFEVWLEQA |
| | | TEIVKEWPVTEAEKKRWLAESLRGPALDLMHIVQADNPSISVEECLE |
| | | AFKQVFGSLESRRTAQVRYLKTYQEEGEKVSAYVLRLETLLRRAVE |
| | | KRAIPRRIADQVRLEQVMAGATLNQMLWCRLRELKDQGPPPSFLEL |
| | | MKVIREEEEEEASFENESIEEPEERDGYGRWNHEGDD |
| 2 | PNMA1 | MAMTLLEDWCRGMDVNSQRALLVWGIPVNCDEAEIEETLQAAMPQ |
| | amino acid | VSYRMLGRMFWREENAKAALLELTGAVDYAAIPREMPGKGGVWK |
| | sequence | VLFKPPTSDAEFLERLHLFLAREGWTVQDVARVLGFQNPTPTPGPEM |
| | | PAEMLNYILDNVIQPLVESIWYKRLTLFSGRDIPGPGEETFDPWLEHT |
| | | NEVLEEWQVSDVEKRRRLMESLRGPAADVIRILKSNNPAITTAECLK |
| | | ALEQVFGSVESSRDAQIKFLNTYQNPGEKLSAYVIRLEPLLQKVVEK |
| | | GAIDKDNVNQARLEQVIAGANHSGAIRRQLWLTGAGEGPAPNLFQL |
| | | LVQIREEEAKEEEEEAEATLLQLGLEGHF |
| 3 | PNMA3 | MPLTLLQDWCRGEHLNTRRCMLILGIPEDCGEDEFEETLQEACRHLG |
| | Q9UL41-1 | RYRVIGRMFRREENAQAILLELAQDIDYALLPREIPGKGGPWEVIVKP |
| | amino acid | RNSDGEFLNRLNRFLEEERRTVSDMNRVLGSDTNCSAPRVTISPEFW |
| | sequence | TWAQTLGAAVQPLLEQMLYRELRVFSGNTISIPGALAFDAWLEHTTE |
| | | MLQMWQVPEGEKRRRLMECLRGPALQVVSGLRASNASITVEECLAA |
| | | LQQVFGPVESHKIAQVKLCKAYQEAGEKVSSFVLRLEPLLQRAVENN |
| | | VVSRRNVNQTRLKRVLSGATLPDKLRDKLKLMKQRRKPPGFLALVK |
| | | LLREEEEWEATLGPDRESLEGLEVAPRPPARITGVGAVPLPASGNSFD |
| | | ARPSQGYRRRRGRGQHRRGGVARAGSRGSRKRKRHTFCYSCGEDG |
| | | HIRVQCINPSNLLLVKQKKQAAVESGNGNWAWDKSHPKSKAK |
| 4 | PNMA4 | MTLRLLEDWCRGMDMNPRKALLIAGISQSCSVAEIEEALQAGLAPLG |
| | (MOAP1) | EYRLLGRMFRRDENRKVALVGLTAETSHALVPKEIPGKGGIWRVIFK |
| | amino acid | PPDPDNTFLSRLNEFLAGEGMTVGELSRALGHENGSLDPEQGMIPEM |
| | sequence | WAPMLAQALEALQPALQCLKYKKLRVFSGRESPEPGEEEFGRWMFH |
| | | TTQMIKAWQVPDVEKRRRLLESLRGPALDVIRVLKINNPLITVDECL |
| | | QALEEVFGVTDNPRELQVKYLTTYQKDEEKLSAYVLRLEPLLQKLV |
| | | QRGAIERDAVNQARLDQVIAGAVHKTIRRELNLPEDGPAPGFLQLLV |
| | | LIKDYEAAEEEEALLQAILEGNFT |
| 5 | PNMA5 | MALTLLEDWCKGMDMDPRKALLIVGIPMECSEVEIQDTVKAGLQPL |
| | amino acid | CAYRVLGRMFRREDNAKAVFIELADTVNYTTLPSHIPGKGGSWEVV |
| | sequence | VKPRNPDDEFLSRLNYFLKDEGRSMTDVARALGCCSLPAESLDAEV |
| | | MPQVRSPPLEPPKESMWYRKLKVFSGTASPSPGEETFEDWLEQVTEI |
| | | MPIWQVSEVEKRRRLLESLRGPALSIMRVLQANNDSITVEQCLDALK |
| | | QIFGDKEDFRASQFRFLQTSPKIGEKVSTFLLRLEPLLQKAVHKSPLSV |
| | | RSTDMIRLKHLLARVAMTPALRGKLELLDQRGCPPNFLELMKLIRDE |
| | | EEWENTEAVMKNKEKPSGRGRGASGRQARAEASVSAPQATVQARS |
| | | FSDSSPQTIQGGLPPLVKRRRLLGSESTRGEDHGQATYPKAENQTPGR |
| | | EGPQAAGEELGNEAGAGAMSHPKPWET |
| 6 | PNMA6A | MAVTMLQDWCRWMGVNARRGLLILGIPEDCDDAEFQESLEAALRP |
| | amino acid | MGHFTVLGKAFREEDNATAALVELDREVNYALVPREIPGTGGPWNV |
| | sequence | VFVPRCSGEEFLGLGRVFHFPEQEGQMVESVAGALGVGLRRVCWLR |
| | | SIGQAVQPWVEAVRCQSLGVFSGRDQPAPGEESFEVWLDHTTEMLH |
| | | VWQGVSERERRRRLLEGLRGTALQLVHALLAENPARTAQDCLAALA |
| | | QVFGDNESQATIRVKCLTAQQQSGERLSAFVLRLEVLLQKAMEKEA |
| | | LARASADRVRLRQMLTRAHLTEPLDEALRKLRMAGRSPSFLEMLGL |
| | | VRESEAWEASLARSVRAQTQEGAGARAGAQAVARASTKVEAVPGG |
| | | PGREPEGLLQAGGQEAEELLQEGLKPVLEECDN |
| 7 | PNMA2 | ALALLEDWCRIMSVDEQKSLMVTGIPADFEEAEIQEVLQETLKSLGR |
| | amino acid | YRLLGKIFRKQENANAVLLELLEDTDVSAIPSEVQGKGGVWKVIFKT |
| | sequence | PNQDTEFLERLNLFLEKEGQTVSGMFRALGQEGVSPATVPCISPELLA |
| | lacking N- | HLLGQAMAHAPQPLLPMRYRKLRVFSGSAVPAPEEESFEVWLEQAT |
| | terminal | EIVKEWPVTEAEKKRWLAESLRGPALDLMHIVQADNPSISVEECLEA |
| | Met | FKQVFGSLESRRTAQVRYLKTYQEEGEKVSAYVLRLETLLRRAVEK |
| | | RAIPRRIADQVRLEQVMAGATLNQMLWCRLRELKDQGPPPSFLELM |
| | | KVIREEEEEEASFENESIEEPEERDGYGRWNHEGDD |
| 8 | PNMA2 | GSGALALLEDWCRIMSVDEQKSLMVTGIPADFEEAEIQEVLQETLKS |
| | amino acid | LGRYRLLGKIFRKQENANAVLLELLEDTDVSAIPSEVQGKGGVWKVI |
| | sequence | FKTPNQDTEFLERLNLFLEKEGQTVSGMFRALGQEGVSPATVPCISPE |
| | after | LLAHLLGQAMAHAPQPLLPMRYRKLRVFSGSAVPAPEEESFEVWLE |
| | expression | QATEIVKEWPVTEAEKKRWLAESLRGPALDLMHIVQADNPSISVEEC |
| | and | LEAFKQVFGSLESRRTAQVRYLKTYQEEGEKVSAYVLRLETLLRRAV |
| | cleavage | EKRAIPRRIADQVRLEQVMAGATLNQMLWCRLRELKDQGPPPSFLEL |
| | | MKVIREEEEEEASFENESIEEPEERDGYGRWNHEGDD |
| 9 | non- | (EAAAK)ₙ |
| | cleavable | |
| | linker | |
| | (n = 1 to 5) | |
| 10 | non- | (EAAAR)ₙ |
| | cleavable | |
| | linker | |
| | (n = 1 to 5) | |
| 11 | non- | (GGGGS)ₙ |
| | cleavable | |
| | linker | |
| | (n = 1 to 10) | |
| 12 | non- | (GGGS)ₙ |
| | cleavable | |
| | linker (n = 1 | |
| | to 10) | |
| 13 | non- | KESGSVSSEQLAQFRSLD |
| | cleavable | |
| | linker | |
| 14 | non- | EGKSSGSGSESKST |
| | cleavable | |
| | linker | |
| 15 | cleavable | GGAANLVRGG |
| | linker | |
| 16 | cleavable | SGRIGFLRTA |
| | linker | |
| 17 | cleavable | SGRSA |
| | linker | |
| 18 | cleavable | GFLG |
| | linker | |
| 19 | cleavable | ALAL |
| | linker | |
| 20 | cleavable | PIC(Et)F-F (where C(Et) indicates S-ethylcysteine) |
| | linker | |
| | cleavable | PR(S/T)(L/I)(S/T) |
| | linker | |
| 22 | cleavable | DEVD |
| | linker | |
| 23 | cleavable | GWEHDG |
| | linker | |
| 24 | cleavable | RPLALWRS |
| | linker | |
| 25 | Nucleotide | ATGCATCACCATCACCATCACGGCTCAGGGTCTGGTAGCGAAAAT |
| | sequence | CTGTACTTCCAGGGGTCAGGT |
| | encoding N- | |
| | terminal tag | |
| 26 | N-terminal | MHHHHHHGSGSGSENLYFQGSG |
| | tag amino | |
| | acid | |
| | sequence | |
| 27 | 6x His tag | HHHHHH |
| 28 | Spacer | GSGSGS |
| 29 | TEV | ENLYFQG |
| | cleavage | |
| | site | |
| 30 | PNMA2- | GCGTTGGCACTGTTAGAAGATTGGTGCCGTATTATGTCTGTGGAC |
| | encoding | GAGCAGAAGTCTCTTATGGTTACTGGGATCCCGGCGGATTTCGAA |
| | NT seq, | GAAGCCGAAATCCAGGAAGTTCTGCAAGAAACATTAAAGAGCTT |
| | lacking N- | GGGCCGGTATCGGCTCTTAGGCAAAATTTTTCGGAAGCAAGAAAA |
| | terminal | TGCTAATGCGGTGTTACTCGAACTGCTCGAGGATACGGATGTTTC |
| | Met | AGCAATCCCATCTGAAGTGCAGGGCAAAGGTGGGGTGTGGAAAG |
| | | TTATCTTCAAGACGCCAAACCAGGATACTGAGTTCCTTGAACGCT |
| | | TGAATCTGTTCTTGGAGAAAGAAGGCCAGACCGTAAGCGGCATGT |
| | | TCCGGGCGCTTGGCCAAGAGGGGGTATCCCCGGCAACGGTACCTT |
| | | GTATTTCCCCTGAGTTGCTCGCGCACTTATTGGGTCAAGCCATGGC |
| | | ACACGCGCCTCAACCTCTCCTGCCGATGCGGTATCGCAAACTGCG |
| | | CGTATTTTCGGGTTCAGCGGTTCCGGCCCCTGAGGAAGAATCATT |
| | | TGAAGTTTGGCTGGAGCAGGCAACCGAGATCGTGAAGGAGTGGC |
| | | CGGTCACAGAGGCCGAAAAGAAACGGTGGTTAGCCGAGAGTCTT |
| | | CGTGGCCCTGCGCTTGACCTTATGCATATTGTACAGGCGGATAAC |
| | | CCTTCTATCTCGGTTGAAGAGTGTCTTGAGGCTTTCAAGCAAGTCT |
| | | TTGGTTCGCTGGAGTCCCGTCGTACTGCCCAGGTCCGGTATCTCAA |
| | | AACGTATCAGGAAGAGGGTGAGAAGGTCTCCGCATACGTCCTGC |
| | | GGTTGGAAACGCTTTTGCGCCGGGCAGTAGAAAAACGCGCAATTC |
| | | CTCGTCGCATTGCCGATCAAGTTCGTTTAGAGCAAGTGATGGCTG |
| | | GCGCAACTTTAAACCAAATGTTATGGTGCCGGTTACGTGAACTGA |
| | | AGGACCAGGGGCCTCCGCCATCTTTTCTTGAGCTGATGAAGGTGA |
| | | TCCGGGAAGAGGAGGAAGAAGAGGCGTCGTTCGAAAACGAGAGT |
| | | ATCGAAGAGCCGGAGGAACGTGATGGGTACGGTCGCTGGAACCA |
| | | CGAGGGCGACGAT |
| 31 | PNMA2- | ATGGCGTTGGCACTGTTAGAAGATTGGTGCCGTATTATGTCTGTG |
| | encoding | GACGAGCAGAAGTCTCTTATGGTTACTGGGATCCCGGCGGATTTC |
| | NT seq | GAAGAAGCCGAAATCCAGGAAGTTCTGCAAGAAACATTAAAGAG |
| | | CTTGGGCCGGTATCGGCTCTTAGGCAAAATTTTTCGGAAGCAAGA |
| | | AAATGCTAATGCGGTGTTACTCGAACTGCTCGAGGATACGGATGT |
| | | TTCAGCAATCCCATCTGAAGTGCAGGGCAAAGGTGGGGTGTGGA |
| | | AAGTTATCTTCAAGACGCCAAACCAGGATACTGAGTTCCTTGAAC |
| | | GCTTGAATCTGTTCTTGGAGAAAGAAGGCCAGACCGTAAGCGGCA |
| | | TGTTCCGGGCGCTTGGCCAAGAGGGGGTATCCCCGGCAACGGTAC |
| | | CTTGTATTTCCCCTGAGTTGCTCGCGCACTTATTGGGTCAAGCCAT |
| | | GGCACACGCGCCTCAACCTCTCCTGCCGATGCGGTATCGCAAACT |
| | | GCGCGTATTTTCGGGTTCAGCGGTTCCGGCCCCTGAGGAAGAATC |
| | | ATTTGAAGTTTGGCTGGAGCAGGCAACCGAGATCGTGAAGGAGT |
| | | GGCCGGTCACAGAGGCCGAAAAGAAACGGTGGTTAGCCGAGAGT |
| | | CTTCGTGGCCCTGCGCTTGACCTTATGCATATTGTACAGGCGGATA |
| | | ACCCTTCTATCTCGGTTGAAGAGTGTCTTGAGGCTTTCAAGCAAGT |
| | | CTTTGGTTCGCTGGAGTCCCGTCGTACTGCCCAGGTCCGGTATCTC |
| | | AAAACGTATCAGGAAGAGGGTGAGAAGGTCTCCGCATACGTCCT |
| | | GCGGTTGGAAACGCTTTTGCGCCGGGCAGTAGAAAAACGCGCAA |
| | | TTCCTCGTCGCATTGCCGATCAAGTTCGTTTAGAGCAAGTGATGG |
| | | CTGGCGCAACTTTAAACCAAATGTTATGGTGCCGGTTACGTGAAC |
| | | TGAAGGACCAGGGGCCTCCGCCATCTTTTCTTGAGCTGATGAAGG |
| | | TGATCCGGGAAGAGGAGGAAGAAGAGGCGTCGTTCGAAAACGAG |
| | | AGTATCGAAGAGCCGGAGGAACGTGATGGGTACGGTCGCTGGAA |
| | | CCACGAGGGCGACGAT |
| 32 | Nucleotide | ATGCATCACCATCACCATCACGGCTCAGGGTCTGGTAGCGAAAAT |
| | sequence | CTGTACTTCCAGGGG |
| | encoding N- | |
| | terminal tag | |
| 33 | N-terminal | MHHHHHHGSGSGSENLYFQG |
| | tag amino | |
| | acid | |
| | sequence | |
| 34 | Arc | GGGGAGCTGGACCACCGGACCAGCGGCGGGCTCCACGCCTACCC |
| | nucleotide | CGGGCCGCGGGGGGGGCAGGTGGCCAAGCCCAACGTGATCCTGC |
| | sequence, | AGATCGGGAAGTGCCGGGCCGAGATGCTGGAGCACGTGCGGCGG |
| | lacking N- | ACGCACCGGCACCTGCTGGCCGAGGTGTCCAAGCAGGTGGAGCG |
| | terminal | CGAGCTGAAGGGGCTGCACCGGTCGGTCGGGAAGCTGGAGAGCA |
| | Met | ACCTGGACGGCTACGTGCCCACGAGCGACTCGCAGCGCTGGAAG |
| | | AAGTCCATCAAGGCCTGCCTGTGCCGCTGCCAGGAGACCATCGCC |
| | | AACCTGGAGCGCTGGGTCAAGCGCGAGATGCACGTGTGGCGCGA |
| | | GGTGTTCTACCGCCTGGAGCGCTGGGCCGACCGCCTGGAGTCCAC |
| | | GGGCGGCAAGTACCCGGTGGGCAGCGAGTCAGCCCGCCACACCG |
| | | TTTCCGTGGGCGTGGGGGGTCCCGAGAGCTACTGCCACGAGGCAG |
| | | ACGGCTACGACTACACCGTCAGCCCCTACGCCATCACCCCGCCCC |
| | | CAGCCGCTGGCGAGCTGCCCGGGCAGGAGCCCGCCGAGGCCCAG |
| | | CAGTACCAGCCGTGGGTCCCCGGCGAGGACGGGCAGCCCAGCCC |
| | | CGGCGTGGACACGCAGATCTTCGAGGACCCTCGAGAGTTCCTGAG |
| | | CCACCTAGAGGAGTACTTGCGGCAGGTGGGCGGCTCTGAGGAGT |
| | | ACTGGCTGTCCCAGATCCAGAATCACATGAACGGGCCGGCCAAG |
| | | AAGTGGTGGGAGTTCAAGCAGGGCTCCGTGAAGAACTGGGTGGA |
| | | GTTCAAGAAGGAGTTCCTGCAGTACAGCGAGGGCACGCTGTCCCG |
| | | AGAGGCCATCCAGCGCGAGCTGGACCTGCCGCAGAAGCAGGGCG |
| | | AGCCGCTGGACCAGTTCCTGTGGCGCAAGCGGGACCTGTACCAGA |
| | | CGCTCTACGTGGACGCGGACGAGGAGGAGATCATCCAGTACGTG |
| | | GTGGGCACCCTGCAGCCCAAGCTCAAGCGTTTCCTGCGCCACCCC |
| | | CTGCCCAAGACCCTGGAGCAGCTCATCCAGAGGGGCATGGAGGT |
| | | GCAGGATGACCTGGAGCAGGCGGCCGAGCCGGCCGGCCCCCACC |
| | | TCCCGGTGGAGGATGAGGCGGAGACCCTCACGCCCGCCCCCAAC |
| | | AGCGAGTCCGTGGCCAGTGACCGGACCCAGCCCGAG |
| 35 | Arc amino | ELDHRTSGGLHAYPGPRGGQVAKPNVILQIGKCRAEMLEHVRRTHR |
| | acid | HLLAEVSKQVERELKGLHRSVGKLESNLDGYVPTSDSQRWKKSIKA |
| | sequence, | CLCRCQETIANLERWVKREMHVWREVFYRLERWADRLESTGGKYP |
| | lacking N- | VGSESARHTVSVGVGGPESYCHEADGYDYTVSPYAITPPPAAGELPG |
| | terminal | QEPAEAQQYQPWVPGEDGQPSPGVDTQIFEDPREFLSHLEEYLRQVG |
| | Met | GSEEYWLSQIQNHMNGPAKKWWEFKQGSVKNWVEFKKEFLQYSEG |
| | | TLSREAIQRELDLPQKQGEPLDQFLWRKRDLYQTLYVDADEEEIIQY |
| | | VVGTLQPKLKRFLRHPLPKTLEQLIQRGMEVQDDLEQAAEPAGPHLP |
| | | VEDEAETLTPAPNSESVASDRTQPE |
| 36 | Arc amino | GELDHRTSGGLHAYPGPRGGQVAKPNVILQIGKCRAEMLEHVRRTH |
| | acid | RHLLAEVSKQVERELKGLHRSVGKLESNLDGYVPTSDSQRWKKSIK |
| | sequence | ACLCRCQETIANLERWVKREMHVWREVFYRLERWADRLESTGGKY |
| | after | PVGSESARHTVSVGVGGPESYCHEADGYDYTVSPYAITPPPAAGELP |
| | expression | GQEPAEAQQYQPWVPGEDGQPSPGVDTQIFEDPREFLSHLEEYLRQV |
| | and | GGSEEYWLSQIQNHMNGPAKKWWEFKQGSVKNWVEFKKEFLQYSE |
| | cleavage | GTLSREAIQRELDLPQKQGEPLDQFLWRKRDLYQTLYVDADEEEIIQ |
| | | YVVGTLQPKLKRFLRHPLPKTLEQLIQRGMEVQDDLEQAAEPAGPHL |
| | | PVEDEAETLTPAPNSESVASDRTQPE |
| 37 | N-terminal | MHHHHHHGSGSGSENLYFQG |
| | amino acid | |
| | sequence | |
| | lacking SG | |
| | linker | |
| 38 | PNMA2 | GALALLEDWCRIMSVDEQKSLMVTGIPADFEEAEIQEVLQETLKSLG |
| | amino acid | RYRLLGKIFRKQENANAVLLELLEDTDVSAIPSEVQGKGGVWKVIFK |
| | sequence | TPNQDTEFLERLNLFLEKEGQTVSGMFRALGQEGVSPATVPCISPELL |
| | after | AHLLGQAMAHAPQPLLPMRYRKLRVFSGSAVPAPEEESFEVWLEQA |
| | expression | TEIVKEWPVTEAEKKRWLAESLRGPALDLMHIVQADNPSISVEECLE |
| | and | AFKQVFGSLESRRTAQVRYLKTYQEEGEKVSAYVLRLETLLRRAVE |
| | cleavage, | KRAIPRRIADQVRLEQVMAGATLNQMLWCRLRELKDQGPPPSFLEL |
| | without | MKVIREEEEEEASFENESIEEPEERDGYGRWNHEGDD |
| | additional | |
| | SG spacer | |
| 39 | Synthetic | EMRRARKRKPRRRQRRKKRGSGQP |
| | nucleic acid | |
| | binding | |
| | domain | |
| 40 | Engineered | ALALLEDWCRIMSVDEQKSLMVTGIPADFEEAEIQEVLQETLKSLGR |
| | PNMA2 | YRLLGKIFRKQENANAVLLELLEDTDVSAIPSEVQGKGGVWKVIFKT |
| | with C- | PNQDTEFLERLNLFLEKEGQTVSGMFRALGQEGVSPATVPCISPELLA |
| | terminal | HLLGQAMAHAPQPLLPMRYRKLRVFSGSAVPAPEEESFEVWLEQAT |
| | RNA | EIVKEWPVTEAEKKRWLAESLRGPALDLMHIVQADNPSISVEECLEA |
| | binding | FKQVFGSLESRRTAQVRYLKTYQEEGEKVSAYVLRLETLLRRAVEK |
| | domain | RAIPRRIADQVRLEQVMAGATLNQMLWCRLRELKDQGPPPSFLELM |
| | (RBD) | KVIREEEEEEAEMRRARKRKPRRRQRRKKRGSGQP |
| 41 | Engineered | MHHHHHHGSGSGSENLYFQGALALLEDWCRIMSVDEQKSLMVTGIP |
| | PNMA2 | ADFEEAEIQEVLQETLKSLGRYRLLGKIFRKQENANAVLLELLEDTD |
| | with C- | VSAIPSEVQGKGGVWKVIFKTPNQDTEFLERLNLFLEKEGQTVSGMF |
| | terminal | RALGQEGVSPATVPCISPELLAHLLGQAMAHAPQPLLPMRYRKLRVF |
| | RBD and N- | SGSAVPAPEEESFEVWLEQATEIVKEWPVTEAEKKRWLAESLRGPAL |
| | terminal | DLMHIVQADNPSISVEECLEAFKQVFGSLESRRTAQVRYLKTYQEEG |
| | 6xHis tag | EKVSAYVLRLETLLRRAVEKRAIPRRIADQVRLEQVMAGATLNQML |
| | and | WCRLRELKDQGPPPSFLELMKVIREEEEEEAEMRRARKRKPRRRQRR |
| | cleavage | KKRGSGQP |
| | site | |
| 42 | Nucleic acid | RRRRGRGQHRRGGVARAGSR |
| | binding | |
| | domain | |
| | from | |
| | PNMA3 | |
| 43 | Engineered | ALALLEDWCRIMSVDEQKSLMVTGIPADFEEAEIQEVLQETLKSLGR |
| | PNMA2 | YRLLGKIFRKQENANAVLLELLEDTDVSAIPSEVQGKGGVWKVIFKT |
| | with C- | PNQDTEFLERLNLFLEKEGQTVSGMFRALGQEGVSPATVPCISPELLA |
| | terminal | HLLGQAMAHAPQPLLPMRYRKLRVFSGSAVPAPEEESFEVWLEQAT |
| | nucleic acid | EIVKEWPVTEAEKKRWLAESLRGPALDLMHIVQADNPSISVEECLEA |
| | binding | FKQVFGSLESRRTAQVRYLKTYQEEGEKVSAYVLRLETLLRRAVEK |
| | domain | RAIPRRIADQVRLEQVMAGATLNQMLWCRLRELKDQGPPPSFLELM |
| | | KVIREEEEEEARRRRGRGQHRRGGVARAGSR |
| 44 | Engineered | MHHHHHHGSGSGSENLYFQGALALLEDWCRIMSVDEQKSLMVTGIP |
| | PNMA2 | ADFEEAEIQEVLQETLKSLGRYRLLGKIFRKQENANAVLLELLEDTD |
| | with C- | VSAIPSEVQGKGGVWKVIFKTPNQDTEFLERLNLFLEKEGQTVSGMF |
| | terminal | RALGQEGVSPATVPCISPELLAHLLGQAMAHAPQPLLPMRYRKLRVF |
| | nucleic acid | SGSAVPAPEEESFEVWLEQATEIVKEWPVTEAEKKRWLAESLRGPAL |
| | binding | DLMHIVQADNPSISVEECLEAFKQVFGSLESRRTAQVRYLKTYQEEG |
| | domain and | EKVSAYVLRLETLLRRAVEKRAIPRRIADQVRLEQVMAGATLNQML |
| | N-terminal | WCRLRELKDQGPPPSFLELMKVIREEEEEEARRRRGRGQHRRGGVA |
| | 6xHis tag | RAGSR |
| | and | |
| | cleavage | |
| | site | |
| 45 | Inverted | PRRRQRRKKRGSGQP |
| | TAT nucleic | |
| | acid binding | |
| | domain | |
| 46 | Engineered | ALALLEDWCRIMSVDEQKSLMVTGIPADFEEAEIQEVLQETLKSLGR |
| | PNMA2 | YRLLGKIFRKQENANAVLLELLEDTDVSAIPSEVQGKGGVWKVIFKT |
| | with C- | PNQDTEFLERLNLFLEKEGQTVSGMFRALGQEGVSPATVPCISPELLA |
| | terminal | HLLGQAMAHAPQPLLPMRYRKLRVFSGSAVPAPEEESFEVWLEQAT |
| | nucleic acid | EIVKEWPVTEAEKKRWLAESLRGPALDLMHIVQADNPSISVEECLEA |
| | binding | FKQVFGSLESRRTAQVRYLKTYQEEGEKVSAYVLRLETLLRRAVEK |
| | domain | RAIPRRIADQVRLEQVMAGATLNQMLWCRLRELKDQGPPPSFLELM |
| | | KVIREEEEEEASFENESIEEPEERDGYGRWNHEGDDPRRRQRRKKRG |
| | | SGQP |
| 47 | Engineered | MHHHHHHGSGSGSENLYFQGALALLEDWCRIMSVDEQKSLMVTGIP |
| | PNMA2 | ADFEEAEIQEVLQETLKSLGRYRLLGKIFRKQENANAVLLELLEDTD |
| | with C- | VSAIPSEVQGKGGVWKVIFKTPNQDTEFLERLNLFLEKEGQTVSGMF |
| | terminal | RALGQEGVSPATVPCISPELLAHLLGQAMAHAPQPLLPMRYRKLRVF |
| | nucleic acid | SGSAVPAPEEESFEVWLEQATEIVKEWPVTEAEKKRWLAESLRGPAL |
| | binding | DLMHIVQADNPSISVEECLEAFKQVFGSLESRRTAQVRYLKTYQEEG |
| | domain and | EKVSAYVLRLETLLRRAVEKRAIPRRIADQVRLEQVMAGATLNQML |
| | N-terminal | WCRLRELKDQGPPPSFLELMKVIREEEEEEASFENESIEEPEERDGYG |
| | 6xHis tag | RWNHEGDDPRRRQRRKKRGSGQP |
| | and | |
| | cleavage | |
| | site | |
| 48 | PNMA2 | ALALLEDWSRIMSVDEQKSLMVTGIPADFEEAEIQEVLQETLKSLGR |
| | C10S/C136 | YRLLGKIFRKQENANAVLLELLEDTDVSAIPSEVQGKGGVWKVIFKT |
| | S/C233S/C3 | PNQDTEFLERLNLFLEKEGQTVSGMFRALGQEGVSPATVPSISPELLA |
| | 10S | HLLGQAMAHAPQPLLPMRYRKLRVFSGSAVPAPEEESFEVWLEQAT |
| | | EIVKEWPVTEAEKKRWLAESLRGPALDLMHIVQADNPSISVEESLEA |
| | | FKQVFGSLESRRTAQVRYLKTYQEEGEKVSAYVLRLETLLRRAVEK |
| | | RAIPRRIADQVRLEQVMAGATLNQMLWSRLRELKDQGPPPSFLELM |
| | | KVIREEEEEEASFENESIEEPEERDGYGRWNHEGDD |
| 49 | C10S/C136S/ | ALALLEDWSRIMSVDEQKSLMVTGIPADFEEAEIQEVLQETLKSLGR |
| | C233S/C310S | YRLLGKIFRKQENANAVLLELLEDTDVSAIPSEVQGKGGVWKVIFKT |
| | with C- | PNQDTEFLERLNLFLEKEGQTVSGMFRALGQEGVSPATVPSISPELLA |
| | terminal | HLLGQAMAHAPQPLLPMRYRKLRVFSGSAVPAPEEESFEVWLEQAT |
| | cysteine | EIVKEWPVTEAEKKRWLAESLRGPALDLMHIVQADNPSISVEESLEA |
| | attached via | FKQVFGSLESRRTAQVRYLKTYQEEGEKVSAYVLRLETLLRRAVEK |
| | flexible | RAIPRRIADQVRLEQVMAGATLNQMLWSRLRELKDQGPPPSFLELM |
| | linker | KVIREEEEEEASFENESIEEPEERDGYGRWNHEGDDGSGSGSGSGSGS |
| | | C |
| 50 | Anti-EGFR | AAQVKLEESGGGSVQTGGSLRLTCAASGRTSRSYGMGWFRQAPGKE |
| | VHH | REFVSGISWRGDSTGYADSVKGRFTISRDNAKNTVDLQMNSLKPEDT |
| | domain | AIYYCAAAAGSAWYGTLYEYDYWGQGTQVTVSS |
| 51 | Anti-EGFR | MHHHHHHGSGSGSENLYFQGAAQVKLEESGGGSVQTGGSLRLTCA |
| | VHH | ASGRTSRSYGMGWFRQAPGKEREFVSGISWRGDSTGYADSVKGRFT |
| | domain with | ISRDNAKNTVDLQMNSLKPEDTAIYYCAAAAGSAWYGTLYEYDYW |
| | N-terminal | GQGTQVTVSSSGSGSGSGSG |
| | 6x-His tag | |
| | and | |
| | cleavage | |
| | site and C- | |
| | terminal | |
| | linker | |
| 52 | PNMA2- | AAQVKLEESGGGSVQTGGSLRLTCAASGRTSRSYGMGWFRQAPGKE |
| | VH | REFVSGISWRGDSTGYADSVKGRFTISRDNAKNTVDLQMNSLKPEDT |
| | | AIYYCAAAAGSAWYGTLYEYDYWGQGTQVTVSSSGSGSGSGSGAL |
| | | ALLEDWCRIMSVDEQKSLMVTGIPADFEEAEIQEVLQETLKSLGRYR |
| | | LLGKIFRKQENANAVLLELLEDTDVSAIPSEVQGKGGVWKVIFKTPN |
| | | QDTEFLERLNLFLEKEGQTVSGMFRALGQEGVSPATVPCISPELLAHL |
| | | LGQAMAHAPQPLLPMRYRKLRVFSGSAVPAPEEESFEVWLEQATEIV |
| | | KEWPVTEAEKKRWLAESLRGPALDLMHIVQADNPSISVEECLEAFKQ |
| | | VFGSLESRRTAQVRYLKTYQEEGEKVSAYVLRLETLLRRAVEKRAIP |
| | | RRIADQVRLEQVMAGATLNQMLWCRLRELKDQGPPPSFLELMKVIR |
| | | EEEEEEASFENESIEEPEERDGYGRWNHEGDD |
| 53 | PNMA2- | MHHHHHHGSGSGSENLYFQGAAQVKLEESGGGSVQTGGSLRLTCA |
| | VH with N- | ASGRTSRSYGMGWFRQAPGKEREFVSGISWRGDSTGYADSVKGRFT |
| | terminal tag | ISRDNAKNTVDLQMNSLKPEDTAIYYCAAAAGSAWYGTLYEYDYW |
| | | GQGTQVTVSSSGSGSGSGSGALALLEDWCRIMSVDEQKSLMVTGIPA |
| | | DFEEAEIQEVLQETLKSLGRYRLLGKIFRKQENANAVLLELLEDTDVS |
| | | AIPSEVQGKGGVWKVIFKTPNQDTEFLERLNLFLEKEGQTVSGMFRA |
| | | LGQEGVSPATVPCISPELLAHLLGQAMAHAPQPLLPMRYRKLRVFSG |
| | | SAVPAPEEESFEVWLEQATEIVKEWPVTEAEKKRWLAESLRGPALDL |
| | | MHIVQADNPSISVEECLEAFKQVFGSLESRRTAQVRYLKTYQEEGEK |
| | | VSAYVLRLETLLRRAVEKRAIPRRIADQVRLEQVMAGATLNQMLWC |
| | | RLRELKDQGPPPSFLELMKVIREEEEEEASFENESIEEPEERDGYGRW |
| | | NHEGDD |
| 55 | Engineered | MALALLEDWCRIMSVDEQKSLMVTGIPADFEEAEIQEVLQETLKSLG |
| | PNMA2 | RYRLLGKIFRKQENANAVLLELLEDTDVSAIPSEVQGKGGVWKVIFK |
| | with C- | TPNQDTEFLERLNLFLEKEGQTVSGMFRALGQEGVSPATVPCISPELL |
| | terminal | AHLLGQAMAHAPQPLLPMRYRKLRVFSGSAVPAPEEESFEVWLEQA |
| | RNA | TEIVKEWPVTEAEKKRWLAESLRGPALDLMHIVQADNPSISVEECLE |
| | binding | AFKQVFGSLESRRTAQVRYLKTYQEEGEKVSAYVLRLETLLRRAVE |
| | domain | KRAIPRRIADQVRLEQVMAGATLNQMLWCRLRELKDQGPPPSFLEL |
| | (RBD) and | MKVIREEEEEEAEMRRARKRKPRRRQRRKKRGSGQP |
| | N-terminal | |
| | Met | |
| 60 | RTL10 | PRGRCRQQGPRIPIWAAANYANAHPWQQMDKASPGVAYTPLVDPW |
| | lacking N | IERPCCGDTVCVRTTMEQKSTASGTCGGKPAERGPLAGHMPSSRPHR |
| | terminal | VDFCWVPGSDPGTFDGSPWLLDRFLAQLGDYMSFHFEHYQDNISRV |
| | Met | CEILRRLTGRAQAWAAPYLDGDLPLPDDYELFCQDLKEVVQDPNSF |
| | | AEYHAVVTCPLPLASSQLPVAPQLPVVRQYLARFLEGLALDMGTAP |
| | | RSLPAAMATPAVSGSNSVSRSALFEQQLTKESTPGPKEPPVLPSSTCSS |
| | | KPGPVEPASSQPEEAAPTPVPRLSESANPPAQRPDPAHPGGPKPQKTE |
| | | EEVLETEGDQEVSLGTPQEVVEAPETPGEPPLSPGF |
| 61 | RTL10 | GPRGRCRQQGPRIPIWAAANYANAHPWQQMDKASPGVAYTPLVDP |
| | amino acid | WIERPCCGDTVCVRTTMEQKSTASGTCGGKPAERGPLAGHMPSSRP |
| | sequence | HRVDFCWVPGSDPGTFDGSPWLLDRFLAQLGDYMSFHFEHYQDNIS |
| | after | RVCEILRRLTGRAQAWAAPYLDGDLPLPDDYELFCQDLKEVVQDPN |
| | expression | SFAEYHAVVTCPLPLASSQLPVAPQLPVVRQYLARFLEGLALDMGTA |
| | and | PRSLPAAMATPAVSGSNSVSRSALFEQQLTKESTPGPKEPPVLPSSTCS |
| | cleavage | SKPGPVEPASSQPEEAAPTPVPRLSESANPPAQRPDPAHPGGPKPQKT |
| | | EEEVLETEGDQEVSLGTPQEVVEAPETPGEPPLSPGF |
| 62 | RTL10 with | MHHHHHHGSGSGSENLYFQGPRGRCRQQGPRIPIWAAANYANAHP |
| | N-terminal | WQQMDKASPGVAYTPLVDPWIERPCCGDTVCVRTTMEQKSTASGT |
| | tag | CGGKPAERGPLAGHMPSSRPHRVDFCWVPGSDPGTFDGSPWLLDRF |
| | | LAQLGDYMSFHFEHYQDNISRVCEILRRLTGRAQAWAAPYLDGDLP |
| | | LPDDYELFCQDLKEVVQDPNSFAEYHAVVTCPLPLASSQLPVAPQLP |
| | | VVRQYLARFLEGLALDMGTAPRSLPAAMATPAVSGSNSVSRSALFE |
| | | QQLTKESTPGPKEPPVLPSSTCSSKPGPVEPASSQPEEAAPTPVPRLSE |
| | | SANPPAQRPDPAHPGGPKPQKTEEEVLETEGDQEVSLGTPQEVVEAP |
| | | ETPGEPPLSPGF |
| 63 | 3xGS- | GSGSGSENLYFQG |
| | AcTEV | |
| 64 | MBP | MKIKTGARILALSALTTMMFSASALAKIEEGKLVIWINGDKGYNGLA |
| | | EVGKKFEKDTGIKVTVEHPDKLEEKFPQVAATGDGPDIIFWAHDRFG |
| | | GYAQSGLLAEITPDKAFQDKLYPFTWDAVRYNGKLIAYPIAVEALSLI |
| | | YNKDLLPNPPKTWEEIPALDKELKAKGKSALMFNLQEPYFTWPLIAA |
| | | DGGYAFKYENGKYDIKDVGVDNAGAKAGLTFLVDLIKNKHMNADT |
| | | DYSIAEAAFNKGETAMTINGPWAWSNIDTSKVNYGVTVLPTFKGQPS |
| | | KPFVGVLSAGINAASPNKELAKEFLENYLLTDEGLEAVNKDKPLGAV |
| | | ALKSYEEELVKDPRIAATMENAQKGEIMPNIPQMSAFWYAVRTAVIN |
| | | AASGRQTVDEALKDAQTNSSSNNNNNNNNNNL |
| 65 | PEG10 | TERRRDELSEEINNLREKVMKQSEENNNLQSQVQKLTEENTTLREQV |
| | lacking N- | EPTPEDEDDDIELRGAAAAAAPPPPIEEECPEDLPEKFDGNPDMLAPF |
| | terminal | MAQCQIFMEKSTRDFSVDRVRVCFVTSMMTGRAARWASAKLERSH |
| | Met | YLMHNYPAFMMEMKHVFEDPQRREVAKRKIRRLRQGMGSVIDYSN |
| | | AFQMIAQDLDWNEPALIDQYHEGLSDHIQEELSHLEVAKSLSALIGQ |
| | | CIHIERRLARAAAARKPRSPPRALVLPHIASHHQVDPTEPVGGARMR |
| | | LTQEEKERRRKLNLCLYCGTGGHYADNCPAKASKSSPAGKLPGPA |
| 66 | PEG10- | GTERRRDELSEEINNLREKVMKQSEENNNLQSQVQKLTEENTTLREQ |
| | 6xHIS | VEPTPEDEDDDIELRGAAAAAAPPPPIEEECPEDLPEKFDGNPDMLAP |
| | Sequence | FMAQCQIFMEKSTRDFSVDRVRVCFVTSMMTGRAARWASAKLERS |
| | | HYLMHNYPAFMMEMKHVFEDPQRREVAKRKIRRLRQGMGSVIDYS |
| | | NAFQMIAQDLDWNEPALIDQYHEGLSDHIQEELSHLEVAKSLSALIG |
| | | QCIHIERRLARAAAARKPRSPPRALVLPHIASHHQVDPTEPVGGARM |
| | | RLTQEEKERRRKLNLCLYCGTGGHYADNCPAKASKSSPAGKLPGPA |
| | | HHHHHH |
| 67 | MBP-3xGS- | MKIKTGARILALSALTTMMFSASALAKIEEGKLVIWINGDKGYNGLA |
| | AcTEV- | EVGKKFEKDTGIKVTVEHPDKLEEKFPQVAATGDGPDIIFWAHDRFG |
| | PEG10- | GYAQSGLLAEITPDKAFQDKLYPFTWDAVRYNGKLIAYPIAVEALSLI |
| | 6xHIS | YNKDLLPNPPKTWEEIPALDKELKAKGKSALMENLQEPYFTWPLIAA |
| | | DGGYAFKYENGKYDIKDVGVDNAGAKAGLTFLVDLIKNKHMNADT |
| | | DYSIAEAAFNKGETAMTINGPWAWSNIDTSKVNYGVTVLPTFKGQPS |
| | | KPFVGVLSAGINAASPNKELAKEFLENYLLTDEGLEAVNKDKPLGAV |
| | | ALKSYEEELVKDPRIAATMENAQKGEIMPNIPQMSAFWYAVRTAVIN |
| | | AASGRQTVDEALKDAQTNSSSNNNNNNNNNNLGSGSGSENLYFQGT |
| | | ERRRDELSEEINNLREKVMKQSEENNNLQSQVQKLTEENTTLREQVE |
| | | PTPEDEDDDIELRGAAAAAAPPPPIEEECPEDLPEKFDGNPDMLAPFM |
| | | AQCQIFMEKSTRDFSVDRVRVCFVTSMMTGRAARWASAKLERSHYL |
| | | MHNYPAFMMEMKHVFEDPQRREVAKRKIRRLRQGMGSVIDYSNAF |
| | | QMIAQDLDWNEPALIDQYHEGLSDHIQEELSHLEVAKSLSALIGQCIH |
| | | IERRLARAAAARKPRSPPRALVLPHIASHHQVDPTEPVGGARMRLTQ |
| | | EEKERRRKLNLCLYCGTGGHYADNCPAKASKSSPAGKLPGPAHHHH |
| | | HH |
| 68 | PEG10 after | GTERRRDELSEEINNLREKVMKQSEENNNLQSQVQKLTEENTTLREQ |
| | expression | VEPTPEDEDDDIELRGAAAAAAPPPPIEEECPEDLPEKFDGNPDMLAP |
| | and | FMAQCQIFMEKSTRDFSVDRVRVCFVTSMMTGRAARWASAKLERS |
| | cleavage | HYLMHNYPAFMMEMKHVFEDPQRREVAKRKIRRLRQGMGSVIDYS |
| | | NAFQMIAQDLDWNEPALIDQYHEGLSDHIQEELSHLEVAKSLSALIG |
| | | QCIHIERRLARAAAARKPRSPPRALVLPHIASHHQVDPTEPVGGARM |
| | | RLTQEEKERRRKLNLCLYCGTGGHYADNCPAKASKSSPAGKLPGPA |
| 69 | RTL10 | MPRGRCRQQGPRIPIWAAANYANAHPWQQMDKASPGVAYTPLVDP |
| | Q7L3V2 | WIERPCCGDTVCVRTTMEQKSTASGTCGGKPAERGPLAGHMPSSRP |
| | | HRVDFCWVPGSDPGTFDGSPWLLDRFLAQLGDYMSFHFEHYQDNIS |
| | | RVCEILRRLTGRAQAWAAPYLDGDLPLPDDYELFCQDLKEVVQDPN |
| | | SFAEYHAVVTCPLPLASSQLPVAPQLPVVRQYLARFLEGLALDMGTA |
| | | PRSLPAAMATPAVSGSNSVSRSALFEQQLTKESTPGPKEPPVLPSSTCS |
| | | SKPGPVEPASSQPEEAAPTPVPRLSESANPPAQRPDPAHPGGPKPQKT |
| | | EEEVLETEGDQEVSLGTPQEVVEAPETPGEPPLSPGF |
| 70 | PEG10 | MTERRRDELSEEINNLREKVMKQSEENNNLQSQVQKLTEENTTLREQ |
| | Q86TG7 | VEPTPEDEDDDIELRGAAAAAAPPPPIEEECPEDLPEKFDGNPDMLAP |
| | | FMAQCQIFMEKSTRDFSVDRVRVCFVTSMMTGRAARWASAKLERS |
| | | HYLMHNYPAFMMEMKHVFEDPQRREVAKRKIRRLRQGMGSVIDYS |
| | | NAFQMIAQDLDWNEPALIDQYHEGLSDHIQEELSHLEVAKSLSALIG |
| | | QCIHIERRLARAAAARKPRSPPRALVLPHIASHHQVDPTEPVGGARM |
| | | RLTQEEKERRRKLNLCLYCGTGGHYADNCPAKASKSSPAGKLPGPA |
| | | VEGPSATGPEIIRSPQDDASSPHLQVMLQIHLPGRHTLFVRAMIDSGA |
| | | SGNFIDHEYVAQNGIPLRIKDWPILVEAIDGRPIASGPVVHETHDLIVD |
| | | LGDHREVLSFDVTQSPFFPVVLGVRWLSTHDPNITWSTRSIVFDSEYC |
| | | RYHCRMYSPIPPSLPPPAPQPPLYYPVDGYRVYQPVRYYYVQNVYTP |
| | | VDEHVYPDHRLVDPHIEMIPGAHSIPSGHVYSLSEPEMAALRDFVAR |
| | | NVKDGLITPTIAPNGAQVLQVKRGWKLQVSYDCRAPNNFTIQNQYP |
| | | RLSIPNLEDQAHLATYTEFVPQIPGYQTYPTYAAYPTYPVGFAWYPV |
| | | GRDGQGRSLYVPVMITWNPHWYRQPPVPQYPPPQPPPPPPPPPPPP |
| | | STL |
| 71 | PEG10 | MTERRRDELSEEINNLREKVMKQSEENNNLQSQVQKLTEENTTLREQ |
| | Q86TG7-2 | VEPTPEDEDDDIELRGAAAAAAPPPPIEEECPEDLPEKFDGNPDMLAP |
| | | FMAQCQIFMEKSTRDFSVDRVRVCFVTSMMTGRAARWASAKLERS |
| | | HYLMHNYPAFMMEMKHVFEDPQRREVAKRKIRRLRQGMGSVIDYS |
| | | NAFQMIAQDLDWNEPALIDQYHEGLSDHIQEELSHLEVAKSLSALIG |
| | | QCIHIERRLARAAAARKPRSPPRALVLPHIASHHQVDPTEPVGGARM |
| | | RLTQEEKERRRKLNLCLYCGTGGHYADNCPAKASKSSPAGNSPAPL |
| 72 | PEG10 | MGPDCPPPPPPPPPNNNNNNNSKHTGHKSACVPNMTERRRDELSEEI |
| | Q86TG7-3 | NNLREKVMKQSEENNNLQSQVQKLTEENTTLREQVEPTPEDEDDDIE |
| | | LRGAAAAAAPPPPIEEECPEDLPEKFDGNPDMLAPFMAQCQIFMEKS |
| | | TRDFSVDRVRVCFVTSMMTGRAARWASAKLERSHYLMHNYPAFM |
| | | MEMKHVFEDPQRREVAKRKIRRLRQGMGSVIDYSNAFQMIAQDLD |
| | | WNEPALIDQYHEGLSDHIQEELSHLEVAKSLSALIGQCIHIERRLARA |
| | | AAARKPRSPPRALVLPHIASHHQVDPTEPVGGARMRLTQEEKERRRK |
| | | LNLCLYCGTGGHYADNCPAKASKSSPAGNSPAPL |
| 73 | PEG10 | MRNKRVLKTKKRRSGRGGQDPGLHPHRSEATAGRSPPTPTVTLGPD |
| | Q86TG7-4 | CPPPPPPPPPNNNNNNNSKHTGHKSACVPNMTERRRDELSEEINNLRE |
| | | KVMKQSEENNNLQSQVQKLTEENTTLREQVEPTPEDEDDDIELRGA |
| | | AAAAAPPPPIEEECPEDLPEKFDGNPDMLAPFMAQCQIFMEKSTRDFS |
| | | VDRVRVCFVTSMMTGRAARWASAKLERSHYLMHNYPAFMMEMKH |
| | | VFEDPQRREVAKRKIRRLRQGMGSVIDYSNAFQMIAQDLDWNEPALI |
| | | DQYHEGLSDHIQEELSHLEVAKSLSALIGQCIHIERRLARAAAARKPR |
| | | SPPRALVLPHIASHHQVDPTEPVGGARMRLTQEEKERRRKLNLCLYC |
| | | GTGGHYADNCPAKASKSSPAGKLPGPAVEGPSATGPEIIRSPQDDASS |
| | | PHLQVMLQIHLPGRHTLFVRAMIDSGASGNFIDHEYVAQNGIPLRIKD |
| | | WPILVEAIDGRPIASGPVVHETHDLIVDLGDHREVLSFDVTQSPFFPV |
| | | VLGVRWLSTHDPNITWSTRSIVFDSEYCRYHCRMYSPIPPSLPPPAPQ |
| | | PPLYYPVDGYRVYQPVRYYYVQNVYTPVDEHVYPDHRLVDPHIEMI |
| | | PGAHSIPSGHVYSLSEPEMAALRDFVARNVKDGLITPTIAPNGAQVLQ |
| | | VKRGWKLQVSYDCRAPNNFTIQNQYPRLSIPNLEDQAHLATYTEFVP |
| | | QIPGYQTYPTYAAYPTYPVGFAWYPVGRDGQGRSLYVPVMITWNPH |
| | | WYRQPPVPQYPPPQPPPPPPPPPPPP |
| 74 | PEG10 | MRNKRVLKTKKRRSGRGGQDPGLHPHRSEATAGRSPPTPTVTLGPD |
| | Q86TG7-5 | CPPPPPPPPPNNNNNNNSKHTGHKSACVPNMTERRRDELSEEINNLRE |
| | | KVMKQSEENNNLQSQVQKLTEENTTLREQVEPTPEDEDDDIELRGA |
| | | AAAAAPPPPIEEECPEDLPEKFDGNPDMLAPFMAQCQIFMEKSTRDFS |
| | | VDRVRVCFVTSMMTGRAARWASAKLERSHYLMHNYPAFMMEMKH |
| | | VFEDPQRREVAKRKIRRLRQGMGSVIDYSNAFQMIAQDLDWNEPALI |
| | | DQYHEGLSDHIQEELSHLEVAKSLSALIGQCIHIERRLARAAAARKPR |
| | | SPPRALVLPHIASHHQVDPTEPVGGARMRLTQEEKERRRKLNLCLYC |
| | | GTGGHYADNCPAKASKSSPAGNSPAPL |
| 75 | +36 eGFP | MGSSHHHHHHSSGLVPRGSHMASGGASKGERLFRGKVPILVELKGD |
| | | VNGHKFSVRGKGKGDATRGKLTLKFICTTGKLPVPWPTLVTTLTYG |
| | | VQCFSRYPKHMKRHDFFKSAMPKGYVQERTISFKKDGKYKTRAEVK |
| | | FEGRTLVNRIKLKGRDFKEKGNILGHKLRYNFNSHKVYITADKRKNG |
| | | IKAKFKIRHNVKDGSVQLADHYQQNTPIGRGPVLLPRNHYLSTRSKL |
| | | SKDPKEKRDHMVLLEFVTAAGIKHGRDERYK |
| 76 | BH3 | LAQLGDYMS |
| | domain | |
| 77 | PNMA5 | ALTLLEDWCKGMDMDPRKALLIVGIPMECSEVEIQDTVKAGLQPLC |
| | lacking N | AYRVLGRMFRREDNAKAVFIELADTVNYTTLPSHIPGKGGSWEVVV |
| | terminal | KPRNPDDEFLSRLNYFLKDEGRSMTDVARALGCCSLPAESLDAEVM |
| | Met | PQVRSPPLEPPKESMWYRKLKVFSGTASPSPGEETFEDWLEQVTEIMP |
| | | IWQVSEVEKRRRLLESLRGPALSIMRVLQANNDSITVEQCLDALKQIF |
| | | GDKEDFRASQFRFLQTSPKIGEKVSTFLLRLEPLLQKAVHKSPLSVRS |
| | | TDMIRLKHLLARVAMTPALRGKLELLDQRGCPPNFLELMKLIRDEEE |
| | | WENTEAVMKNKEKPSGRGRGASGRQARAEASVSAPQATVQARSFS |
| | | DSSPQTIQGGLPPLVKRRRLLGSESTRGEDHGQATYPKAENQTPGRE |
| | | GPQAAGEELGNEAGAGAMSHPKPWET |
| 78 | PNMA5 | GALTLLEDWCKGMDMDPRKALLIVGIPMECSEVEIQDTVKAGLQPL |
| | amino acid | CAYRVLGRMFRREDNAKAVFIELADTVNYTTLPSHIPGKGGSWEVV |
| | sequence | VKPRNPDDEFLSRLNYFLKDEGRSMTDVARALGCCSLPAESLDAEV |
| | after | MPQVRSPPLEPPKESMWYRKLKVFSGTASPSPGEETFEDWLEQVTEI |
| | expression | MPIWQVSEVEKRRRLLESLRGPALSIMRVLQANNDSITVEQCLDALK |
| | and | QIFGDKEDFRASQFRFLQTSPKIGEKVSTFLLRLEPLLQKAVHKSPLSV |
| | cleavage | RSTDMIRLKHLLARVAMTPALRGKLELLDQRGCPPNFLELMKLIRDE |
| | | EEWENTEAVMKNKEKPSGRGRGASGRQARAEASVSAPQATVQARS |
| | | FSDSSPQTIQGGLPPLVKRRRLLGSESTRGEDHGQATYPKAENQTPGR |
| | | EGPQAAGEELGNEAGAGAMSHPKPWET |
| 79 | PNMA5 | MHHHHHHGSGSGSENLYFQGALTLLEDWCKGMDMDPRKALLIVGI |
| | with N- | PMECSEVEIQDTVKAGLQPLCAYRVLGRMFRREDNAKAVFIELADT |
| | terminal tag | VNYTTLPSHIPGKGGSWEVVVKPRNPDDEFLSRLNYFLKDEGRSMTD |
| | | VARALGCCSLPAESLDAEVMPQVRSPPLEPPKESMWYRKLKVFSGT |
| | | ASPSPGEETFEDWLEQVTEIMPIWQVSEVEKRRRLLESLRGPALSIMR |
| | | VLQANNDSITVEQCLDALKQIFGDKEDFRASQFRFLQTSPKIGEKVST |
| | | FLLRLEPLLQKAVHKSPLSVRSTDMIRLKHLLARVAMTPALRGKLEL |
| | | LDQRGCPPNFLELMKLIRDEEEWENTEAVMKNKEKPSGRGRGASGR |
| | | QARAEASVSAPQATVQARSFSDSSPQTIQGGLPPLVKRRRLLGSESTR |
| | | GEDHGQATYPKAENQTPGREGPQAAGEELGNEAGAGAMSHPKPWE |
| | | T |
| 80 | PNMA3 | MPLTLLQDWCRGEHLNTRRCMLILGIPEDCGEDEFEETLQEACRHLG |
| | Q9UL41-2 | RYRVIGRMFRREENAQAILLELAQDIDYALLPREIPGKGGPWEVIVKP |
| | | RNSDGEFLNRLNRFLEEERRTVSDMNRVLGSDTNCSAPRVTISPEFW |
| | | TWAQTLGAAVQPLLEQMLYRELRVFSGNTISIPGALAFDAWLEHTTE |
| | | MLQMWQVPEGEKRRRLMECLRGPALQVVSGLRASNASITVEECLAA |
| | | LQQVFGPVESHKIAQVKLCKAYQEAGEKVSSFVLRLEPLLQRAVENN |
| | | VVSRRNVNQTRLKRVLSGATLPDKLRDKLKLMKQRRKPPGFLALVK |
| | | LLREEEEWEATLGPDRESLEGLEVAPRPPARITGVGAVPLPASGNSFD |
| | | ARPSQGYRRRRGRGQHRRGGVARAGSRGSRKRKRHTFCYSCGEDG |
| | | HIRVQCINPSNLLLAKETKEILEGGEREAQTNSR |
| 81 | PNMA6B/6D | GAVTMLQDWCRWMGVNARRGLLILGIPEDCDDAEFQESLEAALRP |
| | | MGHFTVLGKVFREEDNATAALVELDREVNYALVPREIPGTGGPWNV |
| | | VFVPRCSGEEFLGLGRVFHFPEQEGQMVESVAGALGVGLRRVCWLR |
| | | SIGQAVQPWVEAVRYQSLGVFSGRDQPAPGEESFEVWLDHTTEMLH |
| | | VWQGVSERERRRRLLEGLRGTALQLVHALLAENPARTAQDCLAALA |
| | | QVFGDNESQATIRVKCLTAQQQSGERLSAFVLRLEVLLQKAMEKEA |
| | | LARASADRVRLRQMLTRAHLTEPLDEALRKLRMAGRSPSFLEMLGL |
| | | VRESEAWEASLARSVRAQTQEGAGARAGAQAVARASTKVEAVPGG |
| | | PGREPEGLRQAGGQEAEELLQEGLKPVLEECDN |
| 82 | PNMA6E | MALAMLRDWCRWMGANAERSLLILGIPDDCKEHEFQEAVRAALSPL |
| | A0A0J9YXQ4 | GRYRVLTKHFRKELGAKAALVEFAEYLNRSLIPHQIPGNGGPWKVIF |
| | | LPQVPVIEFQDMPSFPAQPQGQAVAKAAGEGGGAGEAGGVGEVGA |
| | | AGEAGGTGEAGATGEAGAAGEAGGAGEAGGVGEAGAAGEAGGAG |
| | | EAGAAGEGGAAGEAGGAGEAGGVGEAGAAGEAGGAGEAGGVGEA |
| | | GAAGEAGGAGEAGAAGEAGGAGEGRAAGEAGAAGEAGAVGEAGA |
| | | AGEAGAVGEAGAAGEAGAVGEAGGTNVTKAWVQPWRCTLQPVLE |
| | | NRAYRELRPFSRREQPGCEEESFESWVEHAKDMLQLWCHASEREKK |
| | | RWLLESLGGPALEVVSGLLEEDTNLSALDCLAALGQVFRNQDTRMT |
| | | SRLKFLTCTQGPQEGLFAFVVRLEGLLQRAVEKGAVCPALANYLRL |
| | | QQVLSWARPSEALQDTLRGMQLEKRPPGFLGLLRLIREMEAWAAFP |
| | | ARSQQGVAWAAAPVESEDPAAAQASPAQGNASEAGPGAEDAAEAA |
| | | SATKEAARGAPAAGEGESAPAGPEGLGQARPIEVPWGSSPARMSSAV |
| | | WVFPRGLSWGPEGLIQVRGQEARKPPLEGLQTILEEPENEDEDGAGD |
| | | EGQPKSSQGK |
| 83 | PNMA6E | MALAMLRDWCRWMGANAERSLLILGIPDDCKEHEFQEAVRAALSPL |
| | A0A0J9YXH5 | GRREQPGCEEESFESWVEHAKDMLQLWCHASEREKKRWLLESLGGP |
| | | ALEVVSGLLEEDTNLSALDCLAALGQVFRNQDTRMTSRLKFLTCTQ |
| | | GPQEGLFAFVVRLEGLLQRAVEKGAVCPALANYLRLQQVLSWARPS |
| | | EALQDTLRGMQLEKRPPGFLGLLRLIREMEAWAAFPARSQQGVAWA |
| | | AAPVESEDPAAAQASPAQGNASEAGPGAEDAAEAASATKEAARGAP |
| | | AAGEGESAPAGPEGLGQARPIEVPWGSSPARMSSAVWVFPRGLSWG |
| | | PEGLIQVRGQEARKPPLEGLQTILEEPENEDEDGAGDEGQPKSSQGK |
| 84 | PNMA6F | MLQDWCRRMGVNAERSLLILDIPDDCEEHEFQEAVRAALSPLGRYR |
| | | VLIKVFRKELGARAALVEFAEGLNQSLIPRQIAGKGGPWKVISLPQAL |
| | | DAEFQDIPSFPAQPQGQAVARGAGEAGAAGEAGSVGEAGGVNEERS |
| | | AGEDEAGGIGEAGGVGEAGAAGEAGAAGEAGAAGEAGGAGEAGG |
| | | AGEAGGAGEEGGTGEEGGAGEAGGAGEEGGEDEAGAAGEAVGAG |
| | | VVEAWTQSWRQTLRPLVKTMAYRELRPFSGREQPGCVEESFESWLE |
| | | DAKDMLQLWCHASERERRRRLLDSLDGLALDIVSGLLEEDPDFSAQ |
| | | DCLTALGQVFRSRDTWMTSRMKFLTCTQGPQEGLFAFVVRLEGLLQ |
| | | KAVEKGAVHPAMANHLRLRQVLSRARPSEALQDTLRRMQLERRPPD |
| | | FLRLLRLIRDMEAWAASLARSQQGVAWAAAPVESEDPAAAQASPAQ |
| | | GDASEADPGAEDADEAASTTKEAARVAPATGEDENAPAGLEGLGQG |
| | | RSPDAPGGLPARMGSAVDMAPGGPSWEPEGLVQVGGQEAEEPPQEG |
| | | LKPILEESENEDEDGAGEAGKPKSPPGK |
| 85 | PNMA7A | MASIIARVGNSRRLNAPLPPWAHSMLRSLGRSLGPIMASMADRNMK |
| | (ZCCHC12) | LFSGRVVPAQGEETFENWLTQVNGVLPDWNMSEEEKLKRLMKTLR |
| | | GPAREVMRVLQATNPNLSVADFLRAMKLVFGESESSVTAHGKFFNT |
| | | LQAQGEKASLYVIRLEVQLQNAIQAGIIAEKDANRTRLQQLLLGGEL |
| | | SRDLRLRLKDFLRMYANEQERLPNFLELIRMVREEEDWDDAFIKRKR |
| | | PKRSESMVERAVSPVAFQGSPPIVIGSADCNVIEIDDTLDDSDEDVILV |
| | | ESQDPPLPSWGAPPLRDRARPQDEVLVIDSPHNSRAQFPSTSGGSGYK |
| | | NNGPGEMRRARKRKHTIRCSYCGEEGHSKETCDNESDKAQVFENLII |
| | | TLQELTHTEMERSRVAPGEYNDFSEPL |
| 86 | PNMA7B | MASITACVGNSRQQNAPLPPWAHSMLRSLGRSLCPLVVKMAERNM |
| | (ZCCHC18) | KLFSGRVVPAQGKETFENWLIQVNEVLPDWSMSEEEKLKRLMKTLR |
| | | GPAREVMRLLQAANPNLSVADFLRAMKLVFGESESSVTAHGKFFNT |
| | | LQAQGEKASLYVIRLEVQLQNAIQAGILAEKDANQTRLQQLLLGAEL |
| | | NRDLRFRLKHLLRMYANKQERLPNFLELIKMIREEEDWDDAFIKRKR |
| | | PKRSEPIMERAASPVAFQGAQPIAISSADCNCNVIEIDDTLDDSDEDVI |
| | | LVVSLYPSLTPTGAPPFRGRARPLDQVLVIDSPNNSGAQSLSTSGGSG |
| | | YKNDGPGNIRRARKRKYTTRCSYCGEEGHSKETCDNESNKAQVFEN |
| | | LIITLQELTHTEERSKEVPGEHSDASEPQ |
| 87 | PNMA8A | MSKTMAMNLLEDWCRGMEVDIHRSLLVTGIPEDCGQAEIEETLNGV |
| | Q86V59-1 | LSPLGPYRVLNKIFVREENVKAALIEVGEGVNLSTIPREFPGRGGVWR |
| | | VVCRDPTQDAEFLKNLNEFLDAEGRTWEDVVRLLQLNHPTLSQNQH |
| | | QPPENWAEALGVLLGAVVQIIFCMDAEIRSREEARAQEAAEFEEMAA |
| | | WALAAGRKVKKEPGLAAEVGSALKAETPNNWNATEDQHEPTKPLV |
| | | RRAGAKSRSRRKKQKKNSRQEAVPWKKPKGINSNSTANLEDPEVGD |
| | | AESMAISEPIKGSRKPCVNKEELALKKPMAKCAWKGPREPPQDARA |
| | | EAESPGGASESDQDGGHESPPKKKAVAWVSAKNPAPMRKKKKVSL |
| | | GPVSYVLVDSEDGRKKPVMPKKGPGSRREASDQKAPRGQQPAEATA |
| | | STSRGPKAKPEGSPRRATNESRKV |
| 88 | PNMA8A | MSKTMAMNLLEDWCRGMEVDIHRSLLVTGIPEDCGQAEIEETLNGV |
| | Q86V59-2 | LSPLGPYRVLNKIFVREENVKAALIEVGEGVNLSTIPREFPGRGGVWR |
| | | VVCRDPTQDAEFLKNLNEFLDAEGRTWEDVVRLLQLNHPTLSQNQH |
| | | QPPENWAEALGVLLGAVVQIIFCMDAEIRSREEARAQEAAEFEEMAA |
| | | WALAAGRKVKKEPGLAAEVGSALKAETPNNWNATEDQHEPTKPLV |
| | | RRAGAKSRSRRKKQKKNSRQEAVPWKKPKGINSNSTANLEDPEVGD |
| | | AESMAISEPIKGSRKPCVNKEELALKKPMAKCAWKGPREPPQDARA |
| | | EAESPGGASESDQDGGHESPPKKKAVAWVSAKNPAPMRKKKKNPE |
| | | RFDLGDHPY |
| 89 | PNMA8B | MAMSLLQDWCRSLDVDAHRALLVTGIPEGLEQADVEAVLQPTLLPL |
| | Q9ULN7-5 | GTFRLRHMKALMNEKAQAALVEFVEDVNHAAIPREIPGKDGVWRV |
| | | LWKDRAQDTRVLRQMRRLLLDDGPTQAAEAGTPGEAPTPPASETQA |
| | | QDSGEVTGQAGSLLGAARNPRRGRRGRRNRTRRNRLTQKGKKRSR |
| | | GGRPSAPARSEAEDSSDESLGIVIEEIDQGDLSGEEDQSALYATLQAA |
| | | ARELVRQWAPCNSEGEEDGPREFLALVTVTDKSKKEEAEKEPAGAE |
| | | SIRLNTKEDKNGVPDLVALLAVRDTPDEEPVDSDTSESDSQESGDQE |
| | | TEELDNPEFVAIVAYTDPSDPWAREEMLKIASVIESLGWSDEKDKRD |
| | | PLRQVLSVMSKDTNGTRVKVEEAGREVDAVVLRKAGDDGDLRECIS |
| | | TLAQPDLPPQAKKAGRGLFGGWSEHREDEGGLLELVALLAAQDMA |
| | | EVMKEEKENAWEGGKYKYPKGKLGEVLALLAARENMGSNEGSEEA |
| | | SDEQSEEESEDTESEASEPEDRASRKPRAKRARTAPRGLTPAGAPPTA |
| | | SGARKTRAGGRGRGRGVTPEKKAGSRGSAQDDAAGSRKKKGSAGA |
| | | GAHARAGEAKGQAPTGSKAARGKKARRGRRLPPKCR |
| 90 | PNMA8B | MAMSLLQDWCRSLDVDAHRALLVTGIPEGLEQADVEAVLQPTLLPL |
| | Q9ULN7-1 | GTFRLRHMKALMNEKAQAALVEFVEDVNHAAIPREIPGKDGVWRV |
| | | LWKDRAQDTRVLRQMRRLLLDDGPTQAAEAGTPGEAPTPPASETQA |
| | | QDSGEVTGQAGSLLGAARNPRRGRRGRRNRTRRNRLTQKGKKRSR |
| | | GGRPSAPARSEAEDSSDESLGIVIEEIDQGDLSGEEDQSALYATLQAA |
| | | ARELVRQWAPCNSEGGPCPTCPWEGIGGFGCDLTVQTRLSQSYVRD |
| | | TQM |
| 91 | PNMA8C | MLFGVKDIALLEHGCKALEVDSYKSLMILGIPEDCNHEEFEEIIRLPLK |
| | | PLGKFEVAGKAYLEEDKSKAAIIQLTEDINYAVVPREIKGKGGVWRV |
| | | VYMPRKQDIEFLTKLNLFLQSEGRTVEDMARVLRQELCPPATGPREL |
| | | PARKCSVPGLGEKPEAGATVQMDVVPPLDSSEKESKAGVGKRGKRK |
| | | NKKNRRRHHASDKKL |
| 92 | CCDC8 | MLQIGEDVDYLLIPREVRLAGGVWRVISKPATKEAEFRERLTQFLEEE |
| | | GRTLEDVARIMEKSTPHPPQPPKKPKEPRVRRRVQQMVTPPPRLVVG |
| | | TYDSSNASDSEFSDFETSRDKSRQGPRRGKKVRKMPVSYLGSKFLGS |
| | | DLESEDDEELVEAFLRRQEKQPSAPPARRRVNLPVPMFEDNLGPQLS |
| | | KADRWREYVSQVSWGKLKRRVKGWAPRAGPGVGEARLASTAVES |
| | | AGVSSAPEGTSPGDRLGNAGDVCVPQASPRRWRPKINWASFRRRRK |
| | | EQTAPTGQGADIEADQGGEAADSQREEAIADQREGAAGNQRAGAPA |
| | | DQGAEAADNQREEAADNQRAGAPAEEGAEAADNQREEAADNQRA |
| | | EAPADQRSQGTDNHREEAADNQRAEAPADQGSEVTDNQREEAVHD |
| | | QRERAPAVQGADNQRAQARAGQRAEAAHNQRAGAPGIQEAEVSAA |
| | | QGTTGTAPGARARKQVKTVRFQTPGRFSWFCKRRRAFWHTPRLPTL |
| | | PKRVPRAGEARNLRVLRAEARAEAEQGEQEDQL |
| 93 | NusA | MNKEILAVVEAVSNEKALPREKIFEALESALATATKKKYEQEIDVRV |
| | | QIDRKSGDFDTFRRWLVVDEVTQPTKEITLEAARYEDESLNLGDYVE |
| | | DQIESVTFDRITTQTAKQVIVQKVREAERAMVVDQFREHEGEIITGVV |
| | | KKVNRDNISLDLGNNAEAVILREDMLPRENFRPGDRVRGVLYSVRPE |
| | | ARGAQLFVTRSKPEMLIELFRIEVPEIGEEVIEIKAAARDPGSRAKIAV |
| | | KTNDKRIDPVGACVGMRGARVQAVSTELGGERIDIVLWDDNPAQFV |
| | | INAMAPADVASIVVDEDKHTMDIAVEAGNLAQAIGRNGQNVRLASQ |
| | | LSGWELNVMTVDDLQAKHQAEAHAAIDTFTKYLDIDEDFATVLVEE |
| | | GFSTLEELAYVPMKELLEIEGLDEPTVEALRERAKNALATIAQAQEES |
| | | LGDNKPADDLLNLEGVDRDLAFKLAARGVCTLEDLAEQGIDDLADI |
| | | EGLTDEKAGALIMAARNICWFGDEA |
| 94 | Beta- | MKGWLLGVSLTLLGGGSVALAQDDASDRQTREVLDPIVASLMEAQ |
| | lactamase | QIPGMAIALVRPEGTTISHYGAADRETGTPVDDDTLFEIGSLSKTLTA |
| | | TLASLAEVEGKLDFDAPVSRYLPELEGSAFDDISGLNLGTHTGGGLPL |
| | | FVPDEVTDRASLMAWYREWQPTEPIGESRTYSNLGIGLLGLETAASL |
| | | DGEFVPTMRAKVLAPLGMQDTWYDVPEARMADYAMGEDKDGQPT |
| | | RVSPGVLDDEAYGIKTTAADLAKLVRANLHLADVDAELQQAIDATR |
| | | QGHYRVGDMTQALIWEQYSLPVAPETLRAGNGYDMILEPNAAEALE |
| | | PPQSPRDDVWVNKTGSTNGFGGYIVMLPGKHTGLVMLANKNYPND |
| | | ARVEAAYRILSGLGAIDVP |
| 95 | Elongation | MAEITASLVKELRERTGAGMMDCKKALTEANGDIELAIENMRKSGA |
| | factor Ts | IKAAKKAGNVAADGVIKTKIDGNYGIILEVNCQTDFVAKDAGFQAFA |
| | | DKVLDAAVAGKITDVEVLKAQFEEERVALVAKIGENINIRRVAALEG |
| | | DVLGSYQHGARIGVLVAAKGADEELVKHIAMHVAASKPEFIKPEDV |
| | | SAEVVEKEYQVQLDIAMQSGKPKEIAEKMVEGRMKKFTGEVSLTGQ |
| | | PFVMEPSKTVGQLLKEHNAEVTGFIRFEVGEGIEKVETDFAAEVAAM |
| | | SKQS |
| 96 | Peptidyl- | SMKVAKDLVVSLAYQVRTEDGVLVDESPVSAPLDYLHGHGSLISGL |
| | prolyl | ETALEGHEVGDKFDVAVGANDAYGQYDENLVQRVPKDVFMGVDE |
| | isomerase | LQVGMRFLAETDQGPVPVEITAVEDDHVVVDGNHMLAGQNLKFNV |
| | | EVVAIREATEEELAHGHVHGAHDHHHDHDHDGCCGGHGHDHGHE |
| | | HGGEGCCGGKGNGGCGCH |
| 97 | SUMO | MSKHHHHSGHHHTGHHHHSGSHHHTGHINLKVKGQDGNEVFFRIK |
| | | RSTQLKKLMNAYCDRQSVDMTAIAFLFDGRRLRAEQTPDELEMEDG |
| | | DEIDAMLHQTGG |
| 98 | Maltose | MKIEEGKLVIWINGDKGYNGLAEVGKKFEKDTGIKVTVEHPDKLEE |
| | Binding | KFPQVAATGDGPDIIFWAHDRFGGYAQSGLLAEITPDKAFQDKLYPF |
| | Protein | TWDAVRYNGKLIAYPIAVEALSLIYNKDLLPNPPKTWEEIPALDKEL |
| | | KAKGKSALMFNLQEPYFTWPLIAADGGYAFKYENGKYDIKDVGVD |
| | | NAGAKAGLTFLVDLIKNKHMNADTDYSIAEAAFNKGETAMTINGPW |
| | | AWSNIDTSKVNYGVTVLPTFKGQPSKPFVGVLSAGINAASPNKELAK |
| | | EFLENYLLTDEGLEAVNKDKPLGAVALKSYEEELAKDPRIAATMEN |
| | | AQKGEIMPNIPQMSAFWYAVRTAVINAASGRQTVDEALKDAQT |
| 99 | TriggerFactor | MQVSVETTQGLGRRVTITIAADSIETAVKSELVNVAKKVRIDGFRKG |
| | | KVPMNIVAQRYGASVRQDVLGDLMSRNFIDAIIKEKINPAGAPTYVP |
| | | GEYKLGEDFTYSVEFEVYPEVELQGLEAIEVEKPIVEVTDADVDGML |
| | | DTLRKQQATWKEKDGAVEAEDRVTIDFTGSVDGEEFEGGKASDFVL |
| | | AMGQGRMIPGFEDGIKGHKAGEEFTIDVTFPEEYHAENLKGKAAKF |
| | | AINLKKVEERELPELTAEFIKRFGVEDGSVEGLRAEVRKNMERELKS |
| | | AIRNRVKSQAIEGLVKANDIDVPAALIDSEIDVLRRQAAQRFGGNEK |
| | | QALELPRELFEEQAKRRVVVGLLLGEVIRTNELKADEERVKGLIEEM |
| | | ASAYEDPKEVIEFYSKNKELMDNMRNVALEEQAVEAVLAKAKVTE |
| | | KETTFNELMNQQA |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of preparing a capsid comprising:
(a) expressing an endogenous Gag polypeptide conjugate in a host cell or a cell-free expression system, the endogenous Gag polypeptide conjugated to a N-terminal component which prevents, inhibits, or suppresses oligomerization of the endogenous Gag polypeptide;
(b) optionally, isolating the endogenous Gag polypeptide conjugate;
(c) cleaving the N-terminal tag from the endogenous Gag polypeptide conjugate to form cleaved endogenous Gag polypeptide; and
(d) treating the cleaved endogenous Gag polypeptide with an assembly buffer.

2. The method of claim 1, further comprising contacting the endogenous Gag polypeptide with a cargo during step (d), wherein at least a subset of the endogenous Gag capsids comprise the cargo.

3. The method of claim 1, further comprising treating the isolated endogenous Gag polypeptide with a disassembly buffer prior to step (d), thereby producing or maintaining the endogenous Gag polypeptide in a disassembled state.

4. The method of claim 1, wherein less than 20% of the endogenous Gag polypeptide conjugate forms a capsid prior to step (c).

5. A method of preparing a capsid comprising:
(a) cleaving the N-terminal tag from an endogenous Gag polypeptide conjugate to form cleaved endogenous Gag polypeptide, where the endogenous Gag polypeptide conjugate comprises an endogenous Gag polypeptide conjugated to a N-terminal component which prevents, inhibits, or suppresses oligomerization of the endogenous Gag polypeptide;
(b) contacting the cleaved endogenous Gag polypeptide with a cargo; and
(c) treating the cleaved endogenous Gag polypeptide with an assembly buffer, thereby assembling the cleaved endogenous Gag polypeptide into endogenous Gag capsids, wherein at least a subset of the endogenous Gag capsids comprise the cargo.

6. The method of claim 5, further comprising treating the endogenous Gag polypeptide with a disassembly buffer prior to step (b) or (c), thereby producing or maintaining the endogenous Gag polypeptide in a disassembled state.

7. The method of claim 5, wherein less than 20% of the endogenous Gag polypeptide conjugate forms a capsid prior to step (a).

8. The method of claim 5, wherein the N-terminal component comprises (i) a cleavage site directly attached to the endogenous Gag polypeptide and (ii) a bulky solubility tag which prevents, inhibits, or suppresses oligomerization of the endogenous Gag polypeptide.

9. The method of claim 8, wherein the bulky solubility tag comprises a polypeptide.

10. The method of claim 8, wherein the bulky solubility tag increases the solubility of the endogenous Gag polypeptide.

11. The method of claim 8, wherein the bulky solubility tag sterically blocks assembly of the endogenous Gag polypeptide.

12. The method of claim 8, wherein the bulky solubility tag is selected from NusA (SEQ ID NO: 93), beta-lactamase (SEQ ID NO: 94), elongation factor Ts (SEQ ID NO: 95), peptidylprolyl isomerase (SEQ ID NO: 96), SUMO (SEQ ID NO: 97), maltose binding protein (SEQ ID NO: 98), and TriggerFactor (SEQ ID NO: 99).

13. The method of claim 8, wherein the cleavage site is selected from a TEV, HRV3C, thrombin, enterokinase, factor Xa, or carboxypeptidase cleavage site.

14. The method of claim 5, wherein the cleavage site is a TEV cleavage site.

15-16. (canceled)

17. The method of claim 5, wherein the endogenous Gag polypeptide is a PNMA family protein.

18. (canceled)

19. The method of claim 5, wherein the endogenous Gag polypeptide comprises PNMA5 or PNMA2.

20-21. (canceled)

22. The method of claim 6, wherein the disassembly buffer comprises a reducing agent.

23. The method of claim 22, wherein the reducing agent comprises reduced glutathione (GSH), beta mercaptoethanol (β-ME), Dithiothreitol (DTT), or tris(2-carboxyethyl)phosphine (TCEP).

24. (canceled)

25. The method of claim 6, wherein the disassembly buffer comprises a solubilizing agent.

26. The method of claim 6, wherein the disassembly buffer has a pH of about 7-11.

27. The method of claim 6, wherein the disassembly buffer further comprises one or more of a divalent cation.

28. The method of claim 6, wherein the disassembly buffer comprises (a) CHAPS, TCEP, and optionally Tris, (b) TCEP, MgCl₂, and optionally Tris, (c) urea, DTT, optionally NaCl, optionally NaP, and optionally glycerol, or (d) urea, DTT, optionally NaCl, and optionally MgCl₂.

29. The method of claim 5, wherein the assembly buffer contains (a) less than 500 mOsm/kg of salt or (b) about 270-330 mOsm/kg of salt.

30-32. (canceled)

33. The method of claim 5, wherein the assembly buffer comprises glycerol.

34. The method of claim 5, wherein the assembly buffer has a pH of about 6-9.

35. The method of claim 5, wherein the step of treating the endogenous Gag polypeptide to form a capsid comprises incubating in a dialysis cassette with an about 3,000-10,000 Da molecular cutoff.

36-37. (canceled)

38. The method of claim 5, wherein the cargo (a) comprises a nucleic acid, (b) comprises or encodes a gene editing system or a component thereof, (c) polypeptide, (d) a therapeutic agent, (e) an antibody or antigen-binding fragment thereof, a peptidomimetic, a nucleotidomimetic, a drug, a diagnostic tool, an imaging tool, a small molecule, or a combination thereof.

39. A kit comprising (a) a first container containing a composition comprising an endogenous Gag polypeptide conjugate where the endogenous Gag polypeptide conjugate comprises an endogenous Gag polypeptide conjugated to a N-terminal component which prevents, inhibits, or suppresses oligomerization of the endogenous Gag polypeptide; and (b) a second container containing a protease for cleaving the N-terminal component from the endogenous Gag polypeptide.

40-45. (canceled)


SEQUENCE LISTING (101 sequences)

SEQ ID NO: 1 - protein, 364 aa, unidentified
MALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 2 - protein, 353 aa, unidentified
MAMTLLEDWCRGMDVNSQRALLVWGIPVNCDEAEIEETLQAAMPQVSYRMLGRMFWREENAKAALLELTGAVDYAAIPREMPGKGGVWKVLFKPPTSDAEFLERLHLFLAREGWTVQDVARVLGFQNPTPTPGPEMPAEMLNYILDNVIQPLVESIWYKRLTLFSGRDIPGPGEETFDPWLEHTNEVLEEWQVSDVEKRRRLMESLRGPAADVIRILKSNNPAITTAECLKALEQVFGSVESSRDAQIKFLNTYQNPGEKLSAYVIRLEPLLQKVVEKGAIDKDNVNQARLEQVIAGANHSGAIRRQLWLTGAGEGPAPNLFQLLVQIREEEAKEEEEEAEATLLQLGLEGHF

SEQ ID NO: 3 - protein, 463 aa, unidentified
MPLTLLQDWCRGEHLNTRRCMLILGIPEDCGEDEFEETLQEACRHLGRYRVIGRMFRREENAQAILLELAQDIDYALLPREIPGKGGPWEVIVKPRNSDGEFLNRLNRFLEEERRTVSDMNRVLGSDTNCSAPRVTISPEFWTWAQTLGAAVQPLLEQMLYRELRVFSGNTISIPGALAFDAWLEHTTEMLQMWQVPEGEKRRRLMECLRGPALQVVSGLRASNASITVEECLAALQQVFGPVESHKIAQVKLCKAYQEAGEKVSSFVLRLEPLLQRAVENNVVSRRNVNQTRLKRVLSGATLPDKLRDKLKLMKQRRKPPGFLALVKLLREEEEWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLVKQKKQAAVESGNGNWAWDKSHPKSKAK

SEQ ID NO: 4 - protein, 351 aa, unidentified
MTLRLLEDWCRGMDMNPRKALLIAGISQSCSVAEIEEALQAGLAPLGEYRLLGRMFRRDENRKVALVGLTAETSHALVPKEIPGKGGIWRVIFKPPDPDNTFLSRLNEFLAGEGMTVGELSRALGHENGSLDPEQGMIPEMWAPMLAQALEALQPALQCLKYKKLRVFSGRESPEPGEEEFGRWMFHTTQMIKAWQVPDVEKRRRLLESLRGPALDVIRVLKINNPLITVDECLQALEEVFGVTDNPRELQVKYLTTYQKDEEKLSAYVLRLEPLLQKLVQRGAIERDAVNQARLDQVIAGAVHKTIRRELNLPEDGPAPGFLQLLVLIKDYEAAEEEEALLQAILEGNFT

SEQ ID NO: 5 - protein, 448 aa, unidentified
MALTLLEDWCKGMDMDPRKALLIVGIPMECSEVEIQDTVKAGLQPLCAYRVLGRMFRREDNAKAVFIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFLQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSESTRGEDHGQATYPKAENQTPGREGPQAAGEELGNEAGAGAMSHPKPWET

SEQ ID NO: 6 - protein, 399 aa, unidentified
MAVTMLQDWCRWMGVNARRGLLILGIPEDCDDAEFQESLEAALRPMGHFTVLGKAFREEDNATAALVELDREVNYALVPREIPGTGGPWNVVFVPRCSGEEFLGLGRVFHFPEQEGQMVESVAGALGVGLRRVCWLRSIGQAVQPWVEAVRCQSLGVFSGRDQPAPGEESFEVWLDHTTEMLHVWQGVSERERRRRLLEGLRGTALQLVHALLAENPARTAQDCLAALAQVFGDNESQATIRVKCLTAQQQSGERLSAFVLRLEVLLQKAMEKEALARASADRVRLRQMLTRAHLTEPLDEALRKLRMAGRSPSFLEMLGLVRESEAWEASLARSVRAQTQEGAGARAGAQAVARASTKVEAVPGGPGREPEGLLQAGGQEAEELLQEGLKPVLEECDN

SEQ ID NO: 7 - protein, 363 aa, unidentified
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 8 - protein, 366 aa, unidentified
GSGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 9 - protein, 25 aa, unidentified
  VARIANT 1..25: note = This sequence may encompass 1-5 EAAAK repeating units
EAAAKEAAAKEAAAKEAAAKEAAAK

SEQ ID NO: 10 - protein, 25 aa, unidentified
  VARIANT 1..25: note = This sequence may encompass 1-5 EAAAR repeating units
EAAAREAAAREAAAREAAAREAAAR

SEQ ID NO: 11 - protein, 50 aa, unidentified
  VARIANT 1..50: note = This sequence may encompass 1-10 GGGGS repeating units
GGGGSGGGGSGGGGSGGGGSGGGGSGGGGSGGGGSGGGGSGGGGSGGGGS

SEQ ID NO: 12 - protein, 40 aa, unidentified
  VARIANT 1..40: note = This sequence may encompass 1-10 GGGS repeating units
GGGSGGGSGGGSGGGSGGGSGGGSGGGSGGGSGGGSGGGS

SEQ ID NO: 13 - protein, 18 aa, unidentified
KESGSVSSEQLAQFRSLD

SEQ ID NO: 14 - protein, 14 aa, unidentified
EGKSSGSGSESKST

SEQ ID NO: 15 - protein, 10 aa, unidentified
GGAANLVRGG

SEQ ID NO: 16 - protein, 10 aa, unidentified
SGRIGFLRTA

SEQ ID NO: 17 - protein, 5 aa, unidentified
SGRSA

SEQ ID NO: 18 - protein, 4 aa, unidentified
GFLG

SEQ ID NO: 19 - protein, 4 aa, unidentified
ALAL

SEQ ID NO: 20 - protein, 5 aa, unidentified
  SITE 3: note = S-ethylcysteine
PIXFF

SEQ ID NO: 21 - skipped

SEQ ID NO: 22 - protein, 4 aa, unidentified
DEVD

SEQ ID NO: 23 - protein, 6 aa, unidentified
GWEHDG

SEQ ID NO: 24 - protein, 8 aa, unidentified
RPLALWRS

SEQ ID NO: 25 - DNA, 66 bp, unidentified
  source 1..66: mol_type = unassigned DNA
atgcatcaccatcaccatcacggctcagggtctggtagcgaaaatctgtacttccaggggtcaggt

SEQ ID NO: 26 - protein, 22 aa, unidentified
MHHHHHHGSGSGSENLYFQGSG

SEQ ID NO: 27 - protein, 6 aa, unidentified
HHHHHH

SEQ ID NO: 28 - protein, 6 aa, unidentified
GSGSGS

SEQ ID NO: 29 - protein, 7 aa, unidentified
ENLYFQG

SEQ ID NO: 30 - DNA, 1089 bp, unidentified
  source 1..1089: mol_type = unassigned DNA
gcgttggcactgttagaagattggtgccgtattatgtctgtggacgagcagaagtctcttatggttactgggatcccggcggatttcgaagaagccgaaatccaggaagttctgcaagaaacattaaagagcttgggccggtatcggctcttaggcaaaatttttcggaagcaagaaaatgctaatgcggtgttactcgaactgctcgaggatacggatgtttcagcaatcccatctgaagtgcagggcaaaggtggggtgtggaaagttatcttcaagacgccaaaccaggatactgagttccttgaacgcttgaatctgttcttggagaaagaaggccagaccgtaagcggcatgttccgggcgcttggccaagagggggtatccccggcaacggtaccttgtatttcccctgagttgctcgcgcacttattgggtcaagccatggcacacgcgcctcaacctctcctgccgatgcggtatcgcaaactgcgcgtattttcgggttcagcggttccggcccctgaggaagaatcatttgaagtttggctggagcaggcaaccgagatcgtgaaggagtggccggtcacagaggccgaaaagaaacggtggttagccgagagtcttcgtggccctgcgcttgaccttatgcatattgtacaggcggataacccttctatctcggttgaagagtgtcttgaggctttcaagcaagtctttggttcgctggagtcccgtcgtactgcccaggtccggtatctcaaaacgtatcaggaagagggtgagaaggtctccgcatacgtcctgcggttggaaacgcttttgcgccgggcagtagaaaaacgcgcaattcctcgtcgcattgccgatcaagttcgtttagagcaagtgatggctggcgcaactttaaaccaaatgttatggtgccggttacgtgaactgaaggaccaggggcctccgccatcttttcttgagctgatgaaggtgatccgggaagaggaggaagaagaggcgtcgttcgaaaacgagagtatcgaagagccggaggaacgtgatgggtacggtcgctggaaccacgagggcgacgat

SEQ ID NO: 31 - DNA, 1092 bp, unidentified
  source 1..1092: mol_type = unassigned DNA
atggcgttggcactgttagaagattggtgccgtattatgtctgtggacgagcagaagtctcttatggttactgggatcccggcggatttcgaagaagccgaaatccaggaagttctgcaagaaacattaaagagcttgggccggtatcggctcttaggcaaaatttttcggaagcaagaaaatgctaatgcggtgttactcgaactgctcgaggatacggatgtttcagcaatcccatctgaagtgcagggcaaaggtggggtgtggaaagttatcttcaagacgccaaaccaggatactgagttccttgaacgcttgaatctgttcttggagaaagaaggccagaccgtaagcggcatgttccgggcgcttggccaagagggggtatccccggcaacggtaccttgtatttcccctgagttgctcgcgcacttattgggtcaagccatggcacacgcgcctcaacctctcctgccgatgcggtatcgcaaactgcgcgtattttcgggttcagcggttccggcccctgaggaagaatcatttgaagtttggctggagcaggcaaccgagatcgtgaaggagtggccggtcacagaggccgaaaagaaacggtggttagccgagagtcttcgtggccctgcgcttgaccttatgcatattgtacaggcggataacccttctatctcggttgaagagtgtcttgaggctttcaagcaagtctttggttcgctggagtcccgtcgtactgcccaggtccggtatctcaaaacgtatcaggaagagggtgagaaggtctccgcatacgtcctgcggttggaaacgcttttgcgccgggcagtagaaaaacgcgcaattcctcgtcgcattgccgatcaagttcgtttagagcaagtgatggctggcgcaactttaaaccaaatgttatggtgccggttacgtgaactgaaggaccaggggcctccgccatcttttcttgagctgatgaaggtgatccgggaagaggaggaagaagaggcgtcgttcgaaaacgagagtatcgaagagccggaggaacgtgatgggtacggtcgctggaaccacgagggcgacgat

SEQ ID NO: 32 - DNA, 60 bp, unidentified
  source 1..60: mol_type = unassigned DNA
atgcatcaccatcaccatcacggctcagggtctggtagcgaaaatctgtacttccagggg

SEQ ID NO: 33 - protein, 20 aa, unidentified
MHHHHHHGSGSGSENLYFQG

SEQ ID NO: 34 - DNA, 1188 bp, unidentified
  source 1..1188: mol_type = unassigned DNA
ggggagctggaccaccggaccagcggcgggctccacgcctaccccgggccgcggggcgggcaggtggccaagcccaacgtgatcctgcagatcgggaagtgccgggccgagatgctggagcacgtgcggcggacgcaccggcacctgctggccgaggtgtccaagcaggtggagcgcgagctgaaggggctgcaccggtcggtcgggaagctggagagcaacctggacggctacgtgcccacgagcgactcgcagcgctggaagaagtccatcaaggcctgcctgtgccgctgccaggagaccatcgccaacctggagcgctgggtcaagcgcgagatgcacgtgtggcgcgaggtgttctaccgcctggagcgctgggccgaccgcctggagtccacgggcggcaagtacccggtgggcagcgagtcagcccgccacaccgtttccgtgggcgtggggggtcccgagagctactgccacgaggcagacggctacgactacaccgtcagcccctacgccatcaccccgcccccagccgctggcgagctgcccgggcaggagcccgccgaggcccagcagtaccagccgtgggtccccggcgaggacgggcagcccagccccggcgtggacacgcagatcttcgaggaccctcgagagttcctgagccacctagaggagtacttgcggcaggtgggcggctctgaggagtactggctgtcccagatccagaatcacatgaacgggccggccaagaagtggtgggagttcaagcagggctccgtgaagaactgggtggagttcaagaaggagttcctgcagtacagcgagggcacgctgtcccgagaggccatccagcgcgagctggacctgccgcagaagcagggcgagccgctggaccagttcctgtggcgcaagcgggacctgtaccagacgctctacgtggacgcggacgaggaggagatcatccagtacgtggtgggcaccctgcagcccaagctcaagcgtttcctgcgccaccccctgcccaagaccctggagcagctcatccagaggggcatggaggtgcaggatgacctggagcaggcggccgagccggccggcccccacctcccggtggaggatgaggcggagaccctcacgcccgcccccaacagcgagtccgtggccagtgaccggacccagcccgag

SEQ ID NO: 35 - protein, 395 aa, unidentified
ELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 36 - protein, 396 aa, unidentified
GELDHRTSGGLHAYPGPRGGQVAKPNVILQIGKCRAEMLEHVRRTHRHLLAEVSKQVERELKGLHRSVGKLESNLDGYVPTSDSQRWKKSIKACLCRCQETIANLERWVKREMHVWREVFYRLERWADRLESTGGKYPVGSESARHTVSVGVGGPESYCHEADGYDYTVSPYAITPPPAAGELPGQEPAEAQQYQPWVPGEDGQPSPGVDTQIFEDPREFLSHLEEYLRQVGGSEEYWLSQIQNHMNGPAKKWWEFKQGSVKNWVEFKKEFLQYSEGTLSREAIQRELDLPQKQGEPLDQFLWRKRDLYQTLYVDADEEEIIQYVVGTLQPKLKRFLRHPLPKTLEQLIQRGMEVQDDLEQAAEPAGPHLPVEDEAETLTPAPNSESVASDRTQPE

SEQ ID NO: 37 - protein, 20 aa, unidentified
MHHHHHHGSGSGSENLYFQG

SEQ ID NO: 38 - protein, 364 aa, unidentified
GALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 39 - protein, 24 aa, unidentified
EMRRARKRKPRRRQRRKKRGSGQP

SEQ ID NO: 40 - protein, 362 aa, unidentified
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEAEMRRARKRKPRRRQRRKKRGSGQP

SEQ ID NO: 41 - protein, 382 aa, unidentified
MHHHHHHGSGSGSENLYFQGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEAEMRRARKRKPRRRQRRKKRGSGQP

SEQ ID NO: 42 - protein, 20 aa, unidentified
RRRRGRGQHRRGGVARAGSR

SEQ ID NO: 43 - protein, 358 aa, unidentified
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEARRRRGRGQHRRGGVARAGSR

SEQ ID NO: 44 - protein, 378 aa, unidentified
MHHHHHHGSGSGSENLYFQGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEARRRRGRGQHRRGGVARAGSR

SEQ ID NO: 45 - protein, 15 aa, unidentified
PRRRQRRKKRGSGQP

SEQ ID NO: 46 - protein, 378 aa, unidentified
ALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDDPRRRQRRKKRGSGQP

SEQ ID NO: 47 - protein, 398 aa, unidentified
MHHHHHHGSGSGSENLYFQGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDDPRRRQRRKKRGSGQP

SEQ ID NO: 48 - protein, 363 aa, unidentified
ALALLEDWSRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPSISPELLAHLLGQAMAHAPQPLLPMRYRKLRVFSGSAVPAPEEESFEVWLEQATEIVKEWPVTEAEKKRWLAESLRGPALDLMHIVQADNPSISVEESLEAFKQVFGSLESRRTAQVRYLKTYQEEGEKVSAYVLRLETLLRRAVEKRAIPRRIADQVRLEQVMAGATLNQMLWSRLRELKDQGPPPSFLELMKVIREEEEEEASFENESIEEPEERDGYGRWNHEGDD

SEQ ID NO: 49 - protein, 376 aa, unidentified
ALALLEDWSRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPSISPELLAHLLGQAMAHAPQPLLPMRYRKLRVFSGSAVPAPEEESFEVWLEQATEIVKEWPVTEAEKKRWLAESLRGPALDLMHIVQADNPSISVEESLEAFKQVFGSLESRRTAQVRYLKTYQEEGEKVSAYVLRLETLLRRAVEKRAIPRRIADQVRLEQVMAGATLNQMLWSRLRELKDQGPPPSFLELMKVIREEEEEEASFENESIEEPEERDGYGRWNHEGDDGSGSGSGSGSGSC

SEQ ID NO: 50 - protein, 126 aa, unidentified
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSS

SEQ ID NO: 51 - protein, 156 aa, unidentified
MHHHHHHGSGSGSENLYFQGAAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGSGSGSGSG

SEQ ID NO: 52 - protein, 499 aa, unidentified
AAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGSGSGSGSGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 53 - protein, 519 aa, unidentified
MHHHHHHGSGSGSENLYFQGAAQVKLEESGGGSVQTGGSLRLTCAASGRTSRSYGMGWFRQAPGKEREFVSGISWRGDSTGYADSVKGRFTISRDNAKNTVDLQMNSLKPEDTAIYYCAAAAGSAWYGTLYEYDYWGQGTQVTVSSSGSGSGSGSGALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEASFENESIEEPEERDGYGRWNHEGDD

SEQ ID NO: 54 - skipped

SEQ ID NO: 55 - protein, 363 aa, unidentified
MALALLEDWCRIMSVDEQKSLMVTGIPADFEEAEIQEVLQETLKSLGRYRLLGKIFRKQENANAVLLELLEDTDVSAIPSEVQGKGGVWKVIFKTPNQDTEFLERLNLFLEKEGQTVSGMFRALGQEGVSPATVPCISPELLAHLLGQAMAHAPQPLLPMRYRKLRVFSGSAVPAPEEESFEVWLEQATEIVKEWPVTEAEKKRWLAESLRGPALDLMHIVQADNPSISVEECLEAFKQVFGSLESRRTAQVRYLKTYQEEGEKVSAYVLRLETLLRRAVEKRAIPRRIADQVRLEQVMAGATLNQMLWCRLRELKDQGPPPSFLELMKVIREEEEEEAEMRRARKRKPRRRQRRKKRGSGQP

SEQ ID NO: 56 - protein, 9 aa, unidentified
RRRRRRRRR

SEQ ID NO: 57 - skipped

SEQ ID NO: 58 - protein, 6 aa, unidentified
GSGSGS

SEQ ID NO: 59 - skipped

SEQ ID NO: 60 - protein, 363 aa, unidentified
PRGRCRQQGPRIPIWAAANYANAHPWQQMDKASPGVAYTPLVDPWIERPCCGDTVCVRTTMEQKSTASGTCGGKPAERGPLAGHMPSSRPHRVDFCWVPGSDPGTFDGSPWLLDRFLAQLGDYMSFHFEHYQDNISRVCEILRRLTGRAQAWAAPYLDGDLPLPDDYELFCQDLKEVVQDPNSFAEYHAVVTCPLPLASSQLPVAPQLPVVRQYLARFLEGLALDMGTAPRSLPAAMATPAVSGSNSVSRSALFEQQLTKESTPGPKEPPVLPSSTCSSKPGPVEPASSQPEEAAPTPVPRLSESANPPAQRPDPAHPGGPKPQKTEEEVLETEGDQEVSLGTPQEVVEAPETPGEPPLSPGF

SEQ ID NO: 61 - protein, 364 aa, unidentified
GPRGRCRQQGPRIPIWAAANYANAHPWQQMDKASPGVAYTPLVDPWIERPCCGDTVCVRTTMEQKSTASGTCGGKPAERGPLAGHMPSSRPHRVDFCWVPGSDPGTFDGSPWLLDRFLAQLGDYMSFHFEHYQDNISRVCEILRRLTGRAQAWAAPYLDGDLPLPDDYELFCQDLKEVVQDPNSFAEYHAVVTCPLPLASSQLPVAPQLPVVRQYLARFLEGLALDMGTAPRSLPAAMATPAVSGSNSVSRSALFEQQLTKESTPGPKEPPVLPSSTCSSKPGPVEPASSQPEEAAPTPVPRLSESANPPAQRPDPAHPGGPKPQKTEEEVLETEGDQEVSLGTPQEVVEAPETPGEPPLSPGF

SEQ ID NO: 62 - protein, 383 aa, unidentified
MHHHHHHGSGSGSENLYFQGPRGRCRQQGPRIPIWAAANYANAHPWQQMDKASPGVAYTPLVDPWIERPCCGDTVCVRTTMEQKSTASGTCGGKPAERGPLAGHMPSSRPHRVDFCWVPGSDPGTFDGSPWLLDRFLAQLGDYMSFHFEHYQDNISRVCEILRRLTGRAQAWAAPYLDGDLPLPDDYELFCQDLKEVVQDPNSFAEYHAVVTCPLPLASSQLPVAPQLPVVRQYLARFLEGLALDMGTAPRSLPAAMATPAVSGSNSVSRSALFEQQLTKESTPGPKEPPVLPSSTCSSKPGPVEPASSQPEEAAPTPVPRLSESANPPAQRPDPAHPGGPKPQKTEEEVLETEGDQEVSLGTPQEVVEAPETPGEPPLSPGF

SEQ ID NO: 63 - protein, 13 aa, unidentified
GSGSGSENLYFQG

SEQ ID NO: 64 - protein, 407 aa, unidentified
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNL

SEQ ID NO: 65 - protein, 324 aa, unidentified
TERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPA

SEQ ID NO: 66 - protein, 331 aa, unidentified
GTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAHHHHHH

SEQ ID NO: 67 - protein, 750 aa, unidentified
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELVKDPRIAATMENAQKGEIMPNIPQMSAFWYAVRTAVINAASGRQTVDEALKDAQTNSSSNNNNNNNNNNLGSGSGSENLYFQGTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAHHHHHH

SEQ ID NO: 68 - protein, 325 aa, unidentified
GTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPA

SEQ ID NO: 69 - protein, 364 aa, unidentified
MPRGRCRQQGPRIPIWAAANYANAHPWQQMDKASPGVAYTPLVDPWIERPCCGDTVCVRTTMEQKSTASGTCGGKPAERGPLAGHMPSSRPHRVDFCWVPGSDPGTFDGSPWLLDRFLAQLGDYMSFHFEHYQDNISRVCEILRRLTGRAQAWAAPYLDGDLPLPDDYELFCQDLKEVVQDPNSFAEYHAVVTCPLPLASSQLPVAPQLPVVRQYLARFLEGLALDMGTAPRSLPAAMATPAVSGSNSVSRSALFEQQLTKESTPGPKEPPVLPSSTCSSKPGPVEPASSQPEEAAPTPVPRLSESANPPAQRPDPAHPGGPKPQKTEEEVLETEGDQEVSLGTPQEVVEAPETPGEPPLSPGF

SEQ ID NO: 70 - protein, 708 aa, unidentified
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLYYPVDGYRVYQPVRYYYVQNVYTPVDEHVYPDHRLVDPHIEMIPGAHSIPSGHVYSLSEPEMAALRDFVARNVKDGLITPTIAPNGAQVLQVKRGWKLQVSYDCRAPNNFTIQNQYPRLSIPNLEDQAHLATYTEFVPQIPGYQTYPTYAAYPTYPVGFAWYPVGRDGQGRSLYVPVMITWNPHWYRQPPVPQYPPPQPPPPPPPPPPPPSYSTL

SEQ ID NO: 71 - protein, 325 aa, unidentified
MTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGNSPAPL

SEQ ID NO: 72 - protein, 359 aa, unidentified
MGPDCPPPPPPPPPNNNNNNNSKHTGHKSACVPNMTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGNSPAPL

SEQ ID NO: 73 - protein, 784 aa, unidentified
MRNKRVLKTKKRRSGRGGQDPGLHPHRSEATAGRSPPTPTVTLGPDCPPPPPPPPPNNNNNNNSKHTGHKSACVPNMTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGKLPGPAVEGPSATGPEIIRSPQDDASSPHLQVMLQIHLPGRHTLFVRAMIDSGASGNFIDHEYVAQNGIPLRIKDWPILVEAIDGRPIASGPVVHETHDLIVDLGDHREVLSFDVTQSPFFPVVLGVRWLSTHDPNITWSTRSIVFDSEYCRYHCRMYSPIPPSLPPPAPQPPLYYPVDGYRVYQPVRYYYVQNVYTPVDEHVYPDHRLVDPHIEMIPGAHSIPSGHVYSLSEPEMAALRDFVARNVKDGLITPTIAPNGAQVLQVKRGWKLQVSYDCRAPNNFTIQNQYPRLSIPNLEDQAHLATYTEFVPQIPGYQTYPTYAAYPTYPVGFAWYPVGRDGQGRSLYVPVMITWNPHWYRQPPVPQYPPPQPPPPPPPPPPPPSYSTL

SEQ ID NO: 74 - protein, 401 aa, unidentified
MRNKRVLKTKKRRSGRGGQDPGLHPHRSEATAGRSPPTPTVTLGPDCPPPPPPPPPNNNNNNNSKHTGHKSACVPNMTERRRDELSEEINNLREKVMKQSEENNNLQSQVQKLTEENTTLREQVEPTPEDEDDDIELRGAAAAAAPPPPIEEECPEDLPEKFDGNPDMLAPFMAQCQIFMEKSTRDFSVDRVRVCFVTSMMTGRAARWASAKLERSHYLMHNYPAFMMEMKHVFEDPQRREVAKRKIRRLRQGMGSVIDYSNAFQMIAQDLDWNEPALIDQYHEGLSDHIQEELSHLEVAKSLSALIGQCIHIERRLARAAAARKPRSPPRALVLPHIASHHQVDPTEPVGGARMRLTQEEKERRRKLNLCLYCGTGGHYADNCPAKASKSSPAGNSPAPL

SEQ ID NO: 75 - protein, 263 aa, unidentified
MGSSHHHHHHSSGLVPRGSHMASGGASKGERLFRGKVPILVELKGDVNGHKFSVRGKGKGDATRGKLTLKFICTTGKLPVPWPTLVTTLTYGVQCFSRYPKHMKRHDFFKSAMPKGYVQERTISFKKDGKYKTRAEVKFEGRTLVNRIKLKGRDFKEKGNILGHKLRYNFNSHKVYITADKRKNGIKAKFKIRHNVKDGSVQLADHYQQNTPIGRGPVLLPRNHYLSTRSKLSKDPKEKRDHMVLLEFVTAAGIKHGRDERYK

SEQ ID NO: 76 - protein, 9 aa, unidentified
LAQLGDYMS

SEQ ID NO: 77 - protein, 447 aa, unidentified
ALTLLEDWCKGMDMDPRKALLIVGIPMECSEVEIQDTVKAGLQPLCAYRVLGRMFRREDNAKAVFIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFLQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSESTRGEDHGQATYPKAENQTPGREGPQAAGEELGNEAGAGAMSHPKPWET

SEQ ID NO: 78 - protein, 448 aa, unidentified
GALTLLEDWCKGMDMDPRKALLIVGIPMECSEVEIQDTVKAGLQPLCAYRVLGRMFRREDNAKAVFIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFLQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSESTRGEDHGQATYPKAENQTPGREGPQAAGEELGNEAGAGAMSHPKPWET

SEQ ID NO: 79 - protein, 467 aa, unidentified
MHHHHHHGSGSGSENLYFQGALTLLEDWCKGMDMDPRKALLIVGIPMECSEVEIQDTVKAGLQPLCAYRVLGRMFRREDNAKAVFIELADTVNYTTLPSHIPGKGGSWEVVVKPRNPDDEFLSRLNYFLKDEGRSMTDVARALGCCSLPAESLDAEVMPQVRSPPLEPPKESMWYRKLKVFSGTASPSPGEETFEDWLEQVTEIMPIWQVSEVEKRRRLLESLRGPALSIMRVLQANNDSITVEQCLDALKQIFGDKEDFRASQFRFLQTSPKIGEKVSTFLLRLEPLLQKAVHKSPLSVRSTDMIRLKHLLARVAMTPALRGKLELLDQRGCPPNFLELMKLIRDEEEWENTEAVMKNKEKPSGRGRGASGRQARAEASVSAPQATVQARSFSDSSPQTIQGGLPPLVKRRRLLGSESTRGEDHGQATYPKAENQTPGREGPQAAGEELGNEAGAGAMSHPKPWET

SEQ ID NO: 80 - protein, 455 aa, unidentified
MPLTLLQDWCRGEHLNTRRCMLILGIPEDCGEDEFEETLQEACRHLGRYRVIGRMFRREENAQAILLELAQDIDYALLPREIPGKGGPWEVIVKPRNSDGEFLNRLNRFLEEERRTVSDMNRVLGSDTNCSAPRVTISPEFWTWAQTLGAAVQPLLEQMLYRELRVFSGNTISIPGALAFDAWLEHTTEMLQMWQVPEGEKRRRLMECLRGPALQVVSGLRASNASITVEECLAALQQVFGPVESHKIAQVKLCKAYQEAGEKVSSFVLRLEPLLQRAVENNVVSRRNVNQTRLKRVLSGATLPDKLRDKLKLMKQRRKPPGFLALVKLLREEEEWEATLGPDRESLEGLEVAPRPPARITGVGAVPLPASGNSFDARPSQGYRRRRGRGQHRRGGVARAGSRGSRKRKRHTFCYSCGEDGHIRVQCINPSNLLLAKETKEILEGGEREAQTNSR

SEQ ID NO: 81 - protein, 399 aa, unidentified
GAVTMLQDWCRWMGVNARRGLLILGIPEDCDDAEFQESLEAALRPMGHFTVLGKVFREEDNATAALVELDREVNYALVPREIPGTGGPWNVVFVPRCSGEEFLGLGRVFHFPEQEGQMVESVAGALGVGLRRVCWLRSIGQAVQPWVEAVRYQSLGVFSGRDQPAPGEESFEVWLDHTTEMLHVWQGVSERERRRRLLEGLRGTALQLVHALLAENPARTAQDCLAALAQVFGDNESQATIRVKCLTAQQQSGERLSAFVLRLEVLLQKAMEKEALARASADRVRLRQMLTRAHLTEPLDEALRKLRMAGRSPSFLEMLGLVRESEAWEASLARSVRAQTQEGAGARAGAQAVARASTKVEAVPGGPGREPEGLRQAGGQEAEELLQEGLKPVLEECDN

SEQ ID NO: 82 - protein, 647 aa, unidentified
MALAMLRDWCRWMGANAERSLLILGIPDDCKEHEFQEAVRAALSPLGRYRVLTKHFRKELGAKAALVEFAEYLNRSLIPHQIPGNGGPWKVIFLPQVPVIEFQDMPSFPAQPQGQAVAKAAGEGGGAGEAGGVGEVGAAGEAGGTGEAGATGEAGAAGEAGGAGEAGGVGEAGAAGEAGGAGEAGAAGEGGAAGEAGGAGEAGGVGEAGAAGEAGGAGEAGGVGEAGAAGEAGGAGEAGAAGEAGGAGEGRAAGEAGAAGEAGAVGEAGAAGEAGAVGEAGAAGEAGAVGEAGGTNVTKAWVQPWRCTLQPVLENRAYRELRPFSRREQPGCEEESFESWVEHAKDMLQLWCHASEREKKRWLLESLGGPALEVVSGLLEEDTNLSALDCLAALGQVFRNQDTRMTSRLKFLTCTQGPQEGLFAFVVRLEGLLQRAVEKGAVCPALANYLRLQQVLSWARPSEALQDTLRGMQLEKRPPGFLGLLRLIREMEAWAAFPARSQQGVAWAAAPVESEDPAAAQASPAQGNASEAGPGAEDAAEAASATKEAARGAPAAGEGESAPAGPEGLGQARPIEVPWGSSPARMSSAVWVFPRGLSWGPEGLIQVRGQEARKPPLEGLQTILEEPENEDEDGAGDEGQPKSSQGK

SEQ ID NO: 83 - protein, 369 aa, unidentified
MALAMLRDWCRWMGANAERSLLILGIPDDCKEHEFQEAVRAALSPLGRREQPGCEEESFESWVEHAKDMLQLWCHASEREKKRWLLESLGGPALEVVSGLLEEDTNLSALDCLAALGQVFRNQDTRMTSRLKFLTCTQGPQEGLFAFVVRLEGLLQRAVEKGAVCPALANYLRLQQVLSWARPSEALQDTLRGMQLEKRPPGFLGLLRLIREMEAWAAFPARSQQGVAWAAAPVESEDPAAAQASPAQGNASEAGPGAEDAAEAASATKEAARGAPAAGEGESAPAGPEGLGQARPIEVPWGSSPARMSSAVWVFPRGLSWGPEGLIQVRGQEARKPPLEGLQTILEEPENEDEDGAGDEGQPKSSQGK

SEQ ID NO: 84 - protein, 578 aa, unidentified
MLQDWCRRMGVNAERSLLILDIPDDCEEHEFQEAVRAALSPLGRYRVLIKVFRKELGARAALVEFAEGLNQSLIPRQIAGKGGPWKVISLPQALDAEFQDIPSFPAQPQGQAVARGAGEAGAAGEAGSVGEAGGVNEERSAGEDEAGGIGEAGGVGEAGAAGEAGAAGEAGAAGEAGGAGEAGGAGEAGGAGEEGGTGEEGGAGEAGGAGEEGGEDEAGAAGEAVGAGVVEAWTQSWRQTLRPLVKTMAYRELRPFSGREQPGCVEESFESWLEDAKDMLQLWCHASERERRRRLLDSLDGLALDIVSGLLEEDPDFSAQDCLTALGQVFRSRDTWMTSRMKFLTCTQGPQEGLFAFVVRLEGLLQKAVEKGAVHPAMANHLRLRQVLSRARPSEALQDTLRRMQLERRPPDFLRLLRLIRDMEAWAASLARSQQGVAWAAAPVESEDPAAAQASPAQGDASEADPGAEDADEAASTTKEAARVAPATGEDENAPAGLEGLGQGRSPDAPGGLPARMGSAVDMAPGGPSWEPEGLVQVGGQEAEEPPQEGLKPILEESENEDEDGAGEAGKPKSPPGK

SEQ ID NO: 85 - protein, 402 aa, unidentified
MASIIARVGNSRRLNAPLPPWAHSMLRSLGRSLGPIMASMADRNMKLFSGRVVPAQGEETFENWLTQVNGVLPDWNMSEEEKLKRLMKTLRGPAREVMRVLQATNPNLSVADFLRAMKLVFGESESSVTAHGKFFNTLQAQGEKASLYVIRLEVQLQNAIQAGIIAEKDANRTRLQQLLLGGELSRDLRLRLKDFLRMYANEQERLPNFLELIRMVREEEDWDDAFIKRKRPKRSESMVERAVSPVAFQGSPPIVIGSADCNVIEIDDTLDDSDEDVILVESQDPPLPSWGAPPLRDRARPQDEVLVIDSPHNSRAQFPSTSGGSGYKNNGPGEMRRARKRKHTIRCSYCGEEGHSKETCDNESDKAQVFENLIITLQELTHTEMERSRVAPGEYNDFSEPL

SEQ ID NO: 86 - protein, 403 aa, unidentified
MASITACVGNSRQQNAPLPPWAHSMLRSLGRSLCPLVVKMAERNMKLFSGRVVPAQGKETFENWLIQVNEVLPDWSMSEEEKLKRLMKTLRGPAREVMRLLQAANPNLSVADFLRAMKLVFGESESSVTAHGKFFNTLQAQGEKASLYVIRLEVQLQNAIQAGILAEKDANQTRLQQLLLGAELNRDLRFRLKHLLRMYANKQERLPNFLELIKMIREEEDWDDAFIKRKRPKRSEPIMERAASPVAFQGAQPIAISSADCNCNVIEIDDTLDDSDEDVILVVSLYPSLTPTGAPPFRGRARPLDQVLVIDSPNNSGAQSLSTSGGSGYKNDGPGNIRRARKRKYTTRCSYCGEEGHSKETCDNESNKAQVFENLIITLQELTHTEERSKEVPGEHSDASEPQ

SEQ ID NO: 87 - protein, 439 aa, unidentified
MSKTMAMNLLEDWCRGMEVDIHRSLLVTGIPEDCGQAEIEETLNGVLSPLGPYRVLNKIFVREENVKAALIEVGEGVNLSTIPREFPGRGGVWRVVCRDPTQDAEFLKNLNEFLDAEGRTWEDVVRLLQLNHPTLSQNQHQPPENWAEALGVLLGAVVQIIFCMDAEIRSREEARAQEAAEFEEMAAWALAAGRKVKKEPGLAAEVGSALKAETPNNWNATEDQHEPTKPLVRRAGAKSRSRRKKQKKNSRQEAVPWKKPKGINSNSTANLEDPEVGDAESMAISEPIKGSRKPCVNKEELALKKPMAKCAWKGPREPPQDARAEAESPGGASESDQDGGHESPPKKKAVAWVSAKNPAPMRKKKKVSLGPVSYVLVDSEDGRKKPVMPKKGPGSRREASDQKAPRGQQPAEATASTSRGPKAKPEGSPRRATNESRKV

SEQ ID NO: 88 - protein, 378 aa, unidentified
MSKTMAMNLLEDWCRGMEVDIHRSLLVTGIPEDCGQAEIEETLNGVLSPLGPYRVLNKIFVREENVKAALIEVGEGVNLSTIPREFPGRGGVWRVVCRDPTQDAEFLKNLNEFLDAEGRTWEDVVRLLQLNHPTLSQNQHQPPENWAEALGVLLGAVVQIIFCMDAEIRSREEARAQEAAEFEEMAAWALAAGRKVKKEPGLAAEVGSALKAETPNNWNATEDQHEPTKPLVRRAGAKSRSRRKKQKKNSRQEAVPWKKPKGINSNSTANLEDPEVGDAESMAISEPIKGSRKPCVNKEELALKKPMAKCAWKGPREPPQDARAEAESPGGASESDQDGGHESPPKKKAVAWVSAKNPAPMRKKKKNPERFDLGDHPY

SEQ ID NO: 89 - protein, 635 aa, unidentified
MAMSLLQDWCRSLDVDAHRALLVTGIPEGLEQADVEAVLQPTLLPLGTFRLRHMKALMNEKAQAALVEFVEDVNHAAIPREIPGKDGVWRVLWKDRAQDTRVLRQMRRLLLDDGPTQAAEAGTPGEAPTPPASETQAQDSGEVTGQAGSLLGAARNPRRGRRGRRNRTRRNRLTQKGKKRSRGGRPSAPARSEAEDSSDESLGIVIEEIDQGDLSGEEDQSALYATLQAAARELVRQWAPCNSEGEEDGPREFLALVTVTDKSKKEEAEKEPAGAESIRLNTKEDKNGVPDLVALLAVRDTPDEEPVDSDTSESDSQESGDQETEELDNPEFVAIVAYTDPSDPWAREEMLKIASVIESLGWSDEKDKRDPLRQVLSVMSKDTNGTRVKVEEAGREVDAVVLRKAGDDGDLRECISTLAQPDLPPQAKKAGRGLFGGWSEHREDEGGLLELVALLAAQDMAEVMKEEKENAWEGGKYKYPKGKLGEVLALLAARENMGSNEGSEEASDEQSEEESEDTESEASEPEDRASRKPRAKRARTAPRGLTPAGAPPTASGARKTRAGGRGRGRGVTPEKKAGSRGSAQDDAAGSRKKKGSAGAGAHARAGEAKGQAPTGSKAARGKKARRGRRLPPKCR

SEQ ID NO: 90 - protein, 279 aa, unidentified
MAMSLLQDWCRSLDVDAHRALLVTGIPEGLEQADVEAVLQPTLLPLGTFRLRHMKALMNEKAQAALVEFVEDVNHAAIPREIPGKDGVWRVLWKDRAQDTRVLRQMRRLLLDDGPTQAAEAGTPGEAPTPPASETQAQDSGEVTGQAGSLLGAARNPRRGRRGRRNRTRRNRLTQKGKKRSRGGRPSAPARSEAEDSSDESLGIVIEEIDQGDLSGEEDQSALYATLQAAARELVRQWAPCNSEGGPCPTCPWEGIGGFGCDLTVQTRLSQSYVRDTQM

SEQ ID NO: 91 - protein, 204 aa, unidentified
MLFGVKDIALLEHGCKALEVDSYKSLMILGIPEDCNHEEFEEIIRLPLKPLGKFEVAGKAYLEEDKSKAAIIQLTEDINYAVVPREIKGKGGVWRVVYMPRKQDIEFLTKLNLFLQSEGRTVEDMARVLRQELCPPATGPRELPARKCSVPGLGEKPEAGATVQMDVVPPLDSSEKESKAGVGKRGKRKNKKNRRRHHASDKKL

SEQ ID NO: 92 - protein, 538 aa, unidentified
MLQIGEDVDYLLIPREVRLAGGVWRVISKPATKEAEFRERLTQFLEEEGRTLEDVARIMEKSTPHPPQPPKKPKEPRVRRRVQQMVTPPPRLVVGTYDSSNASDSEFSDFETSRDKSRQGPRRGKKVRKMPVSYLGSKFLGSDLESEDDEELVEAFLRRQEKQPSAPPARRRVNLPVPMFEDNLGPQLSKADRWREYVSQVSWGKLKRRVKGWAPRAGPGVGEARLASTAVESAGVSSAPEGTSPGDRLGNAGDVCVPQASPRRWRPKINWASFRRRRKEQTAPTGQGADIEADQGGEAADSQREEAIADQREGAAGNQRAGAPADQGAEAADNQREEAADNQRAGAPAEEGAEAADNQREEAADNQRAEAPADQRSQGTDNHREEAADNQRAEAPADQGSEVTDNQREEAVHDQRERAPAVQGADNQRAQARAGQRAEAAHNQRAGAPGIQEAEVSAAQGTTGTAPGARARKQVKTVRFQTPGRFSWFCKRRRAFWHTPRLPTLPKRVPRAGEARNLRVLRAEARAEAEQGEQEDQL

SEQ ID NO: 93 - protein, 495 aa, unidentified [sequence omitted]

SEQ ID NO: 94 - protein, 388 aa, unidentified
MKGWLLGVSLTLLGGGSVALAQDDASDRQTREVLDPIVASLMEAQQIPGMAIALVRPEGTTISHYGAADRETGTPVDDDTLFEIGSLSKTLTATLASLAEVEGKLDFDAPVSRYLPELEGSAFDDISGLNLGTHTGGGLPLFVPDEVTDRASLMAWYREWQPTEPIGESRTYSNLGIGLLGLETAASLDGEFVPTMRAKVLAPLGMQDTWYDVPEARMADYAMGEDKDGQPTRVSPGVLDDEAYGIKTTAADLAKLVRANLHLADVDAELQQAIDATRQGHYRVGDMTQALIWEQYSLPVAPETLRAGNGYDMILEPNAAEALEPPQSPRDDVWVNKTGSTNGFGGYIVMLPGKHTGLVMLANKNYPNDARVEAAYRILSGLGAIDVP

SEQ ID NO: 95 - protein, 283 aa, unidentified
MAEITASLVKELRERTGAGMMDCKKALTEANGDIELAIENMRKSGAIKAAKKAGNVAADGVIKTKIDGNYGIILEVNCQTDFVAKDAGFQAFADKVLDAAVAGKITDVEVLKAQFEEERVALVAKIGENINIRRVAALEGDVLGSYQHGARIGVLVAAKGADEELVKHIAMHVAASKPEFIKPEDVSAEVVEKEYQVQLDIAMQSGKPKEIAEKMVEGRMKKFTGEVSLTGQPFVMEPSKTVGQLLKEHNAEVTGFIRFEVGEGIEKVETDFAAEVAAMSKQS

SEQ ID NO: 96 - protein, 197 aa, unidentified
SMKVAKDLVVSLAYQVRTEDGVLVDESPVSAPLDYLHGHGSLISGLETALEGHEVGDKFDVAVGANDAYGQYDENLVQRVPKDVFMGVDELQVGMRFLAETDQGPVPVEITAVEDDHVVVDGNHMLAGQNLKFNVEVVAIREATEEELAHGHVHGAHDHHHDHDHDGCCGGHGHDHGHEHGGEGCCGGKGNGGCGCH

SEQ ID NO: 97 - protein, 103 aa, unidentified
MSKHHHHSGHHHTGHHHHSGSHHHTGHINLKVKGQDGNEVFFRIKRSTQLKKLMNAYCDRQSVDMTAIAFLFDGRRLRAEQTPDELEMEDGDEIDAMLHQTGG

SEQ ID NO: 98 - protein, 367 aa, unidentified
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 99 - protein, 432 aa, unidentified
MQVSVETTQGLGRRVTITIAADSIETAVKSELVNVAKKVRIDGFRKGKVPMNIVAQRYGASVRQDVLGDLMSRNFIDAIIKEKINPAGAPTYVPGEYKLGEDFTYSVEFEVYPEVELQGLEAIEVEKPIVEVTDADVDGMLDTLRKQQATWKEKDGAVEAEDRVTIDFTGSVDGEEFEGGKASDFVLAMGQGRMIPGFEDGIKGHKAGEEFTIDVTFPEEYHAENLKGKAAKFAINLKKVEERELPELTAEFIKRFGVEDGSVEGLRAEVRKNMERELKSAIRNRVKSQAIEGLVKANDIDVPAALIDSEIDVLRRQAAQRFGGNEKQALELPRELFEEQAKRRVVVGLLLGEVIRTNELKADEERVKGLIEEMASAYEDPKEVIEFYSKNKELMDNMRNVALEEQAVEAVLAKAKVTEKETTFNELMNQQA

SEQ ID NO: 100 - protein, 20 aa, unidentified
  VARIANT 1..20: note = This sequence may encompass 1-10 GS repeating units
GSGSGSGSGSGSGSGSGSGS

SEQ ID NO: 101 - protein, 20 aa, unidentified
  VARIANT 1..20: note = This sequence may encompass 1-10 SG repeating units
SGSGSGSGSGSGSGSGSGSG